(19) United States
(12) Patent Application Publication
(10) Pub. No.: US 2024/0166618 A1
(43) Pub. Date: May 23, 2024

(54) PHENALKYLAMINES AND METHODS OF MAKING AND USING THE SAME

(71) Applicant: GILGAMESH PHARMACEUTICALS, INC., New York, NY (US)

(72) Inventor: Andrew Carry KRUEGEL, New York, NY (US)

(73) Assignee: GILGAMESH PHARMACEUTICALS, INC., New York, NY (US)

(21) Appl. No.: 18/281,127

(22) PCT Filed: Mar. 14, 2022
(86) PCT No.: PCT/US2022/020196

§ 371 Date: Sep. 8, 2023

Related U.S. Application Data
(60) Provisional application No. 63/160,292, filed on Mar. 12, 2021.

(51) Int. Cl. C07D 317/58; A61K 31/138; A61K 31/145; A61K 31/277; A61K 31/36; A61P 25/22; A61P 25/24; C07C 217/60; C07C 255/59; C07C 323/32

(52) U.S. Cl. CPC C07D 317/58; A61K 31/138; A61K 31/145; A61K 31/277; A61K 31/36; A61P 25/22; A61P 25/24; C07C 217/60; C07C 255/59; C07C 323/32

(57) ABSTRACT

The present disclosure provides phenalkylamine compounds and their use in treating medical disorders, such as psychiatric diseases and disorders. Pharmaceutical compositions and methods of making various phenalkylamine compounds are provided.

### BACKGROUND

Psychiatric illnesses, including depression and anxiety, represent a serious detriment to health and effective human functioning worldwide. Although a number of psychiatric medications are available and extensively prescribed, they fail to deliver relief for many individuals. For those patients who do respond, changes in mood and behavior are often slow to manifest. In recent years, these persistent unmet needs for improved pharmacotherapies to treat psychiatric disorders have led to consideration of previously maligned options. For example, classical serotonergic hallucinogens such as lysergic acid diethylamide (LSD), psilocybin, and dimethyltryptamine (DMT), have been considered as experimental therapeutics for a variety of psychiatric indications.

However, such compounds induce profound hallucinogenic effects, which inhibit normal functioning of individuals so treated. Accordingly, these compounds are currently classified as Schedule I drugs under the Controlled Substances Act due to their high abuse potential, no accepted medical use, and lack of established safety. Such effects are mediated largely through engagement of serotonin receptors. Of particular importance is agonism of the serotonin 2A receptor (5-HT2A), which is responsible for the problematic hallucinogenic activity of these compounds but also thought to be critical for their purported therapeutic effects. Accordingly, compounds of this type that would deliver therapeutic benefits while limiting hallucinogenic activity, and therefore the potential for abuse and adverse events, would be of high therapeutic value.

### SUMMARY

The present disclosure provides, for example, compounds which are modulators of 5-HT2A receptors (5-HT2A), and their use as medicinal agents, processes for their preparation, and pharmaceutical compositions containing them as an active ingredient both alone or in combination with other agents, as well as provides for their use as medicaments and/or in the manufacture of medicaments for the activation of 5-HT2A in warm-blooded animals such as humans. In particular, this disclosure relates to compounds useful for the treatment of psychiatric diseases or disorders. Further, this disclosure provides compounds that induce useful therapeutic effects while exhibiting attenuated or no hallucinogenic effects. Also provided are pharmaceutical compositions comprising at least one disclosed compound and a pharmaceutically acceptable carrier.

### BRIEF DESCRIPTION OF THE DRAWINGS

FIG. 1 is a graph that depicts dose-response curves for 6 selected compounds and DOI in the mouse head twitch response assay. Curves were fit using a Gaussian distribution in GraphPad Prism 9.

FIG. 2 is a graph that depicts total number of head twitches counted over 20 minutes in mice for Compound 23 in the presence or absence of the 5-HT2A receptor antagonist MDL100907. **** p<0.0001

FIG. 3 is a graph that depicts Ex vivo receptor occupancy of Compound 22 and 23 at 15 minutes after drug administration. Both compounds showed significant occupancy of the 5-HT2A receptor but there was no significant difference between the drug-treated groups. * p<0.05, **p<0.01

FIG. 4 is a graph that depicts time immobile in the rat forced swim test 24 hours after drug administration. Both Compound 22 and 23 showed a significant reduction in the time spent immobile. ** p<0.01, **** p<0.0001

FIG. 5 depicts total number of marbles buried in a 30-minute observation period in the marble burying test 30 minutes after drug administration. Both Compound 22 and 23 showed a significant dose-dependent reduction in the total number of marbles buried. Comparisons to vehicle: *** p<0.001, **** p<0.0001

FIG. 6 depicts class and subclass probabilities of Compound 23 and Compound 22 in the SmartCube® (Psychogenics, Inc.). The top panel shows the percent probability that each compound belongs to the classes shown on the left. The bottom panel shows the percent probability that each compound belongs to the sub-classes shown on the left. Compound 23 shows the greatest probability of belonging to the anxiolytic class (yellow), while Compound 22 shows the greatest probability of belonging to the hallucinogen class (magenta). Numbers along the x-axis represent doses in mg/kg.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

The term “alkoxy” as used herein refers to a straight or branched alkyl group attached at oxygen (alkyl-O—). Exemplary alkoxy groups include, but are not limited to, alkoxy groups of 1-6 or 2-6 carbon atoms, referred to herein as C₁-C₆ alkoxy, and C₂-C₆ alkoxy, respectively. Exemplary alkoxy groups include, but are not limited to methoxy, ethoxy, isopropoxy, etc.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁-C₆ alkyl, C₁-C₄ alkyl, and C₁-C₃ alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkenyl” as used herein refers to a straight or branched hydrocarbon with one or more double bonds. Exemplary alkenyl groups include, but are not limited to, straight or branched hydrocarbons of 2-6, 2-4, or 2-3 carbon atoms with one double bond. Exemplary alkenyl groups include, but are not limited to, vinyl, allyl, homoallyl, etc.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The term “cyano” as used herein refers to the radical —CN.

The terms “cycloalkyl” or a “carbocyclic group” as used herein refers to a saturated or partially unsaturated cyclic hydrocarbon group of, for example, 3-6, or 4-6 carbons, referred to herein as C₃-C₆ cycloalkyl or C₄-C₆ cycloalkyl, respectively. Exemplary cycloalkyl groups include, but are not limited to, cyclohexyl, cyclopentyl, cyclopentenyl, cyclobutyl, cyclopropyl, etc.

The term “cycloalkylalkyl” as used herein refers to a saturated straight or branched hydrocarbon substituted with a saturated or partially unsaturated cyclic hydrocarbon group of, for example, 3-6, or 4-6 carbons. Exemplary cycloalkylalkyl groups include, but are not limited to, cyclopropylmethyl, cyclopentylmethyl, 2-cycloproylethyl, etc.

The terms “halo” or “halogen” as used herein refer to F, Cl, Br, or I.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

The terms “heterocyclyl” or “heterocyclic group” are art-recognized and refer to saturated or partially unsaturated, 4-10 membered ring structures, including bridged or fused rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran, dihydrofuran, etc.

The terms “hydroxy” and “hydroxyl” as used herein refers to the radical —OH.

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the disclosure is desirably a mammal in which treatment of psychiatric disease or disorder is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system or animal, (e.g. mammal or human) that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect, such as an amount which results in a decrease in symptoms of a psychiatric disorder.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diasteriomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaemo, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

### I. Phenalkylamine Compounds

In some embodiments, the present disclosure provides a compound having the structure:

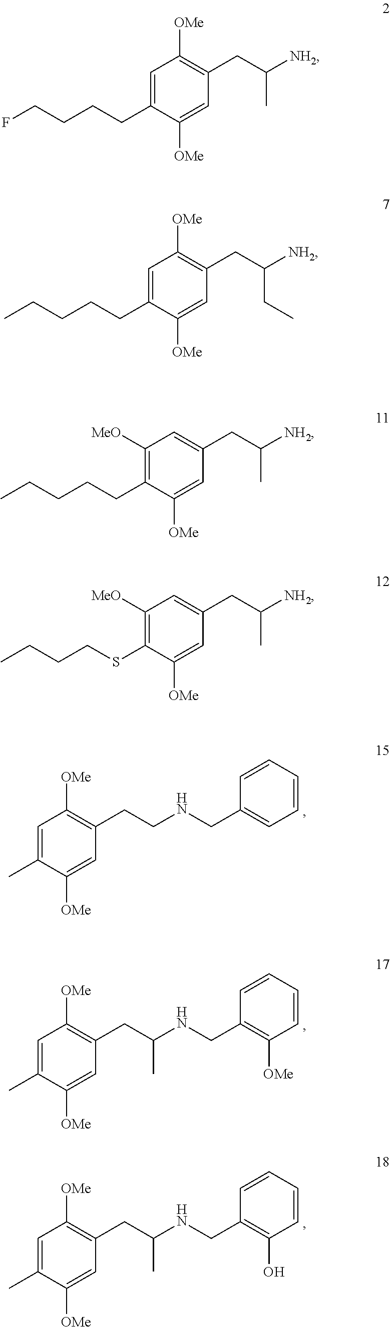

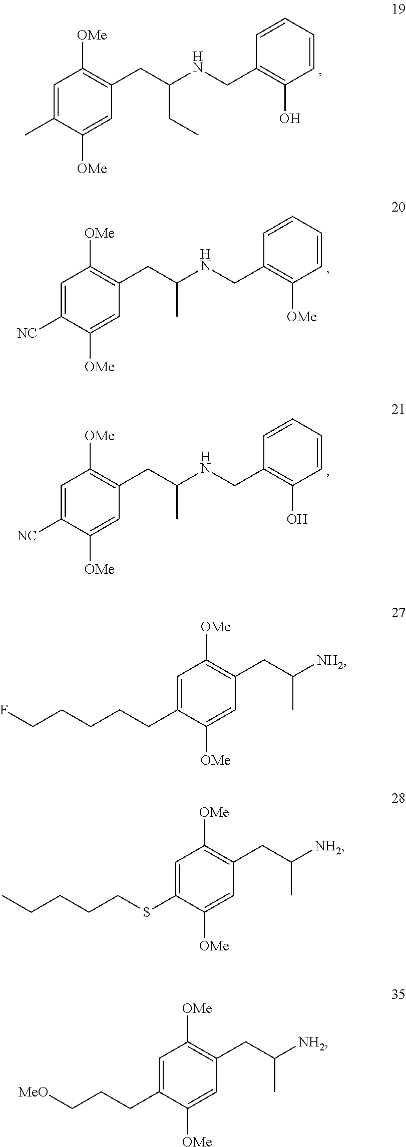 [SMILES: CCC(Cc1cc(OC)c(C)cc1OC)NCc1ccccc1O ; COc1cc(CC(C)NCc2ccccc2OC)c(OC)cc1C#N ; COc1cc(CC(C)NCc2ccccc2O)c(OC)cc1C#N ; COc1cc(CC(C)N)c(OC)cc1CCCCCF ; CCCCCSc1cc(OC)c(CC(C)N)cc1OC ; COCCCc1cc(OC)c(CC(C)N)cc1OC]

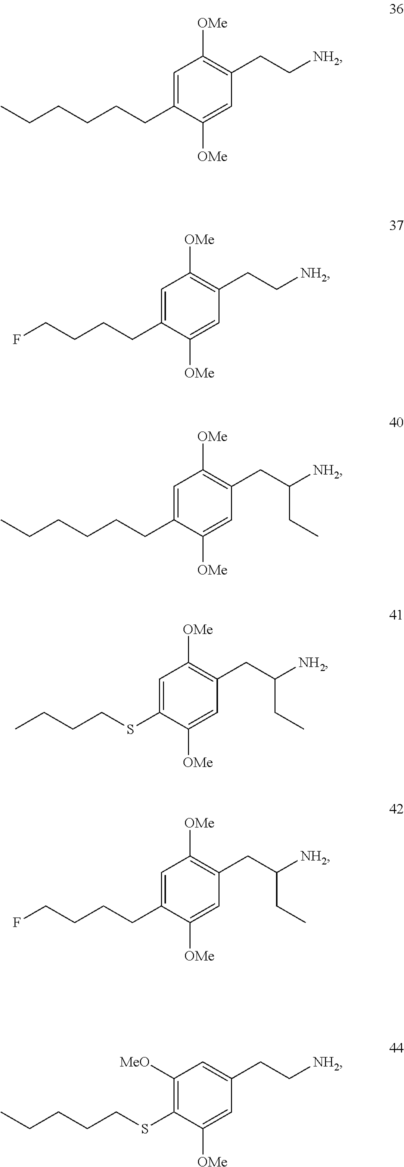

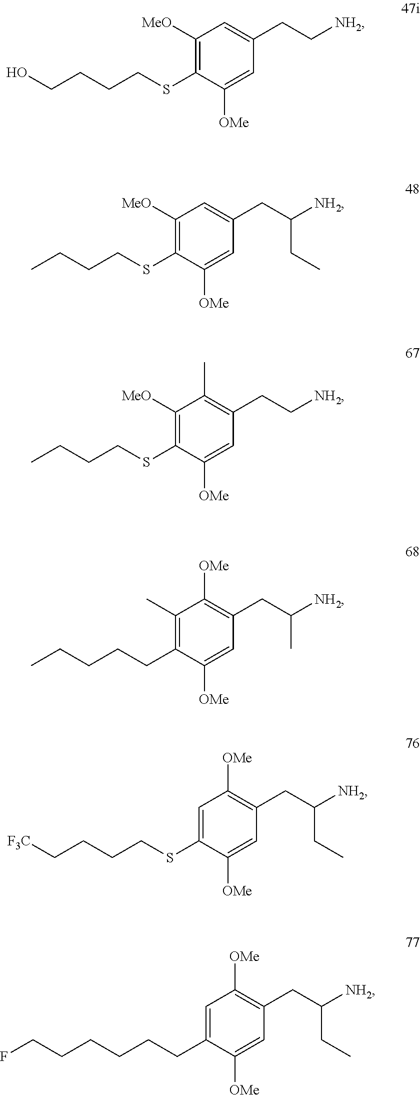

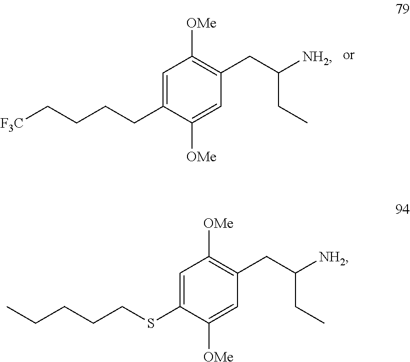

or a pharmaceutically acceptable salt thereof.

In some embodiments, the present disclosure provides a compound having the structure:

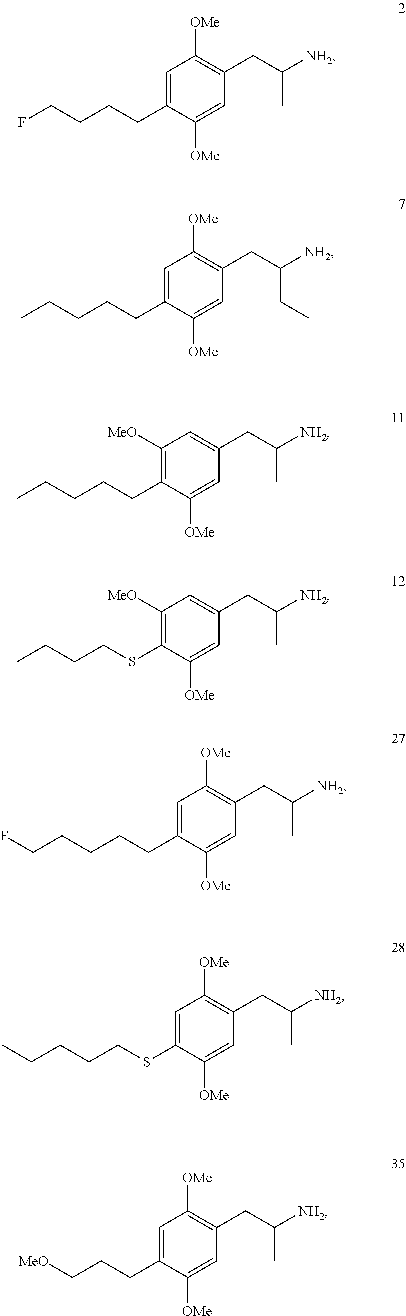

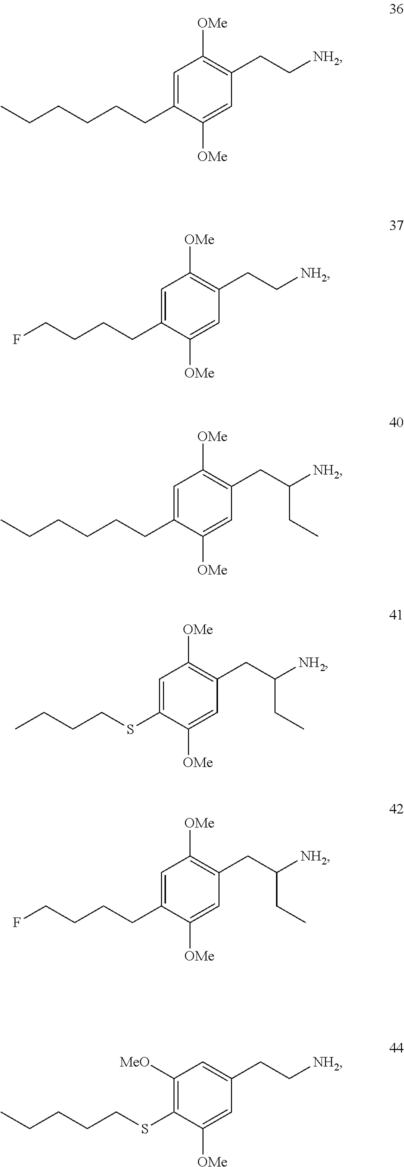

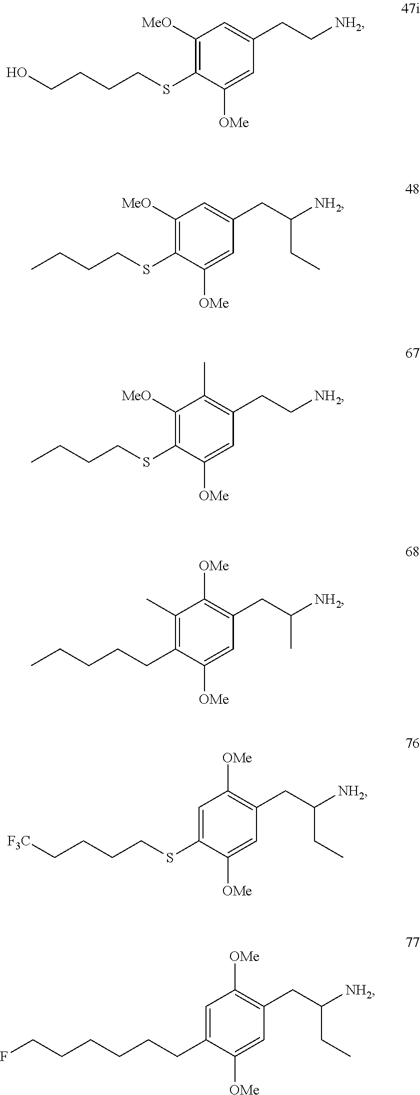

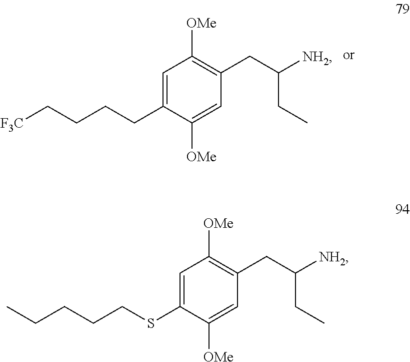

or a pharmaceutically acceptable salt thereof.

In some embodiments, the present disclosure provides a compound having the structure:

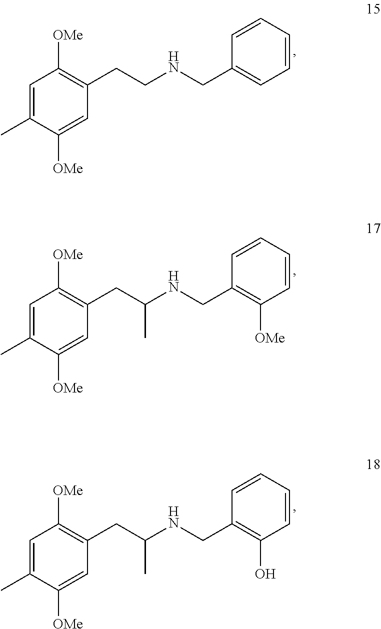 [SMILES: COc1cc(CCNCc2ccccc2)c(OC)cc1C ; COc1cc(CC(C)NCc2ccccc2OC)c(OC)cc1C ; COc1cc(CC(C)NCc2ccccc2O)c(OC)cc1C]

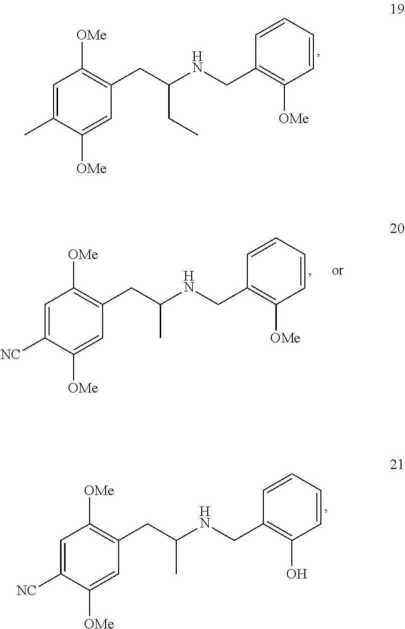 [SMILES: CCC(Cc1cc(OC)c(C)cc1OC)NCc1ccccc1OC ; COc1cc(CC(C)NCc2ccccc2OC)c(OC)cc1C#N ; COc1cc(CC(C)NCc2ccccc2O)c(OC)cc1C#N]

or a pharmaceutically acceptable salt thereof.

In some embodiments, the present disclosure provides a compound having the structure:

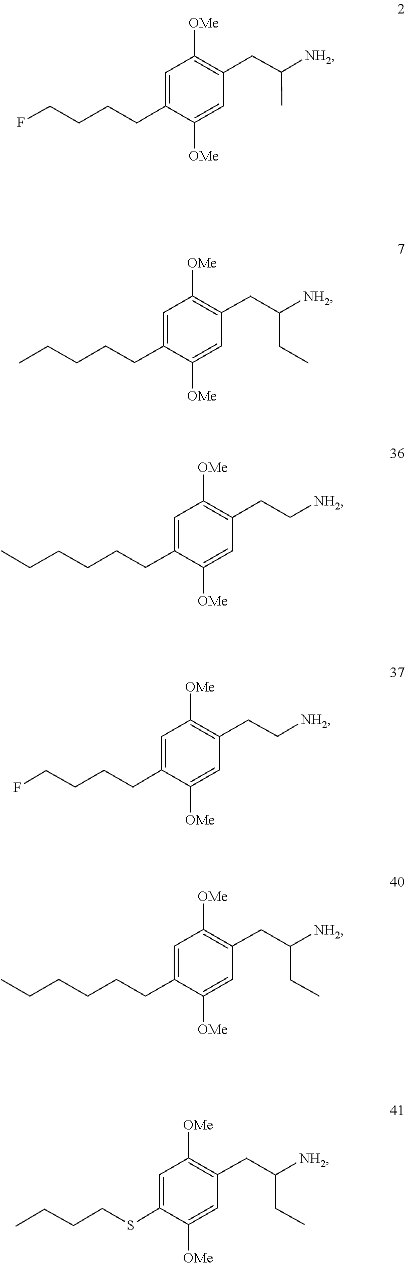

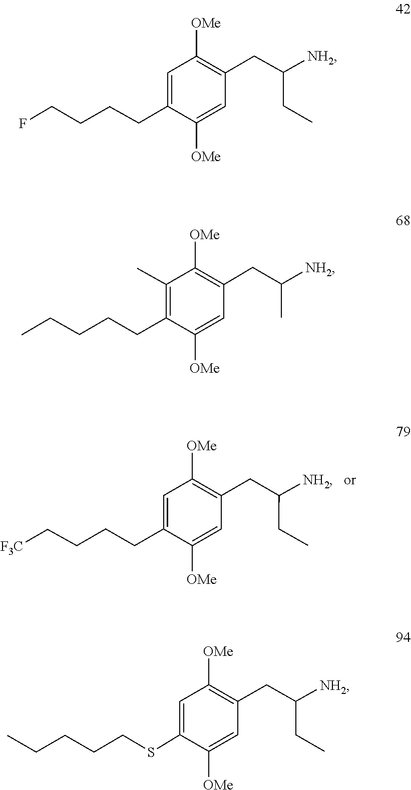 [SMILES: CCC(N)Cc1cc(OC)c(CCCCF)cc1OC ; CCCCCc1c(OC)cc(CC(C)N)c(OC)c1C ; CCC(N)Cc1cc(OC)c(CCCCC(F)(F)F)cc1OC ; CCCCCSc1cc(OC)c(CC(N)CC)cc1OC]

or a pharmaceutically acceptable salt thereof.

In some embodiments, the present disclosure provides a compound having the structure:

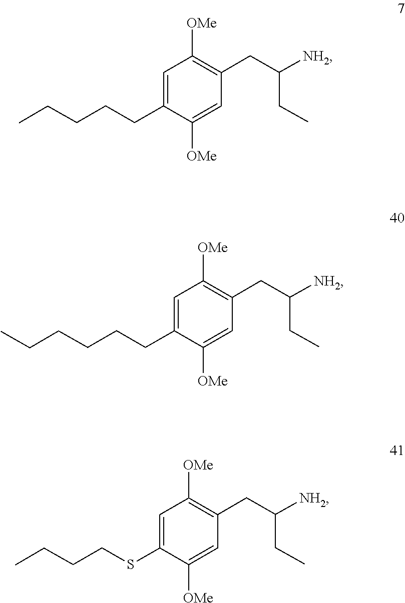

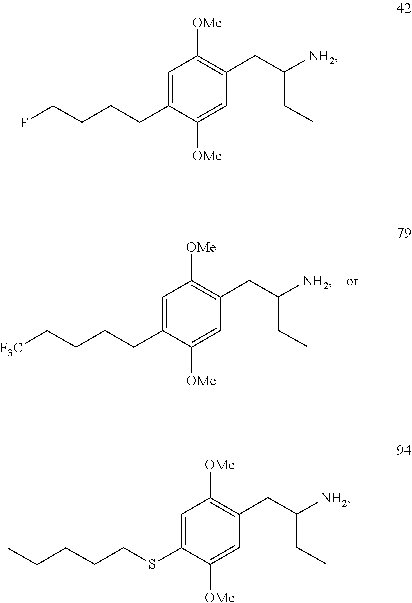

or a pharmaceutically acceptable salt thereof.

In some embodiments, the present disclosure provides a compound of Formula

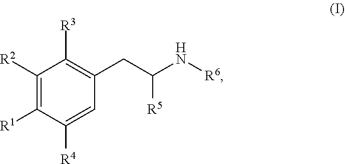

or a pharmaceutically acceptable salt thereof,
wherein
R¹ is C₄-C₈ alkyl, —S(C₄-C₈ alkyl), C₄-C₈ cycloalkylalkyl, —S(C₄-C₈ cycloalkylalkyl), or C₄-C₈ alkoxy,
   wherein R¹ is substituted with one or more substituents, wherein each substituent is fluoro;
R² is hydrogen, hydroxyl, C₁-C₃ alkyl, halo, —CF₃, —OCF₃, C₁-C₃ alkoxy, or —S(C₁-C₃ alkyl);
R³ is hydrogen, hydroxyl, C₁-C₃ alkyl, halo, —CF₃, —OCF₃, C₁-C₃ alkoxy, or —S(C₁-C₃ alkyl); wherein R² and R³ are not both hydrogen;
R⁴ is hydrogen, hydroxyl, C₁-C₃ alkyl, halo, —CF₃, —OCF₃, C₁-C₃ alkoxy, or —S(C₁-C₃ alkyl);
R⁵ is hydrogen or C₁-C₃ alkyl;
R⁶ is hydrogen or benzyl, wherein the phenyl ring of benzyl is optionally substituted with 1-5 instances of R^{6a};
each R^{6a} is independently selected for each occurrence from the group consisting of hydroxyl, C₁-C₆ alkoxy, C₁-C₆ alkyl, and halogen, or
   wherein any two adjacent R^{6a} can be taken together with the atoms on which they are attached to form an optionally substituted C₅-C₇ cycloalkyl or optionally substituted 3-7 membered heterocyclyl ring; and wherein when R¹ is —SCH₂CH₂CH₂CH₂F and R², R⁵, and R⁶ are each hydrogen, then at least one of R³ or R⁴ is other than —OMe.

In some embodiments, the present disclosure provides a compound of Formula (I), wherein
R¹ is C₄-C₈ alkyl, —S(C₄-C₈ alkyl), or C₄-C₈ alkoxy,
   wherein R¹ is substituted with one or more substituents, wherein each substituent is fluoro;
R² is hydrogen, C₁-C₃ alkoxy, or —S(C₁-C₃ alkyl);
R³ is hydrogen, C₁-C₃ alkoxy, or —S(C₁-C₃ alkyl);
   wherein R² and R³ are not both hydrogen;
R⁴ is hydrogen, C₁-C₃ alkoxy, or —S(C₁-C₃ alkyl);
R⁵ is hydrogen or C₁-C₂ alkyl;
R⁶ is hydrogen or benzyl, wherein the phenyl ring of benzyl is optionally substituted with 1-5 instances of R^{6a};
each R^{6a} is independently selected for each occurrence from the group consisting of hydroxyl, C₁-C₆ alkoxy, and halogen, or
   wherein any two adjacent R^{6a} can be taken together with the atoms on which they are attached to form an optionally substituted 3-7 membered heterocyclyl ring; and
wherein when R¹ is —SCH₂CH₂CH₂CH₂F and R², R⁵, and R⁶ are each hydrogen, then at least one of R³ or R⁴ is other than —OMe.

In some embodiments, the present disclosure provides a compound of Formula (I), wherein
R¹ is C₄-C₈ alkyl or —S(C₄-C₈ alkyl),
   wherein R¹ is substituted with one or more substituents, wherein each substituent is fluoro;
R² is hydrogen or C₁-C₃ alkoxy;
R³ is hydrogen or C₁-C₃ alkoxy;
   wherein R² and R³ are not both hydrogen;
R⁴ is hydrogen or C₁-C₃ alkoxy;
R⁵ is hydrogen or C₁-C₂ alkyl;
R⁶ is hydrogen; and
wherein when R¹ is —SCH₂CH₂CH₂CH₂F and R², R⁵, and R⁶ are each hydrogen, then at least one of R³ or R⁴ is other than —OMe.

In some embodiments, the present disclosure provides a compound of Formula (I), wherein
R¹ is C₄-C₈ alkyl or —S(C₄-C₈ alkyl),
   wherein R¹ is substituted with one or more substituents, wherein each substituent is fluoro;
R² is hydrogen, —OMe, or —OEt;
R³ is hydrogen, —OMe, or —OEt;
   wherein R² and R³ are not both hydrogen;
R⁴ is —OMe or —OEt;
R⁵ is hydrogen, Me, or Et;
R⁶ is hydrogen; and
wherein when R¹ is —SCH₂CH₂CH₂CH₂F and R², R⁵, and R⁶ are each hydrogen, then at least one of R³ or R⁴ is other than —OMe.

In some embodiments, the present disclosure provides a compound of Formula (I-a):

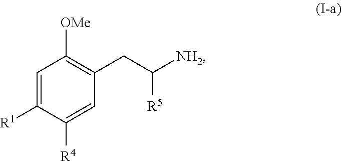

or a pharmaceutically acceptable salt thereof.

In some embodiments, the present disclosure provides a compound of Formula (I-b):

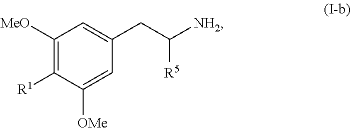

or a pharmaceutically acceptable salt thereof.

In some embodiments, the present disclosure provides a compound of Formula (I-c):

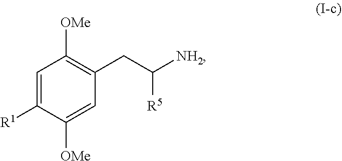

or a pharmaceutically acceptable salt thereof.

In some embodiments, R¹ is —S(C₄-C₈ alkyl) substituted with one or more substituents, wherein each substituent is fluoro.

In some embodiments, R¹ is C₄-C₈ alkyl substituted with one or more substituents, wherein each substituent is fluoro.

In some embodiments, R¹ is substituted with one, two, or three substituents, wherein each substituent is fluoro.

In some embodiments, R¹ is —S(C₄-C₈ alkyl) and substituted with one, two or three substituents, each selected from the group consisting of halogen. In some embodiments, R¹ is —S(C₄ alkyl) and substituted with one, two or three substituents, each selected from the group consisting of halogen. In some embodiments, R¹ is —S(C₅ alkyl) and substituted with one, two or three substituents, each selected from the group consisting of halogen. In some embodiments, R¹ is —S(C₆ alkyl) and substituted with one, two or three substituents, each selected from the group consisting of halogen. In some embodiments, R¹ is —S(C₇ alkyl) and substituted with one, two or three substituents, each selected from the group consisting of halogen.

In some embodiments, R¹ is —S(C₄-C₈ alkyl) and substituted with one halogen. In some embodiments, R¹ is —S(C₄-C₈ alkyl) and substituted with fluoro. In some embodiments, R¹ is —S(C₄-C₈ alkyl) and substituted with three halogens. In some embodiments, R¹ is —S(C₄-C₈ alkyl) and substituted with three fluoro groups.

In some embodiments, R¹ is selected from the group consisting of —SCH₂CH₂CH₂CH₂F, —SCH₂CH₂CH₂CH₂CH₂F, —SCH₂CH₂CH₂CH₂CH₂CH₂F, and —SCH₂CH₂CH₂CH₂CH₂CH₂CH₂F.

In some embodiments, R¹ is selected from the group consisting of —SCH₂CH₂CH₂CF₃, —SCH₂CH₂CH₂CH₂CF₃, —SCH₂CH₂CH₂CH₂CH₂CF₃, and —SCH₂CH₂CH₂CH₂CH₂CH₂CF₃.

In some embodiments, R¹ is —SCH₂CH₂CH₂CH₂F. In some embodiments, R¹ is —SCH₂CH₂CH₂CF₃. In some embodiments, R¹ is —SCH₂CH₂CH₂CH₂CH₂F. In some embodiments, R¹ is —SCH₂CH₂CH₂CH₂CF₃. In some embodiments, R¹ is —SCH₂CH₂CH₂CH₂CH₂CH₂F. In some embodiments, R¹ is —SCH₂CH₂CH₂CH₂CH₂CF₃. In some embodiments, R¹ is —SCH₂CH₂CH₂CH₂CH₂CH₂CH₂F. In some embodiments, R¹ is —SCH₂CH₂CH₂CH₂CH₂CH₂CF₃.

In some embodiments, R¹ is C₄-C₈ alkyl and substituted with one, two or three substituents, each selected from the group consisting of halogen. In some embodiments, R¹ is C₄ alkyl and substituted with one, two or three substituents, each selected from the group consisting of halogen. In some embodiments, R¹ is C₅ alkyl and substituted with one, two or three substituents, each selected from the group consisting of halogen. In some embodiments, R¹ is C₆ alkyl and substituted with one, two or three substituents, each selected from the group consisting of halogen. In some embodiments, R¹ is C₇ alkyl and substituted with one, two or three substituents, each selected from the group consisting of halogen.

In some embodiments, R¹ is C₄-C₈ alkyl and substituted with one halogen. In some embodiments, R¹ is C₄-C₈ alkyl and substituted with fluoro. In some embodiments, R¹ is C₄-C₈ alkyl and substituted with three halogens. In some embodiments, R¹ is C₄-C₈ alkyl and substituted with three fluoro groups.

In some embodiments, R¹ is selected from the group consisting of —CH₂CH₂CH₂CH₂F, —CH₂CH₂CH₂CH₂CH₂F, —CH₂CH₂CH₂CH₂CH₂CH₂F, and —CH₂CH₂CH₂CH₂CH₂CH₂CH₂F.

In some embodiments, R¹ is selected from the group consisting of —CH₂CH₂CH₂CF₃, —CH₂CH₂CH₂CH₂CF₃, —CH₂CH₂CH₂CH₂CH₂CF₃, and —CH₂CH₂CH₂CH₂CH₂CH₂CF₃.

In some embodiments, R¹ is —CH₂CH₂CH₂CH₂F. In some embodiments, R¹ is —CH₂CH₂CH₂CF₃. In some embodiments, R¹ is —CH₂CH₂CH₂CH₂CH₂F. In some embodiments, R¹ is —CH₂CH₂CH₂CH₂CF₃. In some embodiments, R¹ is —CH₂CH₂CH₂CH₂CH₂CH₂F. In some embodiments, R¹ is —CH₂CH₂CH₂CH₂CH₂CF₃. In some embodiments, R¹ is —CH₂CH₂CH₂CH₂CH₂CH₂CH₂F. In some embodiments, R¹ is —CH₂CH₂CH₂CH₂CH₂CH₂CF₃.

In some embodiments, the present disclosure provides a compound selected from the group consisting of

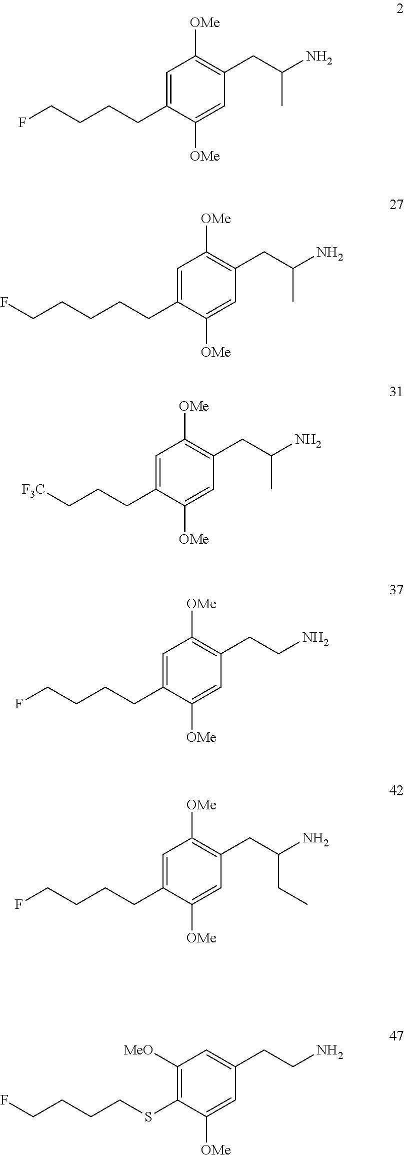 [SMILES: COc1cc(CC(C)N)c(OC)cc1CCCCF ; COc1cc(CC(C)N)c(OC)cc1CCCCCF ; COc1cc(CC(C)N)c(OC)cc1CCCC(F)(F)F ; COc1cc(CCCCF)c(OC)cc1CCN ; CCC(N)Cc1cc(OC)c(CCCCF)cc1OC ; COc1cc(CCN)cc(OC)c1SCCCCF]

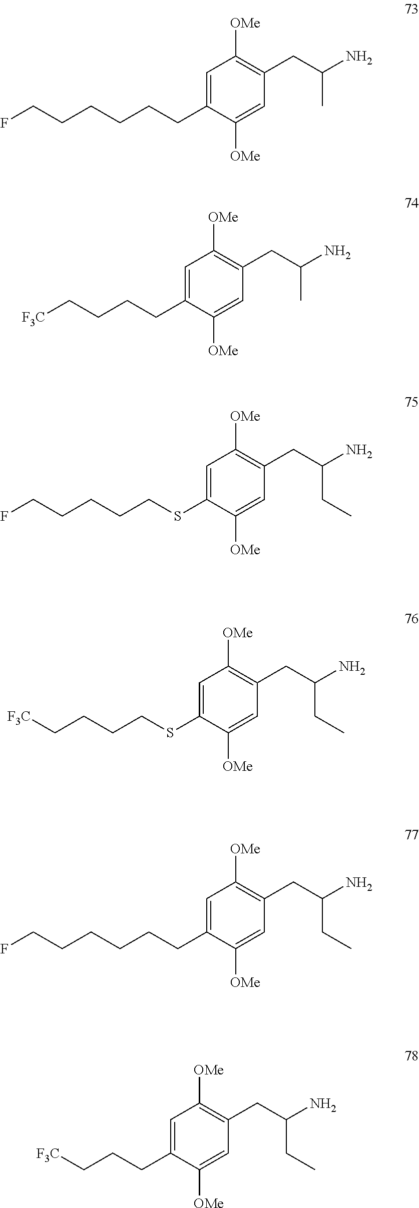 [SMILES: COc1cc(CC(C)N)c(OC)cc1CCCCCCF ; COc1cc(CC(C)N)c(OC)cc1CCCCC(F)(F)F ; CCC(N)Cc1cc(OC)c(SCCCCCF)cc1OC ; CCC(N)Cc1cc(OC)c(SCCCCC(F)(F)F)cc1OC ; CCC(N)Cc1cc(OC)c(CCCCCCF)cc1OC ; CCC(N)Cc1cc(OC)c(CCCC(F)(F)F)cc1OC]

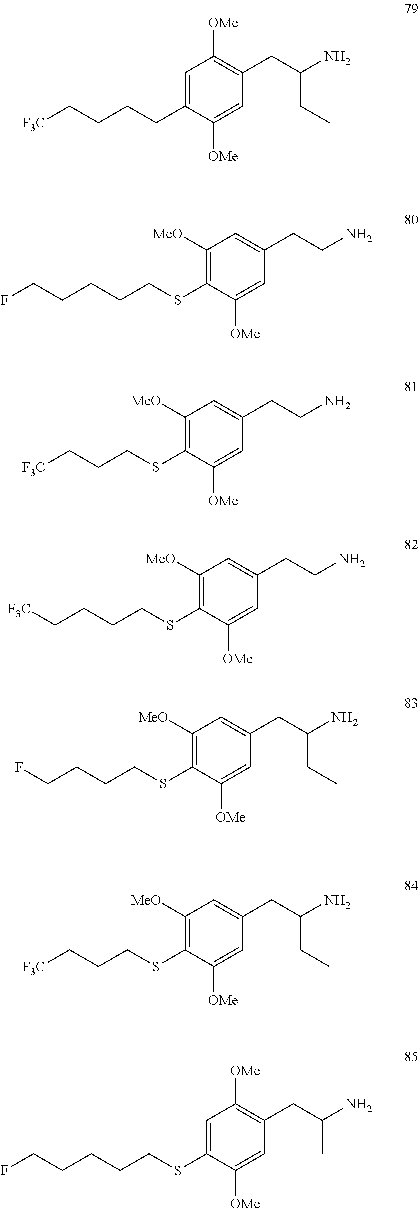

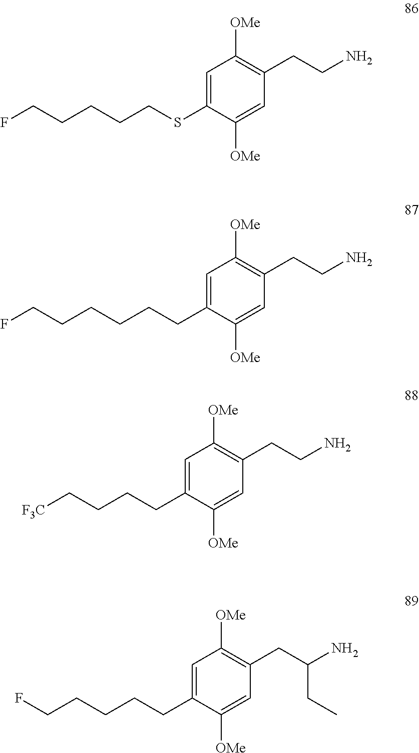 [SMILES: COc1cc(SCCCCCF)c(OC)cc1CCN ; COc1cc(CCCCCCF)c(OC)cc1CCN ; COc1cc(CCCCC(F)(F)F)c(OC)cc1CCN ; CCC(N)Cc1cc(OC)c(CCCCCF)cc1OC]

or a pharmaceutically acceptable salt thereof.

In some embodiments, the present disclosure provides a compound of Formula (II):

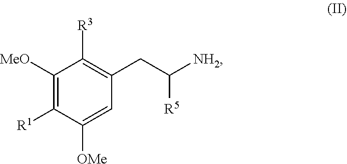

or a pharmaceutically acceptable salt thereof,
wherein
R¹ is C₄-C₈ alkyl or —S(C₄-C₈ alkyl),
   wherein C₄-C₈ alkyl may be optionally substituted by one or more substituents, each independently selected from the group consisting of hydroxyl, oxo, —CN, —NR⁷R⁸, C₁-C₆ alkoxy, C₁-C₃ alkyl, phenyl, 5-6 membered heteroaryl, C₃-C₆ cycloalkyl, and 3-6-membered heterocyclyl;
R³ is selected from the group consisting of hydrogen, halogen, and C₁-C₃ alkyl;
R⁵ is hydrogen or C₁-C₃ alkyl;
R⁷ and R⁸ are independently selected, for each occurrence, from the group consisting of hydrogen and C₁-C₃ alkyl; wherein C₁-C₃ alkyl may optionally be substituted by one or more substituents selected from the group consisting of fluorine, cyano, oxo, and hydroxyl;
or R⁷ and R⁸, together with the nitrogen to which they are attached, may form a 4-6 membered heterocyclic ring, which may have an additional heteroatom selected from O, S, or N;
   wherein the 4-6 membered heterocyclic ring may optionally be substituted by one or more substituents selected from the group consisting of fluorine, cyano, oxo, and hydroxyl; and
      wherein when R¹ is —SCH₂CH₂CH₂CH₃, R³ and R⁵ are not both hydrogen.

In some embodiments, the present disclosure provides a compound of Formula (II), wherein
R¹ is C₄-C₈ alkyl or —S(C₄-C₈ alkyl),
   wherein C₄-C₈ alkyl may be optionally substituted by one or more substituents, each independently selected from the group consisting of hydroxyl, C₁-C₆ alkoxy, and C₃-C₆ cycloalkyl;
R³ is selected from the group consisting of hydrogen, halogen, and C₁-C₃ alkyl;
R⁵ is hydrogen, Me, or Et; and
wherein when R¹ is —SCH₂CH₂CH₂CH₃, R³ and R⁵ are not both hydrogen.

In some embodiments, the present disclosure provides a compound of Formula (II), wherein
R¹ is C₄-C₈ alkyl or —S(C₄-C₈ alkyl),
   wherein C₄-C₈ alkyl is unsubstituted;
R³ is selected from the group consisting of hydrogen, halogen, and Me;
R⁵ is hydrogen, Me, or Et; and
wherein when R¹ is —SCH₂CH₂CH₂CH₃, R³ and R⁵ are not both hydrogen.

In some embodiments, R⁵ is hydrogen.

In some embodiments, R⁵ is C₁-C₂ alkyl.

In some embodiments, R⁵ is Me.

In some embodiments, R⁵ is Et.

In some embodiments, the present disclosure provides a compound of Formula (ITT):

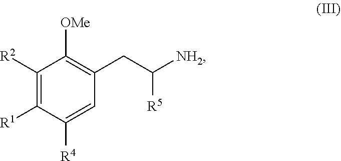

or a pharmaceutically acceptable salt thereof,
wherein
R¹ is selected from the group consisting of C₄-C₈ alkyl, —S(C₄-C₈ alkyl), and —(CH₂)₁₋₂O(C₁-C₃ alkyl),
   wherein C₄-C₈ alkyl may be optionally substituted by one or more substituents, each independently selected from the group consisting of hydroxyl, oxo, —CN, —NR⁷R⁸, C₁-C₆ alkoxy, C₁-C₃ alkyl, phenyl, 5-6 membered heteroaryl, C₃-C₆ cycloalkyl, and C₃-C₆ heterocyclyl;
R² is hydrogen or C₁-C₃ alkyl;
R⁴ is hydrogen or C₁-C₃ alkoxy;
R⁵ is hydrogen or C₁-C₃ alkyl, wherein C₁-C₃ alkyl may be optionally substituted by hydroxyl;
R⁷ and R⁸ are independently selected, for each occurrence, from the group consisting of hydrogen and C₁-C₃ alkyl; wherein C₁-C₃ alkyl may optionally be substituted by one or more substituents selected from the group consisting of fluorine, cyano, oxo, and hydroxyl;
or R⁷ and R⁸, together with the nitrogen to which they are attached, may form a 4-6 membered heterocyclic ring, which may have an additional heteroatom selected from O, S, or N;
   wherein the 4-6 membered heterocyclic ring may optionally be substituted by one or more substituents selected from the group consisting of fluorine, cyano, oxo, and hydroxyl; and
wherein
   when R¹ is —S(n-butyl) or —SCH₂CH₂CH₂CH₂F and R⁴ is —OMe, R² and R⁵ are not both hydrogen; and
   when R¹ is —S(n-butyl), —S(sec-butyl), —S(n-pentyl), n-pentyl, n-hexyl, n-heptyl, or —CH₂CH₂CH₂OMe, R⁴ is —OMe, and R² is hydrogen, R⁵ is not methyl.

In some embodiments, the present disclosure provides a compound of formula (IV):

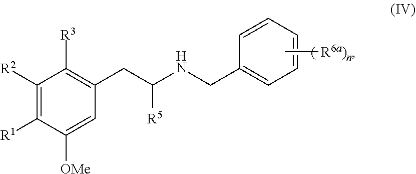

or a pharmaceutically acceptable salt thereof,
wherein
R¹ is selected from the group consisting of —CN, C₄-C₈ alkyl, and —S(C₄-C₈ alkyl),
   wherein C₄-C₈ alkyl may be optionally substituted by one or more substituents, each independently selected from the group consisting of hydroxyl, oxo, —CN, —NR⁷R⁸, C₁-C₆ alkoxy, C₁-C₃ alkyl, phenyl, 5-6 membered heteroaryl, C₃-C₆ cycloalkyl, and C₃-C₆ heterocyclyl;
R² is hydrogen or C₁-C₃ alkoxy;
R³ is hydrogen or C₁-C₃ alkoxy;
wherein R² and R³ are not both hydrogen;
R⁵ is hydrogen or C₁-C₃ alkyl;
each R^{6a} is independently selected for each occurrence from the group consisting of hydroxyl, C₁-C₆ alkoxy, C₁-C₆ alkyl, and halogen, or
   wherein any two adjacent R^{6a} can be taken together with the atoms on which they are attached to form an optionally substituted C₅-C₇ cycloalkyl or optionally substituted 3-7 membered heterocyclyl ring;
R⁷ and R⁸ are independently selected, for each occurrence, from the group consisting of hydrogen and C₁-C₃ alkyl; wherein C₁-C₃ alkyl may optionally be substituted by one or more substituents selected from the group consisting of fluorine, cyano, oxo, and hydroxyl;
or R⁷ and R⁸, together with the nitrogen to which they are attached, may form a 4-6 membered heterocyclic ring, which may have an additional heteroatom selected from O, S, or N;
   wherein the 4-6 membered heterocyclic ring may optionally be substituted by one or more substituents selected from the group consisting of fluorine, cyano, oxo, and hydroxyl.
wherein, when R¹ is —CN, R² is H, R³ is —OMe, and R^{6a} is ortho hydroxyl or ortho —OMe, R⁵ is not hydrogen.

In some embodiments, R¹ is selected from the group consisting of —CN, C₁-C₈ alkyl, and —S(C₄-C₈ alkyl), wherein C₁-C₈ alkyl and C₄-C₈ alkyl may be optionally substituted by one, two, three or more substituents, each independently selected from the group consisting of hydroxyl, fluoro, —CN, —NR⁷R⁸, C₁-C₆ alkoxy, C₁-3 alkyl, phenyl, 5-6 membered heteroaryl, C₃-C₆ cycloalkyl, and C₃-C₆ heterocyclyl. In some embodiments, R¹ is selected from the group consisting of —CN, C₄-C₈ alkyl, and —S(C₄-C₈ alkyl), wherein C₄-C₈ alkyl may be optionally substituted by one, two, three or more substituents, each independently selected from the group consisting of hydroxyl, fluoro, —CN, —NR⁷R⁸, C₁-C₆ alkoxy, C₁-3 alkyl, phenyl, 5-6 membered heteroaryl, C₃-C₆ cycloalkyl, and C₃-C₆ heterocyclyl. In some embodiments, R¹ is selected from the group consisting of —CN, C₁-C₈ alkyl, and —S(C₄-C₈ alkyl), wherein C₁-C₈ alkyl and C₄-C₈ alkyl may be optionally substituted by one, two, three or more substituents, each independently selected from the group consisting of hydroxyl, fluoro, —CN, —NR⁷R⁸, C₁-C₃ alkoxy, C₁-3 alkyl, C₃-C₆ cycloalkyl, and C₃-C₆ heterocyclyl. In some embodiments, R¹ is selected from the group consisting of —CN, C₄-C₈ alkyl, and —S(C₄-C₈ alkyl), wherein C₄-C₈ alkyl may be optionally substituted by one, two, three or more substituents, each independently selected from the group consisting of hydroxyl, fluoro, —CN, —NR⁷R⁸, C₁-C₃ alkoxy, C₁₋₃ alkyl, C₃-C₆ cycloalkyl, and C₃-C₆ heterocyclyl. In some embodiments, R¹ is selected from the group consisting of —CN, C₁-C₈ alkyl, and —S(C₄-C₈ alkyl), wherein C₁-C₈ alkyl and C₄-C₈ alkyl may be optionally substituted with hydroxy or fluoro. In some embodiments, R¹ is selected from the group consisting of —CN, C₄-C₈ alkyl, and —S(C₄-C₈ alkyl), wherein C₄-C₈ alkyl may be optionally substituted with hydroxy or fluoro. In some embodiments, R¹ is C₁-C₈ alkyl or —S(C₄-C₈ alkyl), wherein C₁-C₈ alkyl and C₄-C₈ alkyl may be optionally substituted with fluoro. In some embodiments, R¹ is C₄-C₈ alkyl or —S(C₄-C₈ alkyl), wherein C₄-C₈ alkyl may be optionally substituted with fluoro. In some embodiments, R¹ is selected from the group consisting of —CN, C₁-C₈ alkyl, and —S(C₄-C₈ alkyl). In some embodiments, R¹ is selected from the group consisting of —CN, C₄-C₈ alkyl, and —S(C₄-C₈ alkyl). In some embodiments, R¹ is —CN. In some embodiments, R¹ is C₁-C₈ alkyl or —S(C₄-C₈ alkyl). In some embodiments, R¹ is C₄-C₈ alkyl or —S(C₄-C₈ alkyl). In some embodiments, R¹ is C₁-C₈ alkyl. In some embodiments, R¹ is C₄-C₈ alkyl. In some embodiments, R¹ is selected from the group consisting of methyl, n-pentyl, neopentyl, n-hexyl, and isohexyl. In some embodiments, R¹ is selected from the group consisting of n-pentyl, n-hexyl, and isohexyl. In some embodiments, R¹ is —S(C₄-C₈ alkyl). In some embodiments, R¹ is selected from the group consisting of —S(n-propyl), —S(n-butyl), —S(n-pentyl), —S(neopentyl), —S(n-hexyl), and —S(isohexyl). In some embodiments, R¹ is selected from the group consisting of —S(n-butyl), —S(n-pentyl), —S(n-hexyl), and —S(isohexyl).

In some embodiments, R² is hydrogen or C₁-C₃ alkoxy. In some embodiments, R² is hydrogen or C₁-C₃ alkyl. In some embodiments, R² is hydrogen. In some embodiments, R² is C₁-C₃ alkoxy. In some embodiments, R² is methoxy. In some embodiments, R² is methyl.

In some embodiments, R³ is selected from the group consisting of hydrogen, halogen, and C₁-C₃ alkyl. In some embodiments, R³ is hydrogen or C₁-C₃ alkoxy. In some embodiments, R³ is hydrogen. In some embodiments, R³ is C₁-C₃ alkoxy. In some embodiments, R³ is methoxy. In some embodiments, R³ is methyl. In some embodiments, R³ is bromo.

In some embodiments, R² is hydrogen; and R³ is methoxy. In some embodiments, R² is methoxy; and R³ is hydrogen. In some embodiments, R² and R³ are not both hydrogen.

In some embodiments, R⁴ is hydrogen or C₁-C₃ alkoxy. In some embodiments, R⁴ is hydrogen. In some embodiments, R⁴ is C₁-C₃ alkoxy. In some embodiments, R⁴ is methoxy.

In some embodiments, R⁵ is hydrogen or C₁-C₃ alkyl. In some embodiments, R⁵ is hydrogen. In some embodiments, R⁵ is C₁-C₃ alkyl. In some embodiments, R⁵ is methyl. In some embodiments, R⁵ is ethyl. In some embodiments, R⁵ is hydroxymethyl.

In some embodiments, each R^{6a} is independently selected for each occurrence from the group consisting of hydroxyl, C₁-C₆ alkoxy, and halogen. In some embodiments, each R^{6a} is independently selected for each occurrence from the group consisting of hydroxyl, C₁-C₃ alkoxy, and halogen. In some embodiments, each R^{6a} is independently selected for each occurrence from the group consisting of hydroxyl, —OMe, and fluoro. In some embodiments, each R^{6a} is hydroxyl or C₁-C₆ alkoxy. In some embodiments, each R^{6a} is hydroxyl or C₁-C₃ alkoxy. In some embodiments, each R^{6a} is hydroxyl. In some embodiments, each R^{6a} is C₁-C₆ alkoxy. In some embodiments, each R^{6a} is C₁-C₃ alkoxy. In some embodiments, each R^{6a} is methoxy. In some embodiments, each R^{6a} is fluoro. In some embodiments, any two adjacent R^{6a} can be taken together with the atoms on which they are attached to form an optionally substituted C₅-C₇ cycloalkyl or optionally substituted 3-7 membered heterocyclyl ring. In some embodiments, any two adjacent R^{6a} can be taken together with the atoms on which they are attached to form a methylenedioxy ring.

In some embodiments, R⁶ is selected from the group consisting of

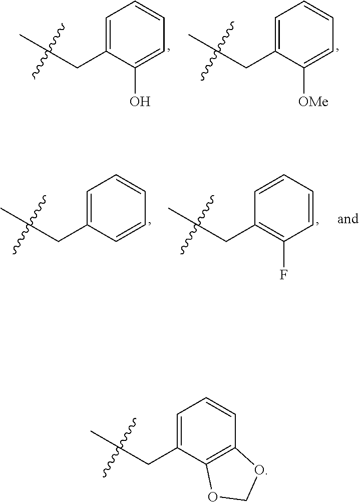 [SMILES: *Cc1ccccc1O ; *Cc1ccccc1OC ; *Cc1ccccc1 ; *Cc1ccccc1F ; *Cc1cccc2c1OCO2]

In some embodiments, the present disclosure provides a compound selected from the group consisting of

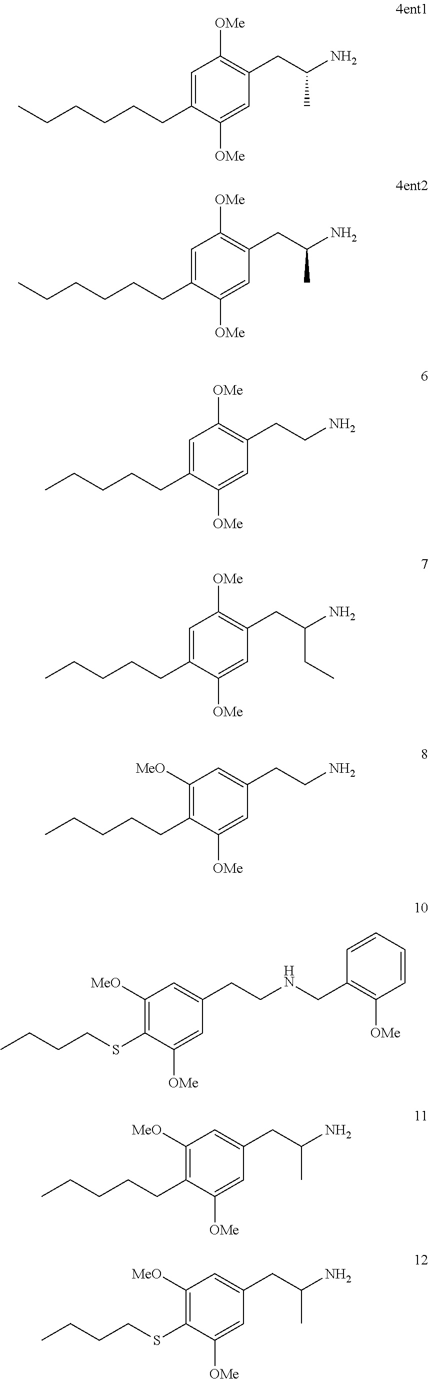

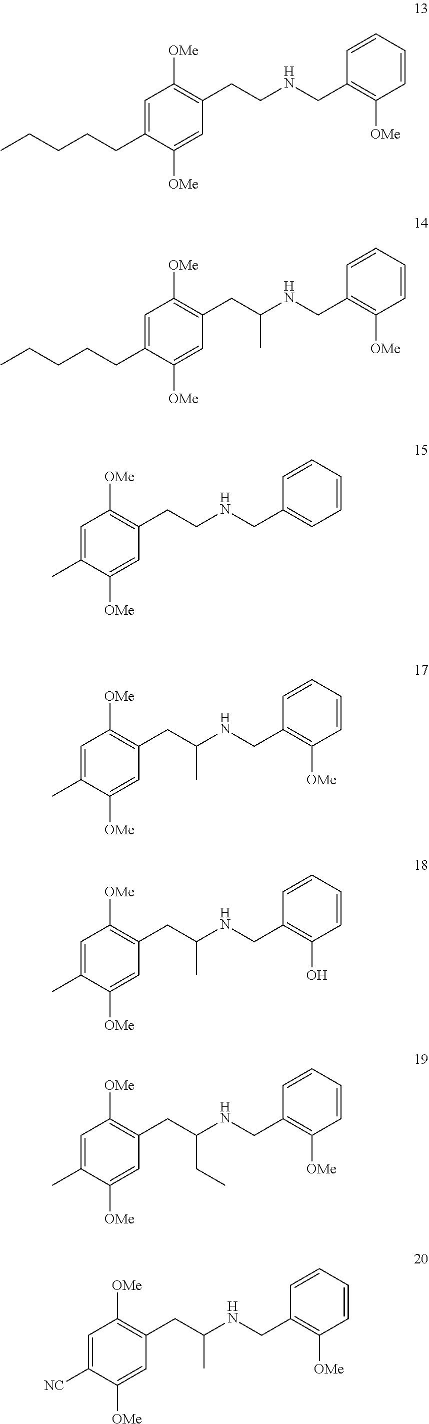

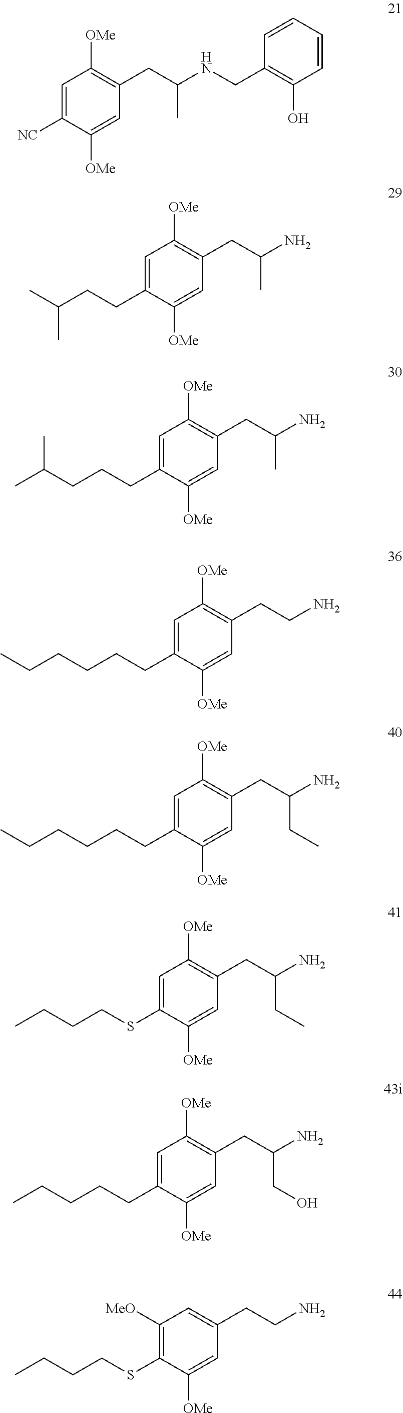

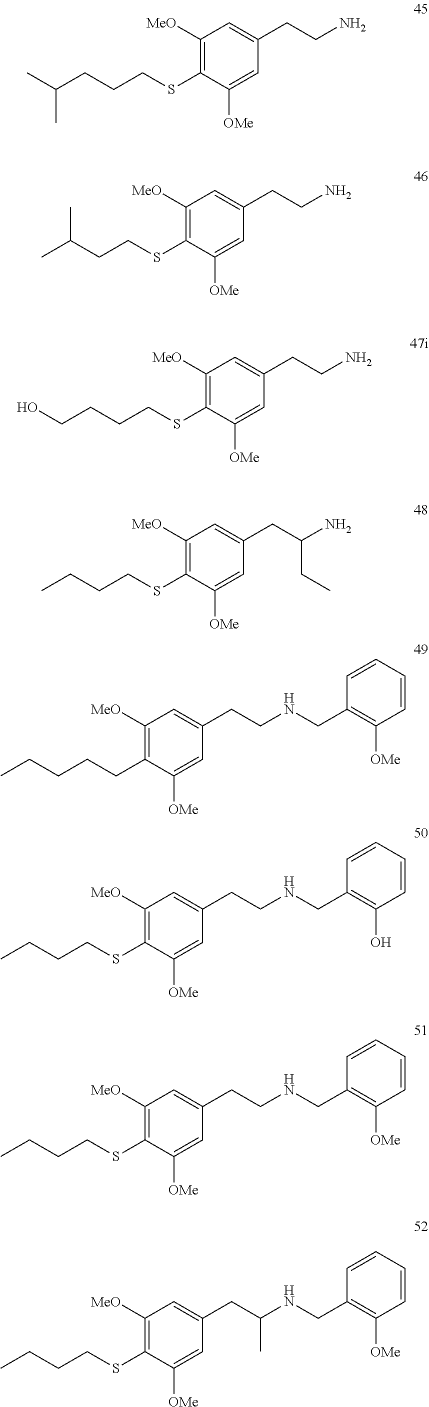

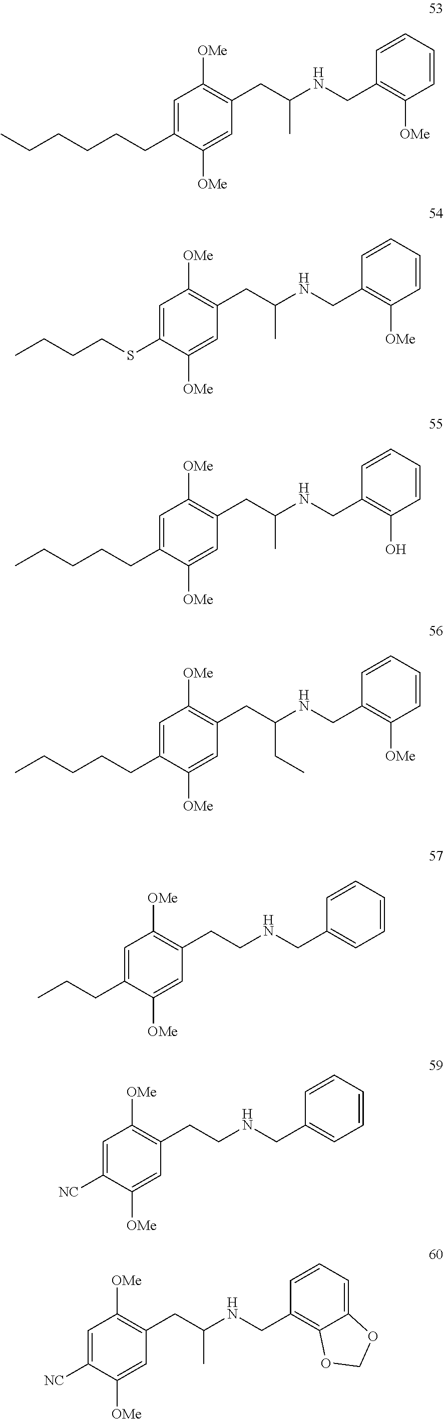

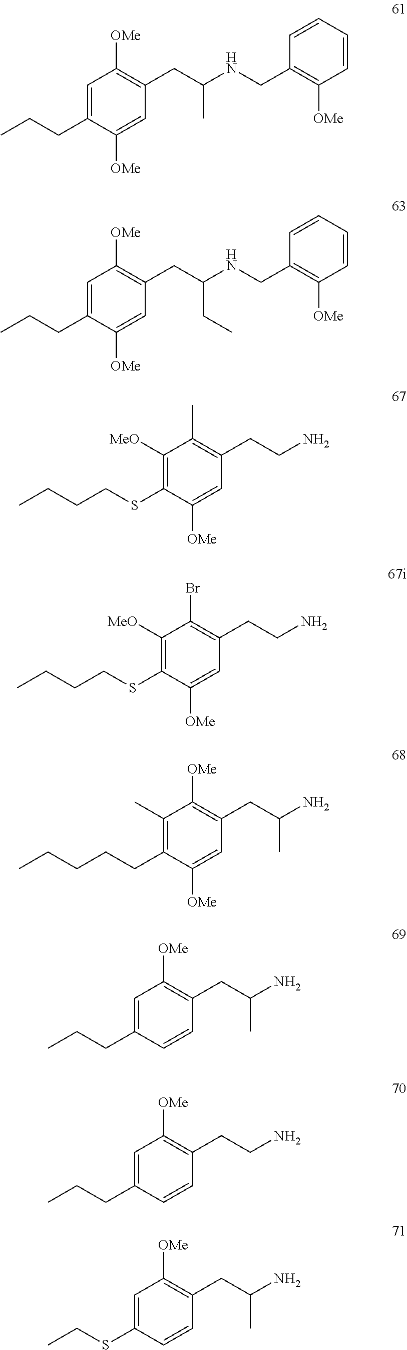

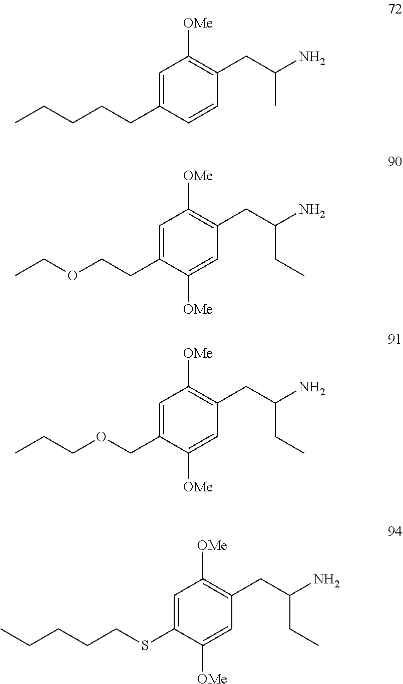

or a pharmaceutically acceptable salt thereof.

Salts of compounds of the present disclosure can be prepared by the reaction of a compound of the present disclosure with an appropriate acid or base in a suitable solvent, or mixture of solvents (such as an ether, for example, diethyl ether, or an alcohol, for example ethanol, or an aqueous solvent) using conventional procedures. Salts of compounds of the present disclosure can be exchanged for other salts by treatment using conventional ion-exchange chromatography procedures.

Where it is desired to obtain a particular enantiomer of a compound of the present disclosure, this may be produced from a corresponding mixture of enantiomers by employing any suitable conventional procedure for resolving enantiomers. For example, diastereomeric derivatives (such as salts) can be produced by reaction of a mixture of enantiomers of a compound of the present disclosure (such as a racemate) and an appropriate chiral compound (such as a chiral acid). The diastereomers can then be separated by any conventional means such as crystallisation, and the desired enantiomer recovered (such as by treatment with a base in the instance where the diastereomer is an acid salt). Alternatively, a racemic mixture of esters can be resolved by kinetic hydrolysis using a variety of biocatalysts (for example, see Patel Steroselective Biocatalysts, Marcel Decker; New York 2000).

In another resolution process, a racemate of a compound of the present disclosure can be separated using chiral High Performance Liquid Chromatography. Alternatively, a particular enantiomer can be obtained by using an appropriate chiral intermediate in one of the processes described above. Chromatography, recrystallisation, and other conventional separation procedures may also be used with intermediates or final products where it is desired to obtain a particular geometric isomer of the compounds disclosure herein.

### II. Methods

Another aspect of the disclosure provides methods of modulating the activity of 5-HT2A. Such methods comprise exposing said receptor to a compound described herein. In some embodiments, the compound utilized by one or more of the foregoing methods is one of the generic, subgeneric, or specific compounds described herein, such as a compound of Formula I, I-a, I-b, I-c, II, III or IV. The ability of compounds described herein to modulate, activate, or inhibit 5-HT2A can be evaluated by procedures known in the art and/or described herein. Another aspect of the disclosure provides methods of treating a disease associated with expression or activity of 5-HT2A in a patient. For example, a contemplated method includes administering a disclosed compound in an amount sufficient to establish activation of 5-HT2A effective to decrease the symptoms of a psychiatric disease or disorder in the patient. Further, treatment with a disclosed compound may also increase neuroplasticity or neurogenesis in a 5-HT2A-dependent manner.

In certain embodiments, the compound utilized by one or more of the foregoing methods is one of the generic, subgeneric, or specific compounds described herein, such as a compound of Formula I, I-a, I-b, I-c, II, III or IV.

In some embodiments, the present disclosure provides a method of treating a psychiatric disease or disorder comprising administering to a patient in need thereof a therapeutically effective amount of a compound of the present disclosure.

In some embodiments, the present disclosure provides a method of treating a psychiatric disease or disorder comprising administering to a patient in need thereof a therapeutically effective amount of a compound selected from the group consisting of:

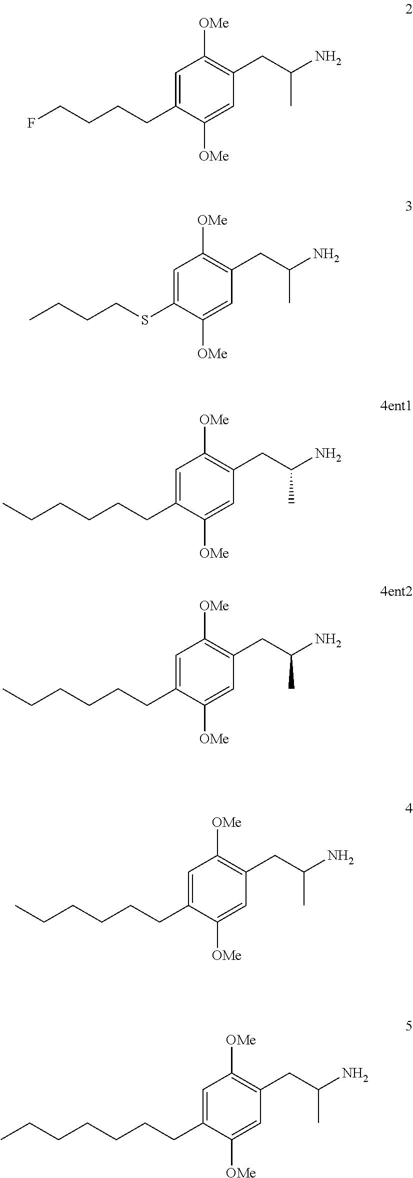 [SMILES: COc1cc(CC(C)N)c(OC)cc1CCCCF ; CCCCSc1cc(OC)c(CC(C)N)cc1OC ; CCCCCCc1cc(OC)c(C[C@@H](C)N)cc1OC ; CCCCCCc1cc(OC)c(C[C@H](C)N)cc1OC ; CCCCCCc1cc(OC)c(CC(C)N)cc1OC ; CCCCCCCc1cc(OC)c(CC(C)N)cc1OC]

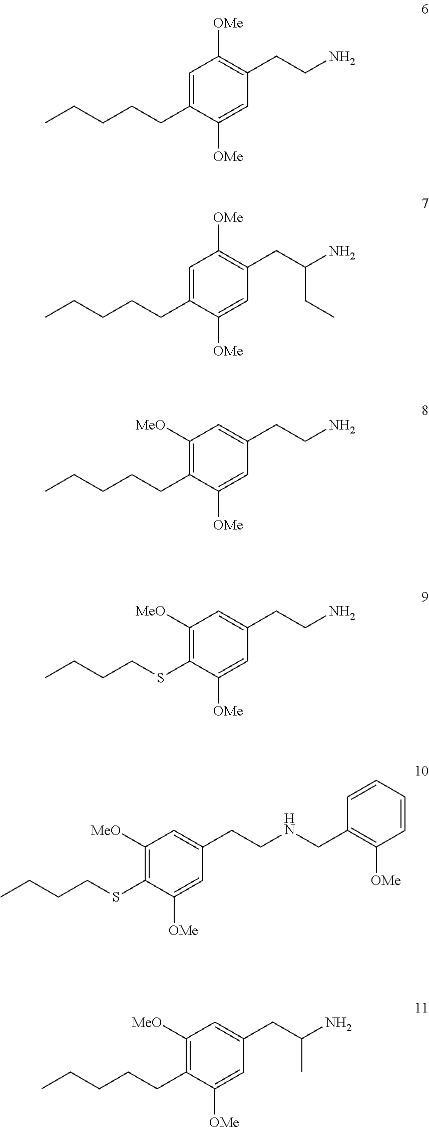

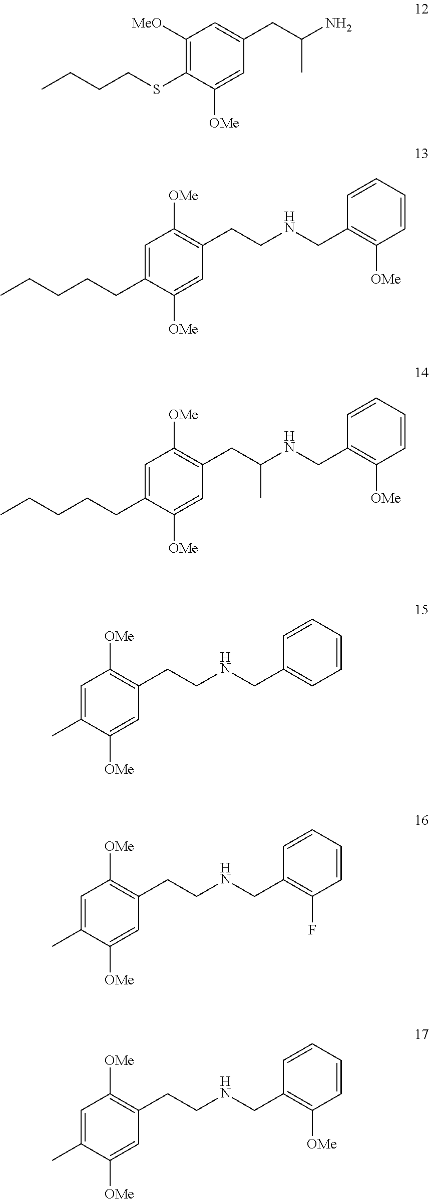 [SMILES: CCCCSc1c(OC)cc(CC(C)N)cc1OC ; CCCCCc1cc(OC)c(CCNCc2ccccc2OC)cc1OC ; CCCCCc1cc(OC)c(CC(C)NCc2ccccc2OC)cc1OC ; COc1cc(CCNCc2ccccc2)c(OC)cc1C ; COc1cc(CCNCc2ccccc2F)c(OC)cc1C ; COc1cc(CCNCc2ccccc2OC)c(OC)cc1C]

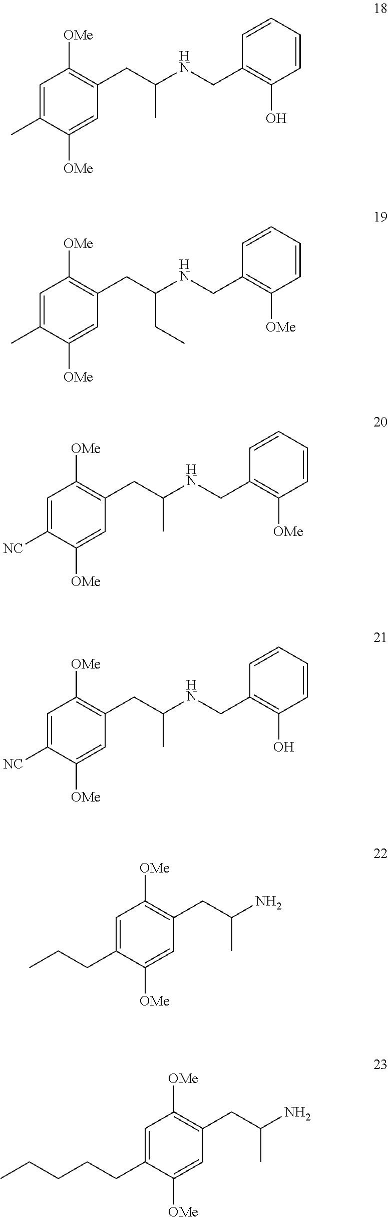 [SMILES: COc1cc(CC(C)NCc2ccccc2O)c(OC)cc1C ; CCC(Cc1cc(OC)c(C)cc1OC)NCc1ccccc1OC ; COc1cc(CC(C)NCc2ccccc2OC)c(OC)cc1C#N ; COc1cc(CC(C)NCc2ccccc2O)c(OC)cc1C#N ; CCCc1cc(OC)c(CC(C)N)cc1OC ; CCCCCc1cc(OC)c(CC(C)N)cc1OC]

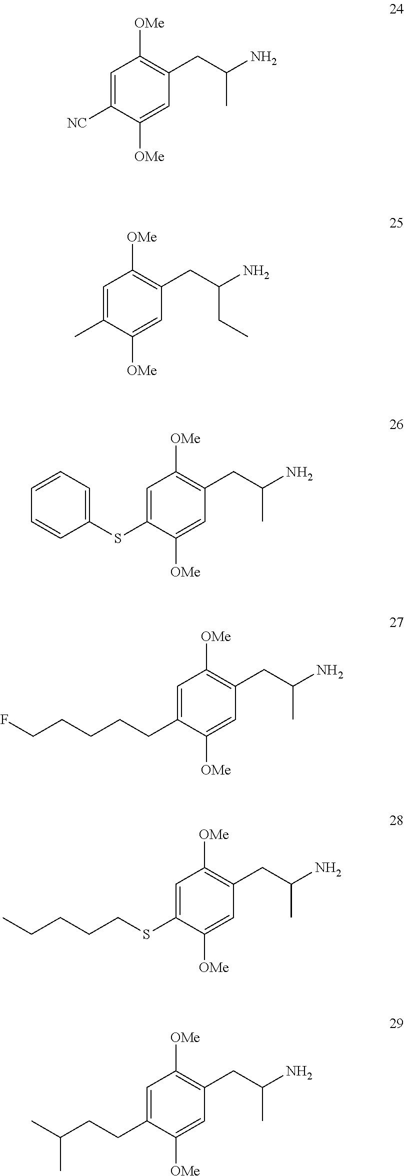 [SMILES: COc1cc(CC(C)N)c(OC)cc1C#N ; CCC(N)Cc1cc(OC)c(C)cc1OC ; COc1cc(Sc2ccccc2)c(OC)cc1CC(C)N ; COc1cc(CC(C)N)c(OC)cc1CCCCCF ; CCCCCSc1cc(OC)c(CC(C)N)cc1OC ; COc1cc(CC(C)N)c(OC)cc1CCC(C)C]

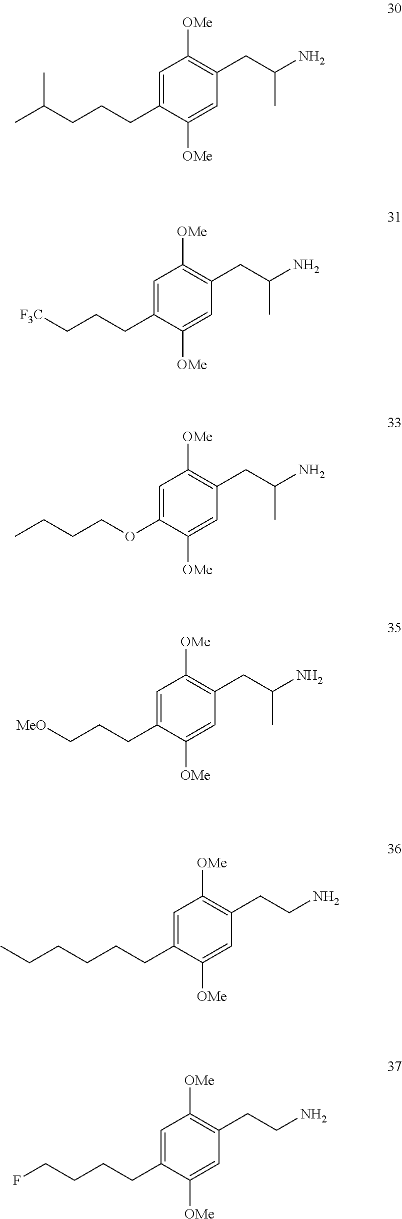 [SMILES: COc1cc(CC(C)N)c(OC)cc1CCCC(C)C ; COc1cc(CC(C)N)c(OC)cc1CCCC(F)(F)F ; CCCCOc1cc(OC)c(CC(C)N)cc1OC ; COCCCc1cc(OC)c(CC(C)N)cc1OC ; CCCCCCc1cc(OC)c(CCN)cc1OC ; COc1cc(CCCCF)c(OC)cc1CCN]

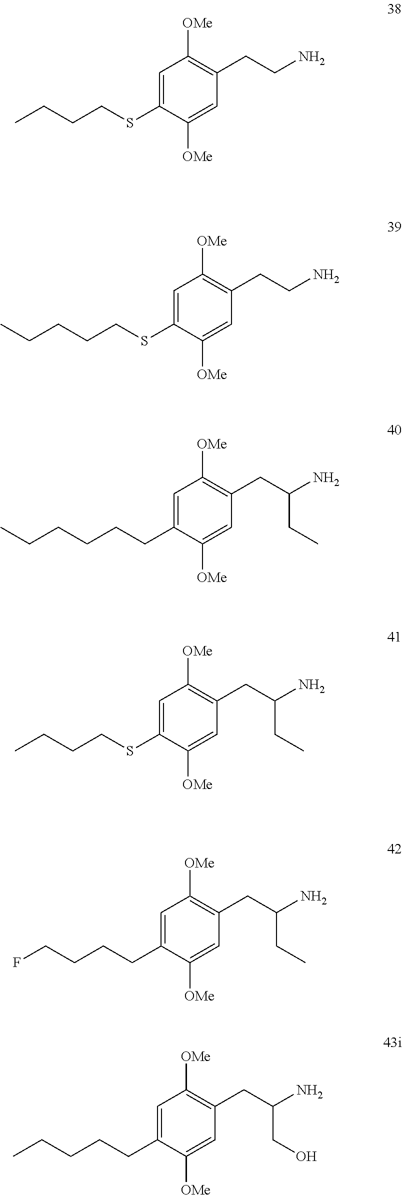

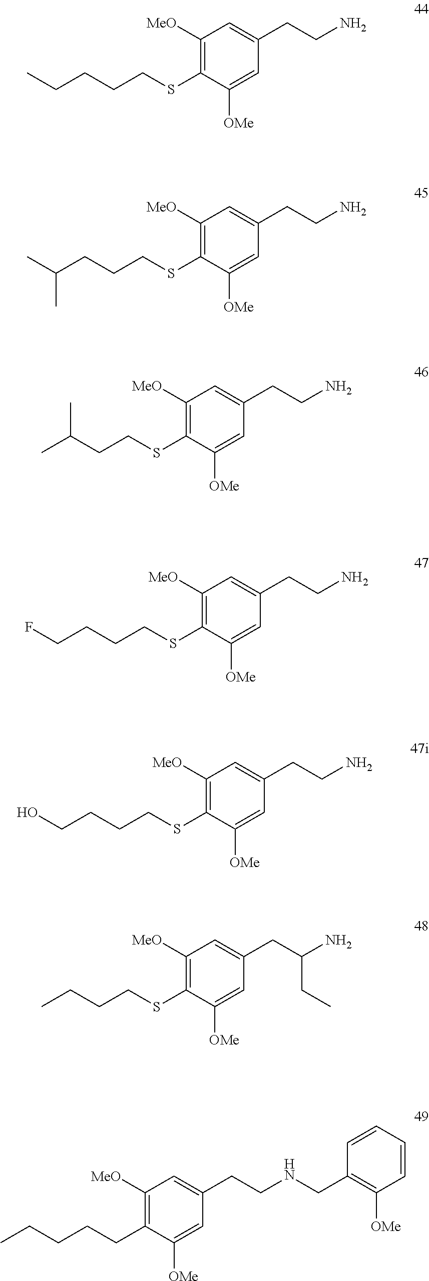

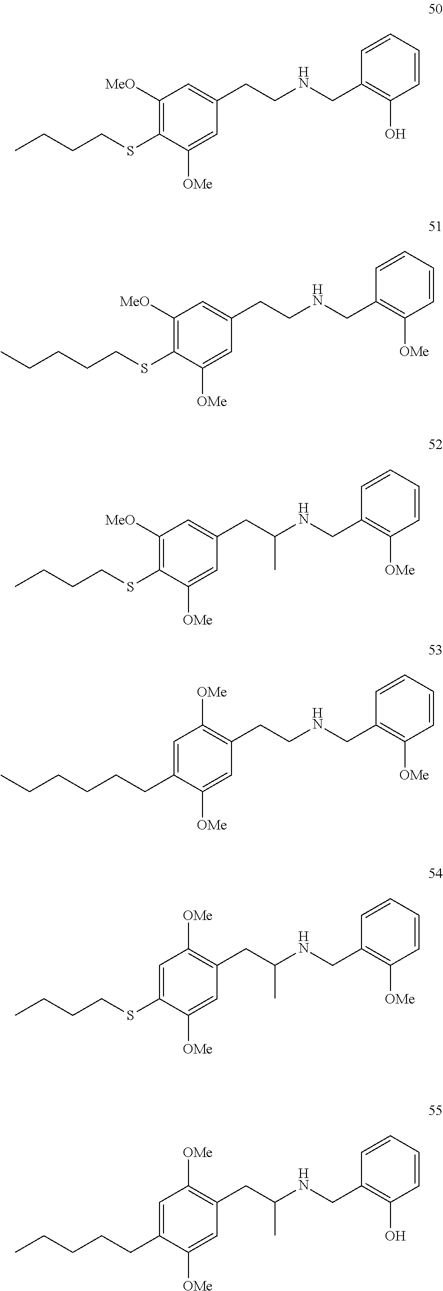 [SMILES: CCCCSc1c(OC)cc(CCNCc2ccccc2O)cc1OC ; CCCCCSc1c(OC)cc(CCNCc2ccccc2OC)cc1OC ; CCCCSc1c(OC)cc(CC(C)NCc2ccccc2OC)cc1OC ; CCCCCCc1cc(OC)c(CCNCc2ccccc2OC)cc1OC ; CCCCSc1cc(OC)c(CC(C)NCc2ccccc2OC)cc1OC ; CCCCCc1cc(OC)c(CC(C)NCc2ccccc2O)cc1OC]

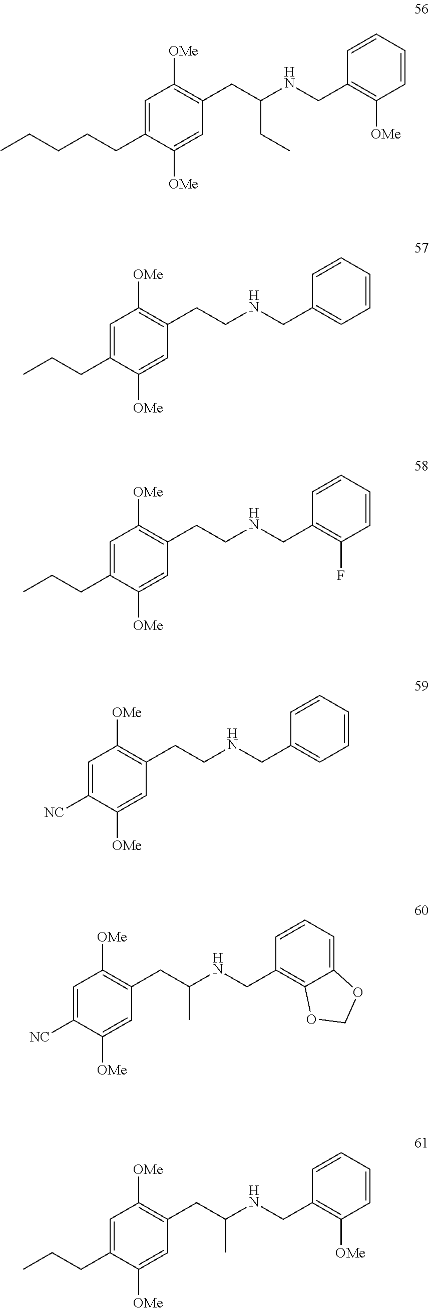 [SMILES: CCCCCc1cc(OC)c(CC(CC)NCc2ccccc2OC)cc1OC ; CCCc1cc(OC)c(CCNCc2ccccc2)cc1OC ; CCCc1cc(OC)c(CCNCc2ccccc2F)cc1OC ; COc1cc(CCNCc2ccccc2)c(OC)cc1C#N ; COc1cc(CC(C)NCc2cccc3c2OCO3)c(OC)cc1C#N ; CCCc1cc(OC)c(CC(C)NCc2ccccc2OC)cc1OC]

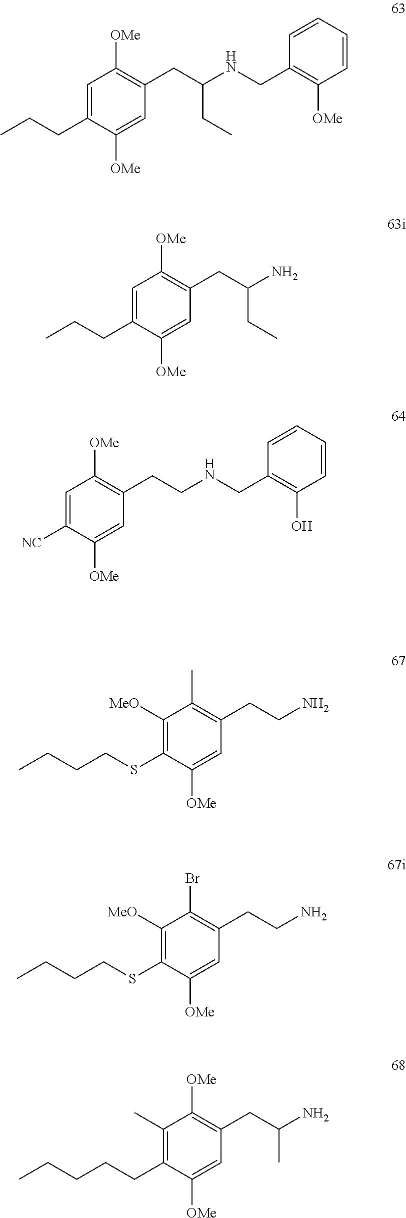

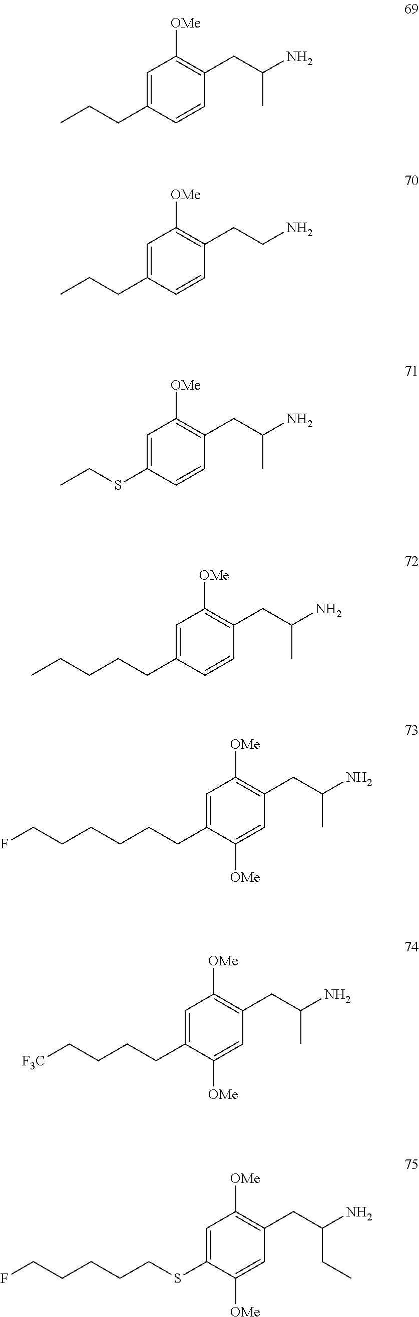

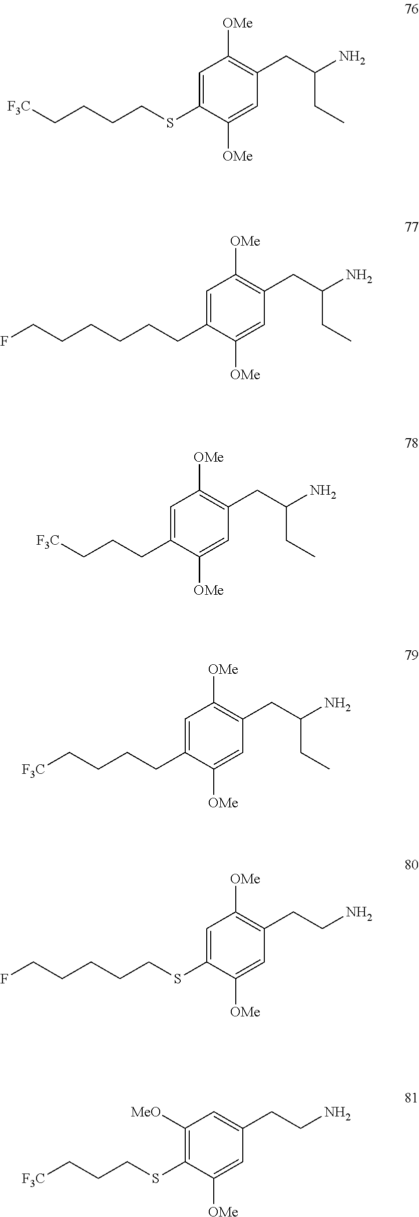 [SMILES: CCC(N)Cc1cc(OC)c(SCCCCC(F)(F)F)cc1OC ; CCC(N)Cc1cc(OC)c(CCCCCCF)cc1OC ; CCC(N)Cc1cc(OC)c(CCCC(F)(F)F)cc1OC ; CCC(N)Cc1cc(OC)c(CCCCC(F)(F)F)cc1OC ; COc1cc(SCCCCCF)c(OC)cc1CCN ; COc1cc(CCN)cc(OC)c1SCCCC(F)(F)F]

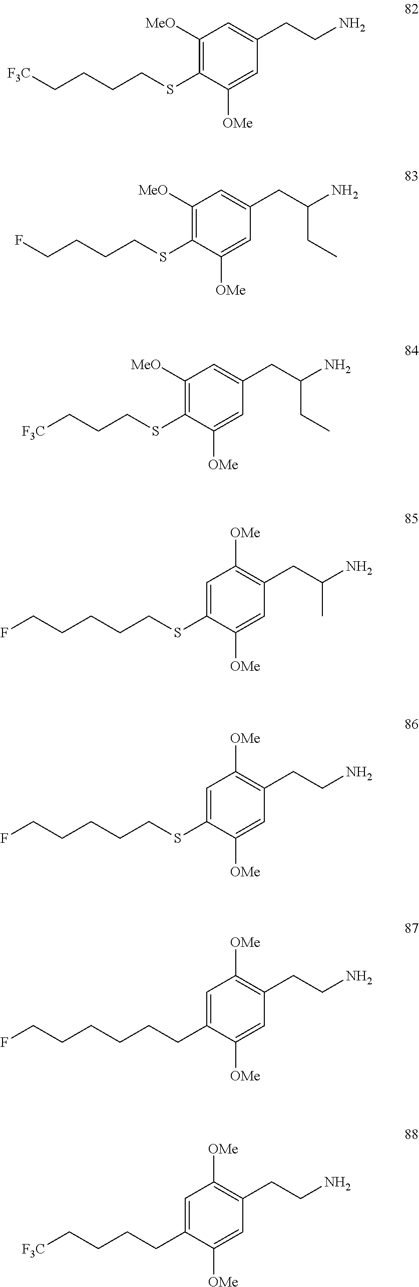

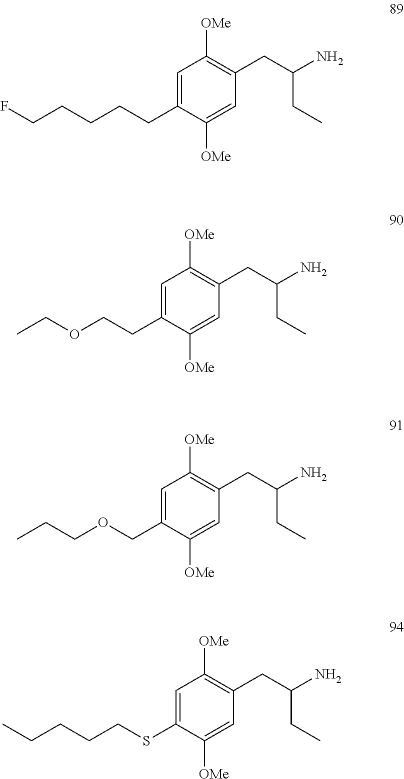

or a pharmaceutically acceptable salt thereof.

In some embodiments, the psychiatric disease or disorder is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, and depressive disorder due to another medical condition.

In some embodiments, the psychiatric disease or disorder is selected from the group consisting of bipolar disorder I, bipolar disorder II, cyclothymic disorder, substance/medication-induced bipolar and related disorder, and bipolar and related disorder due to another medical condition.

In some embodiments, the psychiatric disease or disorder is a substance-related disorder or substance-use disorder.

In some embodiments, the psychiatric disease or disorder is selected from the group consisting of separation anxiety disorder, selective mutism, specific phobia, social anxiety disorder, panic disorder, panic attach, agoraphobia, generalized anxiety disorder, substance/medication-induced anxiety disorder, anxiety disorder due to another medical condition.

In some embodiments, the psychiatric disease or disorder is selected from the group consisting of obsessive-compulsive and related disorders, trauma- and stressor-related disorders, feeding and eating disorders, borderline personality disorder, attention-deficit/hyperactivity disorder, and autism spectrum disorder.

In some embodiments, the psychiatric disorder is a neurocognitive disorder.

In some embodiments, the psychiatric disease or disorder is a treatment-resistant disease or disorder.

The present disclosure further provides a method of enhancing creativity or cognition in a subject, said method comprising administering to said subject a composition comprising an effective amount of a compound of the present disclosure.

In some embodiments, the compounds, methods, and compositions may be used to treat a psychiatric disorder including Depressive Disorders, e.g., Major Depressive Disorder, Persistent Depressive Disorder, Postpartum Depression, Premenstrual Dysphoric Disorder, Seasonal Affective Disorder, Psychotic Depression, Disruptive Mood Dysregulation Disorder, Substance/Medication-Induced Depressive Disorder, and Depressive Disorder Due to Another Medical Condition.

Also provided herein are compounds, methods, and compositions for treating refractory depression, e.g., patients suffering from a depressive disorder that does not, and/or has not, responded to adequate courses of at least one, or at least two, other antidepressant compounds or therapeutics. As used herein “depressive disorder” encompasses refractory depression.

In some embodiments, the compounds, methods, and compositions may be used to treat a psychiatric disorder including Bipolar and Related Disorders, e.g., Bipolar I Disorder, Bipolar II Disorder, Cyclothymic Disorder, Substance/Medication-Induced Bipolar and Related Disorder, and Bipolar and Related Disorder Due to Another Medical Condition.

In some embodiments, the compounds, methods, and compositions may be used to treat a psychiatric disorder including Substance-Related Disorders, e.g., preventing a substance use craving, diminishing a substance use craving, and/or facilitating substance use cessation or withdrawal. Substance use disorders involve abuse of psychoactive compounds such as alcohol, caffeine, cannabis, inhalants, opioids, sedatives, hypnotics, anxiolytics, stimulants, nicotine and tobacco. As used herein “substance” or “substances” are psychoactive compounds which can be addictive such as alcohol, caffeine, cannabis, hallucinogens, inhalants, opioids, sedatives, hypnotics, anxiolytics, stimulants, nicotine and tobacco. For example, the methods and compositions may be used to facilitate smoking cessation or cessation of opioid use.

In some embodiments, the compounds, methods, and compositions may be used to treat a psychiatric disorder including Anxiety Disorders, e.g., Separation Anxiety Disorder, Selective Mutism, Specific Phobia, Social Anxiety Disorder (Social Phobia), Panic Disorder, Panic Attack, Agoraphobia, Generalized Anxiety Disorder, Substance/Medication-Induced Anxiety Disorder, and Anxiety Disorder Due to Another Medical Condition.

In some embodiments, the compounds, methods, and compositions may be used to treat a psychiatric disorder including Obsessive-Compulsive and Related Disorders, e.g., Obsessive-Compulsive Disorder, Body Dysmorphic Disorder, Hoarding Disorder, Trichotillomania (Hair-Pulling Disorder), Excoriation (Skin-Picking) Disorder, Substance/Medication-Induced Obsessive-Compulsive and Related Disorder, and Obsessive-Compulsive and Related Disorder Due to Another Medical Condition.

In some embodiments, the compounds, methods, and compositions may be used to treat a psychiatric disorder including Trauma- and Stressor-Related Disorders, e.g., Reactive Attachment Disorder, Disinhibited Social Engagement Disorder, Posttraumatic Stress Disorder, Acute Stress Disorder, and Adjustment Disorders.

In some embodiments, the compounds, methods, and compositions may be used to treat a psychiatric disorder including Feeding and Eating Disorders, e.g., Anorexia Nervosa, Bulimia Nervosa, Binge-Eating Disorder, Pica, Rumination Disorder, and Avoidant/Restrictive Food Intake Disorder.

In some embodiments, the compounds, methods, and compositions may be used to treat a psychiatric disorder including Neurocognitive Disorders, e.g., Delirium, Major Neurocognitive Disorder, Mild Neurocognitive Disorder, Major or Mild Neurocognitive Disorder Due to Alzheimer's Disease, Major or Mild Frontotemporal Neurocognitive Disorder, Major or Mild Neurocognitive Disorder With Lewy Bodies, Major or Mild Vascular Neurocognitive Disorder, Major or Mild Neurocognitive Disorder Due to Traumatic Brain Injury, Substance/Medication-Induced Major or Mild Neurocognitive Disorder, Major or Mild Neurocognitive Disorder Due to HIV Infection, Major or Mild Neurocognitive Disorder Due to Prion Disease, Major or Mild Neurocognitive Disorder Due to Parkinson's Disease, Major or Mild Neurocognitive Disorder Due to Huntington's Disease, Major or Mild Neurocognitive Disorder Due to Another Medical Condition, and Major or Mild Neurocognitive Disorder Due to Multiple Etiologies.

In some embodiments, the compounds, methods, and compositions may be used to treat a psychiatric disorder including Neurodevelopmental Disorders, e.g., Autism Spectrum Disorder, Attention-Deficit/Hyperactivity Disorder, Stereotypic Movement Disorder, Tic Disorders, Tourette's Disorder, Persistent (Chronic) Motor or Vocal Tic Disorder, and Provisional Tic Disorder.

In some embodiments, the compounds, methods, and compositions may be used to treat a psychiatric disorder including Personality Disorders, e.g. Borderline Personality Disorder.

In some embodiments, the compounds, methods, and compositions may be used to treat a psychiatric disorder including Sexual Dysfunctions, e.g., Delayed Ejaculation, Erectile Disorder, Female Orgasmic Disorder, Female Sexual Interest/Arousal Disorder, Genito-Pelvic Pain/Penetration Disorder, Male Hypoactive Sexual Desire Disorder, Premature (Early) Ejaculation, and Substance/Medication-Induced Sexual Dysfunction.

In some embodiments, the compounds, methods, and compositions may be used to treat a psychiatric disorder including Gender Dysphoria, e.g., Gender Dysphoria.

In some embodiments, the compounds, methods, and compositions may be used to treat a headache disorder. In some embodiments, a headache disorder is a migraine or cluster headaches.

In some embodiments, the compounds, methods, and compositions may be used to treat an inflammatory disorder. In some embodiments, an inflammatory disorder is inflammatory bowel disease, including ulcerative colitis and Crohn's disease. In some embodiments, an inflammatory disorder is inflammatory bowel syndrome. In some embodiments, an inflammatory disorder is an inflammation-related cardiovascular disorder, such as artherosclerosis and coronary artery disease. In some embodiments, an inflammatory disorder is an inflammatory disorder dependent on TNF-α activity.

In some embodiments, the compounds, methods, and compositions may be used to treat high intraocular pressure.

The compounds of the disclosure may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound of this disclosure may be administered orally, subcutaneously, topically, parenterally, by inhalation spray, by vaporization, intranasally, or rectally in dosage unit formulations containing conventional non-toxic pharmaceutically acceptable carriers, adjuvants and vehicles. Parenteral administration may include subcutaneous injections, intravenous or intramuscular injections or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a decrease in symptoms of a psychiatric disorder, is achieved. A treatment regimen can include a corrective phase, during which a dose sufficient to provide symptomatic relief is administered, and can be followed by a maintenance phase, during which a lower dose sufficient to prevent a return of symptoms is administered. A suitable maintenance dose is likely to be found in the lower parts of the dose ranges provided herein, but corrective and maintenance doses can readily be established for individual subjects by those of skill in the art without undue experimentation, based on the disclosure herein. Maintenance doses can be employed to maintain remission in subjects whose symptoms have been previously controlled by other means, including treatments employing other pharmacological agents.

In some embodiments, methods include treating a psychiatric disorder, e.g., a depressive disorder, by administering to a patient in need thereof a pharmaceutical composition including about 0.01 mg to about 400 mg of a compound of the present disclosure. In some embodiments, doses may be, e.g., in the range of about 0.01 to 400 mg, 0.01 to 300 mg, 0.01 to 250 mg, 0.01 to 200 mg, 0.01 to 150 mg, 0.01 to 100 mg, 0.01 to 75 mg, 0.01 to 50 mg, 0.01 to 25 mg, 0.01 to 20 mg, 0.01 to 15 mg, 0.01 to 10 mg, 0.01 to 5 mg, 0.01 to 1 mg, 0.01 to 0.5 mg, 0.01 to 0.1 mg, 0.1 to 400 mg, 0.1 to 300 mg, 0.1 to 250 mg, 0.1 to 200 mg, 0.1 to 150 mg, 0.1 to 100 mg, 0.1 to 75 mg, 0.1 to 50 mg, 0.1 to 25 mg, 0.1 to 20 mg, 0.1 to 15 mg, 0.1 to 10 mg, 0.1 to 5 mg, 0.1 to 1 mg, 10 to 400 mg, 10 to 300 mg, 10 to 250 mg, 10 to 200 mg, 10 to 150 mg, 10 to 100 mg, 10 to 50 mg, 10 to 25 mg, 10 to 15 mg, 20 to 400 mg, 20 to 300 mg, 20 to 250 mg, 20 to 200 mg, 20 to 150 mg, 20 to 100 mg, 20 to 50 mg, 50 to 400 mg, 50 to 300 mg, 50 to 250 mg, 50 to 200 mg, 50 to 150 mg, 50 to 100 mg, 100 to 400 mg, 100 to 300 mg, 100 to 250 mg, 100 to 200 mg, with doses of, e.g., about 0.01 mg, 0.025 mg, 0.05 mg. 0.1 mg, 0.15 mg, 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 10 mg, 15 mg, 20 mg, 25 mg, 30, mg, 35 mg, 40 mg, 45 mg, 50 mg, 75 mg, 100 mg, 125 mg, 150 mg, 175 mg, 200 mg, 225 mg, 250 mg, 275 mg, 300 mg, and 400 mg being examples.

In some embodiments, dosages may include amounts of a compound of the present disclosure or a pharmaceutically acceptable salt thereof in the range of about, e.g., 1 mg to 50 mg, 1 mg to 40 mg, 1 mg to 30 mg, 1 mg to 20 mg, 1 mg to 15 mg, 1 mg to 10 mg, 0.1 mg to 10 mg, 0.1 to 5 mg, or 0.1 to 1 mg, with doses of 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12.5 mg, 15 mg, 17.5 mg, 20 mg, 22.5 mg, 25 mg, 27.5 mg, 30 mg, 35 mg, 40 mg, 45 mg, and 50 mg being specific examples of doses.

Typically, dosages of a compound of the present disclosure or a pharmaceutically acceptable salt thereof, are administered once, twice, three or four times daily, every other day, every three days, twice weekly, once weekly, twice monthly, once monthly, every two months, every 3 months, twice yearly, or once yearly to a patient in need thereof. In some embodiments, the dosage is about, e.g., 0.1-400 mg/administration, 0.1-300 mg/administration, 0.1-250 mg/administration, 0.1-200 mg/administration, 0.1-100 mg/administration, 0.1-50 mg/administration, or 0.1 to 25 mg/administration, for example 300 mg/administration, 250 mg/administration, 200 mg/administration, 150 mg/administration, 100 mg/administration, 75 mg/administration, 50 mg/administration, 25 mg/administration, 20 mg/administration, 10 mg/administration, 5 mg/administration, 2.5 mg/administration, 1 mg/administration, 0.5 mg/administration, 0.25 mg/administration, or 0.1 mg/administration.

In some embodiments, pharmaceutical compositions for parenteral or inhalation, e.g., a spray or mist, administration of a compound of the present disclosure or a pharmaceutically acceptable salt thereof, having a concentration of about 0.005 mg/mL to about 500 mg/mL. In some embodiments, the compositions include a compound of the present disclosure or a pharmaceutically acceptable salt thereof, at a concentration of, e.g., about 5 mg/mL to about 500 mg/mL, about 5 mg/mL to about 100 mg/mL, about 5 mg/mL to about 50 mg/mL, about 1 mg/mL to about 100 mg/mL, about 1 mg/mL to about 50 mg/mL, about 0.1 mg/mL to about 25 mg/mL, about 0.1 mg/mL to about 10 mg/mL, about 0.05 mg/mL to about 10 mg/mL, about 0.05 mg/mL to about 5 mg/mL, about 0.05 mg/mL to about 1 mg/mL, about 0.005 mg/mL to about 1 mg/mL, about 0.005 mg/mL to about 0.25 mg/mL, or about 0.005 mg/mL to about 0.1 mg/mL.

In some embodiments, the composition includes a compound of the present disclosure or a pharmaceutically acceptable salt thereof, at a concentration of, e.g., about 0.05 mg/mL to about 500 mg/mL, about 0.05 mg/mL to about 100 mg/mL, about 0.05 mg/mL to about 50 mg/mL, about 0.05 mg/mL to about 25 mg/mL, about 0.05 mg/mL to about 10 mg/mL, about 0.05 mg/mL to about 5 mg/mL, about 0.005 mg/mL to about 1 mg/mL, about 0.005 mg/mL to about 0.25 mg/mL, about 0.005 mg/mL to about 0.05 mg/mL, or about 0.005 mg/mL to about 0.025 mg/mL. In some embodiments, the pharmaceutical compositions are formulated as a total volume of about, e.g., 0.1 mL, 0.25 mL, 0.5 mL, 1 mL, 2 mL, 5 mL, 10 mL, 20 mL, 25 mL, 50 mL, 100 mL, 200 mL, 250 mL, or 500 mL.

Typically, dosages may be administered to a subject once, twice, three times or four times daily, every other day, every three days, twice weekly, once weekly, twice monthly, once monthly, thrice yearly, twice yearly, or once yearly. In some embodiments, a compound of the present disclosure or a pharmaceutically acceptable salt thereof is administered to a subject once in the morning, or once in the evening. In some embodiments, a compound of the present disclosure or a pharmaceutically acceptable salt thereof is administered to a subject once in the morning, and once in the evening. In some embodiments, a compound of the present disclosure or a pharmaceutically acceptable salt thereof is administered to a subject three times a day (e.g., at breakfast, lunch, and dinner), at a dose, e.g., of 0.5 mg/administration (e.g., 1.5 mg/day).

In some embodiments, a compound of the present disclosure or a pharmaceutically acceptable salt thereof is administered to a subject at a dose of 0.5 mg/day in one or more doses. In some embodiments, a compound of the present disclosure or a pharmaceutically acceptable salt thereof is administered to a subject at a dose of 1 mg/day in one or more doses. In some embodiments, a compound of the present disclosure or a pharmaceutically acceptable salt thereof is administered to a subject at a dose of 2.5 mg/day in one or more doses. In some embodiments, a compound of the present disclosure or a pharmaceutically acceptable salt thereof is administered to a subject at a dose of 5 mg/day in one or more doses. In some embodiments, a compound of the present disclosure or a pharmaceutically acceptable salt thereof is administered to a subject at a dose of 10 mg/day in one or more doses. In some embodiments, a compound of the present disclosure or a pharmaceutically acceptable salt thereof is administered to a subject at a dose of 15 mg/day in one or more doses. In some embodiments, a compound of the present disclosure or a pharmaceutically acceptable salt thereof is administered to a subject at a dose of 20 mg/day in one or more doses. In some embodiments, a compound of the present disclosure or a pharmaceutically acceptable salt thereof is administered to a subject at a dose of 25 mg/day in one or more doses. In some embodiments, a compound of the present disclosure or a pharmaceutically acceptable salt thereof is administered to a subject at a dose of 30 mg/day in one or more doses. In some embodiments, a compound of the present disclosure or a pharmaceutically acceptable salt thereof is administered to a subject at a dose of 40 mg/day in one or more doses. In some embodiments, a compound of the present disclosure or a pharmaceutically acceptable salt thereof is administered to a subject at a dose of 50 mg/day in one or more doses. In some embodiments, a compound of the present disclosure or a pharmaceutically acceptable salt thereof is administered to a subject at a dose of 75 mg/day in one or more doses. In some embodiments, a compound of the present disclosure or a pharmaceutically acceptable salt thereof is administered to a subject at a dose of 100 mg/day in one or more doses.

In some embodiments, the dosage of a compound of the present disclosure or a pharmaceutically acceptable salt thereof is 0.0005-5 mg/kg, 0.001-1 mg/kg, 0.01-1 mg/kg or 0.1-5 mg/kg once, twice, three times or four times daily. For example, in some embodiments, the dosage is 0.0005 mg/kg, 0.001 mg/kg, 0.005 mg/kg, 0.01 mg/kg, 0.025 mg/kg, 0.05 mg/kg, 0.1 mg/kg, 0.15 mg/kg, 0.2 mg/kg, 0.25 mg/kg, 0.3 mg/kg, 0.4 mg/kg, 0.5 mg/kg, 1 mg/kg, 2.5 mg/kg, 5 mg/kg, once, twice, three times, or four times daily. In some embodiments, a subject is administered a total daily dose of 0.01 mg to 500 mg of a compound of the present disclosure or a pharmaceutically acceptable salt thereof once, twice, three times, or four times daily. In some embodiments, the total amount administered to a subject in 24-hour period is, e.g., 0.01 mg, 0.025 mg, 0.05 mg, 0.075 mg, 0.1 mg, 0.125 mg, 0.15 mg, 0.175 mg, 0.2 mg, 0.25 mg, 0.3 mg, 0.4 mg, 0.5 mg, 0.75 mg, 1 mg, 1.5 mg, 2 mg, 2.5 mg, 3 mg, 4 mg, 5 mg, 7.5 mg, 10 mg, 12.5 mg, 15 mg, 17.5 mg 20 mg, 25 mg, 30 mg, 35 mg, 40 mg, 50 mg, 60 mg, 75 mg, 100 mg, 150 mg, 200 mg, 300 mg, 400 mg, 500 mg. In some embodiments, the subject may be started at a low dose and the dosage is escalated. In some embodiments, the subject may be started at a high dose and the dosage is decreased.

In some embodiments, a compound of the present disclosure or a pharmaceutically acceptable salt thereof is administered to a patient under the supervision of a healthcare provider.

In some embodiments, a compound of the present disclosure or a pharmaceutically acceptable salt thereof is administered to a patient under the supervision of a healthcare provider at a clinic specializing in the delivery of psychoactive treatments.

In some embodiments, a compound of the present disclosure is administered to a patient under the supervision of a healthcare provider at a high dose intended to induce a psychedelic experience in the subject, e.g., 5 mg, 7.5 mg, 10 mg, 12.5 mg, 15 mg, 17.5 mg, 20 mg, 25 mg, 30 mg, 35 mg, 40 mg, 50 mg, 60 mg, 70 mg, 80 mg, 90 mg, or 100 mg.

In some embodiments, the administration to a patient of a high dose under the supervision of a healthcare provider occurs periodically in order to maintain a therapeutic effect in the patient, e.g., every three days, twice weekly, once weekly, twice monthly, once monthly, four times yearly, thrice yearly, twice yearly, or once yearly.

In some embodiments, a compound of the present disclosure or a pharmaceutically acceptable salt thereof is administered by a patient on their own at home or otherwise away from the supervision of a healthcare provider.

In some embodiments, a compound of the present disclosure or a pharmaceutically acceptable salt thereof is administered by a patient on their own at home or otherwise away from the supervision of a healthcare provider at a low dose intended to be sub-perceptual or to induce threshold psychoactive effects, e.g., 0.1 mg, 0.25 mg, 0.5 mg, 0.75 mg, 1 mg, 1.5 mg, 2 mg, 2.5 mg, 3 mg, or 4 mg.

In some embodiments, the administration by a patient of a low dose on their own occurs periodically in order to maintain a therapeutic effect in the patient, e.g., daily, every other day, every three days, twice weekly, once weekly, twice monthly, or once monthly,

In some embodiments, a compound of the present disclosure or a pharmaceutically acceptable salt thereof may be administered, e.g., via inhalation or orally, at specified intervals. For example, during treatment a patient may be administered a compound of the present disclosure at intervals of every, e.g., 1 year, 6 months, 4 months, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

### III. Pharmaceutical Compositions and Kits

Another aspect of the disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, intranasal, aerosol, or vaporization administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions of this disclosure may be used in the form of a pharmaceutical preparation, for example, in solid, semisolid or liquid form, which contains one or more of the compounds of the disclosure, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

In another aspect, the disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Advantageously, the disclosure also provides kits for use by a, e.g., a consumer in need of treatment with a disclosed compound. Such kits include a suitable dosage form such as those described above and instructions describing the method of using such dosage form to treat a medical disorder, for example, a psychiatric disease or disorder. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . etc”. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Also contemplated herein are methods and compositions that include a second active agent, or administering a second active agent.

### EXEMPLIFICATION

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment, and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “Intermediates” herein are contemplated as compounds of the disclosure.

### General Procedures

The compounds of the present disclosure may be prepared by techniques well known in organic synthesis and familiar to a practitioner ordinarily skilled in the art. For example, the compounds may be prepared by the chemical transformations described in the following examples. However, these may not be the only means by which to synthesize or obtain the desired compounds.

### ABBREVIATIONS

DOI=1-(4-iodo-2,5-dimethoxyphenyl)propan-2-amine
25D-NBOMe=2-(2,5-dimethoxy-4-methylphenyl)-N-(2-methoxybenzyl)ethan-1-amine
2C-TFM=1-(2,5-dimethoxy-4-(trifluoromethyl)phenyl)propan-2-amine
25CN-NBOH=4-(2-((2-hydroxybenzyl)amino)ethyl)-2,5-dimethoxybenzonitrile
LSD=(6aR,9R)—N,N-diethyl-7-methyl-4,6,6a,7,8,9-hexahydroindolo[4,3-fg]quinoline-9-carboxamide=lysergic acid diethylamide
Mescaline=2-(3,4,5-trimethoxyphenyl)ethan-1-amine
DMT=2-(1H-indol-3-yl)-N,N-dimethylethan-1-amine=N,N-dimethyltryptamine
2C-B=2-(4-bromo-2,5-dimethoxyphenyl)ethan-1-amine
2C-E=2-(4-ethyl-2,5-dimethoxyphenyl)ethan-1-amine
Psilocin=3-(2-(dimethylamino)ethyl)-1H-indol-4-ol=4-hydroxy-N,N-dimethyltryptamine
5-MeO-DMT=2-(5-methoxy-1H-indol-3-yl)-N,N-dimethylethan-1-amine=5-methoxy-N,N-dimethyltryptamine

### Intermediates

### Preparation 1: Preparation of tert-butyl (4-bromo-2,5-dimethoxyphenethyl)carbamate (Intermediate 1)

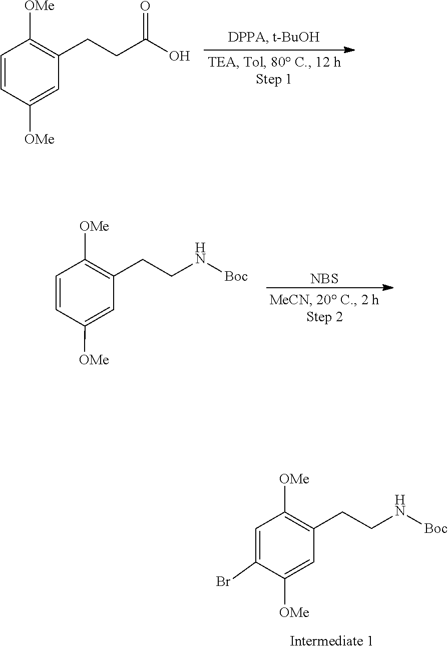 [SMILES: COc1ccc(OC)c(CCC(=O)O)c1 ; COc1ccc(OC)c(CCNC(=O)OC(C)(C)C)c1 ; "NBS" ; COc1cc(CCNC(=O)OC(C)(C)C)c(OC)cc1Br]

### Step 1: Preparation of tert-butyl (2,5-dimethoxyphenethyl)carbamate

To a solution of 3-(2,5-dimethoxyphenyl)propanoic acid (10 g, 47.57 mmol, 1 eq.) in toluene (150 mL) was added DPPA (15.71 g, 57.08 mmol, 12.37 mL, 1.2 eq.) and TEA (9.63 g, 95.14 mmol, 13.24 mL, 2 eq.). The mixture was stirred at 80° C. for 5 h, then t-BuOH (17.63 g, 237.84 mmol, 22.75 mL, 5 eq.) was added to the solution, and the reaction mixture was stirred at 80° C. for 7 h. Upon completion, the solvent was removed in vacuo. The residue was purified by silica gel chromatography (PE:EA 100:1-10:1) to afford tert-butyl (2,5-dimethoxyphenethyl)carbamate (7 g, 24.9 mmol, 52% yield) as a yellow oil. ¹H NMR (400 MHz, CHLOROFORM-d) δ=6.81-6.77 (m, 1H), 6.76-6.71 (m, 2H), 4.73-4.60 (m, 1H), 3.80-3.73 (m, 6H), 3.40-3.29 (m, 2H), 2.84-2.74 (m, 2H), 1.44 (s, 9H).

### Step 2: Preparation of tert-butyl (4-bromo-2,5-dimethoxyphenethyl)carbamate (Intermediate 1)

To a solution of tert-butyl (2,5-dimethoxyphenethyl)carbamate (4 g, 14.22 mmol, 1 eq.) in MeCN (50 mL) was added NBS (3.29 g, 18.48 mmol, 1.3 eq.) at 20° C. The mixture was stirred at 20° C. for 1 h. Upon completion, the mixture was poured into sat. aq. Na₂S₂O₃ soln. (5 mL) and extracted with EA (5 mL×2). The organic layer was washed with brine, dried over Na₂SO₄, filtered, and concentrated. The residue was purified by silica gel chromatography (PE:EA=100:1-8:1) to afford tert-butyl (4-bromo-2,5-dimethoxyphenethyl)carbamate (4.9 g, 13.60 mmol, 96% yield) as a brown oil. ¹H NMR (400 MHz, CHLOROFORM-d) δ=7.05-7.02 (m, 1H), 6.76-6.71 (m, 1H), 4.62 (br s, 1H), 3.87-3.83 (m, 3H), 3.81-3.77 (m, 3H), 3.37-3.28 (m, 2H), 2.81-2.74 (m, 2H), 1.45-1.41 (m, 9H).

### Preparation 2: Preparation of benzyl (1-(4-bromo-2,5-dimethoxyphenyl)propan-2-yl) carbamate (Intermediate 2)

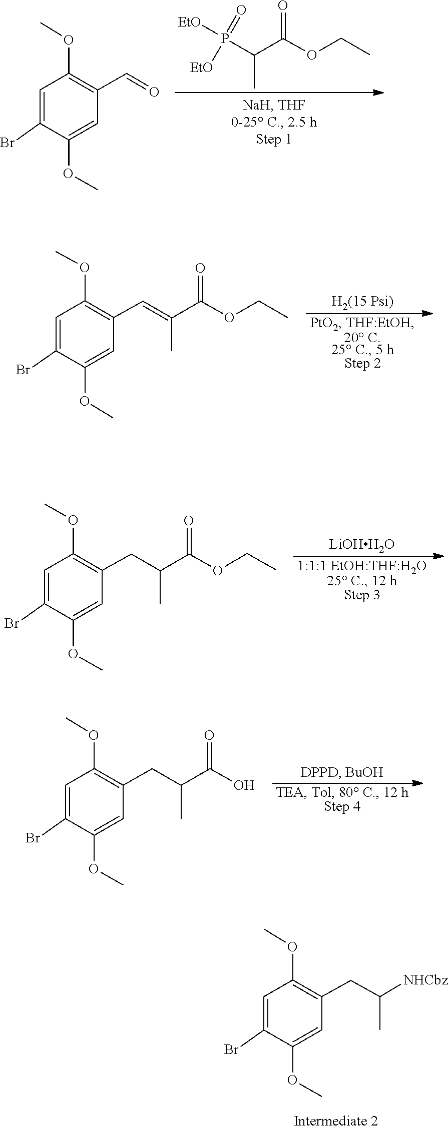

### Step 1: Preparation of ethyl (E)-3-(4-bromo-2,5-dimethoxyphenyl)-2-methylacrylate

To a suspension of NaH (5.39 g, 134.66 mmol, 60% purity, 1.1 eq.) in THF (300 mL) was added dropwise ethyl 2-diethoxyphosphorylpropanoate (32.08 g, 134.66 mmol, 29.43 mL, 1.1 eq.) at 0° C. The resulting solution was stirred at 0° C. for 30 min. Then a solution of 4-bromo-2,5-dimethoxybenzaldehyde (30 g, 122.41 mmol, 1 eq) in THF (50 mL) was added via syringe. The reaction mixture was stirred at 25° C. for 2 h. Upon completion, the mixture was quenched with saturated aqueous NH₄Cl solution (100 mL). The organic layers were separated, and the aqueous phase was extracted with DCM (100 mL×3). The combined organic layer was washed with brine, dried over Na₂SO₄, filtered, and concentrated in vacuo. The residue was purified by column chromatography (SiO₂, Petroleum ether/Ethyl acetate=100/1-0/1) to afford ethyl (E)-3-(4-bromo-2,5-dimethoxyphenyl)-2-methylacrylate (29 g, 88.10 mmol, 72% yield) as a white solid. ¹H NMR (400 MHz, DMSO-d₆) δ ppm 10.29 (s, 1H), 7.59 (s, 1H), 7.29 (s, 1H), 7.04 (s, 1H), 4.23-4.17 (m, 2H), 3.90-3.84 (m, 1H), 3.80 (s, 3H), 3.78 (s, 3H), 1.98 (s, 3H), 1.26 (t, J=6.8 Hz, 3H).

### Step 2: Preparation of ethyl 3-(4-bromo-2,5-dimethoxyphenyl)-2-methylpropanoate

To a solution of ethyl (E)-3-(4-bromo-2,5-dimethoxyphenyl)-2-methylacrylate (14 g, 42.53 mmol, 1 eq.) in EtOH (140 mL) and THF (140 mL) was added PtO₂ (2.80 g, 12.33 mmol, 0.29 eq.) under N₂. The suspension was degassed under vacuum and purged with H₂ several times. The mixture was stirred under H₂ (15 psi) at 15° C. for 1 h. Upon completion, the reaction mixture was filtered, and the filtrate was concentrated to afford ethyl 3-(4-bromo-2,5-dimethoxy-phenyl)-2-methylpropanoate (13 g, crude) as a yellow oil.

### Step 3: Preparation of 3-(4-bromo-2,5-dimethoxyphenyl)-2-methylpropanoic acid

A mixture of ethyl 3-(4-bromo-2,5-dimethoxy-phenyl)-2-methylpropanoate (7 g, 21.14 mmol, 1 eq.) and LiOH·H₂O (1.24 g, 29.59 mmol, 1.4 eq.) in THF (25 mL), H₂O (25 mL), and EtOH (25 mL) was stirred at 25° C. for 12 h. Upon completion, the reaction mixture was quenched by addition of aq. HCl (1M) until pH=6-7 was reached and then the mixture was diluted with H₂O (100 mL) and extracted with EtOAc (200 mL×2). The combined organic layer was washed with brine, dried over Na₂SO₄, filtered, and concentrated to afford 3-(4-bromo-2,5-dimethoxy-phenyl)-2-methylpropanoic acid (6 g, 19.79 mmol, 94% yield) as a white solid.

### Step 4: Preparation of benzyl (1-(4-bromo-2,5-dimethoxyphenyl)propan-2-yl)carbamate (Intermediate 2)

To a solution of 3-(4-bromo-2,5-dimethoxy-phenyl)-2-methylpropanoic acid (15 g, 49.48 mmol, 1 eq.) in toluene (150 mL) was added DPPA (14.98 g, 54.43 mmol, 11.79 mL, 1.1 eq.) and TEA (15.02 g, 148.44 mmol, 20.66 mL, 3 eq.). The mixture was stirred at 15° C. for 1 h, then phenylmethanol (10.70 g, 98.96 mmol, 10.29 mL, 2 eq.) was added dropwise. The resulting mixture was stirred at 80° C. for 12 h. Upon completion, the mixture was quenched with H₂O (100 mL). The layers were separated, and the aqueous phase was extracted with EtOAc (200 mL×3). The combined organic layer was washed with brine (200 mL), dried over Na₂SO₄, filtered, and concentrated in vacuo. The residue was purified by column chromatography (SiO₂, Petroleum ether/Ethyl acetate=100/1-0/1) to afford benzyl (1-(4-bromo-2,5-dimethoxyphenyl)propan-2-yl)carbamate (18.7 g, crude) as a white solid. ¹H NMR (400 MHz, DMSO-d₆) δ ppm 7.35-7.24 (m, 5H), 7.14 (s, 1H), 6.93 (s, 1H), 4.51-4.49 (d, J=5.6 Hz, 2H), 3.83-3.77 (m, 1H), 3.72 (s, 6H), 2.72-2.57 (m, 2H), 1.06 (t, J=6.8 Hz, 3H).

### Preparation 3: Preparation of benzyl (1-(4-bromo-2,5-dimethoxyphenyl)butan-2-yl)carbamate (Intermediate 3)

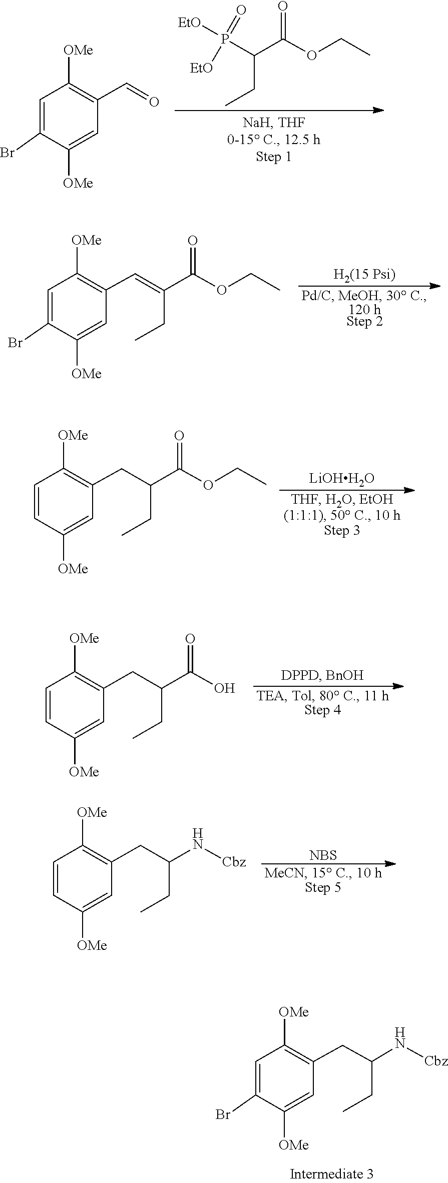

### Step 1: Preparation of ethyl (E)-2-(4-bromo-2,5-dimethoxybenzylidene)butanoate

To a suspension of NaH (1.80 g, 44.9 mmol, 60% purity, 1.1 eq.) in THF (20 mL) was added dropwise ethyl 2-diethoxyphosphorylbutanoate (11.3 g, 44.9 mmol, 10.7 mL, 1.1 eq.). The resulting solution was stirred at 0° C. for 30 min. Then 4-bromo-2,5-dimethoxybenzaldehyde (10 g, 40.8 mmol, 1 eq.) in THF (10 mL) was added. The reaction mixture was stirred at 15° C. for 12 h. Upon completion, the mixture was quenched with saturated aqueous NH₄Cl solution (40 mL). The layers were separated, and the aqueous phase was extracted with DCM (40 mL×3). The combined organic layer was washed with saturated aqueous NaCl solution (20 mL), dried over Na₂SO₄, filtered, and concentrated in vacuo. The residue was purified by column chromatography (SiO₂, Petroleum ether/Ethyl acetate=100/1-0/1) to afford ethyl (E)-2-(4-bromo-2,5-dimethoxybenzylidene)butanoate (10 g, 29.14 mmol, 71% yield) as a white solid. 1H NMR (400 MHz, CHLOROFORM-d) δ 7.70-7.64 (m, 1H), 7.10 (s, 1H), 6.86 (s, 1H), 4.28 (q, J=7.2 Hz, 2H), 3.87-3.84 (m, 3H), 3.83-3.80 (m, 3H), 2.48 (q, J=7.2 Hz, 2H), 1.36 (t, J=7.2 Hz, 3H), 1.17 (t, J=7.6 Hz, 3H).

### Step 2: Preparation of ethyl 2-(2,5-dimethoxybenzyl)butanoate

To a solution of ethyl (E)-2-(4-bromo-2,5-dimethoxybenzylidene)butanoate (7.00 g, 20.40 mmol, 1 eq.) in MeOH (70 mL) was added Pd/C (1.40 g, 140 mmol, 10% Pd, 6.9 eq.) under N₂. The suspension was degassed under vacuum and purged with H₂ several times. The mixture was stirred under H₂ (15 psi) at 30° C. for 120 h. Upon completion, the reaction mixture was filtered and the filtrate concentrated to afford ethyl 2-(2,5-dimethoxybenzyl)butanoate (5.98 g, crude) as a yellow oil. 1H NMR (400 MHz, CHLOROFORM-d) δ 6.81-6.68 (m, 3H), 4.08 (q, J=6.8 Hz, 2H), 3.79 (s, 3H), 3.75 (s, 3H), 2.82 (d, J=7.2 Hz, 2H), 2.72-2.62 (m, 1H), 1.72-1.50 (m, 2H), 1.18 (t, J=7.2 Hz, 3H), 0.97-0.88 (m, 3H).

### Step 3: Preparation of 2-(2,5-dimethoxybenzyl)butanoic acid

To a solution of ethyl 2-(2,5-dimethoxybenzyl)butanoate (5.98 g, 22.45 mmol, 1 eq.) in H₂O (20 mL), THF (20 mL), and EtOH (20 mL) was added LiOH·H₂O (2.83 g, 67.4 mmol, 3 eq.) at 0° C. Then the mixture was stirred at 50° C. for 10 h. Upon completion, the mixture was adjusted pH to 3 with 1M aq. HCl (10 mL). The residue was washed with water (50 mL×2). The aqueous phase was extracted with ethyl acetate (50 mL×2). The combined organic phase was washed with brine (60 mL×1), dried with anhydrous Na₂SO₄, filtered, and concentrated in vacuo to afford 2-(2,5-dimethoxybenzyl)butanoic acid (4.5 g, 18.9 mmol, 84% yield) as a yellow solid. 1H NMR (400 MHz, CHLOROFORM-d) δ 6.80-6.69 (m, 3H), 3.77 (s, 3H), 3.75 (s, 3H), 2.94-2.86 (m, 1H), 2.85-2.77 (m, 1H), 2.75-2.65 (m, 1H), 1.73-1.52 (m, 2H), 0.96 (t, J=7.2 Hz, 3H).

### Step 4: Preparation of benzyl (1-(2,5-dimethoxyphenyl)butan-2-Yl)carbamate

To a solution of 2-(2,5-dimethoxybenzyl)butanoic acid (2.5 g, 10.49 mmol, 1 eq.) in toluene (10 mL) was added DPPA (2.89 g, 10.49 mmol, 2.27 mL, 1 eq.) and TEA (3.19 g, 31.48 mmol, 4.38 mL, 3 eq.). The mixture was stirred at 15° C. for 1 h. Then phenylmethanol (3.40 g, 31.48 mmol, 3.27 mL, 3 eq.) was added. Then the mixture was stirred at 80° C. for 10 h. Upon completion, the mixture was poured into H₂O (50 mL). The layers were separated, and the aqueous phase was extracted with EtOAc (50 mL×3). The combined organic layer was washed with saturated aqueous NaCl solution (50 mL), dried over Na₂SO₄, filtered, and concentrated in vacuo. The residue was purified by column chromatography (SiO₂, Petroleum ether/Ethyl acetate=50/1-0/1) to afford benzyl (1-(2,5-dimethoxyphenyl)butan-2-yl)carbamate (1.8 g, crude) as a white solid. ¹H NMR (400 MHz, CHLOROFORM-d) δ 7.40-7.28 (m, 5H), 6.82-6.64 (m, 3H), 5.04 (s, 2H), 3.87-3.79 (m, 1H), 3.78-3.72 (m, 7H), 2.76 (br d, J=6.8 Hz, 2H), 1.68-1.52 (m, 2H), 0.96 (t, J=7.2 Hz, 3H).

### Step 5: Preparation of benzyl (1-(4-bromo-2,5-dimethoxyphenyl)butan-2-yl)carbamate (Intermediate 3)

To a solution of benzyl (1-(2,5-dimethoxyphenyl)butan-2-yl)carbamate (1.7 g, 4.95 mmol, 1 eq.) in MeCN (20 mL) was added NBS (1.76 g, 9.90 mmol, 2 eq.). The mixture was stirred at 25° C. for 10 h. Upon completion, the residue was added to water (50 mL). The aqueous phase was extracted with ethyl acetate (50 mL×3). The combined organic phase was washed with brine (50 mL×1), dried with anhydrous Na₂SO₄, filtered, and concentrated in vacuo. The residue was purified by column chromatography (SiO₂, Petroleum ether/Ethyl acetate=50/1-0/1) to afford benzyl (1-(4-bromo-2,5-dimethoxyphenyl)butan-2-yl)carbamate (3 g, crude) as a white solid. ¹H NMR (400 MHz, CHLOROFORM-d) δ 7.41-7.28 (m, 5H), 7.03 (s, 1H), 6.74 (s, 1H), 5.11-4.97 (m, 2H), 4.79 (br d, J=8.4 Hz, 1H), 3.78 (br d, J=12.8 Hz, 7H), 2.98 (s, 3H), 2.78 (s, 2H), 1.66-1.41 (m, 3H), 0.97 (t, J=7.2 Hz, 3H).

### EXAMPLES

### Example 1: Preparation of 1-(4-(4-fluorobutyl)-2,5-dimethoxyphenyl)propan-2-amine (2)

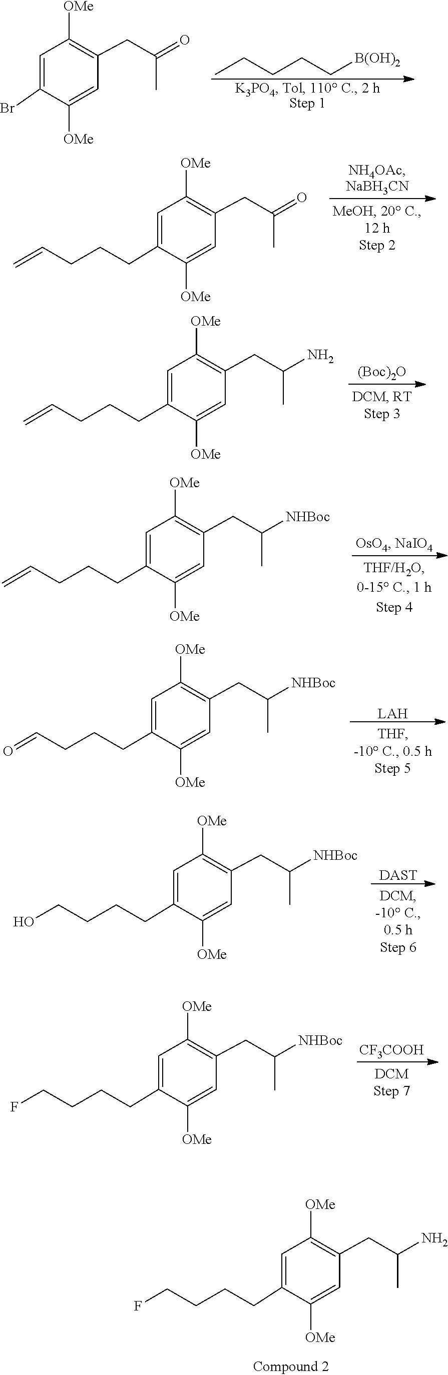

### Step 1: Preparation of 1-(2,5-dimethoxy-4-(pent-4-en-1-yl)phenyl)propan-2-one

A mixture of 1-(4-bromo-2,5-dimethoxyphenyl)propan-2-one (4 g, 14.65 mmol, 1 eq.), pent-4-enylboronic acid (2.00 g, 17.57 mmol, 1.2 eq.), Pd(dppf)Cl₂ (536 mg, 732.3 μmol, 0.05 eq.), and K₃PO₄ (9.33 g, 43.94 mmol, 3 eq.) in toluene (60 mL) was stirred and warmed to 110° C. for 12 h. Upon completion, the mixture was cooled, filtered, and concentrated. The residue was purified by silica gel chromatography (Petroleum ether:Ethyl acetate=40:1-20:1) to afford 1-(2,5-dimethoxy-4-(pent-4-en-1-yl)phenyl)propan-2-one (1.9 g, 7.24 mmol, 50% yield) as a yellow oil.

### Step 2: Preparation of 1-(2,5-dimethoxy-4-(pent-4-en-1-yl)phenyl)propan-2-amine

A mixture of 1-(2,5-dimethoxy-4-(pent-4-en-1-yl)phenyl)propan-2-one (1.5 g, 5.72 mmol, 1 eq.), NH₄OAc (2.20 g, 28.59 mmol, 5 eq.), and NaBH₃CN (719 mg, 11.44 mmol, 2 eq.) in MeOH (20 mL) was stirred at 15° C. for 12 h. Upon completion, the solvent was removed. The residue was basified to pH=8 with sat. aq. NaHCO₃ soln. and extracted with DCM (20 mL×2). The organic layer was washed with brine, dried over Na₂SO₄, filtered, and concentrated to afford 1-(2,5-dimethoxy-4-(pent-4-en-1-yl)phenyl)propan-2-amine (1.9 g, crude) as a yellow oil. LCMS R_{T}=2.257 min, MS cal.: 263.19, [M+H]⁺=264.2).

### Step 3: Preparation of tert-butyl (1-(2,5-dimethoxy-4-(pent-4-en-1-yl)phenyl)propan-2-yl)carbamate

A solution of 1-(2,5-dimethoxy-4-(pent-4-en-1-yl)propan-2-amine (1.3 g, 4.94 mmol, 1 eq.), Boc₂O (1.29 g, 5.92 mmol, 1.36 mL, 1.2 eq.), and TEA (999 mg, 9.87 mmol, 1.37 mL, 2 eq.) in DCM (15 mL) was stirred at 20° C. for 2 h. Upon completion, the solvent was removed. The residue was purified by silica gel chromatography (Petroleum ether/Ethyl acetate=100:1) to afford tert-butyl (1-(2,5-dimethoxy-4-(pent-4-en-1-yl)phenyl)propan-2-yl)carbamate (1 g, 2.75 mmol, 56% yield) as a yellow solid.

### Step 4: Preparation of tert-butyl (1-(2,5-dimethoxy-4-(4-oxobutyl)phenyl)propan-2-yl)carbamate

A mixture of tert-butyl (1-(2,5-dimethoxy-4-(pent-4-en-1-yl)phenyl)propan-2-yl)carbamate (1.5 g, 4.13 mmol, 1 eq.) and NaIO₄ (4.41 g, 20.63 mmol, 1.14 mL, 5 eq.) in THF (30 mL) and H₂O (10 mL) was cooled to 0° C. Then OsO₄ (419.65 mg, 1.65 mmol, 85.64 ul, 0.4 eq.) was added. The mixture was stirred at 20° C. for 1 h. Upon completion, the mixture was poured into EA (30 mL), washed with sat. aq. Na₂SO₃ soln. (50 mL) and brine, dried over Na₂SO₄, filtered, and concentrated to afford tert-butyl (1-(2,5-dimethoxy-4-(4-oxobutyl)phenyl)propan-2-yl)carbamate (1.4 g, crude) as a yellow oil. LCMS R_{T}=1.136 min, MS cal.: 365.22, [M+H−100]⁺=266.3).

### Step 5: Preparation of tert-butyl (1-(4-(4-hydroxybutyl)-2,5-dimethoxyphenyl)propan-2-yl)carbamate

A solution of tert-butyl (1-(2,5-dimethoxy-4-(4-oxobutyl)phenyl)propan-2-yl)carbamate (2.7 g, 7.39 mmol, 1 eq.) in THF (20 mL) was cooled to −10° C. Then LiAlH₄ (561 mg, 14.78 mmol, 2 eq.) was added. The mixture was stirred at −10° C. for 0.5 h. Upon completion, the mixture was quenched with H₂O (0.3 mL) and 30% aq. NaOH (0.3 mL). The mixture was stirred to a smooth dispersion and then filtered and concentrated. The residue was purified by silica gel chromatography (Petroleum ether/Ethyl acetate=10:1-3:1) to afford tert-butyl (1-(4-(4-hydroxybutyl)-2,5-dimethoxyphenyl)propan-2-yl)carbamate (1.2 g, 2.35 mmol, 32% yield, 72% purity) as a yellow solid. LCMS R_{T}=1.071 min, MS cal.: 367.24, [M+H−100]⁺=268.3).

### Step 6: Preparation of tert-butyl (1-(4-(4-fluorobutyl)-2,5-dimethoxyphenyl)propan-2-yl)carbamate

A solution of tert-butyl (1-(4-(4-hydroxybutyl)-2,5-dimethoxyphenyl)propan-2-yl)carbamate (1.2 g, 3.27 mmol, 1 eq.) in DCM (15 mL) was cooled to 0° C. Then DAST (1.05 g, 6.53 mmol, 2 eq.) was added. The mixture was stirred at 0° C. for 0.5 h. Upon completion, the mixture was poured into sat. aq. NaHCO₃ soln. and extracted with DCM (10 mL×2). The organic layer was washed with brine, dried over Na₂SO₄, filtered, and concentrated. The residue was purified by prep-HPLC (column: Phenomenex luna C18 250×50 mm×10 μm; mobile phase: [water (0.04% HCl)-ACN]; B %: 60%-80%, 10 min) to afford tert-butyl (1-(4-(4-fluorobutyl)-2,5-dimethoxyphenyl)propan-2-yl)carbamate (100 mg, 270.66 μmol, 8% yield) as a white solid. ¹H NMR (400 MHz, CHLOROFORM-d6) δ ppm 6.68-6.63 (m, 2H), 4.81-4.69 (m, 1H), 4.56-4.51 (m, 1H), 4.42 (t, J=5.6 Hz, 1H), 3.89 (br s, 1H), 3.81-3.76 (m, 6H), 2.79-2.67 (m, 2H), 2.63 (t, J=7.2 Hz, 2H), 1.82-1.65 (m, 4H), 1.43-1.35 (m, 9H), 1.17-1.11 (m, 3H).

### Step 7: Preparation of 1-(4-(4-fluorobutyl)-2,5-dimethoxyphenyl)propan-2-amine (2)

A solution of tert-butyl (1-(4-(4-fluorobutyl)-2,5-dimethoxyphenyl)propan-2-yl)carbamate (160 mg, 433 μmol, 1 eq.) in DCM (1 mL) and TFA (1 mL) was stirred at 20° C. for 1 h. Upon completion, the solvent was removed. The residue was purified by prep-HPLC (column: Phenomenex luna C18 80×40 mm×3 um; mobile phase: [water (0.04% HCl)-ACN]; B %: 17%-43%, 7 min) to afford 1-(4-(4-fluorobutyl)-2,5-dimethoxyphenyl)propan-2-amine (100 mg, 371.26 μmol, 86% yield, 100% purity, HCl salt) as a white solid. LCMS R_{T}=1.913 min, MS cal.: 269.18, [M+H]⁺=270.1; 1H NMR (400 MHz, CHLOROFORM-d6, HCl salt) δ ppm 8.17 (br s, 3H), 6.68 (d, J=4.0 Hz, 2H), 4.53 (t, J=6.0 Hz, 1H), 4.46-4.35 (m, 1H), 3.79 (s, 3H), 3.77 (s, 3H), 3.67 (s, 1H), 3.06 (dd, J=6.4, 13.2 Hz, 1H), 2.87 (dd, J=7.6, 13.2 Hz, 1H), 2.62 (t, J=7.2 Hz, 2H), 1.82-1.63 (m, 4H), 1.36 (d, J=6.4 Hz, 3H); ¹³C NMR (101 MHz, DMSO-d₆, HCl salt) δ ppm 151.01, 150.74, 129.25, 122.50, 114.00, 113.09, 84.51, 82.91, 55.85, 46.94, 34.81, 29.76, 29.57, 29.18, 25.26, 25.21, 17.87.

### Example 2: Preparation of 1-(4-(butylthio)-2,5-dimethoxyphenyl)propan-2-amine (3)

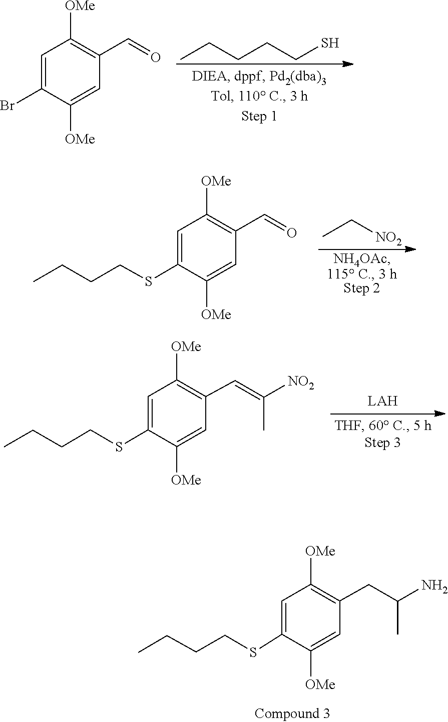

### Step 1: Preparation of 4-(butylthio)-2,5-dimethoxybenzaldehyde

To a solution of 4-bromo-2,5-dimethoxybenzaldehyde (3 g, 12.24 mmol, 1 eq.) and butane-1-thiol (1.44 g, 15.91 mmol, 1.70 mL, 1.3 eq.) in toluene (30 mL) was added DIEA (4.75 g, 36.72 mmol, 6.40 mL, 3 eq.), DPPF (679 mg, 1.22 mmol, 0.1 eq.), and Pd₂(dba)₃ (1.12 g, 1.22 mmol, 0.1 eq.) under N₂. The mixture was stirred and warmed to 110° C. for 3 h. Upon completion, the mixture was filtered and concentrated. The residue was purified by silica gel chromatography (PE:EA=100:1-30:1) to afford 4-(butylthio)-2,5-dimethoxybenzaldehyde (3 g, 11.80 mmol, 96% yield) as a yellow solid. 1H NMR (400 MHz, CHLOROFORM-d) δ ppm 10.38-10.34 (m, 1H), 7.25 (s, 1H), 6.77 (s, 1H), 3.92 (s, 3H), 3.89 (s, 3H), 2.96 (t, J=7.2 Hz, 2H), 1.78-1.71 (m, 2H), 1.56-1.50 (m, 2H), 0.98 (t, J=7.2 Hz, 3H).

### Step 2: Preparation of (E)-butyl(2,5-dimethoxy-4-(2-nitroprop-1-en-1-yl)phenyl)sulfane

To a solution of 4-bromo-2,5-dimethoxybenzaldehyde (3 g, 11.80 mmol, 1 eq.) in nitroethane (17.71 g, 235.9 mmol, 16.86 mL, 20 eq.) was added NH₄OAc (2.73 g, 35.39 mmol, 3 eq.). The mixture was stirred and warmed to 110° C. for 3 h. Upon completion, the solvent was removed. The residue was purified by silica gel chromatography (PE:EA=100:1-30:1) to afford (E)-butyl(2,5-dimethoxy-4-(2-nitroprop-1-en-1-yl)phenyl)sulfane (2.6 g, 8.35 mmol, 71% yield) as a yellow oil. 1H NMR (400 MHz, CHLOROFORM-d) δ ppm 8.28 (s, 1H), 6.81 (s, 1H), 6.79 (s, 1H), 3.87 (d, J=1.2 Hz, 6H), 2.98-2.94 (m, 2H), 2.43 (d, J=1.2 Hz, 3H), 1.74-1.69 (m, 2H), 1.55-1.49 (m, 2H), 0.97 (t, J=7.2 Hz, 3H).

### Step 3: Preparation of 1-(4-(butylthio)-2,5-dimethoxyphenyl)propan-2-amine (3)

To a solution of (E)-butyl(2,5-dimethoxy-4-(2-nitroprop-1-en-1-yl)phenyl)sulfane (2.6 g, 8.35 mmol, 1 eq.) in THF (40 mL) was added LiAlH₄ (1.27 g, 33.40 mmol, 4 eq.) in one portion at 0° C. under N₂. The mixture was stirred at 20° C. for 30 min, then heated to 60° C. and stirred for 4.5 h. Upon completion, the reaction mixture was quenched by dropwise addition of H₂O (1.5 mL) and 30% aq. NaOH soln. (1.5 mL) at 0° C., and then the solids were filtered, and the filtrate concentrated to give a residue. The residue was purified by prep-HPLC (column: Phenomenex luna C18 250×50 mm×15 μm; mobile phase: [water (0.05% HCl)-ACN]; B %: 15%-45%, 20 min) to afford 1-(4-(butylthio)-2,5-dimethoxyphenyl)propan-2-amine (380 mg, 1.19 mmol, 14% yield, 100% purity, HCl salt) as a white solid. LCMS R_{T}=2.069 min, MS cal.: 283.16, [M+H]⁺=284.1; 1H NMR (400 MHz, DMSO-d6, HCl salt) δ ppm 8.26 (br s, 3H), 6.86 (s, 1H), 6.80 (s, 1H), 3.75 (d, J=4.4 Hz, 6H), 3.41-3.34 (m, 1H), 2.96-2.87 (m, 3H), 2.71 (dd, J=8.4, 13.3 Hz, 1H), 1.54 (m, 2H), 1.40 (m, 2H), 1.12 (d, J=6.4 Hz, 3H), 0.88 (t, J=7.2 Hz, 3H); ¹³C NMR (101 MHz, DMSO-d6, HCl salt) δ ppm 151.52, 150.28, 123.82, 122.28, 114.29, 111.12, 56.21, 56.02, 46.82, 34.55, 30.46, 30.27, 21.37, 17.80, 13.49.

### Example 3: Preparation of 1-(4-hexyl-2,5-dimethoxyphenyl)propan-2-amine (4) and its Enantiomers (4ent1 and 4ent2)

### Preparation of Racemate (4)

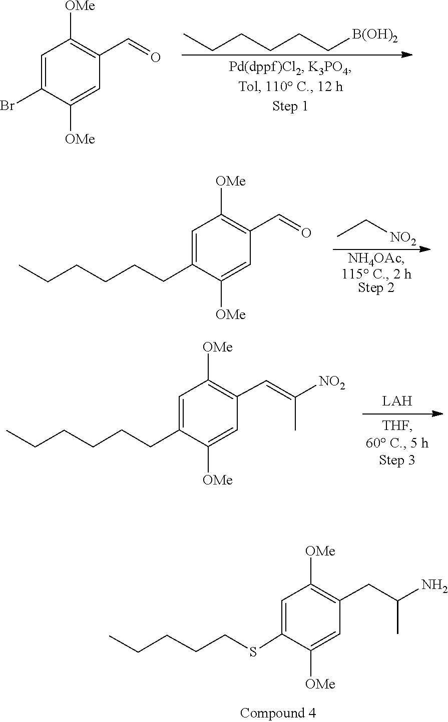

### Step 1: Preparation of 4-hexyl-2,5-dimethoxybenzaldehyde

A mixture of 4-bromo-2,5-dimethoxybenzaldehyde (3 g, 12.24 mmol, 1 eq.), hexylboronic acid (1.59 g, 12.24 mmol, 1 eq.), Pd(dppf)Cl₂ (447 mg, 612 μmol, 0.05 eq.), and K₃PO₄ (5.20 g, 24.48 mmol, 3 eq.) in toluene (30 mL) was stirred at 110° C. for 12 h. Upon completion, the mixture was filtered and concentrated. The residue was purified by silica gel chromatography (PE:EA=100:1-50:1) to afford 4-hexyl-2,5-dimethoxybenzaldehyde (2.6 g, 10.39 mmol, 85% yield) as a yellow oil.

### Step 2: Preparation of (E)-1-hexyl-2,5-dimethoxy-4-(2-nitroprop-1-en-1-yl) benzene

A mixture of 4-hexyl-2,5-dimethoxybenzaldehyde (2.6 g, 10.39 mmol, 1 eq.) and NH₄OAc (1.60 g, 20.78 mmol, 2 eq.) in nitroethane (20 mL) was stirred and warmed to 115° C. for 1 h. Upon completion, the solvent was removed. The residue was purified by silica gel chromatography (PE:EA=80:1-5:1) to afford (E)-1-hexyl-2,5-dimethoxy-4-(2-nitroprop-1-en-1-yl)benzene (2.4 g, 7.81 mmol, 75% yield) as a yellow oil.

### Step 3: Preparation of 1-(4-hexyl-2,5-dimethoxyphenyl)propan-2-amine (4)

A solution of (E)-1-hexyl-2,5-dimethoxy-4-(2-nitroprop-1-en-1-yl)benzene (2.4 g, 7.81 mmol, 1 eq.) in THF (40 mL) was cooled to 0° C. Then LiAlH₄ (1.19 g, 31.23 mmol, 4 eq.) was added. The mixture was warmed to 60° C. and stirred at 60° C. for 5 h. Upon completion, the mixture was cooled to 0° C. Then 2 mL H₂O was added. Then 2 mL 30% aq. NaOH was added. The mixture was stirred to obtain a smooth dispersion and then filtered and concentrated. The residue was purified by prep-HPLC (column: Phenomenex luna C18 250×70 mm×15 μm; mobile phase: [water (0.05% HCl)-ACN]; B %: 25%-55%, 20 min) to afford 1-(4-hexyl-2,5-dimethoxyphenyl)propan-2-amine (450 mg, 1.42 mmol, 18% yield, HCl salt) as a white solid. LCMS R_{T}=2.310 min, MS cal.: 279.42, [M+H]⁺=280.2; 1H NMR (400 MHz, DMSO-d6, HCl salt) δ ppm 8.12 (br s, 3H), 6.79 (s, 2H), 3.73 (s, 6H), 3.50-3.35 (m, 1H), 2.91 (dd, J=5.6, 13.2 Hz, 1H), 2.69 (dd, J=8.4, 13.2 Hz, 1H), 2.54 (s, 2H), 1.62-1.43 (m, 2H), 1.28 (s, 6H), 1.12 (d, J=6.4 Hz, 3H), 0.93-0.80 (m, 3H); ¹³C NMR (101 MHz, DMSO-d6, HCl salt) δ ppm 150.96, 150.71, 129.76, 122.29, 113.97, 113.01, 55.85, 55.84, 46.90, 34.75, 31.08, 29.62, 29.53, 28.60, 22.04, 17.80, 13.92.

### Preparation of Enantiomers (4ent1 and 4ent2)

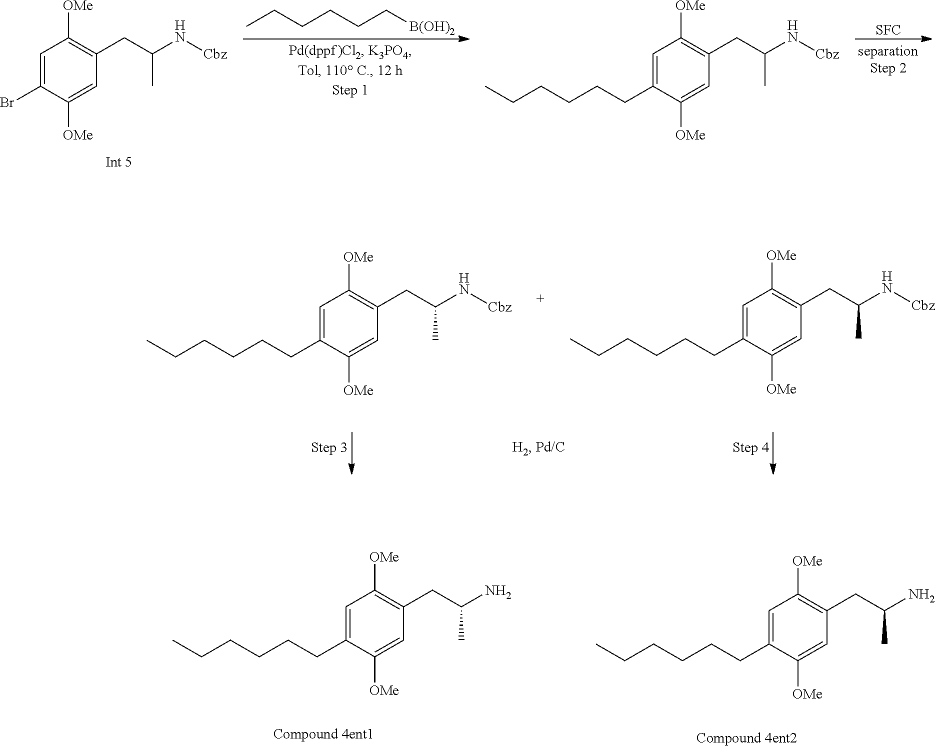

### Step 1: Preparation of benzyl (1-(4-hexyl-2,5-dimethoxyphenyl)propan-2-yl)carbamate

To a solution of benzyl (1-(4-bromo-2,5-dimethoxyphenyl)propan-2-yl)carbamate (0.3 g, 735 μmol, 1 eq.) in toluene (3 mL) was added hexylboronic acid (143 mg, 1.10 mmol, 1.5 eq.), K₃PO₄ (468 mg, 2.20 mmol, 3 eq.) and Pd(dppf)Cl₂ (53.8 mg, 73.5 μmol, 0.1 eq.) under N₂. The mixture was stirred and warmed to 120° C. for 12 h. Upon completion, the reaction was cooled, and brine (30 mL) and DCM (30 mL) were added, the mixture was filtered, and the filtrate was extracted with DCM (30 mL). The organic phase was dried over Na₂SO₄, filtered, and concentrated to give a residue. The residue was purified by column chromatography (SiO₂, PE/EA=5/1) to give benzyl (1-(4-hexyl-2,5-dimethoxyphenyl)propan-2-yl)carbamate (0.2 g, 459 μmol, 63% yield, 95% purity) as a white solid. ¹H NMR (400 MHz, DMSO-d₆) δ ppm 7.42-7.22 (m, 5H), 7.15 (d, J=8.4 Hz, 1H), 6.73 (s, 2H), 4.96 (d, J=5.2 Hz, 2H), 3.85-3.75 (m, 1H), 3.74-3.59 (m, 6H), 2.74-2.58 (m, 2H), 1.49 (d, J=7.6 Hz, 2H), 1.27 (s, 6H), 1.04 (d, J=6.4 Hz, 2H), 1.01-0.97 (m, 1H), 0.90-0.82 (m, 3H).

### Step 2: Separation of benzyl (1-(4-hexyl-2,5-dimethoxyphenyl)propan-2-yl)carbamate enantiomers

Racemic benzyl (1-(4-hexyl-2,5-dimethoxyphenyl)propan-2-yl)carbamate (0.2 g, 483.62 μmol, 1 eq.) was separated by prep-SFC (column: DAICEL CHIRALPAK IG (250 mm×30 mm, 10 μm); mobile phase: A: CO₂, B: 0.1% NH₃H₂O in MeOH; B %=20%, 10 min) to give the first eluting isomer (Cbz-ent1, RT=0.997 min) of benzyl (1-(4-hexyl-2,5-dimethoxyphenyl)propan-2-yl)carbamate (90 mg, 217.6 μmol, 45% yield) as a white solid and the later eluting isomer (Cbz-ent2, RT=1.061 min) of benzyl (1-(4-hexyl-2,5-dimethoxyphenyl)propan-2-yl)carbamate (80 mg, 193.5 μmol, 40% yield) as a white solid. Retention times were determined using the following chiral analytical method: column: Chiralpak IG-3, 50×4.6 mm I.D., 3 μm; mobile phase: A: CO₂, B: MeOH (0.05% IPAm, v/v); gradient: (Time (min)/A %/B %), (0.0/95/5, 0.2/95/5, 1.2/50/50, 2.2/50/50, 2.6/95/5, 3.0/95/5); flow rate: 3.4 mL/min; column temp.: 35° C.; ABPR: 1800 psi. Cbz-ent1, RT=0.997 min (assigned here as the R isomer), ¹H NMR (400 MHz, CHLOROFORM-d) δ ppm 7.38-7.29 (m, 5H), 6.67-6.61 (m, 1H), 5.05 (s, 2H), 3.95 (s, 1H), 3.84-3.68 (m, 6H), 2.83-2.65 (m, 2H), 2.61-2.49 (m, 2H), 1.62-1.56 (m, 2H), 1.39-1.29 (m, 6H), 1.18 (d, J=6.4 Hz, 3H), 0.95-0.86 (m, 3H); Cbz-ent2, RT=1.061 min (assigned here as the S isomer), ¹H NMR (400 MHz, CHLOROFORM-d) δ ppm 7.40-7.28 (m, 5H), 6.67-6.62 (m, 1H), 5.10-5.03 (m, 2H), 4.01-3.89 (m, 1H), 4.01-3.89 (m, 1H), 3.80-3.72 (m, 6H), 2.85-2.65 (m, 2H), 2.61-2.53 (m, 2H), 1.62-1.55 (m, 2H), 1.38-1.28 (m, 6H), 1.18 (d, J=6.4 Hz, 3H), 0.93-0.87 (m, 3H).

### Step 3: Preparation of 1-(4-hexyl-2,5-dimethoxyphenyl)propan-2-amine, enantiomer 1 (4ent1)

To a solution of the early-eluting isomer of benzyl (1-(4-hexyl-2,5-dimethoxyphenyl)propan-2-yl)carbamate (Cbz-ent1, 50 mg, 120.9 μmol, 1 eq.) in MeOH (10 mL) and NH₃·H₂O (1 mL) was added Pd(OH)₂ (34 mg, 241.8 μmol, 2 eq.) under H₂ (15 Psi). The mixture was stirred at 20° C. for 2 h. Upon completion, the reaction was filtered, the filter cake was washed with MeOH (50 mL), and the filtrate was concentrated. The residue was purified by prep-HPLC (column: Waters Xbridge BEH C18 100×30 mm×10 μm; mobile phase: [water (10 mM NH₄HCO₃)-ACN]; B %: 35%-70%, 8 min) to give 1-(4-hexyl-2,5-dimethoxyphenyl)propan-2-amine enantiomer 1 (4ent1, assigned here as the R isomer, 25 mg, 89.47 μmol, 74% yield, 100% purity) as a white solid. Chiral HPLC retention times were determined using the following chiral analytical method: column: Chiralpak IG-3, 100×4.6 mm I.D., 3 μm; mobile phase: A: hexanes, B: iPrOH (0.05% IPAm, v/v); B %=5%; flow rate: 0.5 mL/min; column temp.: 30° C. Chiral HPLC RT=6.055 min; LCMS (ESI+): m/z 280.2, [M+H]⁺; ¹H NMR (400 MHz, DMSO-d6) δ ppm 6.74-6.66 (m, 2H), 3.69 (d, J=3.2 Hz, 6H), 3.05-2.92 (m, 1H), 2.49-2.41 (m, 4H), 1.48 (d, J=7.6 Hz, 2H), 1.27 (d, J=3.2 Hz, 6H), 0.92 (d, J=6.4 Hz, 3H), 0.88-0.81 (m, 3H).

### Step 4: Preparation of 1-(4-hexyl-2,5-dimethoxyphenyl)propan-2-amine, enantiomer 2 (4ent2)

To a solution of the late-eluting isomer of benzyl (1-(4-hexyl-2,5-dimethoxyphenyl)propan-2-yl)carbamate (Cbz-ent2, 50 mg, 120.9 μmol, 1 eq.) in MeOH (10 mL) and NH₃·H₂O (1 mL) was added Pd(OH)₂ (34 mg, 241.8 μmol, 2 eq.) under H₂ (15 Psi), and then the mixture was stirred at 20° C. for 2 h. Upon completion, the reaction was filtered, the filter cake was washed with MeOH (50 mL), and the filtrate was concentrated. The residue was purified by prep-HPLC (column: Waters Xbridge BEH C18 100×30 mm×10 um; mobile phase: [water (10 mM NH₄HCO₃)-ACN]; B %: 35%-80%, 8 min) to give 1-(4-hexyl-2,5-dimethoxyphenyl)propan-2-amine enantiomer 2 (4ent2, assigned here as the S isomer, 10 mg, 35.8 μmol, 30% yield, 100% purity) as a white solid. Chiral HPLC retention times were determined using the following chiral analytical method: column: Chiralpak IG-3, 100×4.6 mm I.D., 3 μm; mobile phase: A: hexanes, B: iPrOH (0.05% IPAm, v/v); B %=5%; flow rate: 0.5 mL/min; column temp.: 30° C. Chiral HPLC RT=6.498 min; LCMS(ESI+): m/z 280.2, [M+H]⁺; 1H NMR (400 MHz, DMSO-d6) δ ppm 6.71 (d, J=9.2 Hz, 2H), 3.70 (d, J=3.2 Hz, 6H), 2.99 (m, 1H), 2.47 (s, 4H), 1.49 (d, J=6.8 Hz, 2H), 1.28 (d, J=3.2 Hz, 6H), 0.93 (d, J=6.4 Hz, 3H), 0.89-0.82 (m, 3H).

### Example 4: Preparation of 1-(4-heptyl-2,5-dimethoxyphenyl)propan-2-amine (5)

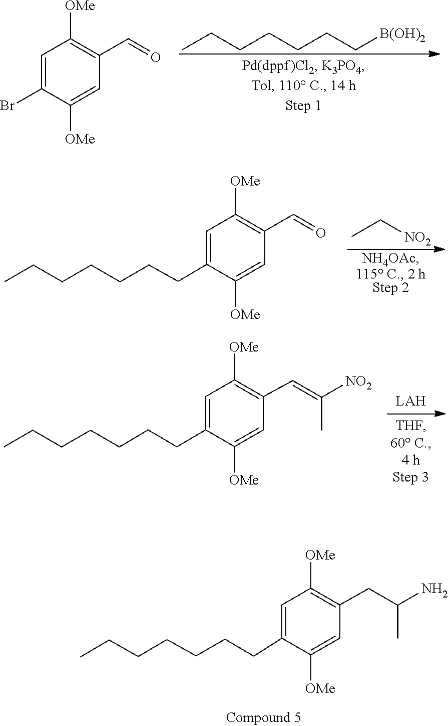

### Step 1: Preparation of 4-heptyl-2,5-dimethoxybenzaldehyde

To a solution of 4-bromo-2,5-dimethoxybenzaldehyde (1.2 g, 4.90 mmol, 1.0 eq.) in toluene (20 mL) was added heptylboronic acid (846.2 mg, 5.88 mmol, 1.2 eq.), K₃PO₄ (3.12 g, 14.69 mmol, 3 eq.), and Pd(dppf)Cl₂ (179.14 mg, 244.83 μmol, 0.05 eq.) under N₂. The mixture was stirred at 110° C. for 14 h. Upon completion, the reaction mixture was filtered and concentrated. The crude product was purified by silica gel chromatography (Petroleum ether/Ethyl acetate=50/1) to give 4-heptyl-2,5-dimethoxybenzaldehyde (1.2 g, 4.54 mmol, 93% yield) as a yellow solid. ¹H NMR (400 MHz, CHLOROFORM-d) δ ppm 10.41 (s, 1H), 7.27-7.29 (m, 1H), 6.80 (s, 1H), 3.90 (s, 3H), 3.83 (s, 3H), 2.60-2.68 (m, 2H), 1.54-1.63 (m, 2H), 1.25-1.38 (m, 8H), 0.89 (t, J=6.8 Hz, 3H).

### Step 2: Preparation of (E)-1-heptyl-2,5-dimethoxy-4-(2-nitroprop-1-en-1-yl)benzene

To a mixture of 4-heptyl-2,5-dimethoxybenzaldehyde (1.2 g, 4.54 mmol, 1.0 eq.) and NH₄OAc (700 mg, 9.08 mmol, 2.0 eq.) was added nitroethane (6.81 g, 90.79 mmol, 6.49 mL, 20.0 eq.) in one portion at 20° C. under N₂. The mixture was stirred at 115° C. for 2 h. Upon completion, the solvent was removed. The crude product was purified by silica gel chromatography (Petroleum ether/Ethyl acetate=50/1) to give (E)-1-heptyl-2,5-dimethoxy-4-(2-nitroprop-1-en-1-yl)benzene (1.05 g, 3.27 mmol, 72% yield) as a yellow oil. ¹H NMR (400 MHz, CHLOROFORM-d) δ ppm 8.30 (s, 1H), 6.77 (d, J=7.6 Hz, 2H), 3.85 (s, 3H), 3.80 (s, 3H), 2.60-2.67 (m, 2H), 2.43 (s, 3H), 1.57-1.63 (m, 2H), 1.27-1.38 (m, 9H), 0.90 (t, J=6.8 Hz, 3H).

### Step 3: Preparation of 1-(4-heptyl-2,5-dimethoxyphenyl)propan-2-amine (5)

To a mixture of (E)-1-heptyl-2,5-dimethoxy-4-(2-nitroprop-1-en-1-yl)benzene (1.05 g, 3.27 mmol, 1 eq.) in THF (20 mL) was added LiAlH₄ (495.91 mg, 13.07 mmol, 4 eq.) in one portion at 0° C. under N₂. The mixture was stirred at 20° C. for 30 min, then heated to 60° C. and stirred for 3.5 h. Upon completion, the mixture was cooled to 0° C. The reaction mixture was quenched by sequential dropwise addition of H₂O (0.5 mL) and 30% aq. NaOH (0.5 mL) at 0° C., filtered, and concentrated to give a residue. The residue was purified by prep-HPLC (column: Phenomenex luna C18 250×50 mm×10 μm; mobile phase: [water (0.04% HCl)-ACN]; B %: 20%-50%, 10 min) to afford 1-(4-heptyl-2,5-dimethoxyphenyl)propan-2-amine (380 mg, 1.15 mmol, 35% yield, 100% purity, HCl salt) as a white solid. LCMS R_{T}=2.420 min, MS cal.: 293.24, [M+H]⁺=294.2; 1H NMR (400 MHz, DMSO-d6, HCl salt) δ ppm 8.15 (br s, 3H), 6.78 (s, 2H), 3.72 (d, J=1.2 Hz, 6H), 3.41-3.36 (m, 1H), 2.91 (dd, J=5.6, 13.2 Hz, 1H), 2.68 (dd, J=8.8, 13.2 Hz, 1H), 2.53-2.50 (m, 2H), 1.54-1.46 (m, 2H), 1.31-1.22 (m, 8H), 1.11 (d, J=6.4 Hz, 3H), 0.88-0.83 (m, 3H); ¹³C NMR (101 MHz, DMSO-d6, HCl salt) δ ppm 150.96, 150.70, 129.75, 122.31, 113.97, 112.99, 55.84, 55.82, 46.91, 34.75, 31.24, 29.62, 29.58, 28.91, 28.51, 22.05, 17.79, 13.92.

### Example 5: Preparation of 2-(2,5-dimethoxy-4-pentylphenyl)ethanamine (6)

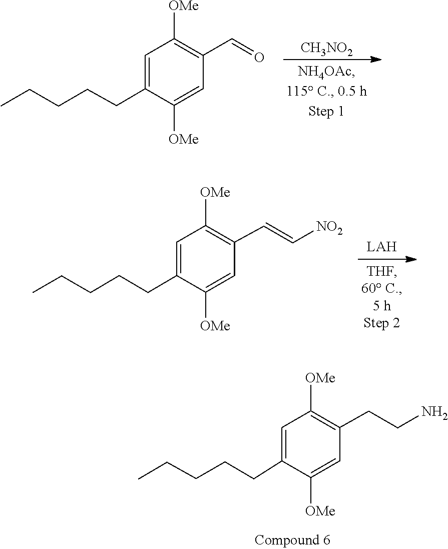

### Step 1: Preparation of 1,4-dimethoxy-2-[(E)-2-nitrovinyl]-5-pentylbenzene

A solution of 2,5-dimethoxy-4-pentyl-benzaldehyde (3 g, 12.70 mmol, 1 eq.) and NH₄OAc (1.96 g, 25.40 mmol, 2 eq.) in nitromethane (13.95 g, 228.60 mmol, 12.35 mL, 18 eq.) was warmed and stirred at 115° C. for 0.5 h. Upon completion, the solvent was removed. The residue was purified by silica gel chromatography (PE:EA=80:1-60:1) to afford 1,4-dimethoxy-2-[(E)-2-nitrovinyl]-5-pentylbenzene (2.89 g, 10.35 mmol, 82% yield) as a yellow oil. ¹H NMR (400 MHz, CHLOROFORM-d) δ ppm 8.145 (d, J=13.6 Hz, 1H), 7.866 (d, J=13.6 Hz, 1H), 6.856 (s, 1H), 6.778 (s, 1H), 3.917 (s, 3H), 3.824 (s, 3H), 2.601-2.672 (m, 2H), 1.534-1.638 (m, 2H), 1.298-1.405 (m, 4H), 0.860-0.966 (m, 3H).

### Step 2: Preparation of 2-(2,5-dimethoxy-4-pentylphenyl)ethanamine (6)

A solution of 1,4-dimethoxy-2-[(E)-2-nitrovinyl]-5-pentylbenzene (2.89 g, 10.35 mmol, 1 eq.) in THF (40 mL) was cooled to 0° C. Then LiAlH₄ (1.57 g, 41.3 mmol, 4 eq.) was added. The mixture was stirred at 60° C. for 5 h. Upon completion, the mixture was cooled to 0° C. Then H₂O (1.6 mL) was added dropwise with stirring followed by 30% aq. NaOH (1.6 mL). The mixture was stirred to a smooth dispersion then filtered and concentrated. The residue was purified by prep-HPLC (column: Phenomenex luna C18 250×50 mm×10 μm; mobile phase: [water (0.04% HCl)-ACN]; B %: 20%-50%, 10 min) to afford 2-(2,5-dimethoxy-4-pentylphenyl)ethanamine (1.5 g, 5.21 mmol, 50% yield, 100% purity, HCl) as a white solid. LCMS R_{T}=2.154 min, MS cal.: 251.19, [M+H]⁺=252.1; 1H NMR (400 MHz, DMSO-d6, HCl salt) δ ppm 8.11 (br s, 3H), 6.78 (d, J=4.4 Hz, 2H), 3.73 (d, J=2.8 Hz, 6H), 2.94 (dd, J=5.2, 8.4 Hz, 2H), 2.87-2.79 (m, 2H), 2.53-2.50 (m, 2H), 1.56-1.45 (m, 2H), 1.34-1.23 (m, 4H), 0.86 (t, J=6.8 Hz, 3H); ¹³C NMR (101 MHz, DMSO-d6, HCl salt) δ ppm 150.81, 150.77, 129.66, 122.84, 113.33, 112.99, 55.85, 38.65, 31.16, 29.58, 29.29, 27.92, 21.94, 13.90.

### Example 6: Preparation of 1-(2,5-dimethoxy-4-pentylphenyl)butan-2-amine (7)

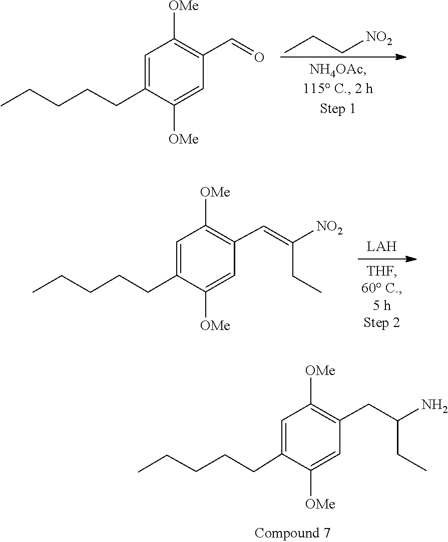

### Step1: Preparation of 1,4-dimethoxy-2-[(E)-2-nitrobut-1-enyl]-5-pentylbenzene

A mixture of 2,5-dimethoxy-4-pentyl-benzaldehyde (1 g, 4.23 mmol, 1 eq.) NH₄OAc (652.37 mg, 8.46 mmol, 2 eq.) in 1-nitropropane (6.79 g, 76.17 mmol, 6.80 mL, 18 eq.) was stirred and warmed to 115° C. for 5 h. Upon completion, the solvent was removed. The residue was purified by silica gel chromatography (PE:EA=80:1-60:1) to afford 1,4-dimethoxy-2-[(E)-2-nitrobut-1-enyl]-5-pentylbenzene (830 mg, 2.70 mmol, 64% yield) as a yellow oil. 1H NMR (400 MHz, CHLOROFORM-d) δ ppm 8.26 (s, 1H), 6.79 (s, 1H), 6.757 (s, 1H), 3.83-3.85 (m, 3H), 3.79-3.81 (m, 3H), 2.869 (d, J=7.2 Hz, 2H), 2.60-2.66 (m, 2H), 1.58-1.62 (m, 2H), 1.34-1.38 (m, 4H), 1.30 (t, J=7.2 Hz, 3H), 0.92 (t, J=6.8 Hz, 4H).

### Step 2: Preparation of 1-(2,5-dimethoxy-4-pentylphenyl)butan-2-amine (7)

A solution of 1,4-dimethoxy-2-[(E)-2-nitrobut-1-enyl]-5-pentylbenzene (830 mg, 2.70 mmol, 1 eq.) in THF (20 mL) was cooled to 0° C. Then LiAlH₄ (409.89 mg, 10.80 mmol, 4 eq.) was added. The mixture was stirred at 60° C. for 5 h. Upon completion, the mixture was cooled to 0° C. Then H₂O (0.6 mL) was added. Then 30% NaOH (0.6 mL) was added. The mixture was stirred to a smooth dispersion then filtered and concentrated. The residue was purified prep-HPLC (column: Phenomenex luna C18 80×40 mm×3 μm; mobile phase: [water (0.04% HCl)-ACN]; B %: 22%-50%, 7 min) to afford 1-(2,5-dimethoxy-4-pentylphenyl)butan-2-amine (350 mg, 1.07 mmol, 40% yield, 96.8% purity, HCl) as a white solid. LCMS R_{T}=2.320 min, MS cal.: 279.42, [M+H]⁺=280.2; 1H NMR (400 MHz, DMSO-d6, HCl salt) δ ppm 8.06-7.90 (m, 3H), 6.82 (s, 1H), 6.78 (s, 1H), 3.77-3.69 (m, 6H), 3.28-3.20 (m, 1H), 2.80 (d, J=6.4 Hz, 2H), 2.54-2.51 (m, 2H), 1.55-1.46 (m, 4H), 1.35-1.25 (m, 4H), 0.89 (td, J=7.2, 18.4 Hz, 6H); ¹³C NMR (101 MHz, DMSO-d6, HCl salt) δ ppm 151.00, 150.71, 129.77, 122.17, 114.06, 112.99, 55.84, 55.81, 52.19, 32.58, 31.16, 29.57, 29.22, 24.73, 21.91, 13.87, 9.40.

### Example 7: Preparation of 2-(3,5-dimethoxy-4-pentylphenyl)ethanamine (8)

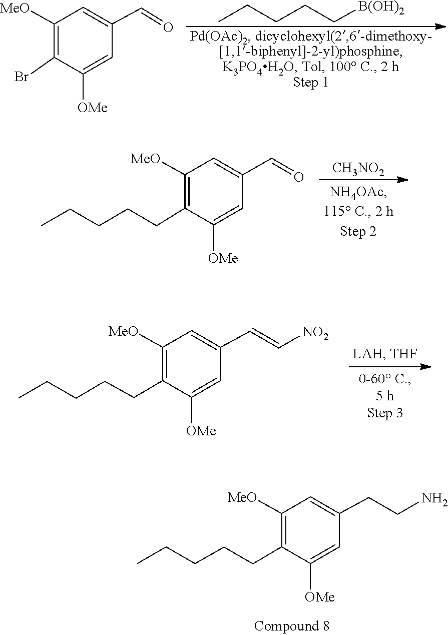

### Step 1: Preparation of 3,5-dimethoxy-4-pentylbenzaldehyde

A mixture of 4-bromo-3,5-dimethoxybenzaldehyde (6 g, 24.48 mmol, 1 eq.), K₃PO₄·H₂O (5.64 g, 24.48 mmol, 1 eq.), pentylboronic acid (4.26 g, 36.72 mmol, 1.5 eq.), dicyclohexyl(2′,6′-dimethoxy-[1,1′-biphenyl]-2-yl)phosphine (2.01 g, 4.90 mmol, 0.2 eq.) and Pd(OAc)₂ (550 mg, 2.45 mmol, 0.1 eq.) in toluene (70 mL) was stirred and warmed to 105° C. for 2 h under N₂. Upon completion, the mixture was filtered and concentrated. The residue was purified by silica gel chromatography (PE:EA=100:1-50:1) to afford 3,5-dimethoxy-4-pentylbenzaldehyde (5.6 g, 23.70 mmol, 97% yield) as a white solid. ¹H NMR (400 MHz, CHLOROFORM-d) δ ppm 9.91 (s, 1H), 6.96-7.12 (m, 2H), 3.89 (s, 6H), 2.62-2.74 (m, 2H), 1.48 (m, 2H), 1.29-1.38 (m, 4H), 0.90 (t, J=6.8 Hz, 3H).

### Step 2: Preparation of (E)-1,3-dimethoxy-5-(2-nitrovinyl)-2-pentylbenzene

A mixture of 3,5-dimethoxy-4-pentylbenzaldehyde (3 g, 12.70 mmol, 1 eq.), NH₄OAc (1.96 g, 25.39 mmol, 2 eq.) in CH₃NO₂ (20 mL), and then the mixture was stirred at 115° C. for 2 h. Upon completion, the solvent was removed. The residue was purified by silica gel chromatography (PE:EA=50:1-0:1) to afford (E)-1,3-dimethoxy-5-(2-nitrovinyl)-2-pentylbenzene (1.2 g, 4.30 mmol, 34% yield) as a white solid. ¹H NMR (400 MHz, CHLOROFORM-d) δ ppm 7.98 (d, J=13.6 Hz, 1H), 7.59 (d, J=13.6 Hz, 1H), 6.69 (s, 2H), 3.86 (s, 6H), 2.63-2.68 (m, 2H), 1.44-1.49 (m, 2H), 1.30-1.36 (m, 4H), 0.88-0.92 (m, 3H).

### Step 3: Preparation of 2-(3,5-dimethoxy-4-pentylphenyl)ethanamine (8)

To a solution of (E)-1,3-dimethoxy-5-(2-nitrovinyl)-2-pentylbenzene (1.2 g, 4.30 mmol, 1 eq.) in THF (15 mL) was added LiAlH₄ (652 mg, 17.18 mmol, 4 eq.) at 0° C. over 10 min. The resulting mixture was stirred at 60° C. for 4 h. Upon completion, the mixture was cooled to 0° C. Then 0.6 mL H₂O was added dropwise with stirring followed by 0.6 mL 30% aq. NaOH. The mixture was stirred to a smooth dispersion then filtered and concentrated. The residue was purified by prep-HPLC (column: Phenomenex luna C18 250×50 mm×10 μm; mobile phase: [water (0.04% HCl)-ACN]; B %: 25%-55%, 10 min) to afford 2-(3,5-dimethoxy-4-pentylphenyl)ethanamine (350 mg, 1.17 mmol, 27% yield, 96.04% purity, HCl) as a white solid. LCMS R_{T}=2.214 min, MS cal.: 251.19, [M+H]⁺=252.1; 1H NMR (400 MHz, DMSO-d6, HCl salt) δ ppm 8.08 (br s, 3H), 6.51 (s, 2H), 3.76 (s, 5H), 3.11-2.98 (m, 2H), 2.91-2.79 (m, 2H), 2.49-2.46 (m, 2H), 1.41-1.20 (m, 6H), 0.85 (t, J=7.2 Hz, 3H); ¹³C NMR (101 MHz, DMSO-d6, HCl salt) δ ppm 157.70, 136.10, 116.26, 104.34, 55.59, 33.41, 31.30, 28.44, 22.19, 21.92, 13.87.

### Example 8: Preparation of 2-(4-(butylthio)-3,5-dimethoxyphenyl)ethanamine (9)

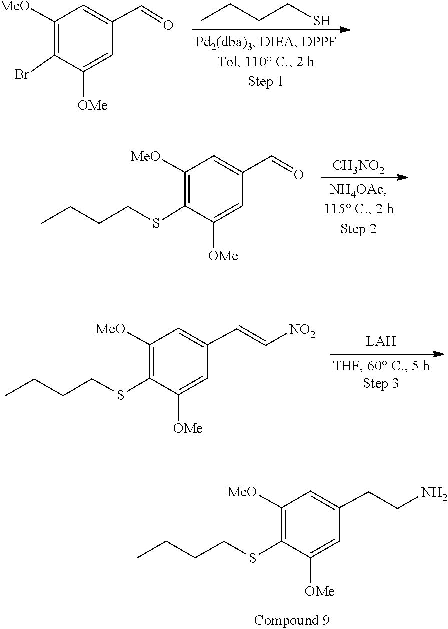

### Step 1: Preparation of 4-(butylthio)-3,5-dimethoxybenzaldehyde

To a mixture of 4-bromo-3,5-dimethoxy-benzaldehyde (5 g, 20.40 mmol, 1 eq.) and butane-1-thiol (2.39 g, 26.52 mmol, 2.84 mL, 1.3 eq.) in toluene (50 mL) was added DIEA (7.91 g, 61.21 mmol, 10.66 mL, 3 eq.), DPPF (1.13 g, 2.04 mmol, 0.1 eq.) and Pd₂(dba)₃ (1.87 g, 2.04 mmol, 0.1 eq.) in one portion at 20° C. under N₂. The mixture was stirred at 110° C. for 2 h. Upon completion, the mixture was filtered and concentrated. The residue was purified by silica gel chromatography (PE:EA=100:1-50:1) to afford 4-(butylthio)-3,5-dimethoxybenzaldehyde (4 g, 15.73 mmol, 77% yield) as brown oil. ¹H NMR (400 MHz, CHLOROFORM-d) δ ppm 9.93 (s, 1H), 7.07 (s, 2H), 3.96 (s, 6H), 2.95 (t, J=7.2 Hz, 2H), 1.54-1.46 (m, 2H), 1.45-1.35 (m, 2H), 0.87 (t, J=7.2 Hz, 3H).

### Step 2: Preparation of (E)-butyl(2,6-dimethoxy-4-(2-nitrovinyl)phenyl)sulfane

A mixture of 4-(butylthio)-3,5-dimethoxybenzaldehyde (3 g, 11.80 mmol, 1 eq.) NH₄OAc (1.82 g, 23.6 mmol, 2 eq.) in 1-nitroethane (14.40 g, 235.9 mmol, 12.74 mL, 20 eq.) was stirred and warmed to 115° C. for 1 h. Upon completion, the solvent was removed. The residue was purified by silica gel chromatography (PE:EA=80:1-60:1) to afford (E)-butyl(2,6-dimethoxy-4-(2-nitrovinyl)phenyl)sulfane (2.6 g, 8.74 mmol, 74% yield) as a yellow oil. ¹H NMR (400 MHz, CHLOROFORM-d) δ ppm 7.97 (d, J=13.6 Hz, 1H), 7.60 (d, J=13.6 Hz, 1H), 6.71 (s, 2H), 3.94 (s, 6H), 2.92 (t, J=7.2 Hz, 2H), 1.54-1.46 (m, 2H), 1.45-1.37 (m, 2H), 0.88 (t, J=7.2 Hz, 3H).

### Step 3: Preparation of 2-(4-(butylthio)-3,5-dimethoxyphenyl)ethanamine (9)

A solution of (E)-butyl(2,6-dimethoxy-4-(2-nitrovinyl)phenyl)sulfane (2.6 g, 8.74 mmol, 1 eq.) in THF (50 mL) was cooled to 0° C. Then LiAlH₄ (1.33 g, 34.97 mmol, 4 eq.) was added. The mixture was warmed to 60° C. and stirred at 60° C. for 5 h. Upon completion, the mixture was cooled to 0° C. Then 1.33 mL H₂O was added. Then 1.33 mL 30% NaOH was added. The mixture was stirred to a smooth dispersion then filtered and concentrated. The residue was purified by prep-HPLC (column: Phenomenex Gemini-NX 80×40 mm×3 μm; mobile phase: [water (10 mM NH₄HCO₃)-ACN]; B %: 20%-40%, 8 min) to afford 2-(4-(butylthio)-3,5-dimethoxyphenyl)ethanamine (1.4 g, 5.20 mmol, 59% yield) as a white solid. LCMS R_{T}=1.834 min, MS cal.: 269.14, [M+H]⁺=270.1; 1H NMR (400 MHz, CHLOROFORM-d) δ ppm 6.4 (s, 2H), 3.85 (s, 6H), 2.97 (br s, 2H), 2.80-2.68 (m, 4H), 1.51-1.32 (m, 4H), 0.84 (t, J=6.8 Hz, 3H); ¹³C NMR (101 MHz, CHLOROFORM-d) δ ppm 160.88, 141.62, 107.99, 104.55, 56.03, 43.11, 40.41, 33.73, 31.59, 21.76, 13.58.

### Example 9: Preparation of 2-(4-(butylthio)-3,5-dimethoxyphenyl)-N-(2-methoxybenzyl)ethanamine (10)

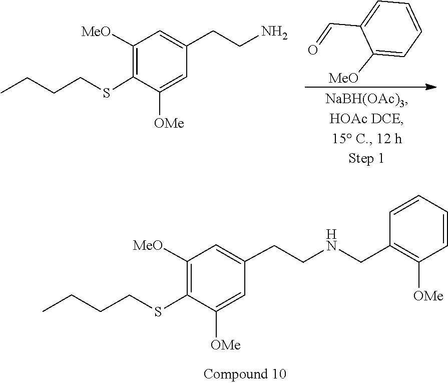 [SMILES: CCCCSc1c(OC)cc(CCN)cc1OC ; COc1ccccc1C=O ; CCCCSc1c(OC)cc(CCNCc2ccccc2OC)cc1OC]

### Step 1: Preparation of 2-(4-(butylthio)-3,5-dimethoxyphenyl)-N-(2-methoxybenzyl)ethanamine 0.10)

To a solution of 2-(4-butylsulfanyl-3,5-dimethoxyphenyl)ethanamine (900 mg, 3.34 mmol, 1 eq.) and 2-methoxybenzaldehyde (363.87 mg, 2.67 mmol, 0.8 eq.) in DCE (10 mL) was added AcOH (0.1 mL). The mixture was stirred at 20° C. for 1 h. Then NaBH(OAc)₃ (1.42 g, 6.68 mmol, 2 eq.) was added. The mixture was stirred at 20° C. for 12 h. Upon completion, the mixture was basified to pH=8 with sat. aq. NaHCO₃ soln. and stirred the extracted with DCM (10 mL×2). The organic layer was washed with brine, dried over Na₂SO₄, filtered and concentrated. The residue was purified by prep-HPLC (column: Phenomenex luna C18 (250×70 mm, 15 μm); mobile phase: [water (0.05% HCl)-ACN]; B %: 20%-50%, 20 min) to afford 2-(4-(butylthio)-3,5-dimethoxyphenyl)-N-(2-methoxybenzyl)ethanamine (310 mg, 796 μmol, 24% yield, HCl) as a white solid. LCMS R_{T}=2.203 min, MS cal.: 389.20, [M+H]⁺=390.1; 1H NMR (400 MHz, DMSO-d6, HCl salt) δ ppm 9.21 (br s, 2H), 7.52-7.48 (m, 1H), 7.44-7.39 (m, 1H), 7.09 (d, J=8.0 Hz, 1H), 7.00 (t, J=7.2 Hz, 1H), 6.57 (s, 2H), 4.13 (s, 2H), 3.83 (s, 3H), 3.79 (s, 6H), 3.17 (d, J=4.0 Hz, 2H), 3.04-2.97 (m, 2H), 2.69 (t, J=6.8 Hz, 2H), 1.36-1.30 (m, 4H), 0.83-0.78 (m, 3H); ¹³C NMR (101 MHz, DMSO-d6, HCl salt) δ ppm 160.51, 157.47, 139.06, 131.45, 130.77, 120.37, 119.74, 111.10, 107.92, 104.84, 55.99, 55.59, 47.27, 44.89, 32.68, 31.75, 31.18, 21.04, 13.50.

### Example 10: Preparation of 1-(3,5-dimethoxy-4-pentylphenyl)propan-2-amine (11)

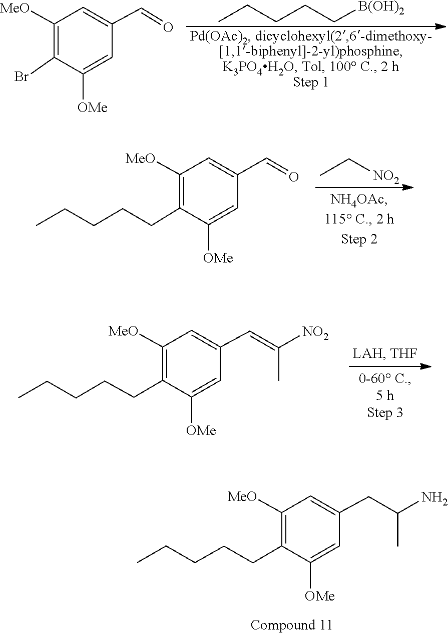

### Step 1: Preparation of 3,5-dimethoxy-4-pentylbenzaldehyde

A mixture of 4-bromo-3,5-dimethoxybenzaldehyde (6 g, 24.48 mmol, 1 eq.), K₃PO₄·H₂O (5.64 g, 24.48 mmol, 1 eq.), pentylboronic acid (4.26 g, 36.72 mmol, 1.5 eq.), dicyclohexyl(2′,6′-dimethoxy-[1,1′-biphenyl]-2-yl)phosphine (2.01 g, 4.90 mmol, 0.2 eq.) and Pd(OAc)₂ (549.66 mg, 2.45 mmol, 0.1 eq.) in toluene (70 mL) was stirred and warmed to 105° C. for 2 h under N₂. Upon completion, the mixture was filtered and concentrated. The residue was purified by silica gel chromatography (PE:EA=100:1-50:1) to afford 3,5-dimethoxy-4-pentylbenzaldehyde (5.6 g, 23.7 mmol, 97% yield) as a white solid. ¹H NMR (400 MHz, CHLOROFORM-d) δ ppm 9.91 (s, 1H), 6.96-7.12 (m, 2H), 3.89 (s, 6H), 2.62-2.74 (m, 2H), 1.48 (m, 2H), 1.29-1.38 (m, 4H), 0.90 (t, J=6.8 Hz, 3H).

### Step 2: Preparation of (E)-1,3-dimethoxy-5-(2-nitroprop-1-en-1-yl)-2-pentylbenzene

To a solution of 3,5-dimethoxy-4-pentylbenzaldehyde (3 g, 12.70 mmol, 1 eq.) in nitroethane (20 mL) was added NH₄OAc (1.96 g, 25.39 mmol, 2 eq.). The mixture was stirred at 115° C. for 2 h. Upon completion, the solvent was removed. The residue was purified by silica gel chromatography (PE:EA=50:1-0:1) to afford (E)-1,3-dimethoxy-5-(2-nitroprop-1-en-1-yl)-2-pentylbenzene (2 g, 6.82 mmol, 54% yield) as a yellow solid. 1H NMR (400 MHz, CHLOROFORM-d) δ ppm 8.08 (s, 1H), 6.60 (s, 2H), 3.84 (s, 6H), 2.62-2.67 (m, 2H), 2.51 (s, 3H), 1.44-1.51 (m, 2H), 1.34 (d, J=3.6 Hz, 4H), 0.90 (t, J=6.8 Hz, 3H).

### Step 3: Preparation of 1-(3,5-dimethoxy-4-pentylphenyl)propan-2-amine (11)

To a mixture of (E)-1,3-dimethoxy-5-(2-nitroprop-1-en-1-yl)-2-pentylbenzene (2 g, 6.82 mmol, 1 eq.) in THF (20 mL) was added LiAlH₄ (1.04 g, 27.27 mmol, 4 eq.) in one portion at 0° C. under N₂. The mixture was stirred at 20° C. for 30 min, then heated to 60° C. and stirred for 4 h. Upon completion, the mixture was cooled to 0° C. and stirred. H₂O (1 mL) was added dropwise. Then 30% aq. NaOH (1 mL) was added dropwise. The mixture was stirred to a smooth dispersion then filtered and concentrated. The residue was purified by prep-HPLC (column: Phenomenex luna C18 250×50 mm×15 μm; mobile phase: [water (0.05% HCl)-ACN]; B %: 15%-45%, 20 min), to afford 1-(3,5-dimethoxy-4-pentylphenyl)propan-2-amine (0.4 g, 1.25 mmol, 18% yield, 94.4% purity, HCl) as a white solid. 368 mg was LCMS R_{T}=2.238 min, MS cal.: 265.20, [M+H]⁺=266.1; 1H NMR (400 MHz, DMSO-d6, HCl salt) δ ppm 8.17 (br s, 3H), 6.49 (s, 2H), 3.75 (s, 6H), 3.49-3.36 (m, 1H), 2.96 (dd, J=5.6, 13.2 Hz, 1H), 2.65 (dd, J=8.4, 13.2 Hz, 1H), 2.50-2.45 (m, 2H), 1.41-1.22 (m, 6H), 1.16 (d, J=6.4 Hz, 3H), 0.85 (t, J=6.8 Hz, 3H); ¹³C NMR (101 MHz, DMSO-d6, HCl salt) δ ppm 157.65, 135.52, 116.29, 104.89, 55.58, 47.91, 40.56, 31.31, 28.42, 22.23, 21.92, 17.82, 13.86.

### Example 11: Preparation of 1-(4-(butylthio)-3,5-dimethoxyphenyl)propan-2-amine (12)

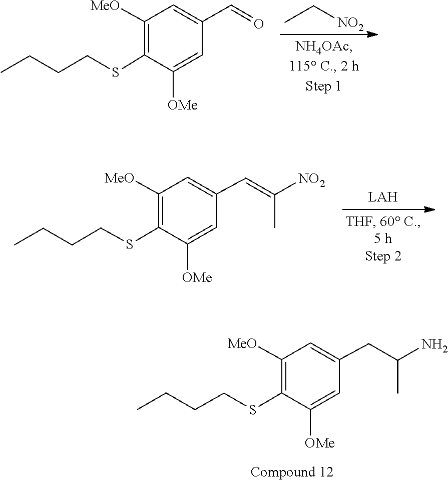

### Step 1: Preparation of (E)-butyl(2,6-dimethoxy-4-(2-nitroprop-1-en-1-yl) phenyl)sulfane

A mixture of 4-(butylthio)-3,5-dimethoxybenzaldehyde (1.5 g, 5.90 mmol, 1 eq.) NH₄OAc (909.19 mg, 11.80 mmol, 2 eq.) in nitroethane (8.85 g, 117.95 mmol, 8.43 mL, 20 eq.) was stirred and warmed to 115° C. for 1 h. Upon completion, the solvent was removed. The residue was purified by silica gel chromatography (PE:EA=80:1-60:1) to afford (E)-butyl(2,6-dimethoxy-4-(2-nitroprop-1-en-1-yl)phenyl)sulfane (1.5 g, 4.82 mmol, 82% yield) as a yellow oil. 1H NMR (400 MHz, CHLOROFORM-d) δ ppm 8.05 (s, 1H), 6.61 (s, 2H), 3.92 (s, 6H), 2.89 (t, J=7.2 Hz, 2H), 2.50 (s, 3H), 1.54-1.47 (m, 2H), 1.45-1.36 (m, 2H), 0.89 (t, J=7.2 Hz, 3H).

### Step 2: Preparation of 1-(4-(butylthio)-3,5-dimethoxyphenyl)propan-2-amine (12)

A solution of (E)-butyl(2,6-dimethoxy-4-(2-nitroprop-1-en-1-yl)phenyl)sulfane (1.1 g, 3.53 mmol, 1 eq.) in THF (30 mL) was cooled to 0° C. Then LiAlH₄ (536 mg, 14.13 mmol, 4 eq.) was added. The mixture was warmed to 60° C. and stirred at 60° C. for 5 h. Upon completion, the mixture was cooled to 0° C. Then 0.54 mL H₂O was added. Then 0.54 mL 30% NaOH was added. The mixture was stirred to a smooth dispersion then filtered and concentrated. The residue was purified by prep-HPLC (column: Kromasil C18 (250×50 mm×10 μm); mobile phase: [water (10 mM NH₄HCO₃)-ACN]; B %: 30%-55%, 10 min) to afford 1-(4-(butylthio)-3,5-dimethoxyphenyl)propan-2-amine (340 mg, 1.20 mmol, 34% yield) as a white solid. LCMS R_{T}=1.929 min, MS cal.: 283.43, [M+H]⁺=284.1; 1H NMR (400 MHz, CHLOROFORM-d) δ ppm 6.40 (s, 2H), 3.86 (s, 6H), 3.25-3.16 (m, 1H), 2.77 (t, J=7.2 Hz, 2H), 2.70 (dd, J=4.8, 13.2 Hz, 1H), 2.47 (dd, J=8.4, 13.2 Hz, 1H), 1.50-1.43 (m, 2H), 1.43-1.34 (m, 2H), 1.14 (d, J=6.4 Hz, 3H), 0.85 (t, J=7.2 Hz, 3H); ¹³C NMR (101 MHz, CHLOROFORM-d6) δ ppm 160.89, 141.55, 108.28, 105.03, 56.09, 48.28, 47.09, 33.79, 31.66, 23.55, 21.79, 13.60.

### Example 12: Preparation of 2-(2,5-dimethoxy-4-pentylphenyl)-N-[(2-methoxyphenyl)methyl]ethanamine (13)

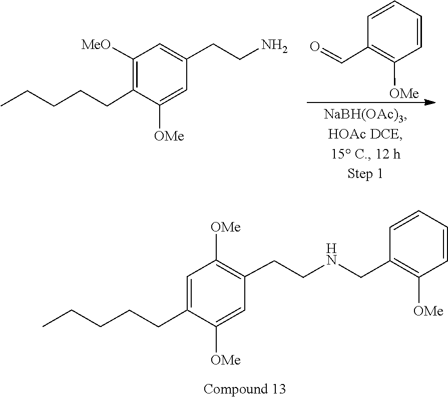 [SMILES: CCCCCc1c(OC)cc(CCN)cc1OC ; COc1ccccc1C=O ; CCCCCc1cc(OC)c(CCNCc2ccccc2OC)cc1OC]

### Step 1: Preparation of 2-(2,5-dimethoxy-4-pentylphenyl)-N-[(2-methoxyphenyl)methyl]ethanamine (13

A solution of 2-(2,5-dimethoxy-4-pentylphenyl)ethanamine (1.1 g, 4.38 mmol, 1 eq.), 2-methoxybenzaldehyde (476.64 mg, 3.50 mmol, 0.8 eq.), and AcOH (52.56 mg, 875.23 μmol, 50.06 uL, 0.2 eq.) in DCE (10 mL) was stirred at 15° C. for 1 h. Then NaBH(OAc)₃ (1.85 g, 8.75 mmol, 2 eq.) was added. The mixture was stirred at 15° C. for 12 h. Upon completion, the mixture was basified to pH=8 with sat. aq. NAHCO₃ soln. the stirred and extracted with DCM (10 mL×2). The organic layer was washed with brine, dried over Na₂SO₄, filtered and concentrated. The residue was purified by prep-HPLC (column: Phenomenex luna C18 (250×70 mm, 15 μm); mobile phase: [water (0.05% HCl)-ACN]; B %: 35%-65%, 20 min) to afford 2-(2,5-dimethoxy-4-pentylphenyl)-N-[(2-methoxyphenyl)methyl]ethanamine (360 mg, 867.41 μmol, 20% yield, 98.3% purity, HCl) as a white solid. LCMS R_{T}=2.527 min, MS cal.: 371.25, [M+H]⁺=372.2; 1H NMR (400 MHz, DMSO-d6, HCl salt) δ ppm 9.10-8.99 (m, 2H), 7.49-7.39 (m, 2H), 7.09 (d, J=8.4 Hz, 1H), 7.00 (t, J=7.2 Hz, 1H), 6.78 (s, 2H), 4.13 (s, 2H), 3.83 (s, 3H), 3.72 (d, J=4.0 Hz, 6H), 3.08-3.01 (m, 2H), 2.96-2.89 (m, 2H), 2.52 (s, 2H), 1.55-1.45 (m, 2H), 1.32-1.26 (m, 4H), 0.86 (t, J=6.8 Hz, 3H); ¹³C NMR (101 MHz, DMSO-d6, HCl salt) δ ppm 157.48, 150.81, 150.77, 131.44, 130.77, 129.81, 122.63, 120.36, 119.69, 113.23, 113.09, 111.09, 55.86, 55.58, 46.23, 44.89, 31.15, 29.57, 29.29, 26.32, 21.93, 13.90.

### Example 13: Preparation of 1-(2,5-dimethoxy-4-pentylphenyl)-N-(2-methoxybenzyl)propan-2-amine (14)

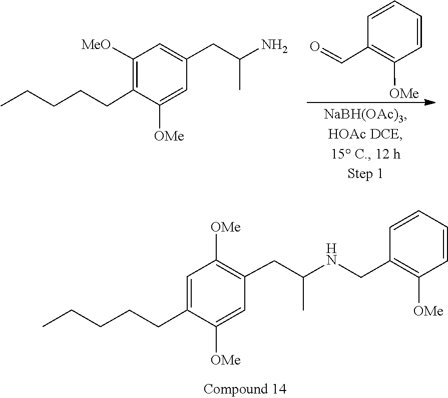 [SMILES: CCCCCc1c(OC)cc(CC(C)N)cc1OC ; COc1ccccc1C=O ; CCCCCc1cc(OC)c(CC(C)NCc2ccccc2OC)cc1OC]

### Step 1: Preparation of 1-(2,5-dimethoxy-4-pentylphenyl)-N-(2-methoxybenzyl)propan-2-amine

A solution of 1-(2,5-dimethoxy-4-pentylphenyl)propan-2-amine (700 mg, 2.64 mmol, 1 eq.), 2-methoxybenzaldehyde (251.38 mg, 1.85 mmol, 0.7 eq.), and AcOH (15.84 mg, 263.76 μmol, 15.09 uL, 0.1 eq.) in DCE (10 mL) was stirred at 20° C. for 1 h. Then NaBH(OAc)₃ (1.12 g, 5.28 mmol, 2 eq.) was added. The mixture was stirred at 20° C. for 12 h. Upon completion, the mixture was basified to pH=8 with sat. aq. NaHCO₃ soln. and extracted with DCM (10 mL×2). The organic layer was washed with brine, dried over Na₂SO₄, filtered and concentrated. The residue was purified by prep-HPLC (column: Phenomenex luna C18 (250×70 mm, 15 μm); mobile phase: [water (0.05% HCl)-ACN]; B %: 35%-65%, 20 min) to afford 1-(2,5-dimethoxy-4-pentylphenyl)-N-(2-methoxybenzyl)propan-2-amine (400 mg, 948 μmol, 36% yield, 100% purity, HCl) as a white solid. LCMS R_{T}=2.546 min, MS cal.: 385.54, [M+H]⁺=386.2; 1H NMR (400 MHz, DMSO-d6, HCl salt) δ ppm 8.97-8.90 (m, 1H), 8.71-8.63 (m, 1H), 7.50-7.41 (m, 2H), 7.12-7.09 (m, 1H), 7.04-6.99 (m, 1H), 6.82-6.75 (m, 2H), 4.22-4.14 (m, 2H), 3.87-3.65 (m, 9H), 3.42-3.35 (m, 1H), 3.12 (dd, J=4.4, 13.2 Hz, 1H), 2.74-2.68 (m, 1H), 2.55-2.51 (m, 2H), 1.55-1.47 (m, 2H), 1.35-1.24 (m, 4H), 1.22-1.11 (m, 3H), 0.93-0.80 (m, 3H); ¹³C NMR (101 MHz, DMSO-d6, HCl salt) δ ppm 157.50, 150.85, 150.77, 131.36, 130.77, 129.96, 122.18, 120.37, 119.85, 113.89, 113.09, 111.05, 55.90, 55.83, 55.54, 53.56, 42.89, 33.10, 31.14, 29.56, 29.22, 21.90, 15.63, 13.87.

### Example 14: Preparation of N-benzyl-2-(2,5-dimethoxy-4-methyl-phenyl)ethanamine (15)

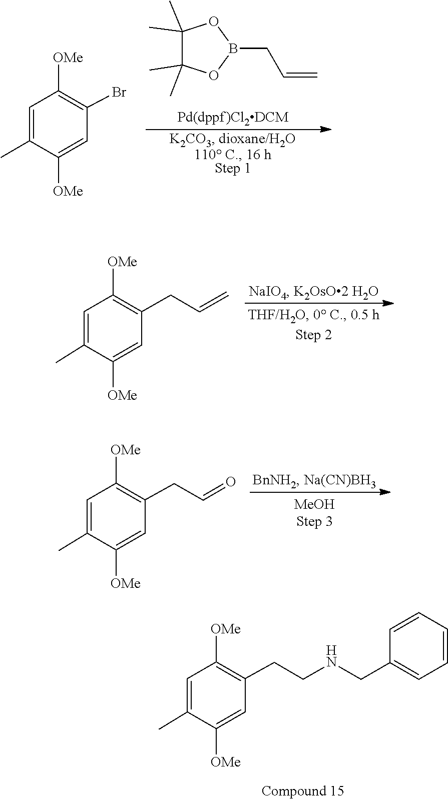

### Step 1: Preparation of 1-allyl-2,5-dimethoxy-4-methylbenzene

A mixture of 1-bromo-2,5-dimethoxy-4-methylbenzene (4 g, 17.31 mmol, 1 eq.), 2-allyl-4,4,5,5-tetramethyl-1,3,2-dioxaborolane (4.36 g, 25.96 mmol, 1.5 eq.), Pd(dppf)Cl₂·CH₂Cl₂ (1.41 g, 1.73 mmol, 0.1 eq.), and K₂CO₃ (7.18 g, 51.93 mmol, 3 eq.) in dioxane (50 mL) and H₂O (5 mL) was stirred and warmed to 110° C. for 12 h. Upon completion, the mixture was filtered and concentrated. The residue was purified by silica gel chromatography (PE:EA=20:1-5:1) to afford 1-allyl-2,5-dimethoxy-4-methylbenzene (2.7 g, 14.04 mmol, 81% yield) as a yellow oil. 1H NMR (400 MHz, CDCl₃-d) δ ppm 6.70 (s, 1H), 6.65 (s, 1H), 6.04-5.94 (m, 1H), 5.10-5.01 (m, 2H), 3.78 (s, 6H), 3.36 (d, J=6.4 Hz, 2H), 2.22 (s, 3H).

### Step 2: Preparation of 2-(2,5-dimethoxy-4-methylphenyl)acetaldehyde

A mixture of 1-allyl-2,5-dimethoxy-4-methylbenzene (2.7 g, 14.04 mmol, 1 eq.), NaIO₄ (9.01 g, 42.13 mmol, 3 eq.) in THF (30 mL) and H₂O (10 mL) was cooled to 0° C. Then potassium osmate(VI) dihydrate (1.03 g, 2.81 mmol, 0.2 eq.) was added. The mixture was stirred at 0° C. for 10 min. Upon completion, the mixture was poured into sat. aq. Na₂SO₃ soln. and extracted with EA (20 mL×2). The organic layer was washed with brine, dried over Na₂SO₄, filtered and concentrated. The residue was purified by silica gel chromatography (PE:EA=1:1) to afford 2-(2,5-dimethoxy-4-methylphenyl)acetaldehyde (0.6 g, 3.09 mmol, 22% yield) as yellow solid. 1H NMR (400 MHz, CDCl₃-d) δ ppm 9.69-9.66 (m, 1H), 6.75 (s, 1H), 6.63 (s, 1H), 3.79 (s, 3H), 3.78 (s, 3H), 3.62 (d, J=2.0 Hz, 2H), 2.24 (s, 3H).

### Step 3: Preparation of N-benzyl-2-(2,5-dimethoxy-4-methylphenyl)ethanamine (15)

A solution of 2-(2,5-dimethoxy-4-methylphenyl)acetaldehyde (500 mg, 2.57 mmol, 1 eq.) and phenylmethanamine (413.77 mg, 3.86 mmol, 1.5 eq.) in MeOH (10 mL) was stirred at 20° C. for 1 h. Then NaBH₃CN (323.54 mg, 5.15 mmol, 2 eq.) was added. The mixture was stirred at 20° C. for 12 h. Upon completion, the solvent was removed. The residue was dissolved with DCM (20 mL), washed with water and brine, dried over Na₂SO₄, filtered and concentrated. The residue was purified by prep-HPLC (column: HUAPU C18 250×50 mm×10 μm; mobile phase: [water (0.05% HCl)-ACN]; B %: 10%-40%, 20 min) to afford N-benzyl-2-(2,5-dimethoxy-4-methylphenyl)ethanamine (113 mg, 395.96 μmol, 15% yield, HCl) as a white solid. LCMS R_{T}=1.988 min, MS cal.: 285.17, [M+H]⁺=286.1; 1H; 1H NMR (400 MHz, DMSO-d₆, HCl salt) δ ppm 9.29 (br s, 2H), 7.55 (dd, J=1.6, 7.6 Hz, 2H), 7.48-7.39 (m, 3H), 6.82 (s, 1H), 6.76 (s, 1H), 4.16 (s, 2H), 3.74-3.69 (m, 6H), 3.10-2.99 (m, 2H), 2.99-2.88 (m, 2H), 2.13 (s, 3H); ¹³C NMR (101 MHz, DMSO-d₆, HCl salt) δ ppm 151.27, 150.87, 132.22, 130.26, 129.13, 128.86, 125.18, 122.70, 114.20, 113.05, 56.06, 55.89, 50.02, 46.34, 26.67, 16.21.

### Example 15: Preparation of 2-(2,5-dimethoxy-4-methylphenyl)-N-(2-fluorobenzyl)ethanamine (16)

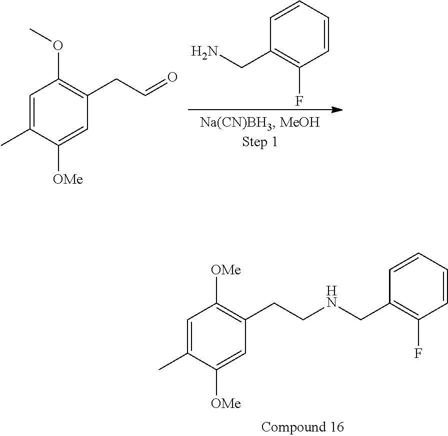 [SMILES: COc1cc(CC=O)c(OC)cc1C ; NCc1ccccc1F ; COc1cc(CCNCc2ccccc2F)c(OC)cc1C]

### Step 1: Preparation of 2-(2,5-dimethoxy-4-methylphenyl)-N-(2-fluorobenzyl)ethanamine (16)

A solution of 2-(2,5-dimethoxy-4-methylphenyl)acetaldehyde (800 mg, 4.12 mmol, 1 eq.) and (2-fluorophenyl)methanamine (618.54 mg, 4.94 mmol, 562.31 uL, 1.2 eq.) in MeOH (10 mL) was stirred at 20° C. for 1 h. Then NaBH₃CN (517.66 mg, 8.24 mmol, 2 eq.) was added. The mixture was stirred at 20° C. for 12 h. Upon completion, the solvent was removed. The residue was dissolved with DCM (20 mL), washed with H₂O and brine, dried over Na₂SO₄, filtered and concentrated. The residue was purified by prep-HPLC (column: Phenomenex luna C18 250×50 mm×10 μm; mobile phase: [water (0.04% HCl)-ACN]; B %: 5%-35%, 10 min) to afford 2-(2,5-dimethoxy-4-methylphenyl)-N-(2-fluorobenzyl)ethanamine (130 mg, 399.01 umol, 10% yield, 100% purity, HCl) as a white solid. LCMS R_{T}=2.014 min, MS cal.: 303.16, [M+H]⁺=304.1; 1H; 1H NMR (400 MHz, DMSO-d₆, HCl salt) δ ppm 9.22 (br s, 2H), 7.67 (t, J=7.2 Hz, 1H), 7.53-7.46 (m, 1H), 7.34-7.27 (m, 2H), 6.83 (s, 1H), 6.78 (s, 1H), 4.22 (s, 2H), 3.72 (d, J=4.0 Hz, 6H), 3.14-3.07 (m, 2H), 2.96-2.89 (m, 2H), 2.13 (s, 3H); ¹³C NMR (101 MHz, DMSO-d₆, HCl salt) δ ppm 150.85, 150.45, 132.09, 132.05, 131.26, 131.18, 124.79, 124.46, 124.42, 122.20, 115.43, 115.22, 113.80, 112.69, 55.64, 55.47, 46.33, 42.90, 42.86, 26.19, 15.67.

### Example 16: Preparation of 1-(2,5-dimethoxy-4-methylphenyl)-N-(2-methoxybenzyl)propan-2-amine (17)

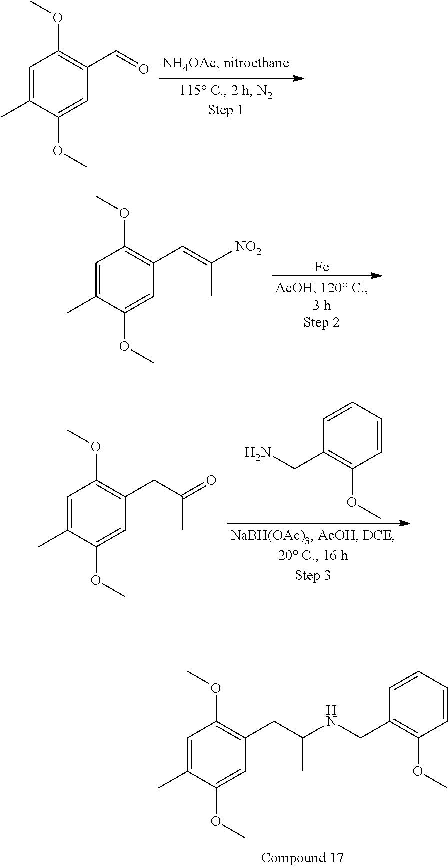

### Step 1: Preparation of (E)-1,4-dimethoxy-2-methyl-5-(2-nitroprop-1-en-1-yl)benzene

To a mixture of 2,5-dimethoxy-4-methylbenzaldehyde (3 g, 16.65 mmol, 1 eq.) eq.) in 1-nitroethane (21.25 g, 283.02 mmol, 20.23 mL, 17 eq.) was added NH₄OAc (2.57 g, 33.30 mmol, 2 eq.) in one portion at 20° C. under N₂. The mixture was stirred at 115° C. for 2 h. Upon completion, the reaction mixture was concentrated to give a residue. The residue was purified by column chromatography (SiO₂, Petroleum ether/Ethyl acetate=100/1-10/1) to afford 1,4-dimethoxy-2-methyl-5-[(E)-2-nitroprop-1-enyl]benzene (2.2 g, 9.27 mmol, 56% yield) as a yellow oil. ¹H NMR (400 MHz, DMSO-d₆) δ ppm 2.21 (s, 4H) 2.38 (s, 4H) 3.77 (s, 4H) 3.81 (s, 4H) 6.95 (s, 1H) 7.01 (s, 1H) 8.15 (s, 1H).

### Step 2: Preparation of 1-(2,5-dimethoxy-4-methylphenyl)propan-2-one

To a mixture of 1,4-dimethoxy-2-methyl-5-[(E)-2-nitroprop-1-enyl]benzene (2.2 g, 9.27 mmol, 1 eq.) in AcOH (40 mL) was added iron (517.84 mg, 9.27 mmol, 1 eq.) in one portion at 20° C. under N₂. The mixture was stirred at 120° C. for 3 h. Upon completion, the mixture was filtered by suction through a bed of wet Celite. The solids were washed with 100 mL of H₂O and 100 mL of EA. The pH was adjusted to ˜8 by added Na₂CO₃, and extracted with EA (3×50 mL). The organic layers were dried over MgSO₄, filtered, and concentrated to give crude 1-(2,5-dimethoxy-4-methylphenyl)propan-2-one (1.9 g, 9.12 mmol, 98% yield) as yellow solid. The crude was used into the next step without further purification. 1H NMR (400 MHz, DMSO-d6) δ ppm 2.06 (s, 3H) 2.14 (s, 3H) 3.60 (s, 2H) 3.67 (s, 3H) 3.70 (s, 3H) 6.74 (s, 1H) 6.80 (s, 1H)

### Step 3: Preparation of 1-(2,5-dimethoxy-4-methylphenyl)-N-(2-methoxybenzyl)propan-2-amine (17)

To a solution of 1-(2,5-dimethoxy-4-methylphenyl)propan-2-one (600 mg, 2.88 mmol, 1 eq.) and (2-methoxyphenyl)methanamine (395.23 mg, 2.88 mmol, 372.86 uL, 1 eq.) in DCE (20 mL) was added AcOH (346.03 mg, 5.76 mmol, 329.56 uL, 2 eq.) in one portion under N₂. After addition, the mixture was stirred at this temperature for 30 min, and then NaBH(OAc)₃ (1.22 g, 5.76 mmol, 2 eq.) was added in one portion at 20° C. The resulting mixture was stirred at 20° C. for 16 h. Upon completion, the mixture was washed with aq. NaHCO₃ (20 mL) and extracted with DCM (10 mL×3). The combined organic layers were dried over Na₂SO₄, filtered, and concentrated. The residue was purified by prep-HPLC (column: Phenomenex luna C18 250×50 mm×10 μm; mobile phase: [water (0.04% HCl)-ACN]; B %: 25%-55%, 10 min). Then the solution after prep-HPLC was basified to pH=8 with sat. aq. NaHCO₃ soln. and extracted with EA (20 mL×2). The organic layer was washed with brine, dried over Na₂SO₄, filtered, and concentrated to give 1-(2,5-dimethoxy-4-methylphenyl)-N-(2-methoxybenzyl)propan-2-amine (420 mg, 1.52 mmol, 53% yield, 100% purity) as a yellow oil. LCMS R_{T}=2.068 min, MS cal.: 329.20, [M+H]⁺=330.1; 1H NMR (400 MHz, CHLOROFORM-d) δ ppm 7.12-7.23 (m, 1H), 6.87 (t, J=7.2 Hz, 1H), 6.76 (d, J=8.4 Hz, 1H), 6.65 (s, 1H), 6.60 (s, 1H), 3.70-3.89 (m, 5H), 3.68 (s, 3H), 3.61 (s, 3H), 2.82-2.93 (m, 1H), 2.60-2.75 (m, 2H), 2.22 (s, 3H), 1.92-2.16 (m, 1H), 1.13 (d, J=6.0 Hz, 3H); ¹³C NMR (101 MHz, DMSO-d6) δ ppm 157.52, 151.36, 151.19, 129.70, 128.33, 127.84, 125.72, 124.62, 119.98, 113.74, 113.48, 109.76, 55.85, 54.72, 51.54, 46.88, 38.07, 20.23, 16.09.

### Example 17: Preparation of 2-(((1-(2,5-dimethoxy-4-methylphenyl)propan-2-yl)amino)methyl)phenol (18)

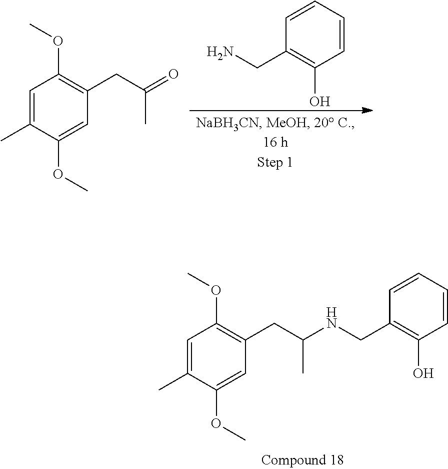 [SMILES: COc1cc(CC(C)=O)c(OC)cc1C ; NCc1ccccc1O ; COc1cc(CC(C)NCc2ccccc2O)c(OC)cc1C]

### Step 1: Preparation of 2-(((1-(2,5-dimethoxy-4-methylphenyl)propan-2-yl)amino)methyl)phenol (18)

To a solution of 1-(2,5-dimethoxy-4-methylphenyl)propan-2-one (560 mg, 2.69 mmol, 1 eq.) in MeOH (10 mL) was added 2-(aminomethyl)phenol (331.16 mg, 2.69 mmol, 62.14 uL, 1 eq.) in one portion under N₂. After addition, the mixture was stirred at this temperature for 30 min, and then NaBH₃CN (168.98 mg, 2.69 mmol, 1 eq.) was added in one portion at 20° C. The resulting mixture was stirred at 20° C. for 4 h. Upon completion, the reaction was washed by aq. NaHCO₃ (20 mL) and extracted with DCM (10 mL×3). The combined organic layers were dried over Na₂SO₄, filtered, and concentrated to give a residue. The residue was purified by prep-HPLC (column: Phenomenex luna C18 250×50 mm×10 μm; mobile phase: [water (0.04% HCl)-ACN]; B %: 20%-50%, 10 min) to afford 2-(((1-(2,5-dimethoxy-4-methylphenyl)propan-2-yl)amino)methyl)phenol (340 mg, 1.05 mmol, 39% yield, 100% purity, HCl) as a white solid. LCMS R_{T}=2.028 min, MS cal.: 315.18, [M+H]⁺=316.1; 1H NMR (400 MHz, DMSO-d₆, HCl salt) δ ppm 10.30 (s, 1H), 8.99 (br s, 1H), 8.78 (br s, 1H), 7.45 (dd, J=7.6, 1.2 Hz, 1H), 7.23-7.29 (m, 1H), 6.99 (d, J=8.0 Hz, 1H), 6.85-6.90 (m, 1H), 6.84 (s, 1H), 6.76 (s, 1H), 4.11-4.21 (m, 2H), 3.74 (s, 3H), 3.71 (s, 3H), 3.15 (dd, J=13.2, 4.0 Hz, 1H), 2.68-2.78 (m, 1H), 2.15 (s, 3H), 1.18 (d, J=6.4 Hz, 3H); ¹³C NMR (101 MHz, DMSO-d₆, HCl salt) δ ppm 156.55, 151.51, 151.24, 131.97, 130.69, 125.49, 122.80, 119.46, 118.79, 115.92, 114.51, 113.94, 56.26, 53.79, 43.03, 33.53, 16.47, 16.05.

### Example 18: Preparation of 1-(2,5-dimethoxy-4-methyl-phenyl)-N-[(2-methoxyphenyl)methyl]butan-2-amine (19)

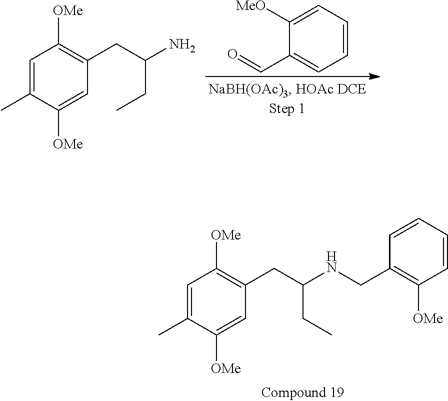 [SMILES: CCC(N)Cc1cc(OC)c(C)cc1OC ; COc1ccccc1C=O ; CCC(Cc1cc(OC)c(C)cc1OC)NCc1ccccc1OC]

### Step 1: Preparation of 1-(2,5-dimethoxy-4-methylphenyl)-N-[(2-methoxyphenyl)methyl]butan-2-amine (19)

A solution of 1-(2,5-dimethoxy-4-methylphenyl)butan-2-amine (1.1 g, 4.93 mmol, 1 eq.), 2-methoxybenzaldehyde (536.52 mg, 3.94 mmol, 0.8 eq.), and AcOH (59.16 mg, 985.18 μmol, 56.34 uL, 0.2 eq.) in DCE (20 mL) was stirred at 15° C. for 1 h. Then NaBH(OAc)₃ (2.09 g, 9.85 mmol, 2 eq.) was added. The mixture was stirred at 15° C. for 12 h. Upon completion, the mixture was basified to pH=8 with sat. aq. NaHCO₃ soln. and extracted with DCM (10 mL×2). The organic layer was washed with brine, dried over Na₂SO₄, filtered and concentrated. The residue was purified by prep-HPLC (column: Phenomenex luna C18 (250×70 mm, 15 μm); mobile phase: [water (0.05% HCl)-ACN]; B %: 20%-50%, 20 min) to afford 1-(2,5-dimethoxy-4-methylphenyl)-N-[(2-methoxyphenyl)methyl]butan-2-amine (400 mg, 1.05 mmol, 21% yield, 100% purity, HCl) as a white solid. LCMS R_{T}=2.179 min, MS cal.: 343.46, [M+H]⁺=344.1; 1H NMR (400 MHz, DMSO-d6, HCl salt) δ ppm 8.90-8.78 (m, 2H), 7.50 (dd, J=1.2, 7.6 Hz, 1H), 7.45-7.38 (m, 1H), 7.08 (d, J=8.0 Hz, 1H), 7.00 (t, J=7.6 Hz, 1H), 6.82 (s, 1H), 6.78 (s, 1H), 4.15 (t, J=5.2 Hz, 2H), 3.79 (s, 3H), 3.71 (s, 3H), 3.67 (s, 3H), 3.26-3.19 (m, 1H), 3.01 (dd, J=5.2, 13.6 Hz, 1H), 2.88 (dd, J=8.8, 13.2 Hz, 1H), 2.13 (s, 3H), 1.67-1.57 (m, 2H), 0.89 (t, J=7.6 Hz, 3H); ¹³C NMR (101 MHz, DMSO-d6, HCl salt) δ ppm 157.55, 151.02, 150.75, 131.65, 130.75, 125.05, 122.19, 120.35, 119.62, 113.98, 113.41, 110.95, 58.38, 55.75, 55.73, 55.47, 43.10, 30.55, 22.48, 15.97, 9.17.

### Examples 19: Preparation of 2,5-dimethoxy-4-(2-((2-methoxybenzyl)amino)propyl)benzonitrile (20)

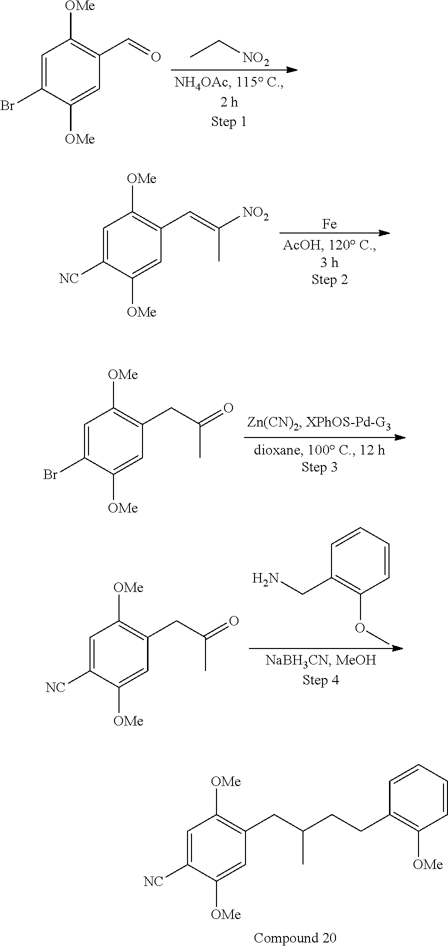

### Step 1: Preparation of 1-bromo-2,5-dimethoxy-4-[(E)-2-nitroprop-1-en-1-yl]benzene

A mixture of 4-bromo-2,5-dimethoxybenzaldehyde (10 g, 40.80 mmol, 1 eq.) and NH₄OAc (6.29 g, 81.61 mmol, 2 eq.) in nitroethane (52.07 g, 693.68 mmol, 49.59 mL, 17 eq.) was stirred and warmed to 115° C. for 2 h. Upon completion, the solvent was removed. The residue was purified by silica gel chromatography (PE:EA=60:1-40:1) to afford 1-bromo-2,5-dimethoxy-4-[(E)-2-nitroprop-1-en-1-yl]benzene (12 g, 39.72 mmol, 97% yield) as a yellow solid. 1H NMR (400 MHz, CHLOROFORM-d) δ ppm 8.17 (s, 1H), 7.17 (s, 1H), 6.83 (s, 1H), 3.88 (s, 3H), 3.85 (s, 3H), 2.39 (s, 3H).

### Step 2: Preparation of 1-(4-bromo-2,5-dimethoxyphenyl)propan-2-one

A mixture of 1-bromo-2,5-dimethoxy-4-[(E)-2-nitroprop-1-en-1-yl]benzene (9 g, 29.79 mmol, 1 eq.) and Fe (9.98 g, 178.74 mmol, 6 eq.) in AcOH (100 mL) was stirred and warmed to 120° C. for 8 h. Upon completion, the mixture was filtered and concentrated. The residue was basified to pH=9 with sat. aq. Na₂CO₃ soln. and extracted with EA (50 mL×2). The organic layer was washed with brine, dried over Na₂SO₄, filtered and concentrated. The residue was purified by silica gel chromatography (PE:EA=20:1-5:1) to afford 1-(4-bromo-2,5-dimethoxyphenyl)propan-2-one (4.7 g, 17.21 mmol, 58% yield) as a yellow solid. ¹H NMR (400 MHz, CHLOROFORM-d) δ ppm 7.07 (s, 1H), 6.72 (s, 1H), 3.84 (s, 3H), 3.77 (s, 3H), 3.65 (s, 2H), 2.16 (s, 3H).

### Step 3: Preparation of 4-acetonyl-2,5-dimethoxybenzonitrile

A mixture of 1-(4-bromo-2,5-dimethoxyphenyl)propan-2-one (2.6 g, 9.52 mmol, 1 eq.), Zn(CN)₂ (782.5 mg, 6.66 mmol, 0.7 eq.) and XPhOS-Pd-G₃ (1.21 g, 1.43 mmol, 0.15 eq.) in dioxane (30 mL) was stirred and warmed to 100° C. for 12 h. Upon completion, the mixture was filtered and concentrated. The residue was purified by silica gel chromatography (PE:EA=10:1-3:1) to afford 4-acetonyl-2,5-dimethoxybenzonitrile (1.8 g, 8.21 mmol, 86% yield) as a yellow solid. 1H NMR (400 MHz, CHLOROFORM-d) δ ppm 7.01 (s, 1H), 6.78 (s, 1H), 3.89 (s, 3H), 3.79 (s, 3H), 3.74 (s, 2H), 2.22 (s, 3H).

### Step 4: Preparation of 2,5-dimethoxy-4-(2-((2-methoxybenzyl)amino)propyl)benzonitrile (20)

A solution of 4-acetonyl-2,5-dimethoxybenzonitrile (700 mg, 3.19 mmol, 1 eq.) and (2-methoxyphenyl)methanamine (656.40 mg, 4.79 mmol, 619.25 uL, 1.5 eq.) in MeOH (10 mL) was stirred at 15° C. for 1 h. Then NaBH₃CN (401.30 mg, 6.38 mmol, 2 eq.) was added. The mixture was stirred at 15° C. for 12 h. Upon completion, the solvent was removed. The residue was dissolved with DCM (20 mL), washed with H₂O and brine, dried over Na₂SO₄, filtered and concentrated. The residue was purified by prep-HPLC (column: Kromasil C18 (250×50 mm×10 μm); mobile phase: [water (10 mM NH₄HCO₃)-ACN]; B %: 15%-55%, 20 min) to afford 2,5-dimethoxy-4-(2-((2-methoxybenzyl)amino)propyl)benzonitrile (340 mg, 982.10 μmol, 31% yield, 98.33% purity) as a yellow solid. LCMS R_{T}=1.952 min, MS cal.: 340.42, [M+H]⁺=341.1; 1H NMR (400 MHz, CHLOROFORM-d) δ ppm 7.24-7.17 (m, 1H), 7.12 (d, J=7.2 Hz, 1H), 6.93-6.90 (m, 1H), 6.87 (t, J=7.2 Hz, 1H), 6.78 (d, J=8.4 Hz, 1H), 6.74-6.71 (m, 1H), 3.86 (s, 0.5H), 3.82 (s, 3.5H), 3.70-3.67 (m, 3.5H), 3.65 (s, 3.5H), 2.93-2.84 (m, 1H), 2.77 (dd, J=7.6, 13.2 Hz, 1H), 2.66 (dd, J=5.6, 12.8 Hz, 1H), 1.90-1.84 (m, 1H), 1.10 (d, J=6.0 Hz, 3H); ¹³C NMR (101 MHz, CHLOROFORM-d) δ ppm 157.85, 155.78, 151.78, 136.48, 130.12, 128.45, 128.43, 120.47, 117.03, 114.86, 114.37, 110.34, 99.07, 56.64, 56.13, 55.25, 51.47, 47.21, 38.97, 20.70.

### Example 20: Preparation of 4-(2-((2-hydroxybenzyl)amino)propyl)-2,5-dimethoxybenzonitrile (21)

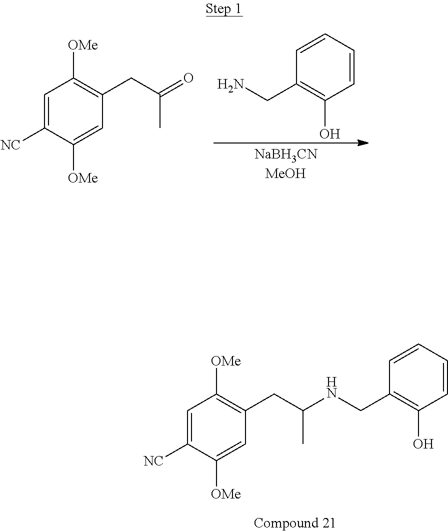 [SMILES: COc1cc(CC(C)=O)c(OC)cc1C#N ; NCc1ccccc1O ; COc1cc(CC(C)NCc2ccccc2O)c(OC)cc1C#N]

### Step 1: Preparation of 4-(2-((2-hydroxybenzyl)amino)propyl)-2,5-dimethoxybenzonitrile (21)

A solution of 4-acetonyl-2,5-dimethoxybenzonitrile (700 mg, 3.19 mmol, 1 eq.) and 2-(aminomethyl)phenol (589.82 mg, 4.79 mmol, 1.5 eq.) in MeOH (10 mL) was stirred at 15° C. for 1 h. Then NaBH₃CN (401.30 mg, 6.39 mmol, 2 eq.) was added. The mixture was stirred at 15° C. for 12 h. Upon completion, the solvent was removed. The residue was dissolved with DCM (10 mL), washed with H₂O and brine, dried over Na₂SO₄, filtered and concentrated. The residue was purified by prep-HPLC (column: Welch Xtimate C18 250×70 mm×10 μm; mobile phase: [water (0.05% NH₃H₂O−10 mM NH₄HCO₃)-ACN]; B %: 32%-62%, 30 min) to afford 4-(2-((2-hydroxybenzyl)amino)propyl)-2,5-dimethoxybenzonitrile (350 mg, 1.04 mmol, 33% yield, 97.28% purity) as a yellow solid. LCMS R_{T}=1.840 min, MS cal.: 326.39, [M+H]⁺=327.1; 1H NMR (400 MHz, CHLOROFORM-d) δ ppm 7.15 (t, J=7.6 Hz, 1H), 6.97 (s, 2H), 6.84-6.70 (m, 3H), 4.08-3.99 (m, 1H), 3.97-3.84 (m, 4H), 3.76 (s, 3H), 3.12-3.03 (m, 1H), 2.94-2.86 (m, 1H), 2.69 (dd, J=6.4, 12.8 Hz, 1H), 1.20-1.11 (m, 3H); ¹³C NMR (101 MHz, CHLOROFORM-d) δ ppm 158.12, 155.57, 151.28, 134.92, 128.63, 128.09, 122.76, 118.90, 116.58, 116.35, 114.65, 114.29, 99.36, 56.40, 55.84, 52.55, 50.11, 38.33, 20.00.

### Example 21: Preparation of 1-(2,5-dimethoxy-4-propylphenyl)propan-2-amine (22)

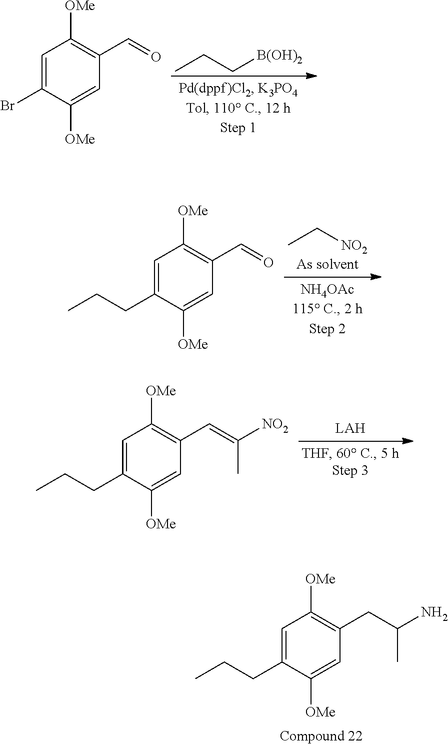

### Step 1: Preparation of 2,5-dimethoxy-4-propylbenzaldehyde

To a solution of 4-bromo-2,5-dimethoxybenzaldehyde (3 g, 12.24 mmol, 1 eq.) and propylboronic acid (1.61 g, 18.36 mmol, 1.5 eq.) in toluene (50 mL) was added K₃PO₄ (7.80 g, 36.72 mmol, 3 eq.) and Pd(dppf)Cl₂ (447.85 mg, 612.07 μmol, 0.05 eq.) under N₂. The mixture was stirred at 110 C for 2 h. Upon completion, the mixture was filtered and concentrated. The residue was purified by silica gel chromatography (PE:EA=100:1-50:1) to afford 2,5-dimethoxy-4-propyl-benzaldehyde (2 g, 9.60 mmol, 79% yield) was obtained as a light yellow solid. ¹H NMR (400 MHz, CHLOROFORM-d) δ ppm 10.42 (s, 1H), 7.29 (s, 1H), 6.81 (s, 1H), 3.91 (s, 3H), 3.84 (s, 3H), 2.67-2.62 (m, 2H), 1.68-1.61 (m, 2H), 0.99 (t, J=7.6 Hz, 3H).

### Step 2: Preparation of 1,4-dimethoxy-2-[(E)-2-nitroprop-1-en-1-yl]-5-propylbenzene

A mixture of 2,5-dimethoxy-4-propyl-benzaldehyde (2 g, 9.60 mmol, 1 eq.) in nitroethane (14.41 g, 192.00 mmol, 13.73 mL, 20 eq.) was treated with NH₄OAc (1.48 g, 19.20 mmol, 2 eq.) in one portion at 20° C. under N₂. The mixture was stirred and warmed to 115° C. for 2 h. Upon completion, the solvent was removed. The residue was purified by silica gel chromatography (PE:EA=80:1-60:1) to afford 1,4-dimethoxy-2-[(E)-2-nitroprop-1-en-1-yl]-5-propylbenzene (1.7 g, 6.41 mmol, 67% yield) as a yellow solid. 1H NMR (400 MHz, CHLOROFORM-d) δ ppm 8.29 (s, 1H), 6.78 (s, 1H), 6.76 (s, 1H), 3.85 (s, 3H), 3.81 (s, 3H), 2.65-2.60 (m, 2H), 2.43 (d, J=0.8, 3H), 1.67-1.61 (m, 2H), 0.99 (t, J=7.2 Hz, 3H).

### Step 3: Preparation of 1-(2,5-dimethoxy-4-propylphenyl)propan-2-amine (22)

To a solution of 1,4-dimethoxy-2-[(E)-2-nitroprop-1-en-1-yl]-5-propylbenzene (1.7 g, 6.41 mmol, 1 eq.) in THF (30 mL) was added LiAlH₄ (973 mg, 25.63 mmol, 4 eq.) in one portion at 0° C. under N. The mixture was stirred at 20° C. for 30 min, then heated to 60° C. and stirred for 4.5 h. Upon completion, the reaction mixture was quenched by dropwise addition of H₂O (1 mL) then 30% of aq. NaOH (1 mL) at 0° C. After stirring to a smooth dispersion, the solids were filtered, and the filtrate concentrated to give a residue. The residue was purified by prep-HPLC (column: Phenomenex luna C18 250×50 mm×15 μm; mobile phase: [water (0.05% HCl)-ACN]; B %: 15%-45%, 20 min) to afford 1-(2,5-dimethoxy-4-propylphenyl)propan-2-amine (420 mg, 1.53 mmol, 24% yield, 100% purity, HCl) as a white solid. LCMS R_{T}=1.930 min, MS cal.: 237.17, [M+H]⁺=238.1; 1H NMR (400 MHz, DMSO-d₆, HCl salt) δ ppm 8.15 (br s, 3H), 6.78 (d, J=2.0 Hz, 2H), 3.75-3.70 (m, 6H), 3.43-3.37 (m, 1H), 2.91 (dd, J=5.6, 13.1 Hz, 1H), 2.69 (dd, J=8.8, 13.2 Hz, 1H), 2.50-2.46 (m, 2H), 1.53 (m, 2H), 1.11 (d, J=6.4 Hz, 3H), 0.89 (t, J=7.2 Hz, 3H); ¹³C NMR (101 MHz, DMSO-d₆, HCl salt) δ ppm 150.95 150.76, 129.51, 122.38, 113.98, 113.11, 55.84, 46.93, 34.78, 31.78, 22.77, 17.84, 13.96.

### Example 22: Preparation of 1-(2,5-dimethoxy-4-pentylphenyl)propan-2-amine (23)

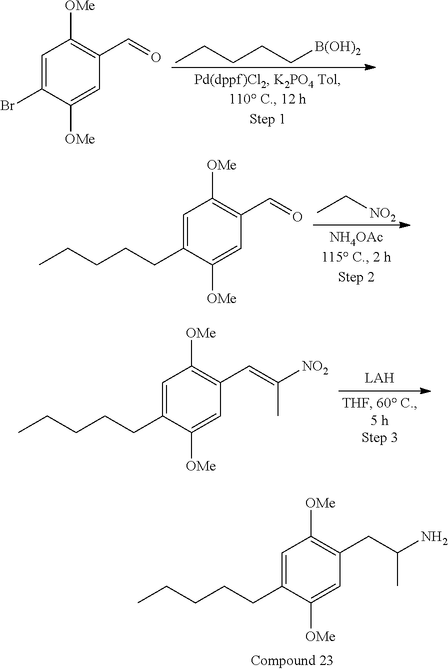

### Step 1: Preparation of 2,5-dimethoxy-4-pentylbenzaldehyde

A mixture of 4-bromo-2,5-dimethoxybenzaldehyde (6 g, 24.48 mmol, 1 eq.), pentylboronic acid (4.26 g, 36.72 mmol, 1.5 eq.), Pd(dppf)Cl₂ (895.71 mg, 1.22 mmol, 0.05 eq.), and K₃PO₄ (15.59 g, 73.45 mmol, 3 eq.) in toluene (100 mL) was stirred and warmed to 110° C. for 12 h. Upon completion, the mixture was filtered and concentrated. The residue was purified by silica gel chromatography (PE:EA=100:1-50:1) to afford 2,5-dimethoxy-4-pentylbenzaldehyde (5.1 g, 21.58 mmol, 88% yield) as a yellow oil. ¹H NMR (400 MHz, CHLOROFORM-d) δ ppm 10.4 (s, 1H), 7.27 (s, 1H), 6.79 (s, 1H), 3.89 (s, 3H), 3.82 (s, 3H), 2.59-2.68 (m, 2H), 1.52-1.65 (m, 2H), 1.26-1.41 (m, 4H), 0.91 (t, J=6.8 Hz, 3H).

### Step 2: Preparation of 1,4-dimethoxy-2-[(E)-2-nitroprop-1-en-1-yl]-5-pentylbenzene

A mixture of 2,5-dimethoxy-4-pentylbenzaldehyde (1.2 g, 5.08 mmol, 1 eq.) and NH₄OAc (782.87 mg, 10.16 mmol, 2 eq.) in nitroethane (9.53 g, 126.95 mmol, 9.08 mL, 25 eq.) was stirred at 115° C. for 1 h. Upon completion, the solvent was removed. The residue was purified by silica gel chromatography (PE:EA=80:1-60:1) to afford 1,4-dimethoxy-2-[(E)-2-nitroprop-1-en-1-yl]-5-pentylbenzene (1.2 g, 4.09 mmol, 81% yield) as a yellow oil. ¹H NMR (400 MHz, CHLOROFORM-d) δ ppm 8.30 (s, 1H), 6.79-6.77 (m, 1H), 6.76 (s, 1H), 3.85 (s, 3H), 3.81 (s, 3H), 2.66-2.61 (m, 2H), 2.43 (s, 3H), 1.65-1.57 (m, 2H), 1.40-1.33 (m, 4H), 0.92 (t, J=6.8 Hz, 3H).

### Step 3: Preparation of 1-(2,5-dimethoxy-4-pentylphenyl)propan-2-amine (23)

A solution of 1,4-dimethoxy-2-[(E)-2-nitroprop-1-en-1-yl]-5-pentylbenzene (1.2 g, 4.09 mmol, 1 eq.) in THF (20 mL) was cooled to 0° C. Then LiAlH₄ (621.02 mg, 16.36 mmol, 4 eq.) was added. The mixture was warmed to 60° C. and stirred at 60° C. for 5 h. Upon completion, the mixture was cooled to 0° C. Then 0.6 mL H₂O was added. Then 0.6 mL 30% aq. NaOH was added. The mixture was stirred to a smooth dispersion then filtered and concentrated. The residue was purified by prep-HPLC (column: Phenomenex luna C18 250×50 mm×10 μm; mobile phase: [water (0.04% HCl)-ACN]; B %: 15%-45%, 10 min) to afford 1-(2,5-dimethoxy-4-pentylphenyl)propan-2-amine (1.08 g, 4.07 mmol, 100% yield, HCl) as a white solid. LCMS R_{T}=2.163 min, MS cal.: 265.39, [M+H]⁺=266.1; 1H NMR (400 MHz, DMSO-d6, HCl salt) δ ppm 8.02 (br s, 3H), 6.78 (d, J=6.4 Hz, 2H), 3.73 (s, 6H), 3.42-3.35 (m, 1H), 2.88 (dd, J=5.6, 12.8 Hz, 1H), 2.68 (dd, J=8.4, 13.2 Hz, 1H), 2.54-2.51 (m, 2H), 1.51 (td, J=7.2, 14.8 Hz, 2H), 1.34-1.24 (m, 4H), 1.10 (d, J=6.4 Hz, 3H), 0.86 (t, J=6.8 Hz, 3H); ¹³C NMR (101 MHz, DMSO-d6, HCl salt) δ ppm 150.96, 150.71, 129.81, 122.22, 113.98, 113.01, 55.85, 55.82, 46.93, 34.77, 31.15, 29.57, 29.23, 21.91, 17.85, 13.88.

### Example 23: Preparation of 4-(2-aminopropyl)-2,5-dimethoxybenzonitrile (24)

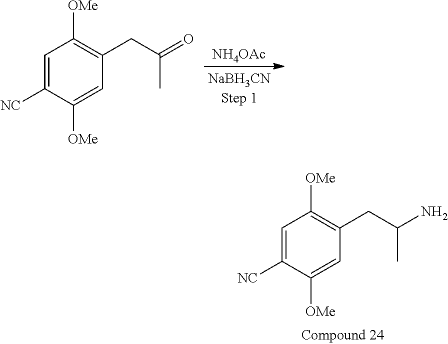 [SMILES: COc1cc(CC(C)=O)c(OC)cc1C#N ; COc1cc(CC(C)N)c(OC)cc1C#N ; "NH4OAc"]

### Step 1: Preparation of 4-(2-aminopropyl)-2,5-dimethoxybenzonitrile (24)

A mixture of 4-acetonyl-2,5-dimethoxybenzonitrile (700 mg, 3.19 mmol, 1 eq.) and NH₄OAc (739 mg, 9.58 mmol, 3 eq.) in MeOH (10 mL) was stirred at 15° C. for 1 h. Then NaBH₃CN (401.3 mg, 6.39 mmol, 2 eq.) was added and the mixture was stirred at 15° C. for 12 h. Upon completion, the solvent was removed. The residue was dissolved with DCM (20 mL), washed with H₂O and brine, dried over Na₂SO₄, filtered, and concentrated. The residue was purified by prep-HPLC (column: Kromasil C18 (250×50 mm×10 μm); mobile phase: [water (10 mM NH₄HCO₃)-ACN]; B %: 10%-50%, 10 min) to afford 4-(2-aminopropyl)-2,5-dimethoxybenzonitrile (160 mg, 726.4 μmol, 23% yield, 100% purity) as a yellow solid. LCMS R_{T}=1.552 min, MS cal.: 220.27, [M+H]⁺=221.1; 1H NMR (400 MHz, CHLOROFORM-d) δ ppm 6.98 (s, 1H), 6.80 (s, 1H), 3.89 (s, 3H), 3.80 (s, 3H), 3.28-3.19 (m, 1H), 2.77 (dd, J=5.2, 12.8 Hz, 1H), 2.58 (dd, J=8.0, 12.8 Hz, 1H), 1.39-1.31 (m, 2H), 1.13 (d, J=6.4 Hz, 3H); ¹³C NMR (101 MHz, CHLOROFORM-d) δ ppm 155.65, 151.49, 136.09, 116.70, 114.67, 114.34, 99.18, 56.47, 55.98, 46.90, 41.65, 23.86.

### Example 24: Preparation of 1-(2,5-dimethoxy-4-methylphenyl)butan-2-amine (25)

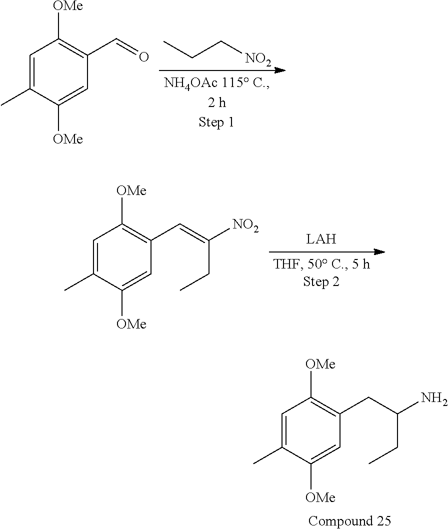

### Step 1: Preparation of 1,4-dimethoxy-2-methyl-5-[(E)-2-nitrobut-1-en-1-Yl]benzene

A mixture of 2,5-dimethoxy-4-methylbenzaldehyde (2 g, 11.10 mmol, 1 eq.) and NH₄OAc (1.71 g, 22.20 mmol, 2 eq.) in 1-nitropropane (16.81 g, 188.7 mmol, 16.84 mL, 17 eq.) was stirred at 115° C. for 2 h. Upon completion, the reaction mixture was concentrated to give a residue. The residue was purified by column chromatography (SiO₂, Petroleum ether/Ethyl acetate=100/1-15/1) to afford 1,4-dimethoxy-2-methyl-5-[(E)-2-nitrobut-1-en-1-yl]benzene (2.1 g, 8.36 mmol, 75% yield) as a yellow solid. 1H NMR (400 MHz, CHLOROFORM-d) δ ppm 8.24 (s, 1H), 6.78 (d, J=2.8 Hz, 2H), 3.82 (d, J=8.4 Hz, 6H), 2.86 (q, J=7.2 Hz, 2H), 2.28 (s, 3H), 1.30-1.26 (m, 3H).

### Step 2: Preparation of 1-(2,5-dimethoxy-4-methylphenyl)butan-2-amine (25)

A solution of 1,4-dimethoxy-2-methyl-5-[(E)-2-nitrobut-1-en-1-yl]benzene (2.7 g, 10.75 mmol, 1 eq.) in THF (40 mL) was cooled to 0° C. Then LiAlH₄ (1.63 g, 42.98 mmol, 4 eq.) was added. The mixture was warmed to 60° C. and stirred at 60° C. for 5 h. Upon completion, the mixture was cooled to 0° C. Then 2 mL (H₂O) was added. Then 2 mL 30% aq. NaOH was added. The mixture was stirred to a smooth dispersion then filtered and concentrated. The residue was purified by prep-HPLC (column: Phenomenex luna C18 250×50 mm×10 μm; mobile phase: [water (0.04% HCl)-ACN]; B %: 10%-40%, 10 min) to afford 1-(2,5-dimethoxy-4-methylphenyl)butan-2-amine (1.4 g, 5.20 mmol, 48% yield, 96.4% purity, HCl) as a white solid. LCMS R_{T}=1.786 min, MS cal.: 223.31, [M+H]⁺=224.1; 1H NMR (400 MHz, DMSO-d6, HCl salt) δ ppm 8.05 (br. s, 3H), 6.82 (s, 2H), 3.73 (d, J=2.4 Hz, 6H), 3.22 (d, J=5.6 Hz, 1H), 2.89-2.75 (m, 2H), 2.13 (s, 3H), 1.57-1.44 (m, 2H), 0.99-0.81 (m, 3H); ¹³C NMR (101 MHz, DMSO-d6, HCl salt) δ ppm 150.96, 150.88, 124.88, 122.13, 113.93, 113.69, 55.82, 55.68, 52.22, 32.55, 24.69, 15.99, 9.45.

### Example 25: Preparation of 1-(2,5-dimethoxy-4-(phenylthio)phenyl)propan-2-amine (26)

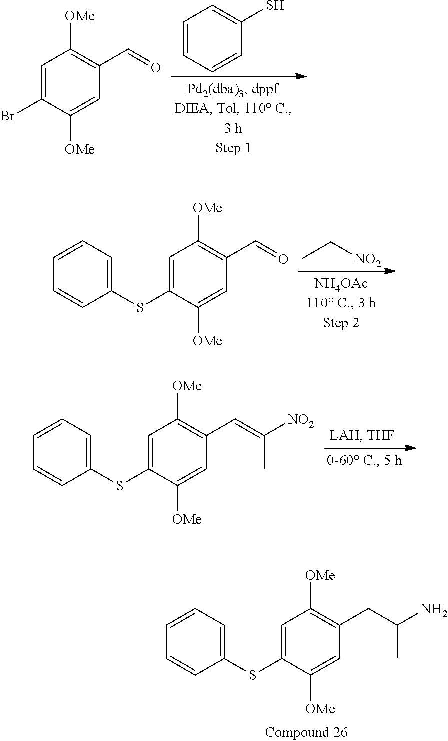

### Step 1: Preparation of 2,5-dimethoxy-4-(phenylthio)benzaldehyde

To a solution of 4-bromo-2,5-dimethoxybenzaldehyde (3 g, 12.24 mmol, 1 eq.) in Tol (30 mL) was added benzenethiol (2.70 g, 24.48 mmol, 2.50 mL, 2 eq.), Pd₂(dba)₃ (1.68 g, 1.84 mmol, 0.15 eq.), Xantphos (1.06 g, 1.84 mmol, 0.15 eq.), and DIEA (7.91 g, 61.21 mmol, 10.66 mL, 5 eq.). The solution was stirred at 110° C. for 3 h. Upon completion, the mixture was filtered and concentrated. The residue was purified by silica gel chromatography (PE:EA=40:1-10:1) to afford 2,5-dimethoxy-4-(phenylthio)benzaldehyde (3 g, 10.94 mmol, 89% yield) as yellow solid. ¹H NMR (400 MHz, CHLOROFORM-d) δ ppm 10.32 (s, 1H), 7.58 (dd, J=3.2, 6.8 Hz, 2H), 7.50-7.44 (m, 3H), 7.29 (s, 1H), 6.29 (s, 1H), 3.93 (s, 3H), 3.57 (s, 3H).

### Step 2: Preparation of (E)-(2,5-dimethoxy-4-(2-nitroprop-1-en-1-yl)phenyl)(phenyl)sulfane

To a solution of 2,5-dimethoxy-4-(phenylthio)benzaldehyde (3 g, 10.94 mmol, 1 eq.) in nitroethane (16.42 g, 218.71 mmol, 15.64 mL, 20 eq.) was added NH₄OAc (2.53 g, 32.81 mmol, 3 eq.). The solution was stirred at 110° C. for 3 h. Upon completion, the solvent was removed. The residue was purified by silica gel chromatography (PE:EA=50:1-20:1) to afford (E)-(2,5-dimethoxy-4-(2-nitroprop-1-en-1-yl)phenyl)(phenyl)sulfane (2.6 g, 7.85 mmol, 72% yield) as a yellow solid. ¹H NMR (400 MHz, CHLOROFORM-d) δ ppm 8.23 (s, 1H), 7.54-7.49 (m, 2H), 7.45-7.39 (m, 3H), 6.82 (s, 1H), 6.43 (s, 1H), 3.89 (s, 3H), 3.58 (s, 3H), 2.44-2.41 (m, 3H).

### Step 3: Preparation of 1-(2,5-dimethoxy-4-(phenylthio)phenyl)propan-2-amine (26)

To a solution of (E)-(2,5-dimethoxy-4-(2-nitroprop-1-en-1-yl)phenyl)(phenyl)sulfane (2.6 g, 7.85 mmol, 1 eq.) in THF (50 mL) was added LiAlH₄ (1.19 g, 31.38 mmol, 4 eq.) at 0° C. Then solution was stirred at 20° C. for 30 mins. Then the mixture was stirred at 60° C. for 4 h. Upon completion, the mixture was cooled to 0° C. Then 1.2 mL H₂O was added. Then 30% NaOH (1.2 mL) was added. The mixture was stirred to a smooth dispersion then filtered and concentrated. The residue was purified by prep-HPLC (column: Phenomenex luna c18 250 mm×100 mm×10 μm; mobile phase: [water (0.05% HCl)-ACN]; B %: 25%-45%, 25 min) to afford 1-(2,5-dimethoxy-4-(phenylthio)phenyl)propan-2-amine (410 mg, 1.21 mmol, 15% yield, 100% purity, HCl) as a white solid. LCMS R_{T}=2.052 min, MS cal.: 303.42, [M+H]⁺=304.1; 1H NMR (400 MHz, DMSO-d6, HCl salt) δ ppm 7.96 (br s, 3H), 7.38-7.22 (m, 5H), 6.99 (s, 1H), 6.75 (s, 1H), 3.75 (s, 3H), 3.61 (s, 3H), 3.47-3.39 (m, 1H), 2.90 (dd, J=6.4, 13.2 Hz, 1H), 2.75 (dd, J=8.0, 13.2 Hz, 1H), 1.14 (d, J=6.4 Hz, 3H); ¹³C NMR (101 MHz, DMSO-d6, HCl salt) δ ppm 151.67, 151.44, 134.69, 129.63, 129.36, 126.81, 125.57, 120.51, 115.14, 115.11, 56.36, 55.82, 46.79, 34.78, 18.07.

### Example 26: Preparation of 1-(4-(5-fluoropentyl)-2,5-dimethoxyphenyl)propan-2-amine (27)

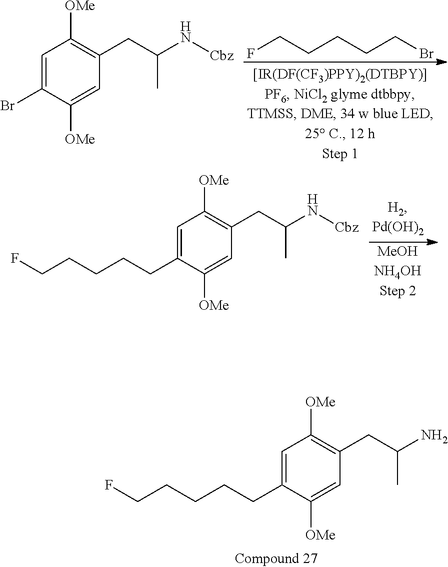

### Step 1: Preparation of benzyl (1-(4-(5-fluoropentyl)-2,5-dimethoxyphenyl)propan-2-yl)carbamate

To a solution of benzyl (1-(4-bromo-2,5-dimethoxyphenyl)propan-2-yl)carbamate (1.5 g, 3.67 mmol, 1 eq.) and 1-bromo-5-fluoropentane (2.48 g, 14.70 mmol, 4 eq.) in DME (10 mL) was added bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (41.22 mg, 36.74 μmol, 0.01 eq.), dichloronickel 1,2-dimethoxyethane (4.04 mg, 18.37 μmol, 0.005 eq.), Na₂CO₃ (778.79 mg, 7.35 mmol, 2 eq.), 4-tert-butyl-2-(4-tert-butyl-2-pyridyl)pyridine (4.93 mg, 18.37 μmol, 0.005 eq.), and bis(trimethylsilyl)silyl-trimethylsilane (913.56 mg, 3.67 mmol, 1 eq.), and the reaction was stirred at 25° C. for 12 h while illuminated with blue light (34 W LED). Upon completion, the mixture was filtered and concentrated. The residue was purified by prep-HPLC (column: Phenomenex luna C18 250×50 mm×15 μm; mobile phase: [water (0.04% HCl)-ACN]; B %: 56%-86%; 20 min) to afford benzyl (1-(4-(5-fluoropentyl)-2,5-dimethoxyphenyl)propan-2-yl)carbamate (800 mg, 1.92 mmol, 52% yield) as a yellow oil. 1H NMR (400 MHz, CHLOROFORM-d) δ=7.55-7.22 (m, 5H), 6.79-6.49 (m, 2H), 5.06 (br s, 2H), 4.58-4.46 (m, 1H), 4.43-4.30 (m, 1H), 3.95 (br d, J=6.0 Hz, 1H), 3.86-3.62 (m, 6H), 2.90-2.64 (m, 2H), 2.64-2.44 (m, 2H), 1.84-1.67 (m, 2H), 1.62 (br t, J=7.6 Hz, 2H), 1.52-1.42 (m, 2H), 1.32 (br s, 1H), 1.18 (d, J=6.4 Hz, 3H).

### Step 2: Preparation of 1-(4-(5-fluoropentyl)-2,5-dimethoxyphenyl)propan-2-amine (27)

To a solution of benzyl (1-(4-(5-fluoropentyl)-2,5-dimethoxyphenyl)propan-2-yl)carbamate (600 mg, 1.44 mmol, 1 eq.) in MeOH (10 mL) and NH₃·H₂O (1 mL) was added Pd(OH)₂ (800 mg) under N₂. The suspension was degassed under vacuum and purged with H₂ several times. The mixture was stirred under H₂ (15 psi) at 25° C. for 1 h. Upon completion, the reaction mixture was filtered, and the filtrate was concentrated. The residue was purified by prep-HPLC (column: Phenomenex luna C18 250×50 mm×15 μm; mobile phase: [water (0.04% HCl)-ACN]; B %: 15%-45%; 8 min) to afford 1-(4-(5-fluoropentyl)-2,5-dimethoxyphenyl)propan-2-amine (320 mg, 1.13 mmol, HCl) as a white solid. LCMS R_{T}=2.098 min, MS cal.: 283.38, [M+H]⁺=284.1; 1H NMR (400 MHz, CHLOROFORM-d, HCl salt) δ=8.32 (br s, 3H), 6.69 (d, J=9.2 Hz, 2H), 4.51 (t, J=6.4 Hz, 1H), 4.39 (t, J=6.4 Hz, 1H), 3.80 (d, J=8.0 Hz, 6H), 3.70 (br s, 1H), 3.18-3.02 (m, 1H), 2.97-2.83 (m, 1H), 2.66-2.50 (m, 2H), 1.61 (br d, J=8.0 Hz, 4H), 1.52-1.43 (m, 2H), 1.41 (d, J=6.4 Hz, 3H); ¹³C NMR (101 MHz, CHLOROFORM-d, HCl salt) δ=151.31, 151.21, 130.97, 121.78, 114.35, 112.96, 83.34, 56.17, 55.92, 48.52, 36.56, 30.37, 30.18, 29.64, 25.10, 25.04, 18.50.

### Example 27: Preparation of 1-(2,5-dimethoxy-4-(pentylthio)phenyl)propan-2-amine (28)

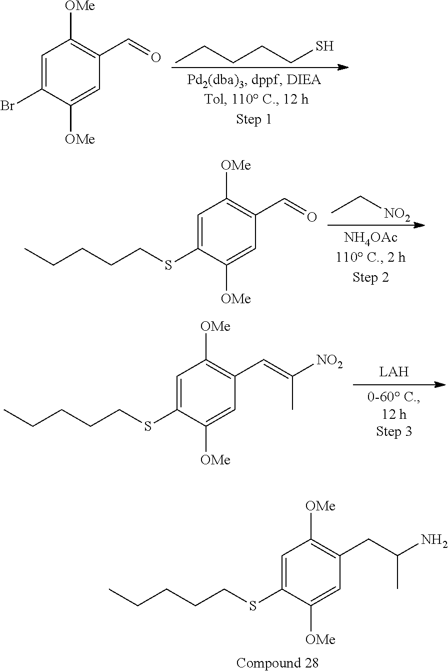

### Step 1: Preparation of 2,5-dimethoxy-4-(pentylthio)benzaldehyde

To a solution of 4-bromo-2,5-dimethoxybenzaldehyde (5 g, 20.40 mmol, 1 eq.) and pentane-1-thiol (2.76 g, 26.52 mmol, 1.3 eq.) in toluene (50 mL) was added DIEA (7.91 g, 61.21 mmol, 10.66 mL, 3 eq.), Pd₂(dba)₃ (1.87 g, 2.04 mmol, 0.1 eq.) and DPPF (1.13 g, 2.04 mmol, 0.1 eq.) under N₂. The mixture was stirred and warmed to 110° C. for 12 h. Upon completion, the mixture was filtered, and concentrated to give a residue. The residue was purified by column chromatography (SiO₂, Petroleum ether/Ethyl acetate=100/1-30/1) to give 2,5-dimethoxy-4-(pentylthio)benzaldehyde (3.5 g, 11.31 mmol, 55% yield) as a yellow solid. 1H NMR (400 MHz, CHLOROFORM-d) δ=10.36 (s, 1H), 7.25 (s, 1H), 6.77 (s, 1H), 3.91 (d, J=11.6 Hz, 6H), 2.96 (t, J 7.6 Hz, 2H), 1.77 (m, J=7.2 Hz, 2H), 1.54-1.45 (m, 2H), 1.44-1.34 (m, 2H), 0.93 (t, J=7.2 Hz, 3H).

### Step 2: Preparation of (E)-(2,5-dimethoxy-4-(2-nitroprop-1-en-1-yl)phenyl)(pentyl)sulfane

To a solution of 2,5-dimethoxy-4-(pentylthio)benzaldehyde (1.7 g, 6.33 mmol, 1 eq.) in nitroethane (9.51 g, 126.7 mmol, 9.06 mL, 20 eq.) was added NH₄OAc (1.46 g, 19.00 mmol, 3 eq.). The mixture was warmed and stirred at 110° C. for 2 h. Upon completion, the mixture was filtered, and concentrated to give a residue. The residue was purified by column chromatography (SiO₂, Petroleum ether/Ethyl acetate=100/1-50/1) to give (E)-(2,5-dimethoxy-4-(2-nitroprop-1-en-1-yl)phenyl)(pentyl)sulfane (0.5 g, 1.48 mmol, 23% yield) as a yellow solid. ¹H NMR (400 MHz, CHLOROFORM-d) δ=8.29 (s, 1H), 6.79 (d, J=6.4 Hz, 2H), 3.87 (d, J=1.6 Hz, 6H), 2.95 (t, J=7.6 Hz, 2H), 2.43 (s, 3H), 1.74 (m, J=7.6 Hz, 2H), 1.52-1.44 (m, 2H), 1.41-1.33 (m, 2H), 0.92 (t, J=7.2 Hz, 3H).

### Step 3: Preparation of 1-(2,5-dimethoxy-4-(pentylthio)phenyl)propan-2-amine (28)

To a solution of (E)-(2,5-dimethoxy-4-(2-nitroprop-1-en-1-yl)phenyl)(pentyl)sulfane (1.3 g, 3.99 mmol, 1 eq.) in THF (20 mL) was added LiAlH₄ (606.48 mg, 15.98 mmol, 4 eq.) in one portion at 0° C. under N₂. The mixture was stirred at 20° C. for 30 min, then heated to 60° C. and stirred for 12 h. Upon completion, the mixture was cooled to 0° C. The reaction mixture was quenched by addition H₂O 1 mL and 30% of aq. NaOH 1 mL at 0° C., and then filtered, and concentrated to give a residue. The residue was purified by prep-HPLC (column: Phenomenex luna C18 250×50 mm×15 μm; mobile phase: [water (0.05% HCl)-ACN]; B %: 20%-50%; 23 min) to afford 1-(2,5-dimethoxy-4-(pentylthio)phenyl)propan-2-amine (380 mg, 1.12 mmol, 28% yield, 98.7% purity, HCl) as a white solid. LCMS R_{T}=2.258 min, MS cal.: 297.46, [M+H]⁺=298.1; 1H NMR (400 MHz, DMSO-d6, HCl salt) δ=8.06 (br s, 3H), 6.83 (d, J=10.8 Hz, 2H), 3.76 (d, J=5.2 Hz, 6H), 3.33 (s, 1H), 2.93-2.84 (m, 3H), 2.70 (dd, J=8.4, 13.2 Hz, 1H), 1.58 (m, J=7.2 Hz, 2H), 1.43-1.25 (m, 4H), 1.11 (d, J=6.4 Hz, 3H), 0.91-0.81 (m, 3H); ¹³C NMR (101 MHz, DMSO-d6, HCl salt) δ=151.51, 150.27, 123.95, 122.11, 114.27, 111.14, 56.21, 56.02, 46.84, 34.58, 30.54, 30.40, 28.00, 21.63, 17.86, 13.81.

### Example 28: Preparation of 1-(4-isopentyl-2,5-dimethoxyphenyl)propan-2-amine (29)

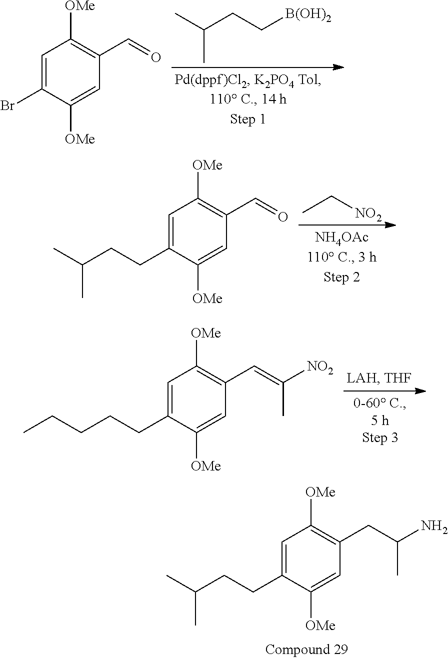

### Step 1: Preparation of 4-isopentyl-2,5-dimethoxybenzaldehyde

To a solution of 4-bromo-2,5-dimethoxybenzaldehyde (2 g, 8.16 mmol, 1 eq.) and isopentylboronic acid (946.40 mg, 8.16 mmol, 1 eq.) in toluene (20 mL) was added K₃PO₄ (5.20 g, 24.48 mmol, 3 eq.) and Pd(dppf)Cl₂ (298.57 mg, 408.05 μmol, 0.05 eq.). The mixture was stirred and warmed to 110° C. for 12 h. Upon completion, the mixture was filtered, and concentrated to remove solvent. The residue was purified by column chromatography (SiO₂, Petroleum ether/Ethyl acetate=100/0 to 4/1) to afford 4-isopentyl-2,5-dimethoxybenzaldehyde (1.7 g, 7.19 mmol, 88% yield) as a yellow oil. ¹H NMR (400 MHz, CHLOROFORM-d) δ ppm 10.97-10.86 (m, 1H), 7.78 (s, 1H), 7.31 (s, 1H), 4.41 (s, 3H), 4.34 (s, 3H), 3.20-3.12 (m, 2H), 2.17-2.10 (m, 1H), 2.03-1.93 (m, 2H), 1.48 (s, 3H), 1.47 (s, 3H).

### Step 2: Preparation of 1-isopentyl-2,5-dimethoxy-4-[(E)-2-nitroprop-1-en-1-yl]benzene

To a solution of 4-isopentyl-2,5-dimethoxybenzaldehyde (1.7 g, 7.19 mmol, 1 eq.) in 1-nitroethane (15.75 g, 209.81 mmol, 15 mL, 29.18 eq.) was added NH₄OAc (1.11 g, 14.38 mmol, 2 eq.). The mixture was stirred and warmed to 110° C. for 1 h. Upon completion, the mixture was concentrated to remove solvent. The residue was purified by column chromatography (SiO₂, Petroleum ether/Ethyl acetate=100/1-50/1) to afford 1-isopentyl-2,5-dimethoxy-4-[(E)-2-nitroprop-1-en-1-yl]benzene (1.4 g, 4.77 mmol, 66% yield) as a yellow oil. 1H NMR (400 MHz, CHLOROFORM-d) δ ppm 8.30 (s, 1H), 6.77 (d, J=6.4 Hz, 2H), 3.85 (s, 3H), 3.81 (s, 3H), 2.67-2.61 (m, 2H), 2.43 (s, 3H), 1.63 (td, J=6.6, 13.4 Hz, 1H), 1.51-1.46 (m, 2H), 0.97 (d, J=6.6 Hz, 6H).

### Step 3: Preparation of 1-(4-isopentyl-2,5-dimethoxyphenyl)propan-2-amine (29)

To a solution of 1-isopentyl-2,5-dimethoxy-4-[(E)-2-nitroprop-1-en-1-yl]benzene (1.4 g, 4.77 mmol, 1 eq.) in THF (30 mL) was added LiAlH₄ (724.52 mg, 19.09 mmol, 4 eq.) at 0° C. The mixture was stirred and warmed to 60° C. for 5 h. Upon completion, the mixture was cooled to 0° C. Then 0.7 mL H₂O was added dropwise with stirring followed by dropwise addition of 30% aq. NaOH (0.7 mL). The mixture was stirred to a smooth dispersion then filtered and concentrated. The residue was purified by prep-HPLC (column: Phenomenex luna C18 250×50 mm×10 μm; mobile phase: [water (0.04% HCl)-ACN]; B %: 20%-50%, 10 min) to afford 1-(4-isopentyl-2,5-dimethoxyphenyl)propan-2-amine (350 mg, 1.13 mmol, 24% yield, 97.9% purity, HCl) as a white solid. LCMS R_{T}=2.264 min, MS cal.: 265.39, [M+H]⁺=266.1; 1H NMR (400 MHz, DMSO-d₆, HCl salt) δ ppm 8.14-7.91 (m, 3H), 6.78 (d, J=6.9 Hz, 2H), 3.75-3.71 (m, 6H), 3.42-3.35 (m, 1H), 2.88 (dd, J=5.7, 13.2 Hz, 1H), 2.68 (dd, J=8.4, 13.2 Hz, 1H), 2.56-2.51 (m, 2H), 1.59-1.48 (m, 1H), 1.43-1.36 (m, 2H), 1.10 (d, J=6.5 Hz, 3H), 0.91 (d, J=6.5 Hz, 6H); ¹³C NMR (101 MHz, DMSO-d₆, HCl salt) δ ppm 151.01, 150.68, 129.97, 122.24, 114.02, 112.89, 55.90, 55.83, 46.93, 38.93, 34.77, 27.51, 27.47, 22.44, 17.84, 11.29.

### Example 29: Preparation of 1-(2,5-dimethoxy-4-(4-methylpentyl)phenyl)propan-2-amine (30)

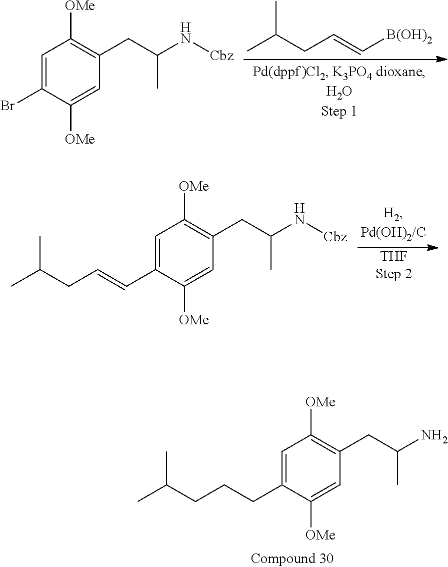

### Step 1: Preparation of benzyl 1-[2-[2,5-dimethoxy-4-[(E)-4-methylpent-1-en-1-yl]phenyl]propan-2-yl]carbamate

A mixture of benzyl (1-(4-bromo-2,5-dimethoxyphenyl)propan-2-yl)carbamate (500 mg, 1.22 mmol, 1 eq.), [(E)-4-methylpent-1-en-1-yl]boronic acid (235.09 mg, 1.84 mmol, 1.5 eq.), K₃PO₄ (779.84 mg, 3.67 mmol, 3 eq.), and Pd(dppf)Cl₂ (179.21 mg, 244.93 μmol, 0.2 eq.) in dioxane (20 mL) and H₂O (2 mL) was de-gassed and then heated to 80° C. for 3 h under N₂. Upon completion, the mixture was filtered and concentrated. The residue was purified by prep-TLC (SiO₂, PE:EA=5:1) to give product benzyl 1-[2-[2,5-dimethoxy-4-[(E)-4-methylpent-1-en-1-yl]phenyl]propan-2-yl]carbamate (340 mg, 67% Y) as a white solid. ¹H NMR (400 MHz, CHLOROFORM-d) δ=7.40-7.27 (m, 5H), 6.91 (s, 1H), 6.69-6.61 (m, 2H), 6.23-6.12 (m, 1H), 5.15-4.97 (m, 3H), 4.08-3.89 (m, 1H), 3.86-3.67 (m, 6H), 2.88-2.64 (m, 2H), 2.18-2.07 (m, 2H), 1.73 (td, J=6.8, 13.3 Hz, 1H), 1.17 (br d, J=6.4 Hz, 2H), 0.95 (d, J=6.4 Hz, 6H).

### Step 2: Preparation of 1-(2,5-dimethoxy-4-(4-methylpentyl)phenyl)propan-2-amine (30)

To a solution of benzyl 1-[2-[2,5-dimethoxy-4-[(E)-4-methylpent-1-en-1-yl]phenyl]propan-2-yl]carbamate (500 mg, 1.21 mmol, 1 eq.) in THF (20 mL) was added Pd(OH)₂/C under N₂. The suspension was degassed under vacuum and purged with H₂ several times. The mixture was stirred under H₂ (15 psi) at 20° C. for 1 h. Upon completion, the reaction mixture was filtered, and the filtrate was concentrated. The residue was purified by prep-HPLC (column: Phenomenex luna C18 250×50 mm×15 μm; mobile phase: [water (0.04% HCl)-ACN]; B %: 25%-45%; 8 min) to give 1-(2,5-dimethoxy-4-(4-methylpentyl)phenyl)propan-2-amine (68 mg, 243.4 μmol, 20% yield, 100% purity, HCl salt) as a white solid. LCMS R_{T}=2.347 min, MS cal.: 279.22, [M+H]⁺=280.1; 1H NMR (400 MHz, DMSO-d6, HCl salt) δ=7.95-7.61 (m, 3H), 6.81 (s, 1H), 6.76 (s, 1H), 3.74 (d, J=2.4 Hz, 6H), 3.45-3.36 (m, 1H), 2.85 (dd, J=6.0, 13.2 Hz, 1H), 2.69-2.67 (m, 1H), 2.60-2.53 (m, 2H), 1.60-1.46 (m, 3H), 1.26-1.17 (m, 2H), 1.11 (d, J=6.8 Hz, 3H), 0.86 (d, J=6.4 Hz, 6H); ¹³C NMR (101 MHz, DMSO-d6, HCl salt) δ=151.46, 151.21, 130.38, 122.63, 114.47, 113.52, 56.36, 56.32, 47.48, 38.84, 35.29, 30.36, 27.92, 27.73, 22.99, 18.42.

### Example 30: Preparation of 1-(2,5-dimethoxy-4-(4,4,4-trifluorobutyl)phenyl)propan-2-amine (31)

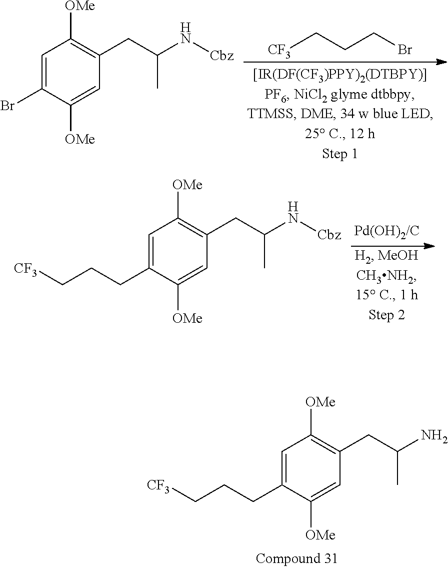 [SMILES: COc1cc(CC(C)NC(=O)OCc2ccccc2)c(OC)cc1Br ; FC(F)(F)CCCBr ; COc1cc(CC(C)NC(=O)OCc2ccccc2)c(OC)cc1CCCC(F)(F)F ; "H2, MeOH CH3·NH2, 15° C., 1 hStep 2" ; COc1cc(CC(C)N)c(OC)cc1CCCC(F)(F)F]

### Step 1: Preparation of benzyl (1-(2,5-dimethoxy-4-(4,4,4-trifluorobutyl)phenyl)propan-2-yl)carbamate

A mixture of benzyl (1-(4-bromo-2,5-dimethoxyphenyl)propan-2-yl)carbamate (500 mg, 1.22 mmol, 1 eq.), 4-bromo-1,1,1-trifluorobutane (935.57 mg, 4.90 mmol, 4 eq.), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (13.74 mg, 12.25 μmol, 0.01 eq.), dichloronickel 1,2-dimethoxyethane (1.35 mg, 6.12 μmol, 0.005 eq.), Na₂CO₃ (259.60 mg, 2.45 mmol, 2 eq.), dtbbpy (1.64 mg, 6.12 μmol, 0.005 eq.), and TTMSS (304.52 mg, 1.22 mmol, 377.81 uL, 1 eq.) in DME (4 mL) was degassed and purged with Ar 3 times. Then the mixture was stirred at 25° C. for 12 h under an Ar atmosphere while illuminated with blue light (34 W LED). Upon completion, the reaction mixture was filtered, and the filtrate was concentrated. The residue was purified by prep-HPLC (column: Phenomenex luna C18 250×50 mm×15 μm; mobile phase: [water (0.04% HCl)-ACN]; B %: 52%-82%; 20 min) to afford benzyl (1-(2,5-dimethoxy-4-(4,4,4-trifluorobutyl)phenyl)propan-2-yl)carbamate (780 mg, crude) as a white solid. ¹H NMR (400 MHz, CHLOROFORM-d) δ 7.38-7.29 (m, 5H), 6.63 (s, 2H), 5.05 (br s, 2H), 4.02-3.89 (m, 1H), 3.82-3.70 (m, 6H), 2.88-2.60 (m, 4H), 2.19-1.98 (m, 2H), 1.90-1.78 (m, 2H), 1.18 (d, J=6.4 Hz, 3H)

### Step 2: Preparation of 1-(2,5-dimethoxy-4-(4,4,4-trifluorobutyl)phenyl)propan-2-amine (31)

To a solution of benzyl (1-(2,5-dimethoxy-4-(4,4,4-trifluorobutyl)phenyl)propan-2-yl)carbamate (580 mg, 1.32 mmol, 1 eq.) in MeOH (30 mL) and CH₃NH₂ (3 mL, 30% purity) was added Pd(OH)₂ (1 g, 7.12 mmol, 5.40 eq.). The mixture was stirred at 15° C. for 2 h under H₂ (15 psi). Upon completion, the reaction mixture was filtered, and the filtrate was concentrated to afford 1-(2,5-dimethoxy-4-(4,4,4-trifluorobutyl)phenyl)propan-2-amine (500 mg) as a white solid. 1H NMR (400 MHz, CHLOROFORM-d) δ 6.69-6.62 (m, 2H), 3.78 (d, J=1.2 Hz, 6H), 3.27-3.16 (m, 1H), 2.78-2.63 (m, 3H), 2.52 (dd, J=8.0, 12.9 Hz, 1H), 2.19-2.05 (m, 2H), 1.91-1.80 (m, 2H), 1.14 (d, J=6.4 Hz, 3H)³C NMR (101 MHz, CHLOROFORM-d) δ 151.57, 151.17, 128.69, 127.73, 126.65, 114.04, 113.03, 56.05, 55.99, 47.30, 40.96, 33.50, 33.23, 29.28, 23.56, 22.30, 22.28.

### Example 31: Preparation of 1-(4-butoxy-2,5-dimethoxyphenyl)propan-2-amine (33)

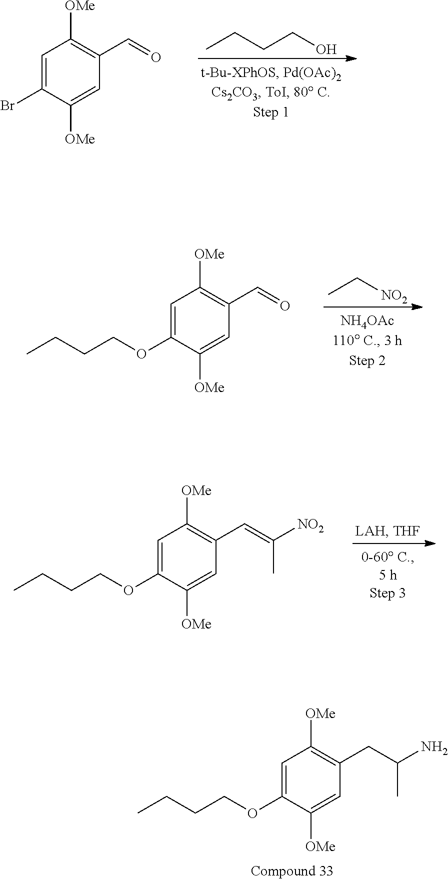

### Step 1: Preparation of 4-butoxy-2,5-dimethoxybenzaldehyde

A mixture of 4-bromo-2,5-dimethoxybenzaldehyde (2 g, 8.16 mmol, 1 eq.), butan-1-ol (1.21 g, 16.32 mmol, 1.49 mL, 2 eq.), Pd(OAc)₂ (183.22 mg, 816.09 μmol, 0.1 eq.), t-BuXphos (693.09 mg, 1.63 mmol, 0.2 eq.), and Cs₂CO₃ (7.98 g, 24.48 mmol, 3 eq.) in toluene (20 mL) was degassed and purged with N₂ 3 times, and then the mixture was stirred at 80° C. for 16 h under a N₂ atmosphere. Upon completion, the reaction mixture was poured into H₂O (20 mL). The mixture was extracted with ethyl acetate (20 mL×2). The organic phase was washed with brine (10 mL), dried over anhydrous Na₂SO₄, filtered, and concentrated in vacuo to give crude product. The crude product was purified by silica gel chromatography (Petroleum ether/Ethyl acetate=100:1-1:1) to give 4-butoxy-2,5-dimethoxybenzaldehyde (1 g, 4.20 mmol, 510% yield) as a yellow solid.

### Step 2: Preparation of (E)-1-butoxy-2,5-dimethoxy-4-(2-nitroprop-1-en-1-yl)benzene

To a solution of 4-butoxy-2,5-dimethoxybenzaldehyde (0.9 g, 3.78 mmol, 1 eq.) in nitroethane (10 mL) was added NH₄OAc (873.41 mg, 11.33 mmol, 3 eq.). The mixture was stirred at 110° C. for 0.5 h. Upon completion, the reaction was concentrated to give crude product. The crude product was purified by chromatography on silica gel eluted with petroleum ether:ethyl acetate (100:1-0:1) to give (E)-1-butoxy-2,5-dimethoxy-4-(2-nitroprop-1-en-1-yl)benzene (1 g, 3.39 mmol, 90% yield) as a yellow solid.

### Step 3: Preparation of 1-(4-butoxy-2,5-dimethoxyphenyl)propan-2-amine (33)

A mixture of (E)-1-butoxy-2,5-dimethoxy-4-(2-nitroprop-1-en-1-yl)benzene (1 g, 3.39 mmol, 1 eq.) in THF (10 mL) was degassed and purged with N₂ 3 times, LiAlH₄ (514.06 mg, 13.54 mmol, 4 eq.) was added at 0° C., and then the mixture was stirred at 60° C. for 6 h under a N₂ atmosphere. Upon completion, the reaction mixture was quenched by dropwise addition of H₂O (1 mL) and 30% aq. NaOH (1 mL) at 0° C., and after solids formed, the mixture was filtered, and the filtrate concentrated to give a residue. The crude product was purified by prep-HPLC (column: Phenomenex luna C18 250×50 mm×10 μm; mobile phase: [water (0.04% HCl)-ACN]; B %: 20%-50%; 10 min) to give 1-(4-butoxy-2,5-dimethoxyphenyl)propan-2-amine (520 mg, 1.84 mmol, 54% yield, 94.7% purity, HCl salt) as a white solid. ¹HNMR (400 MHz, DMSO-d₆, HCl salt) δ ppm 8.02-8.35 (m, 3H), 6.77-6.84 (m, 1H), 6.62-6.68 (m, 1H), 3.93-4.01 (m, 2H), 3.82-3.92 (m, 3H), 3.72-3.78 (m, 3H), 3.66-3.71 (m, 3H), 2.80-2.91 (m, 1H), 2.58-2.70 (m, 1H), 1.61-1.75 (m, 2H), 1.36-1.50 (m, 2H), 1.05-1.17 (m, 3H), 0.86-0.98 (m, 3H); ¹³C NMR (101 MHz, DMSO-d₆, HCl salt) δ ppm 151.59, 147.99, 142.65, 116.02, 115.60, 99.32, 68.17, 56.46, 56.09, 47.05, 34.30, 30.98, 18.82, 17.77, 13.76.

### Example 32: Preparation of 1-(2,5-dimethoxy-4-(3-methoxypropyl)phenyl)propan-2-amine (35)

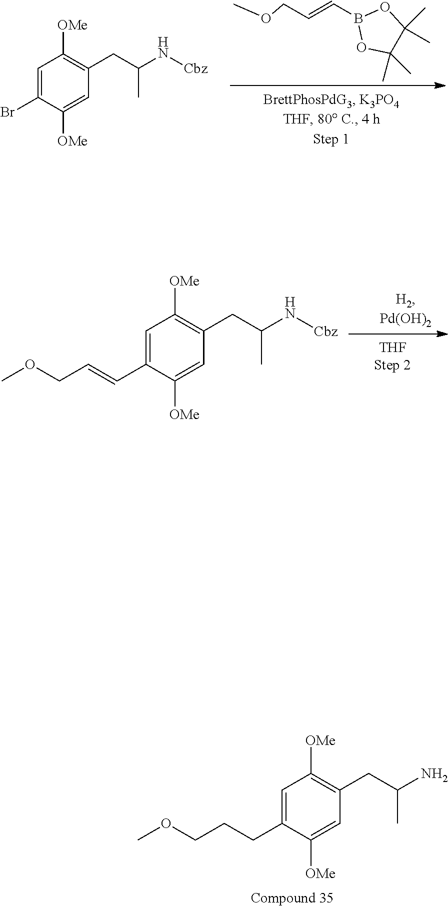

### Step 1: Preparation of benzyl (1-(2,5-dimethoxy-4-((E)-3-methoxyprop-1-en-1-yl)phenyl)propan-2-yl)carbamate

To a mixture of benzyl (1-(4-bromo-2,5-dimethoxyphenyl)propan-2-yl)carbamate (1.5 g, 3.67 mmol, 1 eq.) and 2-[(E)-3-methoxyprop-1-en-1-yl]-4,4,5,5-tetramethyl-1,3,2-dioxaborolane (2.18 g, 11.02 mmol, 2.34 mL, 3 eq.) in THF (30 mL) was added K₃PO₄ (1.56 g, 7.35 mmol, 2 eq.) and BrettPhosPdG3 (333.04 mg, 367.39 μmol, 0.1 eq.) in one portion at 25b° C. under N₂. The mixture was stirred at 80° C. for 4 h. Upon completion, the mixture was filtered and concentrated. The residue was purified by column chromatography (SiO₂, Petroleum ether/Ethyl acetate=100/1-0/1) to afford benzyl (1-(2,5-dimethoxy-4-((E)-3-methoxyprop-1-en-1-yl)phenyl)propan-2-yl)carbamate (600 mg, 1.50 mmol, 41% yield) as a yellow oil. ¹H NMR (400 MHz, CHLOROFORM-d) δ=7.39-7.28 (m, 5H), 6.95 (s, 2H), 6.66 (br s, 1H), 6.27 (td, J=6.4, 16.0 Hz, 1H), 5.05 (s, 3H), 4.12 (dd, J=1.2, 6.4 Hz, 2H), 3.98 (br s, 1H), 3.89-3.69 (m, 6H), 3.40 (s, 3H), 2.73 (br d, J=5.6 Hz, 2H), 1.18 (d, J=6.4 Hz, 3H).

### Step 2: Preparation of 1-(2,5-dimethoxy-4-(3-methoxypropyl) phenyl) propan-2-amine (35)

To a solution of benzyl (1-(2,5-dimethoxy-4-((E)-3-methoxyprop-1-en-1-yl)phenyl)propan-2-yl)carbamate (300 mg, 751 μmol, 1 eq.) in THF (4 mL) was added Pd(OH)₂/C under N₂. The suspension was degassed under vacuum and purged with H₂ several times. The mixture was stirred under H₂ (15 psi) at 25° C. for 1 h. Upon completion, the reaction mixture was filtered, and the filtrate was concentrated. The residue was purified by prep-HPLC (column: Phenomenex luna C18 250×50 mm×15 μm; mobile phase: [water (0.04% HCl)-ACN]; B %: 10%-40%; 7 min) to afford 1-(2,5-dimethoxy-4-(3-methoxypropyl)phenyl)propan-2-amine (151 mg, 497 μmol, 66% yield, HCl) as a white solid. LCMS R_{T}=1.820 min, MS cal.: 267.36, [M+H]⁺=268.1; 1H NMR (400 MHz, CHLOROFORM-d, HCl salt) δ=8.33 (br s, 3H), 6.70 (s, 2H), 3.79 (d, J=5.2 Hz, 6H), 3.70 (br d, J=1.6 Hz, 1H), 3.40 (t, J=6.4 Hz, 2H), 3.36 (s, 3H), 3.09 (br dd, J=6.0, 13.1 Hz, 1H), 2.90 (br dd, J=7.6, 13.2 Hz, 1H), 2.72-2.58 (m, 2H), 1.85 (dd, J=6.8, 8.4 Hz, 2H), 1.40 (br d, J=6.4 Hz, 3H); ¹³C NMR (101 MHz, CHLOROFORM-d, HCl salt) δ=151.33, 151.29, 130.32, 122.02, 114.34, 113.08, 72.32, 58.54, 56.16, 55.90, 48.40, 36.55, 29.76, 27.00, 18.43

### Example 33: Preparation of 2-(4-hexyl-2,5-dimethoxyphenyl)ethanamine (36)

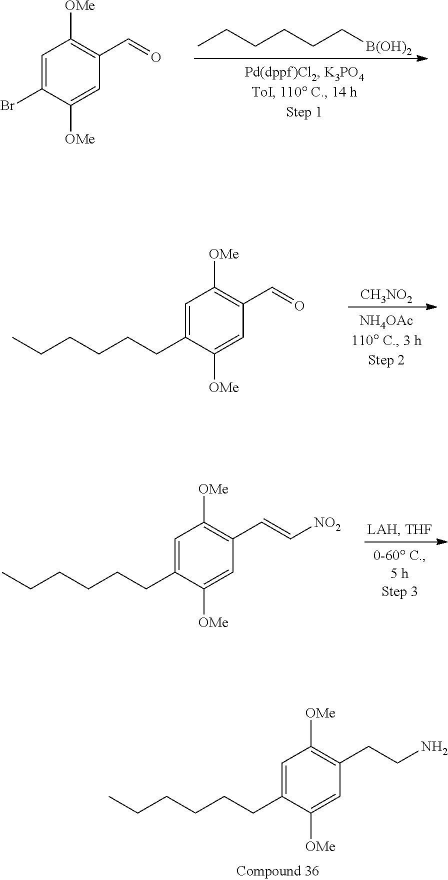

### Step 1: Preparation of 4-hexyl-2,5-dimethoxybenzaldehyde

To a solution of 4-bromo-2,5-dimethoxybenzaldehyde (3 g, 12.24 mmol, 1 eq.) and hexylboronic acid (1.59 g, 12.24 mmol, 1 eq.) in toluene (50 mL) was added Pd(dppf)Cl₂ (448 mg, 0.612 mmol, 0.1 eq.) and K₃PO₄ (5.2 g, 24.48 mmol, 2 eq.) under N₂. The mixture was stirred at 110° C. for 12 h. Upon completion, the mixture was filtered, and concentrated to give a residue. The residue was purified by column chromatography (SiO₂, Petroleum ether/Ethyl acetate=100/1-30/1) to give 4-hexyl-2,5-dimethoxybenzaldehyde (2.6 g, 10.32 mmol, 84% yield) as a yellow solid. 1H NMR (400 MHz, CHLOROFORM-d) δ=10.36 (s, 1H), 7.25 (s, 1H), 6.77 (s, 1H), 3.91 (d, J=11.6 Hz, 6H), 2.56 (t, J=7.6 Hz, 2H), 1.77 (s, 3H), 1.54-1.45 (s, 6H), 0.93 (t, J=7.2 Hz, 3H).

### Step 2: Preparation of (E)-1-hexyl-2,5-dimethoxy-4-(2-nitrovinyl)benzene

To a solution of 4-hexyl-2,5-dimethoxybenzaldehyde (1 g, 3.99 mmol, 1 eq.) in nitromethane (11.3 g, 185.12 mmol, 10 mL, 20 eq.) was added NH₄OAc (615.84 g, 7.99 mmol, 3 eq.). The mixture was stirred and warmed to 110° C. for 2 h. Upon completion, the mixture was filtered, and concentrated to give a residue. The residue was purified by column chromatography (SiO₂, Petroleum ether/Ethyl acetate=100/1-50/1) to give (E)-1-hexyl-2,5-dimethoxy-4-(2-nitrovinyl)benzene (1 g, 3.44 mmol, 84% yield) as a yellow solid. ¹H NMR (400 MHz, CHLOROFORM-d) δ=8.29 (d, J=12.8, 1H), 7.79 (d, 1H), 6.89 (d, 1H), 6.75 (d, J=8.4 Hz, 1H), 3.95 (s, 3H), 3.63 (s, J=8, Hz, 2H), 1.65 (m, 2H), 1.52-1.44 (m, 6H), 0.92 (t, J=7.2 Hz, 3H).

### Step 3: Preparation of 2-(4-hexyl-2,5-dimethoxyphenyl)ethanamine (36)

To a solution of (E)-1-hexyl-2,5-dimethoxy-4-(2-nitrovinyl)benzene (1 g, 3.41 mmol, 1 eq.) in THF (20 mL) was added LiAlH₄ (517.46 mg, 13.64 mmol, 4 eq.) in one portion at 0° C. under N₂. The mixture was stirred at 20° C. for 30 min, then heated to 60° C. and stirred for 11.5 h. Upon completion, the mixture was cooled to 0° C. The reaction mixture was quenched by dropwise addition of H₂O (1 mL) followed by 30% of aq. NaOH (1 mL) at 0° C., and then after stirring the solids were filtered, and the filtrate concentrated to give a residue. The residue was purified by prep-HPLC (column: Phenomenex luna C18 250×50 mm×15 μm; mobile phase: [water (0.05% HCl)-ACN]; B %: 20%-50%; 23 min) to afford 2-(4-hexyl-2,5-dimethoxyphenyl)ethanamine (270 mg, 0.99 mmol, 29% yield, 97% purity, HCl) as a white solid. LCMS R_{T}=2.258 min, MS cal.: 297.46, [M+H]⁺=298.1; 1H NMR (400 MHz, DMSO-d6, HCl salt) δ=7.89 (br. s, 3H), 6.83 (d, J 10.8 Hz, 2H), 3.76 (d, J 5.2 Hz, 6H), 2.89 (t, J 7.2 Hz, 2H), 2.65 (t, J 8.0 Hz, 2H), 2.51 (s, 2H), 1.48 (d, J 7.2 Hz, 2H), 1.43-1.25 (d, J 2.8 Hz, 6H), 0.75 (t, J 6.4 Hz, 3H); ¹³C NMR (101 MHz, DMSO-d6, HCl salt) δ=151.51, 150.27, 123.95, 122.11, 114.27, 111.14, 56.21, 56.02, 46.84, 34.58, 30.54, 30.40, 28.00, 21.63, 17.86, 13.81.

### Example 34: Preparation of 2-(4-(4-fluorobutyl)-2,5-dimethoxyphenyl)ethanamine (37)

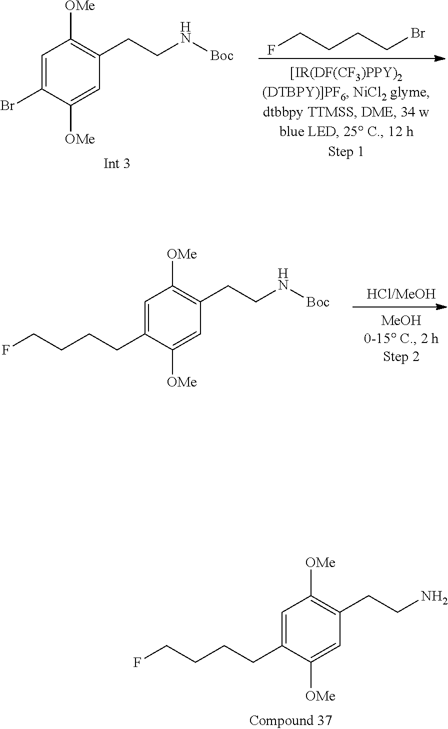 [SMILES: COc1cc(CCNC(=O)OC(C)(C)C)c(OC)cc1Br ; FCCCCBr ; COc1cc(CCNC(=O)OC(C)(C)C)c(OC)cc1CCCCF ; COc1cc(CCCCF)c(OC)cc1CCN]

### Step 1: Preparation of tert-butyl (4-(4-fluorobutyl)-2,5-dimethoxyphenethyl)carbamate

A mixture of tert-butyl (4-bromo-2,5-dimethoxyphenethyl)carbamate (500 mg, 1.39 mmol, 1 eq.), 1-bromo-4-fluorobutane (860.58 mg, 5.55 mmol, 597.62 uL, 4 eq.), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (15.57 mg, 13.88 μmol, 0.01 eq.), dichloronickel 1,2-dimethoxyethane (1.52 mg, 6.94 μmol, 0.005 eq.), Na₂CO₃ (294.22 mg, 2.78 mmol, 2 eq.), dtbbpy (1.86 mg, 6.94 μmol, 0.005 eq.), and TTMSS (345.13 mg, 1.39 mmol, 428.20 uL, 1 eq.) in DME (4 mL) was degassed and purged with Ar 3 times, and then the mixture was stirred at 25° C. for 10 h under an Ar atmosphere while illuminated with blue light (34 W LED). Upon completion, the reaction mixture was filtered, and the filtrate was concentrated. The residue was purified by prep-HPLC (column: Phenomenex luna C18 250×50 mm×15 μm; mobile phase: [water (0.04% HCl)-ACN]; B %: 47%-77%; 20 min) to afford tert-butyl (4-(4-fluorobutyl)-2,5-dimethoxyphenethyl)carbamate (870 mg, crude) as a white solid. 1H NMR (400 MHz, CHLOROFORM-d) δ 6.69-6.63 (m, 2H), 4.76-4.62 (m, 1H), 4.54 (t, J=6.0 Hz, 1H), 4.42 (t, J=5.6 Hz, 1H), 3.78 (d, J=4.0 Hz, 6H), 3.40-3.27 (m, 2H), 2.78 (t, J=6.8 Hz, 2H), 2.63 (t, J=7.2 Hz, 2H), 1.84-1.64 (m, 4H), 1.44 (s, 9H)

### Step 2: Preparation of 2-(4-(4-fluorobutyl)-2,5-dimethoxyphenyl)ethanamine (37)

To a solution of tert-butyl (4-(4-fluorobutyl)-2,5-dimethoxyphenethyl)carbamate (750 mg, 2.11 mmol, 1 eq.) in MeOH (20 mL) was added HCl/MeOH (4 M, 60 mL, 113.74 eq.) at 0° C. The mixture was stirred at 15° C. for 2 h. Upon completion, the mixture was concentrated to afford 2-(4-(4-fluorobutyl)-2,5-dimethoxyphenyl)ethanamine (500 mg, HCl salt) as a white solid. 1H NMR (400 MHz, DMSO-d6, HCl salt) δ 7.83 (br s, 3H), 6.79 (s, 2H), 4.53-4.49 (m, 1H), 4.41-4.37 (m, 1H), 3.74 (d, J=3.2 Hz, 6H), 3.00-2.93 (m, 2H), 2.81 (br d, J=8.4 Hz, 2H), 2.58-2.53 (m, 3H), 1.73-1.56 (m, 4H); ¹³C NMR (101 MHz, DMSO-d6, HCl salt) δ 151.33, 151.27, 129.68, 123.40, 113.86, 113.58, 83.37, 56.33, 30.04, 29.64, 28.48, 25.73.

### Example 35: Preparation of 2-(4-(butylthio)-2,5-dimethoxyphenyl)ethanamine (38)

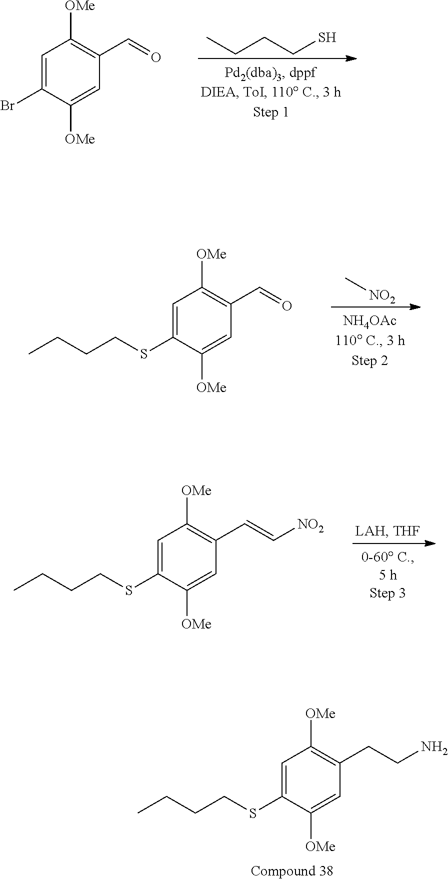

### Step 1: Preparation of 4-(butylthio)-2,5-dimethoxybenzaldehyde

To a solution of 4-bromo-2,5-dimethoxybenzaldehyde (5 g, 20.40 mmol, 1 eq.) and butane-1-thiol (2.76 g, 30.60 mmol, 3.28 mL, 1.5 eq.) in toluene (50 mL) was added DIEA (7.91 g, 61.21 mmol, 10.66 mL, 3 eq.), DPPF (1.13 g, 2.04 mmol, 0.1 eq.), and Pd₂(dba)₃ (1.87 g, 2.04 mmol, 0.1 eq.) under N. The mixture was stirred and warmed to 110° C. for 3 h. Upon completion, the mixture was filtered, and concentrated to give a residue. The residue was purified by column chromatography (SiO₂, Petroleum ether/Ethyl acetate=100/1-30/1) to give 4-(butylthio)-2,5-dimethoxybenzaldehyde (4 g, 15.73 mmol, 77% yield) as a gray solid. ¹H NMR (400 MHz, CHLOROFORM-d) δ=10.35 (s, 1H) 7.24 (s, 1H), 6.76 (s, 1H), 3.89-3.92 (d, J=12.4 Hz, 6H), 2.94-2.98 (t, J=7.2 Hz, 2H), 1.72-1.76 (m, 2H), 1.52-1.55 (m, 2H), 0.95-0.99 (m, 2H).

### Step 2: Preparation of (E)-butyl(2,5-dimethoxy-4-(2-nitrovinyl)phenyl)sulfane

To a solution of 4-(butylthio)-2,5-dimethoxybenzaldehyde (2.5 g, 9.83 mmol, 1 eq.) in nitromethane (12.0 g, 196.6 mmol, 10.62 mL, 20 eq.) was added NH₄OAc (2.27 g, 29.49 mmol, 3 eq.). The mixture was stirred and warmed to 110° C. for 3 h. Upon completion, the mixture was filtered, and concentrated to give a residue. The residue was purified by column chromatography (SiO₂, Petroleum ether/Ethyl acetate=100/1-3/1) to give (E)-butyl(2,5-dimethoxy-4-(2-nitrovinyl)phenyl)sulfane (1 g, 3.36 mmol, 34% yield) as a yellow solid. ¹H NMR (400 MHz, CHLOROFORM-d) δ=8.14 (s, 1H), 7.27 (s, 1H), 6.83 (s, 1H), 6.77 (s, 1H), 3.94 (s, 3H), 3.86 (s, 3H), 2.95-2.98 (t, J=14.8 Hz, 2H), 1.71-1.73 (m, 2H), 1.51-1.53 (m, 2H), 0.96-0.99 (t, J=14.8 Hz, 3H).

### Step 3: Preparation of 2-(4-(butylthio)-2,5-dimethoxyphenyl)ethanamine (38)

To a solution of (E)-butyl(2,5-dimethoxy-4-(2-nitrovinyl)phenyl)sulfane (1 g, 3.36 mmol, 1 eq.) in THF (20 mL) was added LiAlH₄ (510.48 mg, 13.45 mmol, 4 eq.) at 0° C. under N₂. The mixture was heated to 60° C. and stirred for 3 h at 60° C. under N₂. Upon completion, the reaction mixture was quenched by addition of water 0.5 mL at 0° C. and a solution of 15% NaOH (1.5 mL) at 0° C., and then diluted with water (0.5 mL) at 0° C. The mixture was stirred vigorously, filtered, and the filtrate was concentrated to give a residue. The residue was purified by prep-HPLC (column: Phenomenex luna C18 250×50 mm×15 μm; mobile phase: [water (0.05% HCl)-ACN]; B %: 20%-50%; 23 min) to afford 2-(4-(butylthio)-2,5-dimethoxyphenyl)ethanamine (0.26 g, 825 μmol, 25% yield, 97% purity, HCl) as a white solid. LCMS R_{T}=2.105 min, MS cal.: 269.40, [M+H]⁺=270.1; 1H NMR (400 MHz, DMSO-d6, HCl salt) δ=8.02 (br s, 3H), 6.83 (d, J=11.6 Hz, 2H), 3.76 (s, 6H), 2.83-2.90 (m, 6H), 1.41-1.54 (m, 4H), 0.88 (s, 3H); ¹³C NMR (101 MHz, DMSO-d6, HCl salt) δ=151.42, 150.42, 123.78, 122.74, 113.74, 111.19, 56.26, 56.11, 38.64, 30.51, 27.83, 21.42, 13.58.

### Example 36: Preparation of 2-(2,5-demethoxy-4-(pentylthio)phenyl)ethanamine (39)

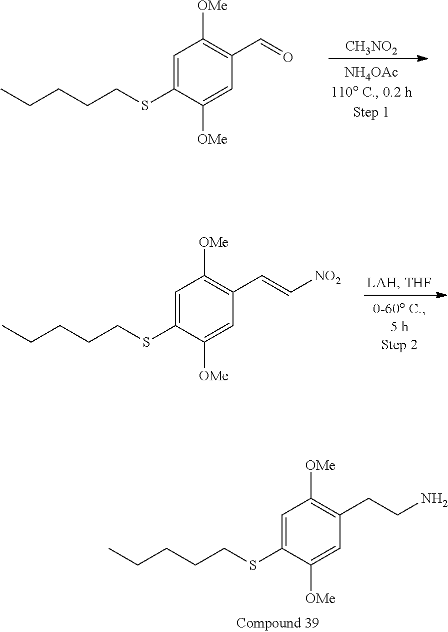 [SMILES: CCCCCSc1cc(OC)c(C=O)cc1OC ; "CH3NO2" ; CCCCCSc1cc(OC)c(/C=C/[N+](=O)[O-])cc1OC ; CCCCCSc1cc(OC)c(CCN)cc1OC]

### Step 1: Preparation of (E)-(2,5-dimethoxy-4-(2-nitrovinyl)phenyl)(pentyl)sulfane

To a solution of 2,5-dimethoxy-4-(pentylthio)benzaldehyde (1.8 g, 6.71 mmol, 1 eq.) in nitromethane (8.19 g, 134.14 mmol, 7.25 mL, 20 eq.) was added NH₄OAc (1.55 g, 20.12 mmol, 3 eq.). The mixture was stirred and warmed to 110° C. for 0.2 h. Upon completion, the mixture was filtered, and concentrated to give a residue. The residue was purified by column chromatography (SiO₂, Petroleum ether/Ethyl acetate=100/1-50/1) to give (E)-(2,5-dimethoxy-4-(2-nitrovinyl)phenyl)(pentyl)sulfane (1 g, 3.21 mmol, 48% yield) as a yellow solid. ¹H NMR (400 MHz, CHLOROFORM-d) δ=8.13 (d, J=13.6 Hz, 1H), 7.84 (d, J=13.6 Hz, 1H), 6.87-6.75 (m, 2H), 3.91 (d, J 18.8 Hz, 6H), 2.99-2.92 (m, 1H), 2.96 (t, J=7.6 Hz, 1H), 1.75 (m, J=7.4 Hz, 2H), 1.53-1.33 (m, 4H), 0.96-0.90 (m, 1H), 0.93 (t, J=7.2 Hz, 2H).

### Step 2: Preparation of 2-(2,5-dimethoxy-4-(pentylthio)phenyl)ethanamine (39)

To a solution of (E)-(2,5-dimethoxy-4-(2-nitrovinyl)phenyl)(pentyl)sulfane (1 g, 3.21 mmol, 1 eq.) in THF (15 mL) was added LiAlH₄ (487.54 mg, 12.85 mmol, 4 eq.) in one portion at 0° C. under N₂. The mixture was stirred at 20° C. for 30 min, then heated to 60° C. and stirred for 12 h. Upon completion, the mixture was cooled to 0° C. The reaction mixture was quenched by addition of H₂O (1 mL) and 30% aq. NaOH (1 mL) at 0° C., and then filtered, and concentrated to give a residue. The residue was purified by prep-HPLC (column: Phenomenex luna C18 250×50 mm×15 μm; mobile phase: [water (0.04% HCl)-ACN]; B %: 20%-50%; 10 min) to afford 2-(2,5-dimethoxy-4-(pentylthio)phenyl)ethanamine (340 mg, 1.20 mmol, 37% yield, 100% purity, HCl) as a white solid. LCMS R_{T}=2.201 min, MS cal.: 283.43, [M+H]⁺=284.1; 1H NMR (400 MHz, DMSO-d6, HCl salt) δ=8.01 (br s, 3H), 6.83 (d, J=10.4 Hz, 2H), 3.76 (d, J=6.8 Hz, 6H), 3.00-2.87 (m, 4H), 2.85-2.78 (m, 2H), 1.57 (m, J=7.2 Hz, 2H), 1.43-1.35 (m, 2H), 1.33-1.27 (m, 2H), 0.86 (t, J=7.2 Hz, 3H); ¹³C NMR (101 MHz, DMSO-d6, HCl salt) δ=151.35, 150.37, 123.75, 122.69, 113.67, 111.18, 56.21, 56.06, 38.57, 30.59, 30.43, 28.05, 27.78, 21.68, 13.86.

### Example 37: Preparation of 1-(4-hexyl-2,5-dimethoxyphenyl)butan-2-amine (40)

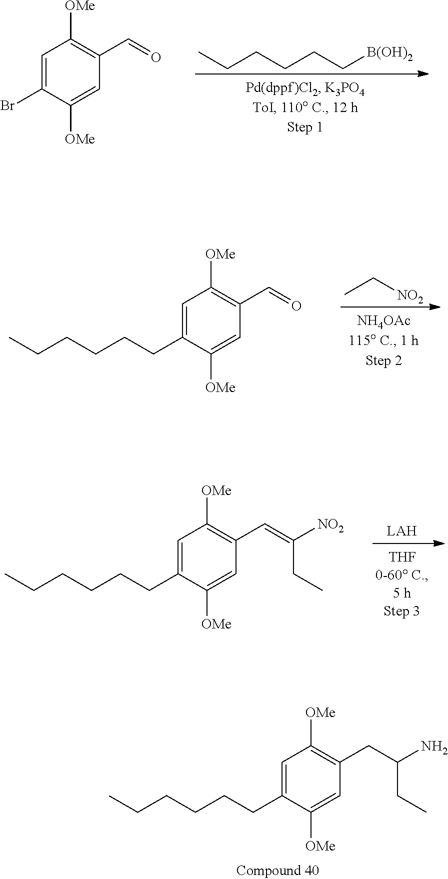

### Step 1: Preparation of 4-hexyl-2,5-dimethoxybenzaldehyde

4-bromo-2,5-dimethoxybenzaldehyde (5 g, 20.4 mmol, 1 eq.), hexylboronic acid (2.65 g, 20.4 mmol, 1 eq.), Pd(dppf)Cl₂ (746 mg, 1.02 mmol, 0.05 eq.), and K₃PO₄ (8.66 g, 40.8 mmol, 2 eq.) in toluene (50 mL) was de-gassed and then warmed to 110° C. for 12 h under N₂. Upon completion, the mixture was filtered, and concentrated and the residue was purified by silica gel chromatography (Petroleum ether/Ethyl acetate=100:1-50:1) to give 4-hexyl-2,5-dimethoxybenzaldehyde (4.2 g, 16.8 mmol, 82% yield) as a yellow solid. 1H NMR (400 MHz, CHLOROFORM-d) δ ppm 10.40 (s, 1H), 7.27 (s, 1H), 6.80 (s, 1H), 3.90 (s, 3H), 3.83 (s, 3H), 2.69-2.60 (m, 2H), 1.67-1.53 (m, 2H), 1.42-1.27 (m, 6H), 0.94-0.85 (m, 3H).

### Step 2: Preparation of (E)-1-hexyl-2,5-dimethoxy-4-(2-nitrobut-1-en-1-yl)benzene

A mixture of 4-hexyl-2,5-dimethoxybenzaldehyde (1.8 g, 7.2 mmol, 1 eq.) and NH₄OAc (1.11 g, 14.4 mmol, 2 eq.) in 1-nitropropane (24.9 g, 279.8 mmol, 25.0 mL, 38.9 eq.) was warmed to 115° C. for 1 h. Upon completion, the mixture was concentrated. The residue was purified by silica gel chromatography (Petroleum ether/Ethyl acetate=30:1) to give (E)-1-hexyl-2,5-dimethoxy-4-(2-nitrobut-1-en-1-yl)benzene (1 g, 3.1 mmol, 43% yield) as a yellow solid. 1H NMR (400 MHz, CHLOROFORM-d) δ ppm 8.26 (s, 1H), 6.78 (d, J=15.6 Hz, 2H), 3.83 (dd, J=1.2, 14.4 Hz, 6H), 2.87 (m, 2H), 2.69-2.58 (m, 1H), 2.69-2.58 (m, 1H), 1.67-1.51 (m, 3H), 1.45-1.25 (m, 11H), 0.97-0.85 (m, 3H).

### Step 3: Preparation of 1-(4-hexyl-2,5-dimethoxyphenyl)butan-2-amine (40)

A solution of (E)-1-hexyl-2,5-dimethoxy-4-(2-nitrobut-1-en-1-yl)benzene (1 g, 3.1 mmol, 1 eq.) in THF (10 mL) was cooled to 0° C. Then LiAlH₄ (473 mg, 12.45 mmol, 4 eq.) was added. The mixture was warmed to 60° C. and stirred at 60° C. for 5 h. Upon completion, the mixture was cooled to 0° C. Then (0.5 mL) H₂O was added dropwise. Then (0.5 mL) 30% aq. NaOH was added dropwise. After stirring until a filterable solid formed, the mixture was filtered, and the filtrate concentrated. The residue was purified by prep-HPLC (column: Phenomenex luna C18 250×50 mm×10 μm; mobile phase: [water (0.04% HCl)-ACN]; B %: 20%-50%, 10 min) to afford 1-(4-hexyl-2,5-dimethoxyphenyl)butan-2-amine (380 mg, 1.15 mmol, 37% yield, 100% purity, HCl) as a white solid. LCMS R_{T}=2.439 min, MS cal.: 293.24, [M+H]⁺=294.2; 1H NMR (400 MHz, DMSO-d6, HCl salt) δ ppm 7.96 (br s, 3H), 6.80 (d, J=10.8 Hz, 2H), 3.73 (s, 6H), 3.33 (s, 12H), 3.28-3.18 (m, 1H), 2.79 (d, J=6.8 Hz, 2H), 2.53 (s, 1H), 2.56-2.52 (m, 1H), 1.57-1.44 (m, 4H), 1.28 (s, 6H), 0.96-0.81 (m, 6H); ¹³C NMR (101 MHz, DMSO-d6, HCl salt) δ ppm 151.01, 150.71, 129.78, 122.16, 114.05, 113.01, 55.85, 52.22, 32.64, 31.12, 29.67, 29.57, 28.66, 24.78, 22.08, 13.97, 9.45.

### Example 38: Preparation of 1-(4-(butylthio)-2,5-dimethoxyphenyl)butan-2-amine (41)

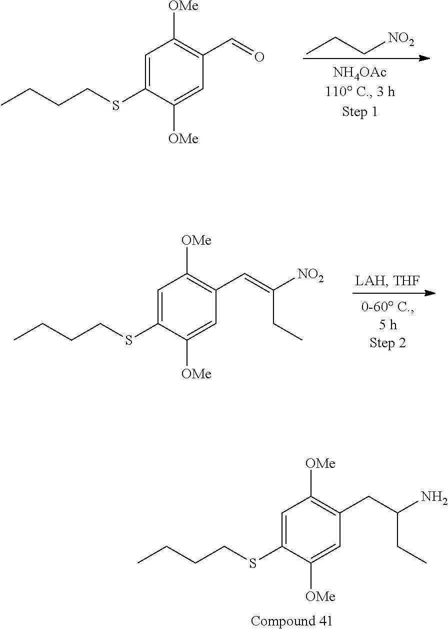

### Step 1: Preparation of (E)-butyl(2,5-dimethoxy-4-(2-nitrobut-1-en-1-yl)phenyl)sulfane

To a solution of 4-(butylthio)-2,5-dimethoxybenzaldehyde (1.5 g, 5.90 mmol, 1 eq.) in 1-nitropropane (10.5 g, 118 mmol, 10.5 mL, 20 eq.) was added NH₄OAc (1.36 g, 17.7 mmol, 3 eq.). The mixture was warmed to 110° C. and stirred for 3 h. Upon completion, the mixture was filtered, and concentrated to give a residue that was purified by column chromatography (SiO₂, Petroleum ether/Ethyl acetate=100/1-30/1) to give (E)-butyl(2,5-dimethoxy-4-(2-nitrobut-1-en-1-yl)phenyl)sulfane (1.0 g, 3.1 mmol, 52% yield) as a yellow oil. ¹H NMR (400 MHz, CHLOROFORM-d) δ ppm 8.24 (s, 1H), 7.27 (s, 1H), 6.79 (s, 1H), 3.89 (s, 3H), 3.87 (s, 3H), 2.95-2.98 (m, 2H), 2.85-2.94 (m, 2H), 1.69-1.75 (m, 2H), 1.50-1.58 (m, 2H), 1.28-1.32 (m, 3H), 0.95-0.99 (m, 3H).

### Step 2: Preparation of 1-(4-(butylthio)-2,5-dimethoxyphenyl)butan-2-amine (41)

To a solution of (E)-butyl(2,5-dimethoxy-4-(2-nitrobut-1-en-1-yl)phenyl)sulfane (1 g, 3.1 mmol, 1 eq.) in THF (20 mL) was added LiAlH₄ (467 mg, 12.3 mmol, 4 eq.) at 0° C. under N₂. The mixture was heated up to 60° C. and stirred for 5 h at 60° C. under N₂. Upon completion, the stirred reaction mixture was quenched by the sequential dropwise addition at 0° C. of water (0.5 mL), a solution of 30% NaOH (0.5 mL), and water (0.5 mL) at 0° C. The mixture was stirred until a smooth solid formed and was filtered, and the filtrate concentrated to give a residue. The residue was purified by prep-HPLC (column: Phenomenex luna C18 (250×70 mm×15 μm); mobile phase: [water (0.05% HCl)-ACN]; B %: 20%-50%, 23 min) to afford 1-(4-(butylthio)-2,5-dimethoxyphenyl)butan-2-amine (0.32 g, 1.1 mmol, 35% yield, HCl) as a white solid. LCMS R_{T}=2.155 min, MS cal.: 297.46, [M+H]⁺=298.1; 1H NMR (400 MHz, DMSO-d6, HCl salt) δ ppm 8.05 (br s, 3H), 6.89 (s, 1H), 6.81 (s, 1H), 3.75 (s, 6H) 3.22-3.25 (m, 1H), 2.89-2.92 (m, 2H), 2.8-2.82 (m, 2H), 1.40-1.57 (m, 6H), 0.87-0.93 (m, 6H); ¹³C NMR (101 MHz, DMSO-d6, HCl salt) δ ppm 151.57, 150.23, 123.97, 121.95, 114.36, 111.01, 56.21, 52.13, 38.64, 32.42, 30.44, 24.85, 21.37, 13.51, 9.44.

### Example 39: Preparation of 1-(4-(4-fluorobutyl)-2,5-dimethoxyphenyl)butan-2-amine (42)

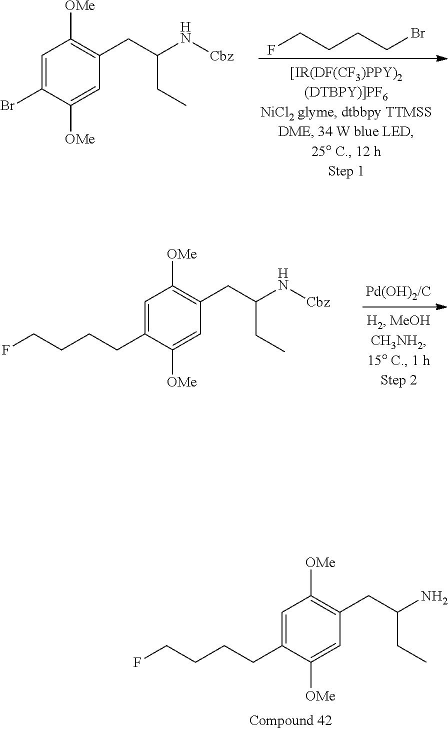 [SMILES: CCC(Cc1cc(OC)c(Br)cc1OC)NC(=O)OCc1ccccc1 ; FCCCCBr ; CCC(Cc1cc(OC)c(CCCCF)cc1OC)NC(=O)OCc1ccccc1 ; CCC(N)Cc1cc(OC)c(CCCCF)cc1OC]

### Step 1: Preparation of benzyl (1-(4-(4-fluorobutyl)-2,5-dimethoxyphenyl)butan-2-yl)carbamate

A stirred mixture of benzyl (1-(4-bromo-2,5-dimethoxyphenyl)butan-2-yl)carbamate (500 mg, 1.18 mmol, 1 eq.), 1-bromo-4-fluorobutane (734 mg, 4.74 mmol, 510 uL, 4 eq.), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (13.3 mg, 11.8 μmol, 0.01 eq.), dichloronickel-1,2-dimethoxyethane (1.3 mg, 5.9 μmol, 0.005 eq.), Na₂CO₃ (250.97 mg, 2.37 mmol, 2 eq.), dtbbpy (1.6 mg, 5.9 μmol, 0.005 eq.), and TTMSS (294 mg, 1.2 mmol, 366 uL, 1 eq.) in DME (4 mL) was degassed and purged with Ar 3 times. Then the mixture was stirred at 25° C. for 10 h under an Ar atmosphere while illuminated with blue light (34 W LED). Upon completion, the reaction mixture was filtered, and the filtrate concentrated. The residue was purified by prep-HPLC ([water (0.04% HCl)-ACN]; B %: 52%-82%, 20 min) to afford benzyl (1-(4-(4-fluorobutyl)-2,5-dimethoxyphenyl)butan-2-yl)carbamate (610 mg, 1.5 mmol, 41% yield) as a white solid. ¹H NMR (400 MHz, CHLOROFORM-d) δ=7.27 (s, 5H), 6.69-6.61 (m, 2H), 5.33-5.29 (m, 2H), 5.06-5.01 (m, 2H), 4.57-4.50 (m, 1H), 3.80-3.69 (m, 5H), 2.79-2.71 (m, 2H), 2.66-2.58 (m, 2H), 1.84-1.61 (m, 6H), 0.99-0.92 (m, 3H).

### Step 2: Preparation of 1-(4-(4-fluorobutyl)-2,5-dimethoxyphenyl)butan-2-amine (42)

To a solution of benzyl (1-(4-(4-fluorobutyl)-2,5-dimethoxyphenyl)butan-2-yl)carbamate (510 mg, 1.22 mmol, 1 eq.) in MeOH (30 mL) and CH₃NH₂ (3 mL, 30% purity) was added Pd(OH)₂ (1 g, 7.12 mmol, 5.8 eq.). The mixture was stirred at 15° C. for 1 h under H₂ (15 psi). Upon completion, the reaction mixture was filtered, and the filtrate concentrated to afford 1-(4-(4-fluorobutyl)-2,5-dimethoxyphenyl)butan-2-amine (300 mg, 1.0 mmol, 83% yield, 96% purity) as a yellow oil. ¹H NMR (400 MHz, CHLOROFORM-d) δ=6.70-6.65 (m, 2H), 4.57-4.51 (m, 1H), 4.45-4.40 (m, 1H), 3.78 (s, 6H), 3.02-2.93 (m, 1H), 2.88-2.79 (m, 1H), 2.68-2.60 (m, 2H), 2.52-2.42 (m, 1H), 1.83-1.74 (m, 2H), 1.71 (br dd, J=3.2, 6.3 Hz, 2H), 1.64-1.48 (m, 1H), 1.48-1.33 (m, 1H), 1.00 (t, J=7.2 Hz, 3H); ¹³C NMR (101 MHz, CHLOROFORM-d) δ=151.56, 151.21, 114.04, 113.06, 84.97, 83.34, 56.09, 53.15, 30.32, 30.12, 29.74, 10.63.

### Example 40: Preparation of 2-amino-3-(2,5-dimethoxy-4-pentylphenyl)propan-1-ol (43i)

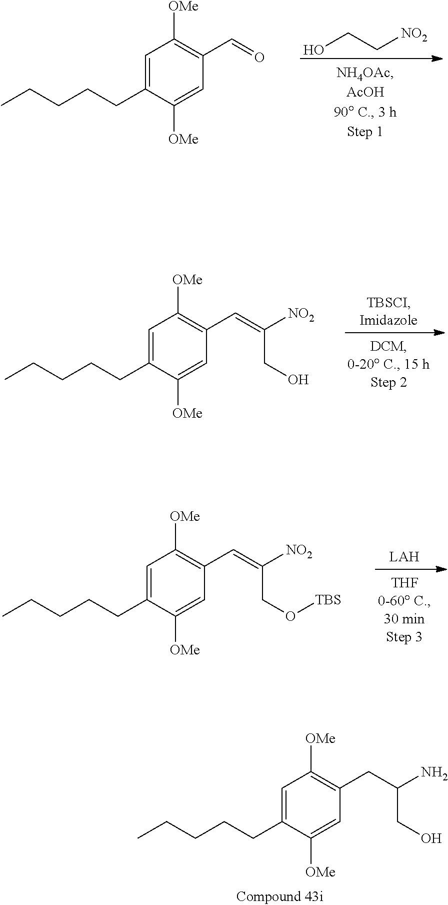

### Step 1: Preparation of (E)-3-(2,5-dimethoxy-4-pentylphenyl)-2-nitroprop-2-en-1-ol

A mixture of 2,5-dimethoxy-4-pentylbenzaldehyde (3 g, 12.7 mmol, 1 eq.) and 2-nitroethanol (8.09 g, 89 mmol, 6.3 mL, 7 eq.) in AcOH (20 mL) was treated with NH₄OAc (1.96 g, 25.4 mmol, 2 eq.) and stirred at 20° C. Then the mixture was warmed and stirred at 90° C. for 2.5 h. The reaction mixture was partially concentrated and poured into ice water (20 mL) and extracted with EtOAc (20 mL×3). The combined with organic layers were washed with brine (20 mL), dried over Na₂SO₄, filtered, and concentrated. The residue was purified by column chromatography (SiO₂, Petroleum ether:Ethyl acetate=10:1) to afford (E)-3-(2,5-dimethoxy-4-pentylphenyl)-2-nitroprop-2-en-1-ol (2 g, 6.47 mmol, 51% yield) as an orange oil. ¹H NMR (400 MHz, CHLOROFORM-d) δ ppm 8.46 (s, 1H), 7.11 (s, 1H), 6.76 (s, 1H), 4.71 (s, 2H), 3.86 (s, 3H), 3.83 (s, 3H), 2.68-2.60 (m, 2H), 1.65-1.55 (m, 2H), 1.42-1.32 (m, 4H), 0.92 (br t, J=6.8 Hz, 3H).

### Step 2: Preparation of (E)-tert-butyl((3-(2,5-dimethoxy-4-pentylphenyl)-2-nitroallyl)oxy)dimethylsilane

To a solution of (E)-3-(2,5-dimethoxy-4-pentylphenyl)-2-nitroprop-2-en-1-ol (1.5 g, 4.9 mmol, 1 eq.) and imidazole (660 mg, 9.7 mmol, 2 eq.) in DCM (20 mL) was added TBSCl (877 mg, 5.82 mmol, 713 uL, 1.2 eq.) at 0° C. After addition, the mixture was stirred at 20° C. for 15 h. The mixture was filtered, to remove the insoluble solids. The filtrate was concentrated in vacuo to give a residue that was purified by column chromatography on silica gel (Petroleum ether:Ethyl acetate=10:1) to give (E)-tert-butyl((3-(2,5-dimethoxy-4-pentylphenyl)-2-nitroallyl)oxy)dimethylsilane (0.8 g, 1.9 mmol, 39% yield) as a yellow oil. ¹H NMR (400 MHz, CHLOROFORM-d) δ ppm 8.48 (s, 1H), 7.28 (s, 1H), 6.75 (s, 1H), 4.80 (s, 2H), 3.85 (s, 3H), 3.82 (s, 3H), 2.68-2.60 (m, 2H), 1.66-1.57 (m, 2H), 1.41-1.32 (m, 4H), 0.92 (s, 9H), 0.95-0.89 (m, 3H), 0.16 (s, 6H).

### Step 3: Preparation of 2-amino-3-(2,5-dimethoxy-4-pentylphenyl)propan-1-ol (43i)

A solution of (E)-tert-butyl((3-(2,5-dimethoxy-4-pentylphenyl)-2-nitroallyl)oxy)dimethylsilane (1.1 g, 2.6 mmol, 1 eq.) in THF (10 mL) was degassed and purged with N₂ 3 times. To this stirred solution cooled to 0° C. was added LiAlH₄ (394 mg, 10.4 mmol, 4 eq.), and the stirred mixture was warmed to 60° C. for 30 min under a N₂ atmosphere. After cooling, the mixture was quenched sequentially dropwise with (0.4 mL) water, (0.4 mL) 15% NaOH solution, and (0.4 mL) water. After stirring to a smooth granular mixture, the solids were filtered, and the filtrate was concentrated. The residue was purified by prep-HPLC ([A: 10 mM NH₄HCO₃ in H₂O; B: ACN] B %: 25%-45%, 8 min) to afford 2-amino-3-(2,5-dimethoxy-4-pentylphenyl) propan-1-ol (300 mg, 1.05 mmol, 40% yield, 98% purity) as an off white solid. LCMS R_{T}=2.116 min, MS cal.: 281.20, [M+H]⁺=282.1; 1H NMR (400 MHz, CHLOROFORM-d) δ ppm 6.69 (s, 1H), 6.65 (s, 1H), 3.79 (s, 3H), 3.78 (s, 3H), 3.54 (dd, J=4.0, 10.8 Hz, 1H), 3.36 (dd, J=6.4, 10.8 Hz, 1H), 3.15-3.05 (m, 1H), 2.76 (dd, J=6.0, 13.3 Hz, 1H), 2.65-2.54 (m, 3H), 1.85 (br s, 3H), 1.63-1.52 (m, 2H), 1.41-1.29 (m, 4H), 0.96-0.87 (m, 3H); ¹³C NMR (101 MHz, CHLOROFORM-d) δ ppm 151.39, 151.34, 130.46, 124.55, 113.99, 113.01, 66.30, 56.17, 56.12, 53.36, 35.15, 31.84, 30.22, 29.85, 22.59, 14.08.

### Example 41: Preparation of 2-(3,5-dimethoxy-4-(pentylthio)phenyl)ethanamine (44)

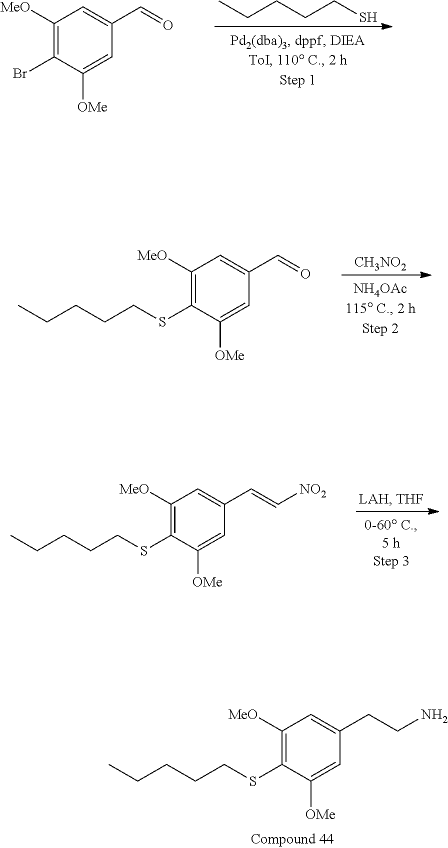

### Step 1: Preparation of 3,5-dimethoxy-4-(pentylthio)benzaldehyde

To a mixture of 4-bromo-3,5-dimethoxybenzaldehyde (3 g, 12.2 mmol, 1 eq.) and pentane-1-thiol (1.66 g, 15.9 mmol, 1.3 eq.) in toluene (30 mL) was added DIEA (4.75 g, 36.7 mmol, 6.40 mL, 3 eq.), DPPF (679 mg, 1.22 mmol, 0.1 eq.), and Pd₂(dba)₃ (1.12 g, 1.22 mmol, 0.1 eq.) in one portion at 20° C. under N₂. The mixture was warmed and stirred at 110° C. for 2 h. Upon completion, the reaction mixture was cooled to rt then filtered, and the filtrate was concentrated. The residue was purified by silica gel chromatography (Petroleum Ether:EA=100:1-50:1) to afford 3,5-dimethoxy-4-(pentylthio)benzaldehyde (3 g, 11.2 mmol, 91% yield) as a brown oil. ¹H NMR (400 MHz, CHLOROFORM-d) δ=9.88 (s, 1H), 7.05-7.00 (m, 2H), 3.92 (s, 6H), 2.90 (t, J=7.2 Hz, 2H), 1.52-1.42 (m, 2H), 1.39-1.17 (m, 4H), 0.81 (t, J=7.2 Hz, 3H)

### Step 2: Preparation of (E)-(2,6-dimethoxy-4-(2-nitrovinyl)phenyl)(pentyl)sulfane

A mixture of 3,5-dimethoxy-4-(pentylthio)benzaldehyde (3 g, 11.2 mmol, 1 eq.) and NH₄OAc (1.72 g, 22.4 mmol, 2 eq.) in nitromethane (20.5 g, 335 mmol, 30 eq.) was stirred at 115° C. for 2 h. Upon completion, the reaction was cooled and concentrated. The residue was purified by silica gel chromatography (Petroleum Ether:EA=80:1-60:1) to afford (E)-(2,6-dimethoxy-4-(2-nitrovinyl)phenyl)(pentyl)sulfane (1.5 g, 4.82 mmol, 43% yield) as a yellow oil. 1H NMR (400 MHz, CHLOROFORM-d) δ=8.00-7.92 (m, 1H), 7.60 (d, J=13.6 Hz, 1H), 6.71 (s, 2H), 3.99-3.91 (m, 6H), 2.97-2.85 (m, 2H), 1.58-1.47 (m, 2H), 1.41-1.24 (m, 4H), 0.91-0.82 (m, 3H)

### Step 3: Preparation of 2-(3,5-dimethoxy-4-(pentylthio)phenyl)ethanamine (44)

A solution of LiAlH₄ (585 mg, 15.4 mmol, 6 eq.) in THF (60 mL) was stirred at 0° C. Then (E)-(2,6-dimethoxy-4-(2-nitrovinyl)phenyl)(pentyl)sulfane (800 mg, 2.57 mmol, 1 eq.) was added as a solution in THF (5 mL). The mixture was warmed and stirred at 60° C. for 5 h. Upon completion, the mixture was cooled to 0° C. To the reaction mixture was added sequentially dropwise (0.58 mL) H₂O, (0.58 mL) 30% aq. NaOH, then (0.58 mL) H₂O. After stirring until a smooth granular solid formed, the reaction mixture was filtered, and the filtrate was concentrated. The residue was purified by prep-HPLC ([water (0.04% HCl)-ACN]; B %: 10%-40%, 10 min) to afford 2-(3,5-dimethoxy-4-(pentylthio)phenyl)ethanamine (170 mg, 599 μmol, 12% yield) as a white solid. LCMS R_{T}=2.077 min, MS cal.: 283.42, [M+H]⁺=284.1; 1H NMR (400 MHz, CHLOROFORM-d, HCl salt) δ=8.37 (br s, 3H), 6.48 (s, 2H), 3.89 (s, 6H), 3.37-3.20 (m, 2H), 3.16-3.03 (m, 2H), 2.83-2.72 (m, 2H), 1.54-1.43 (m, 2H), 1.40-1.24 (m, 4H), 0.91-0.82 (m, 3H); ¹³C NMR (101 MHz, CHLOROFORM-d, HCl salt) δ=161.26, 137.41, 104.87, 56.37, 40.83, 34.41, 34.33, 34.22, 34.07, 30.93, 29.32, 22.28, 13.99.

### Example 42: Preparation of 2-(3,5-dimethoxy-4-((4-methylpentyl)thio)phenyl)ethanamine (45)

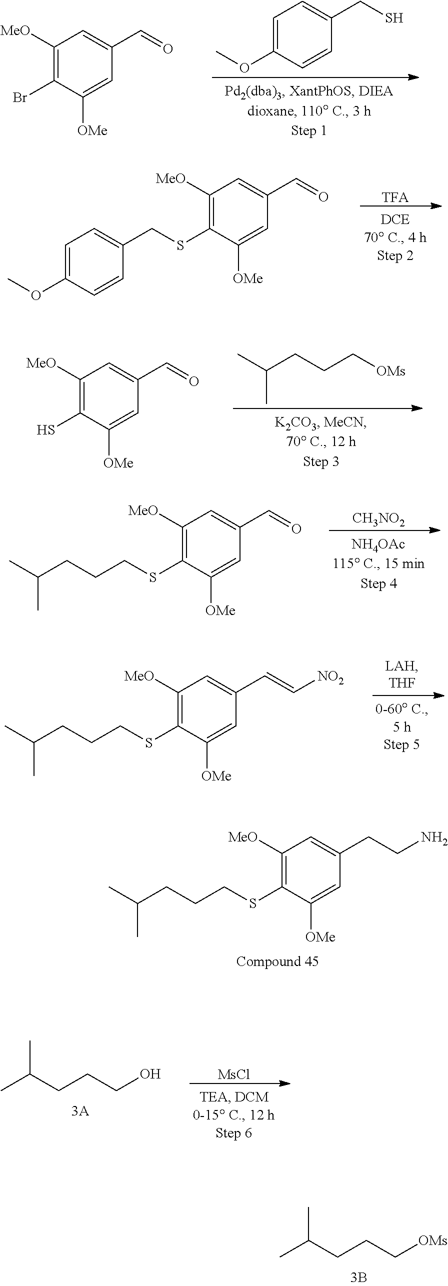

### Step 1: Preparation of 3,5-dimethoxy-4-((4-methoxybenzyl)thio)benzaldehyde

To a mixture of (4-methoxyphenyl)methanethiol (5.66 g, 36.7 mmol, 5.1 mL, 2 eq.), 4-bromo-3,5-dimethoxybenzaldehyde (4.5 g, 18.4 mmol, 1 eq.), and DIEA (4.75 g, 36.7 mmol, 6.4 mL, 2 eq.) in dioxane (100 mL) was added Xantphos (1.06 g, 1.84 mmol, 0.1 eq.) and Pd₂(dba)₃ (1.68 g, 1.84 mmol, 0.1 eq.) in one portion at 15° C. under N₂. The mixture was heated to 110° C. and stirred for 3 h. Upon completion, the reaction mixture was cooled and filtered, and the filtrate was concentrated. The residue was purified by prep-TLC (SiO₂, PE:EA=10:1-0:1) to give 3,5-dimethoxy-4-((4-methoxybenzyl)thio)benzaldehyde (4.5 g, 14.1 mmol, 77% yield) as a yellow solid. ¹H NMR (400 MHz, CHLOROFORM-d) δ=9.91 (s, 1H), 7.17-7.10 (m, 2H), 7.03 (s, 2H), 6.78-6.70 (m, 2H), 4.10 (s, 2H), 3.92 (s, 6H), 3.76 (s, 3H).

### Step 2: Preparation of 4-mercapto-3,5-dimethoxybenzaldehyde

To a solution of 3,5-dimethoxy-4-((4-methoxybenzyl)thio)benzaldehyde (2 g, 6.28 mmol, 1 eq.) in DCE (10 mL) was added TFA (14.3 g, 125.6 mmol, 9.3 mL, 20 eq.) at 0° C. The mixture was warmed and stirred at 70° C. for 1 h. Upon completion, the solvent was removed. The crude product 4-mercapto-3,5-dimethoxybenzaldehyde (2.07 g, crude), a black solid, was used in the next step without further purification. ¹H NMR (400 MHz, CHLOROFORM-d) δ=9.96 (s, 1H), 7.05 (s, 2H), 3.80 (s, 6H).

### Step 3: Preparation of 3,5-dimethoxy-4-((4-methylpentyl)thio)benzaldehyde

To a solution of crude 4-mercapto-3,5-dimethoxybenzaldehyde (1.87 g, 9.43 mmol, 1 eq.) in DMF (100 mL) was added 4-methylpentyl methanesulfonate (3.40 g, 18.9 mmol, 2 eq.) and K₂CO₃ (11.7 g, 84.9 mmol, 9 eq.) under a N₂ atmosphere. The mixture was warmed and stirred at 100° C. for 12 h. Upon completion, the reaction mixture was cooled and diluted with H₂O (300 mL) and extracted with EA (300 mL×3). The combined organic layer was washed with brine (300 mL×2), dried over Na₂SO₄, filtered, and concentrated to give a residue. The residue was purified by silica gel chromatography (SiO₂, Petroleum ether/Ethyl acetate=15/1-0/1) to give 3,5-dimethoxy-4-((4-methylpentyl)thio)benzaldehyde (620 mg) as a yellow oil. 1H NMR (400 MHz, CHLOROFORM-d) δ=9.94 (s, 1H), 7.08 (s, 2H), 4.02-3.92 (m, 6H), 3.68-3.61 (m, 8H), 2.96-2.89 (m, 2H), 1.63-1.54 (m, 14H), 1.32-1.19 (m, 15H), 0.90 (d, J=6.4 Hz, 28H), 0.84 (d, J=6.4 Hz, 5H).

### Step 4: Preparation of (E)-(2,6-dimethoxy-4-(2-nitrovinyl)phenyl)(4-methylpentyl)sulfane

To a mixture of 3,5-dimethoxy-4-((4-methylpentyl)thio)benzaldehyde (613 mg, 2.17 mmol, 1 eq.) in nitromethane (6.62 g, 108.5 mmol, 5.9 mL, 50 eq.) was added NH₄OAc (335 mg, 4.34 mmol, 2 eq.) in one portion at 20° C. under N₂. The mixture was warmed and stirred at 115° C. for 15 min, then cooled and concentrated to give a residue. This was purified by column chromatography (SiO₂, Petroleum ether/Ethyl acetate=15/1-0:1) to give (E)-(2,6-dimethoxy-4-(2-nitrovinyl)phenyl)(4-methylpentyl)sulfane (113 mg, 313 μmol, 14% yield, crude) as a yellow solid. 1H NMR (400 MHz, CHLOROFORM-d) δ=7.97 (br d, J=14.0 Hz, 1H), 7.64-7.61 (m, 1H), 7.43 (td, J=1.6, 2.8 Hz, 1H), 6.71 (s, 2H), 3.94 (s, 6H), 3.78 (br s, 2H), 2.96-2.84 (m, 3H), 1.60-1.53 (m, 5H), 1.26 (s, 3H), 0.85 (d, J=6.4 Hz, 6H).

### Step 5: Preparation of 2-(3,5-dimethoxy-4-((4-methylpentyl)thio)phenyl)ethanamine (45)

A dispersion of LiAlH₄ (660 mg, 17.4 mmol, 20 eq.) was stirred in THF (20 mL) and warmed to 80° C. under N₂. Then (E)-(2,6-dimethoxy-4-(2-nitrovinyl)phenyl)(4-methylpentyl)sulfane (283 mg, 870 μmol, 1 eq.) in THF (5 mL) was added dropwise. The mixture was stirred at 80° C. for 15 min. Upon completion, the reaction mixture was cooled and quenched dropwise with H₂O (0.6 mL) and aq. NaOH (3M) (0.6 mL), at 0° C. After stirring to a smooth dispersion, the solids were filtered, and the filtrate was concentrated to give a crude product. The residue was purified by prep-HPLC (column: Phenomenex luna C18 80×40 mm×3 μm; mobile phase: [water (0.04% HCl)-ACN]; B %: 25%-33%, 7 min) to give desired compound 2-(3,5-dimethoxy-4-((4-methylpentyl)thio)phenyl)ethanamine (18 mg, 69 μmol, 8% yield, 89% purity) as a white solid. LCMS R_{T}=2.163 min, MS cal.: 297.1, [M+H]⁺=298.1; 1H NMR (400 MHz, DMSO-d6, HCl salt) δ=8.10-7.79 (m, 3H), 6.60-6.51 (m, 2H), 3.87-3.76 (m, 6H), 3.13-3.00 (m, 2H), 2.92-2.79 (m, 2H), 2.67 (t, J=7.2 Hz, 2H), 1.50-1.40 (m, 1H), 1.40-1.30 (m, 2H), 1.27-1.18 (m, 2H), 0.80 (d, J=6.8 Hz, 6H); ¹³C NMR (101 MHz, DMSO-d6, HCl salt) δ=161.00, 139.52, 105.36, 56.43, 40.66, 37.68, 33.96, 33.78, 27.56, 27.35, 22.90.

### Step 6: Preparation of 4-methylpentyl methanesulfonate

To a mixture of 4-methylpentan-1-ol (5 g, 49 mmol, 6.17 mL, 1 eq.) and Et₃N (9.9 g, 98 mmol, 13.6 mL, 2 eq.) in DCM (100 mL) was added MsCl (8.41 g, 73.4 mmol, 5.7 mL, 1.5 eq.) dropwise at 0° C. under N₂. The mixture was stirred at 15° C. for 12 h, then the reaction mixture was quenched by addition of H₂O (50 mL) at 15° C. The product was extracted with DCM (100 mL×3). The combined organic layer was washed with brine 300 mL (100 mL×3), dried over Na₂SO₄, filtered, and concentrated to give 4-methylpentyl methanesulfonate (7.1 g, 39.4 mmol, 81% yield) as a yellow oil. 1H NMR (400 MHz, CHLOROFORM-d) δ=4.21 (t, J=6.8 Hz, 2H), 3.01 (s, 3H), 1.88-1.68 (m, 2H), 1.65-1.53 (m, 1H), 1.41-1.22 (m, 2H), 0.91 (d, J=6.8 Hz, 6H).

### Example 43: Preparation of 2-(4-(isopentylthio)-3,5-dimethoxyphenyl)ethanamine (46)

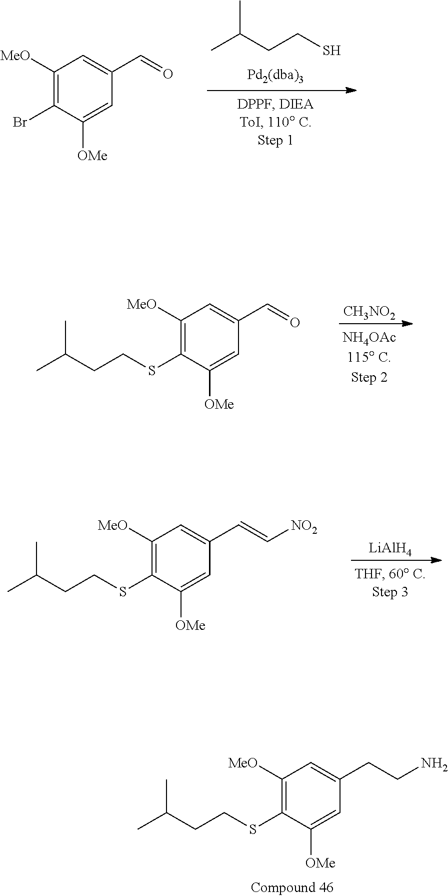

### Step 1: Preparation of 4-(isopentylthio)-3,5-dimethoxybenzaldehyde

To a mixture of 4-bromo-3,5-dimethoxybenzaldehyde (2 g, 8.16 mmol, 1 eq.) and 3-methylbutane-1-thiol (1.11 g, 10.6 mmol, 1.32 mL, 1.3 eq.) in toluene (20 mL) was added DIEA (3.16 g, 24.5 mmol, 4.3 mL, 3 eq.), DPPF (452 mg, 817 μmol, 0.1 eq.), and Pd₂(dba)₃ (747 mg, 816 μmol, 0.1 eq.) in one portion at 20° C. under N₂. The mixture was warmed and stirred at 110° C. for 2 h. Upon completion, the mixture was filtered, and concentrated to give a residue. The residue was purified by column chromatography (SiO₂, Petroleum ether: Ethyl acetate=100:1-10:1) to give 4-(isopentylthio)-3,5-dimethoxybenzaldehyde (1.6 g, 5.96 mmol, 73% yield) as a brown oil. ¹H NMR (400 MHz, CHLOROFORM-d) δ ppm 9.94 (s, 1H), 7.20 (s, 2H), 3.89 (s, 6H), 2.90 (t, J=7.6 Hz, 2H), 1.58-1.62 (m, 1H), 1.24-1.29 (m, 2H), 0.82 (d, J=6.8 Hz, 6H).

### Step 2: Preparation of (E)-(2,6-dimethoxy-4-(2-nitrovinyl)phenyl)(isopentyl)sulfane

A mixture of 4-(isopentylthio)-3,5-dimethoxybenzaldehyde (1.8 g, 6.71 mmol, 1 eq.) and NH₄OAc (1.03 g, 13.4 mmol, 2 eq.) in nitromethane (8.19 g, 134 mmol, 7.25 mL, 20 eq.) was stirred and warm d to 115° C. for 1 h. Upon completion, the solvent was removed to give a residue. The residue w as purified by column chromatography (SiO₂, Petroleum ether:Ethyl acetate=100:1-10:1) to give (E)-(2,6-dimethoxy-4-(2-nitrovinyl)phenyl)(isopentyl)sulfane (1.17 g, 3.76 mmol, 56% yield) as a bronze solid. ¹H NMR (400 MHz, CHLOROFORM-d) δ ppm 7.97 (d, J=8.0 Hz, 1H), 7.63 (d, J=8.0 Hz, 1H), 6.71 (s, 2H), 3.92 (s, 6H), 2.92 (t, J=8.0 Hz, 2H), 1.65-1.74 (m, 1H), 1.37-1.43 (m, 2H), 0.87 (d, J=6.8 Hz, 6H).

### Step 3: Preparation of 2-(4-(isopentylthio)-3,5-dimethoxyphenyl)ethanamine (46)

A solution of (E)-(2,6-dimethoxy-4-(2-nitrovinyl)phenyl)(isopentyl)sulfane (1.17 g, 3.76 mmol, 1 eq.) in THF (20 mL) was cooled to 0° C. To the resulting solution was added LiAlH₄ (570 mg, 15 mmol, 4 eq.) in one portion at 0° C. under N₂. The mixture was stirred at 0° C. for 5 min, then heated to 60° C. and stirred for 5 h. Upon completion, the mixture was cooled to 0° C. Then (0.6 mL) H₂O was added dropwise and (0.6 mL) 30% aq. NaOH was added dropwise. The solids that formed were filtered, and the filtrate concentrated. The residue was purified by prep-HPLC (HCl condition) to give 2-(4-(isopentylthio)-3,5-dimethoxyphenyl)ethanamine (210 mg, 657 μmol, 18% yield, HCl) as a white solid. 1H NMR (400 MHz, DMSO-d₆, HCl salt) δ ppm 8.07 (br s, 3H), 6.58 (s, 2H), 3.79 (s, 6H), 3.07 (s, 2H), 2.93-2.82 (m, 2H), 2.73-2.66 (m, 2H), 1.65 (m, 1H), 1.31-1.20 (m, 2H), 0.81 (d, J=6.4 Hz, 6H); ¹³C NMR (101 MHz, DMSO-d₆, HCl salt) δ ppm 160.45, 139.06, 107.92, 104.95, 55.97, 38.16, 33.41, 31.04, 26.52, 22.13.

### Example 44: Preparation of 2-(4-((4-fluorobutyl)thio)-3,5-dimethoxyphenyl)ethanamine (47)

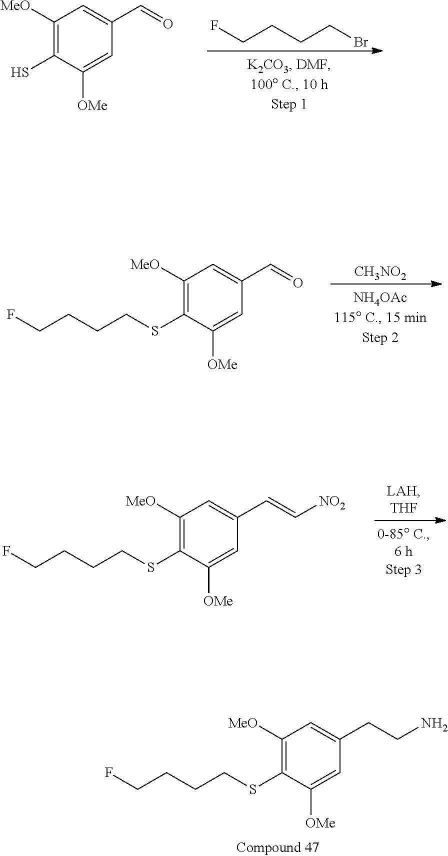

### Step 1: Preparation of 4-((4-fluorobutyl)thio)-3,5-dimethoxybenzaldehyde

To a mixture of 4-mercapto-3,5-dimethoxybenzaldehyde (1.5 g, 7.57 mmol, 1 eq.) and 1-bromo-4-fluorobutane (1.76 g, 11.4 mmol, 1.22 mL, 1.5 eq.) in DMF (100 mL) was added K₂CO₃ (10.46 g, 75.7 mmol, 10 eq.) in one portion at 25° C. under N₂. The mixture was heated to 100° C. and stirred for 12 h. Upon completion, the reaction mixture was diluted with H₂O (200 mL) and extracted with EA (200 mL×3). The combined organic layer was washed with brine (200 mL×2), dried over Na₂SO₄, filtered, and concentrated to give a residue. The residue was purified by column chromatography (SiO₂, Petroleum ether/Ethyl acetate=20/1-5:1) to give 4-((4-fluorobutyl)thio)-3,5-dimethoxybenzaldehyde (176 mg, 582 μmol, 8% yield, 90% purity) as a yellow oil. ¹H NMR (400 MHz, CHLOROFORM-d) δ=9.96 (s, 1H), 7.13-7.04 (m, 2H), 4.49 (t, J=6.0 Hz, 1H), 4.40-4.34 (m, 1H), 4.16-4.10 (m, 1H), 3.98 (s, 6H), 3.04-2.96 (m, 2H), 2.94-2.88 (m, 1H), 1.90-1.74 (m, 5H), 1.69-1.60 (m, 2H), 1.56 (br s, 2H), 1.33-1.21 (m, 2H).

### Step 2: Preparation of (E)-(2,6-dimethoxy-4-(2-nitrovinyl)phenyl)(4-fluorobutyl)sulfane

To a mixture of 4-((4-fluorobutyl)thio)-3,5-dimethoxybenzaldehyde (200 mg, 734 μmol, 1 eq.) in nitromethane (2.24 g, 36.7 mmol, 2 mL, 50 eq.) was added NH₄OAc (113 mg, 1.5 mmol, 2 eq.) in one portion at 20° C. under N₂. The mixture was warmed and stirred at 115° C. for 15 min. Upon completion, the solvent was removed. The residue was purified by prep-TLC (SiO₂, PE:EA=5:1) to give (E)-(2,6-dimethoxy-4-(2-nitrovinyl)phenyl)(4-fluorobutyl)sulfane (124 mg, 354 μmol, 48% yield, 90% purity) as a yellow solid. ¹H NMR (400 MHz, CHLOROFORM-d) δ=7.97-7.84 (m, 1H), 7.52 (d, J=13.7 Hz, 1H), 6.64 (s, 2H), 4.44-4.40 (m, 1H), 4.31-4.27 (m, 1H), 4.10-4.03 (m, 2H), 3.90-3.84 (m, 6H), 2.93-2.84 (m, 2H), 1.76-1.65 (m, 4H), 1.60-1.53 (m, 2H).

### Step 3: Preparation of 2-(4-((4-fluorobutyl)thio)-3,5-dimethoxyphenyl)ethanamine (47)

LiAlH₄ (241 mg, 6.3 mmol, 20 eq.) was carefully added to THF (15 mL) under N₂ then warmed to 80° C. A solution of (E)-(2,6-dimethoxy-4-(2-nitrovinyl)phenyl)(4-fluorobutyl)sulfane (100 mg, 317 μmol, 1 eq.) in THF (2 mL) was added dropwise to the LiAlH₄ solution. The mixture was stirred at 85° C. for 6 h. Upon completion, the reaction was cooled to 0° C. To the stirred reaction mixture was sequentially added H₂O (0.3 mL) dropwise at 0° C., followed by 30% NaOH (0.3 mL). After a smooth dispersion formed, the reaction mixture was filtered, and the filtrate was concentrated. The residue was purified by prep-HPLC (column: Welch Xtimate C18 100×25 mm s 3 um; mobile phase: [water (0.04% HCl)-ACN]; B %: 1%-20%, 8 min) to provide the desired compound 2-(4-((4-fluorobutyl)thio)-3,5-dimethoxyphenyl)ethanamine (8.5 mg, 33 μmol, 11% yield, 96% purity) as a white solid. LCMS R_{T}=1.827 min, MS cal.: 287.14, [M+H]⁺=288.1; 1H NMR (400 MHz, DMSO-d6, HCl salt) δ=8.04-7.87 (m, 3H), 6.58 (s, 2H), 4.45 (br t, J=6.0 Hz, 1H), 4.33 (br t, J=6.0 Hz, 1H), 3.80 (s, 6H), 3.13-3.03 (m, 2H), 2.91-2.81 (m, 2H), 2.76-2.68 (m, 2H), 1.79-1.72 (m, 1H), 1.70-1.64 (m, 1H), 1.42 (td, J=7.2, 14.7 Hz, 2H); ¹³C NMR (101 MHz, DMSO-d6, HCl salt) δ=161.07, 139.71, 107.94, 105.43, 84.76, 83.15, 56.47, 39.13, 33.95, 33.12, 29.27, 29.07, 25.39.

### Example 45: Preparation of 4-((4-(2-aminoethyl)-2,6-dimethoxyphenyl)thio)butan-1-ol (47i)

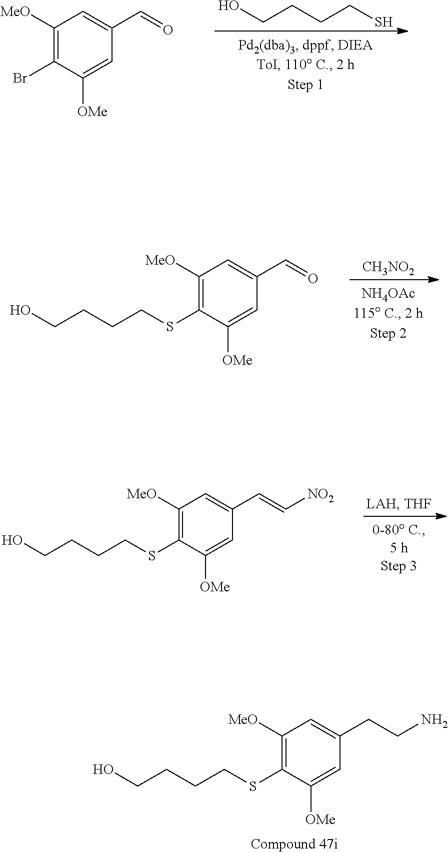

### Step 1: Preparation of 4-((4-hydroxybutyl)thio)-3,5-dimethoxybenzaldehyde

A mixture of 4-bromo-3,5-dimethoxybenzaldehyde (3 g, 12.2 mmol, 1 eq.), 4-sulfanylbutan-1-ol (1.7 g, 15.9 mmol, 1.3 eq.), DPPF (679 mg, 1.22 mmol, 0.1 eq.), DIEA (4.75 g, 36.7 mmol, 6.40 mL, 3 eq.), and Pd₂(dba)₃ (1.12 g, 1.22 mmol, 0.1 eq.) in toluene (20 mL) was degassed and purged with N₂ 3 times. The stirred mixture was warmed and stirred at 110° C. for 2 h under a N₂ atmosphere. Upon completion, the reaction mixture was filtered, and the filtrate was concentrated. The residue was treated with water (100 mL) and extracted with ethyl acetate (100 mL×2). The combined organic phase was washed with brine (100 mL×1), dried over anhydrous Na₂SO₄, filtered, and concentrated. The residue was purified by column chromatography (SiO₂, PE/EA=20/1-0/1) to afford 4-((4-hydroxybutyl)thio)-3,5-dimethoxybenzaldehyde (2.8 g, 10.4 mmol, 85% yield) as a yellow oil. ¹H NMR (400 MHz, CHLOROFORM-d) δ=9.93 (s, 1H), 7.07 (s, 2H), 3.96 (s, 6H), 3.63 (t, J=6.4 Hz, 2H), 2.98 (t, J=7.2 Hz, 2H), 1.72-1.57 (m, 5H)

### Step 2: Preparation of (E)-4-((2,6-demethoxy-4-(2-nitrovinyl)phenyl)thio)butan-1-ol

To a solution of 4-((4-hydroxybutyl)thio)-3,5-dimethoxybenzaldehyde (3.13 g, 11.6 mmol, 1 eq.) in CH₃NO₂ (15 mL) was added NH₄OAc (1.78 g, 23.2 mmol, 2 eq.). The mixture was stirred at 115° C. for 15 min. Upon completion, the solvent was removed to give a residue. The residue was purified by column chromatography (SiO₂, PE/EA=20/1-0/1) to afford (E)-4-((2,6-dimethoxy-4-(2-nitrovinyl)phenyl)thio)butan-1-ol (1.09 g, 3.5 mmol, 30% yield) as a yellow solid. ¹H NMR (400 MHz, CHLOROFORM-d) δ 7.96 (d, J=13.6 Hz, 1H), 7.60 (d, J=13.6 Hz, 1H), 6.71 (s, 2H), 3.94 (s, 6H), 3.65 (t, J=6.4 Hz, 2H), 2.95 (t, J=7.2 Hz, 2H), 1.72-1.65 (m, 3H), 1.65-1.57 (m, 3H)

### Step 3: Preparation of 4-((4-(2-aminoethyl)-2,6-dimethoxyphenyl)thio)butan-1-ol (47i)

To a solution of (E)-4-((2,6-dimethoxy-4-(2-nitrovinyl)phenyl)thio)butan-1-ol (300 mg, 957 μmol, 1 eq.) in THF (15 mL) was added LiAlH₄ (218 mg, 5.7 mmol, 6 eq.) at 0° C. The mixture was warmed to and stirred at 80° C. for 5 h. Upon completion, the mixture was cooled to 0° C. To the reaction mixture was added water dropwise (0.2 mL) and it was stirred for 5 min. Then (0.2 mL) 30% aq. NaOH was added dropwise and stirred. After stirring the resulting dispersion was filtered, and the filtrate was concentrated. The residue was purified by prep-HPLC (column: Phenomenex Gemini-NX C18 75×30 mm×3 μm; mobile phase: [water (10 mM NH₄HCO₃)-ACN]; B %: 5%-30%, 8 min) to afford 4-((4-(2-aminoethyl)-2,6-dimethoxyphenyl)thio)butan-1-ol (25 mg) as a colorless oil. ¹H NMR (400 MHz, METHANOL-d4) δ 6.54 (s, 2H), 4.86 (s, 6H), 3.87-3.83 (m, 6H), 3.50 (t, J=6.4 Hz, 2H), 2.94-2.88 (m, 2H), 2.79-2.71 (m, 4H), 1.67-1.56 (m, 2H), 1.54-1.43 (m, 2H); ¹³C NMR (101 MHz, CHLOROFORM-d) δ 160.80, 142.93, 107.23, 105.32, 67.50, 60.76, 56.35, 43.87, 33.62, 31.96, 26.27, 25.61.

### Example 46: Preparation of 1-(4-(butylthio)-3,5-dimethoxyphenyl)butan-2-amine (48)

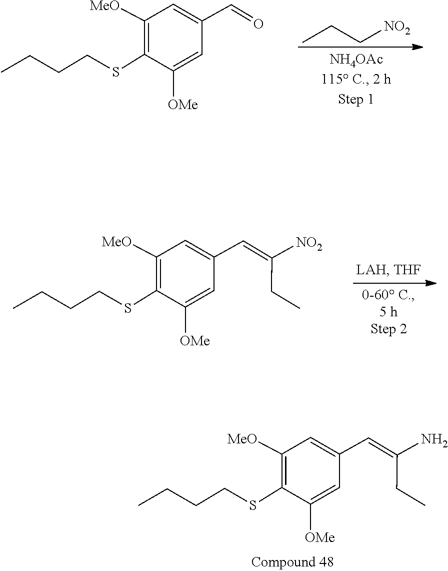

### Step 1: Preparation of (E)-butyl(2,6-dimethoxy-4-(2-nitrobut-1-en-1-yl)phenyl)sulfane

A stirred mixture of 4-(butylthio)-3,5-dimethoxybenzaldehyde (1.8 g, 7.1 mmol, 1 eq.) and NH₄OAc (1.09 g, 14.2 mmol, 2 eq.) in 1-nitropropane (14 mL) was degassed and purged with N₂ 3 times, and then the mixture was stirred at 100° C. for 2 h under a N₂ atmosphere. Upon completion, the solvent was removed. The residue was purified by column chromatography to afford (E)-butyl(2,6-dimethoxy-4-(2-nitrobut-1-en-1-yl)phenyl)sulfane (0.7 g, 2.0 mmol, 29% yield, 95% purity) as a yellow oil. ¹H NMR (400 MHz, CHLOROFORM-d) δ ppm 7.99 (s, 1H), 6.61 (s, 2H), 3.92 (s, 6H), 2.92-2.87 (m, 4H), 1.53-1.40 (m, 4H), 1.32 (t, J=7.6 Hz, 3H), 0.88 (t, J=7.6 Hz, 3H).

### Step 2: Preparation of 1-(4-(butylthio)-3,5-dimethoxyphenyl)butan-2-amine (48)

A stirred solution of (E)-butyl(2,6-dimethoxy-4-(2-nitrobut-1-en-1-yl)phenyl)sulfane (0.7 g, 2.15 mmol, 1 eq.) in THF (10 mL) was degassed and purged with N₂ 3 times, and then LiAlH₄ (327 mg, 8.6 mmol, 4 eq.) was added at 0° C. The mixture was then warmed to 60° C. for 5 h under a N₂ atmosphere. Upon completion, the reaction mixture was quenched by dropwise addition of water (0.3 mL) at 0° C. and then dropwise a solution of 30% aq. NaOH (0.4 mL) at 0° C. After stirring, the resulting dispersion was filtered, and the filtrate was concentrated to give a residue. The residue was purified by prep-HPLC (column: Phenomenex luna C18 250×50 mm×10 μm; mobile phase: [water (0.04% HCl)-ACN]; B %: 25%-55%, 10 min) to afford 1-(4-(butylthio)-3,5-dimethoxyphenyl)butan-2-amine (280 mg, 815 μmol, 38% yield, 97% purity, HCl) as an off-white solid. LCMS R_{T}=2.043 min, MS cal.: 297.46, [M+H]⁺=298.1; 1H NMR (400 MHz, DMSO-d₆, HCl salt) δ ppm 8.08 (br s, 3H), 6.60 (s, 2H), 3.79 (s, 6H), 3.33 (s, 3H), 2.97-2.75 (m, 2H), 2.74-2.54 (m, 2H), 1.67-1.44 (m, 2H), 1.34 (s, 4H), 0.94 (t, J=7.2 Hz, 3H), 0.88-0.73 (m, 3H); ¹³C NMR (101 MHz, DMSO-d₆, HCl salt); 6 ppm 160.90, 138.84, 108.46, 106.09, 56.48, 53.36, 41.24, 41.11, 38.68, 33.17, 31.68, 25.34, 21.55, 13.98, 9.90.

### Example 47: Preparation of 2-(3,5-demethoxy-4-pentylphenyl)-N-(2-methoxybenzyl)ethanamine (49)

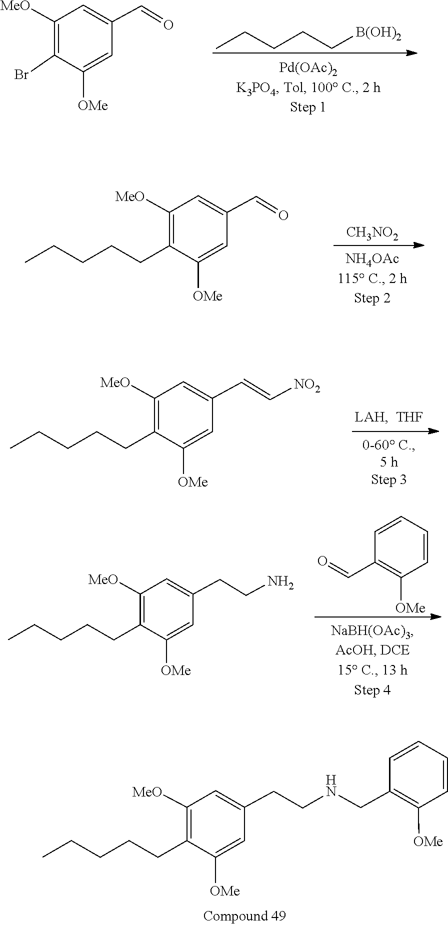

### Step 1: Preparation of 3,5-dimethoxy-4-pentylbenzaldehyde

A mixture of 4-bromo-3,5-dimethoxybenzaldehyde (3.0 g, 12.2 mmol, 1 eq.), K₃PO₄ (7.8 g, 36.7 mmol, 3 eq.), pentylboronic acid (2.13 g, 18.4 mmol, 1.5 eq.), dicyclohexyl-[2-(2,6-dimethoxyphenyl)phenyl]phosphane (1.01 g, 2.45 mmol, 0.2 eq.), and Pd(OAc)₂ (275 mg, 1.22 mmol, 0.1 eq.) in toluene (25 mL) was stirred and warmed to 105° C. for 2 h under N₂. The mixture was stirred at 80° C. for 12 h. Upon completion, the reaction mixture was filtered, and the filtrate was concentrated. The residue was purified by column chromatography (SiO₂, Petroleum ether/Ethyl acetate=100/1-0/1) to afford 3,5-dimethoxy-4-pentylbenzaldehyde (2 g, 8.5 mmol, 69% yield) as a yellow oil. ¹H NMR (400 MHz, CHLOROFORM-d) δ=9.91 (s, 1H), 7.06 (s, 2H), 3.89 (s, 6H), 2.73-2.64 (m, 2H), 1.55-1.42 (m, 2H), 1.34 (br dd, J=3.6, 7.2 Hz, 4H), 0.94-0.86 (m, 3H).

### Step 2: Preparation of (E)-1,3-dimethoxy-5-(2-nitrovinyl)-2-pentylbenzene

A mixture of 3,5-dimethoxy-4-pentylbenzaldehyde (2 g, 8.5 mmol, 1 eq.) and NH₄OAc (1.30 g, 16.9 mmol, 2 eq.) in nitromethane (15.5 g, 254 mmol, 13.7 mL, 30 eq.) was stirred at 115° C. for 2 h. Upon completion, the reaction mixture was concentrated to give a residue that was purified on silica gel chromatography (PE:EA=80:1-60:1) to afford (E)-1,3-dimethoxy-5-(2-nitrovinyl)-2-pentylbenzene (1.7 g, 6.1 mmol, 72% yield) as a yellow oil. ¹H NMR (400 MHz, CHLOROFORM-d) δ=7.97 (d, J=13.6 Hz, 1H), 7.59 (d, J=13.6 Hz, 1H), 6.69 (s, 2H), 3.86 (s, 6H), 2.73-2.59 (m, 2H), 1.52-1.42 (m, 2H), 1.33 (br d, J=3.2 Hz, 4H), 0.90 (t, J=6.8 Hz, 3H).

### Step 3: Preparation of 2-(3,5-dimethoxy-4-pentylphenyl)ethanamine

A solution of (E)-1,3-dimethoxy-5-(2-nitrovinyl)-2-pentylbenzene (600 mg, 2.15 mmol, 1 eq.) in THF (20 mL) was cooled to 0° C. Then LiAlH₄ (489 mg, 12.9 mmol, 6 eq.) was added. The mixture was stirred then warmed to 60° C. for 5 h. Upon completion, the mixture was cooled to 0° C. Then (0.5 mL) H₂O was added dropwise and the mixture stirred. Then (0.5 mL) 30% aq. NaOH was added and the mixture stirred. After stirring to a smooth dispersion, the solids were filtered, and the filtrate concentrated to afford 2-(3,5-dimethoxy-4-pentylphenyl)ethanamine (500 mg, 89% yield) as a white solid. This material was used directly in the next step.

### Step 4: Preparation of 2-(3,5-dimethoxy-4-pentylphenyl)-N-(2-methoxybenzyl)ethanamine (49)

To a solution of 2-(3,5-dimethoxy-4-pentylphenyl)ethanamine (400 mg, 1.6 mmol, 1 eq.) and 2-methoxybenzaldehyde (65 mg, 477 μmol, 0.3 eq.) in DCE (10 mL) was added AcOH (9.56 mg, 159 μmol, 0.1 eq.). The mixture was stirred at 15° C. for 1 h. Then NaBH(OAc)₃ (1.01 g, 4.77 mmol, 3 eq.) was added and the mixture was stirred at 15° C. for 12 h. Upon completion, the mixture was basified to pH=8 with sat. aq. NaHCO₃ soln., stirred, then extracted with DCM (10 mL×2). The organic layer was washed with brine (15 mL), dried over Na₂SO₄, filtered, and concentrated. The residue was purified by prep-HPLC ([water (0.04% HCl)-ACN]; B %: 24%-54%. 20 min) to afford 2-(3,5-dimethoxy-4-pentylphenyl)-N-(2-methoxybenzyl)ethanamine (260 mg, 24% yield, HCl) as a white solid. LCMS R_{T}=2.494 min, MS cal.: 371.51, [M+H]⁺=372.1; 1H NMR (400 MHz, CHLOROFORM-d, HCl salt) δ=9.54-9.23 (m, 2H), 7.43-7.30 (m, 2H), 7.03-6.92 (m, 1H), 6.81 (d, J=8.4 Hz, 1H), 6.30 (s, 2H), 4.16 (br s, 2H), 3.79-3.69 (m, 6H), 3.63 (s, 3H), 3.10 (br s, 4H), 2.64-2.51 (m, 2H), 1.50-1.38 (m, 2H), 1.37-1.24 (m, 4H), 0.89 (br t, J=6.8 Hz, 3H); ¹³C NMR (101 MHz, CHLOROFORM-d, HCl salt) δ=156.66, 130.12, 129.41, 119.22, 108.53, 102.28, 53.90, 30.10, 26.95, 20.80, 20.62, 12.15.

### Example 48: Preparation of 2-(((4-(butylthio)-3,5-dimethoxyphenethyl)amino)methyl)phenol (50)

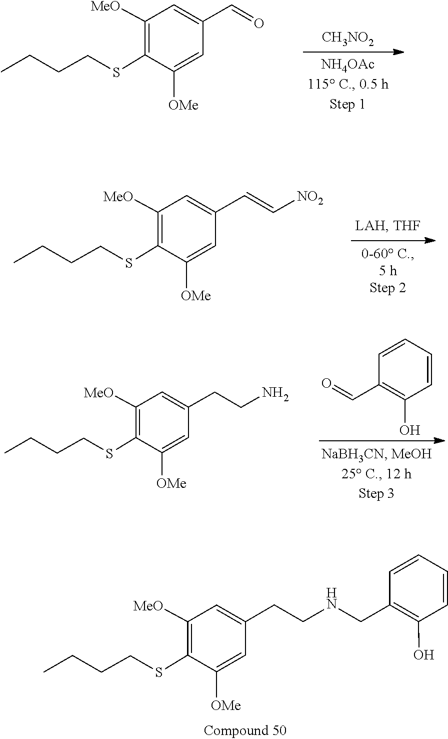

### Step 1: Preparation of (E)-butyl(2,6-dimethoxy-4-(2-nitrovinyl)phenyl)sulfane

To a mixture of 4-(butylthio)-3,5-dimethoxybenzaldehyde (3 g, 11.8 mmol, 1 eq.) in nitromethane (13 mL) was added NH₄OAc (1.82 g, 23.6 mmol, 2 eq.), and the mixture was warmed and stirred at 115° C. for 0.5 h under a N₂ atmosphere. Upon completion, the reaction mixture was concentrated to give a residue that was purified by column chromatography (SiO₂, Petroleum ether/Ethyl acetate=50/1) to afford (E)-butyl(2,6-dimethoxy-4-(2-nitrovinyl)phenyl)sulfane (2.2 g, 6.66 mmol, 56% yield, 90% purity) as a yellow oil. ¹H NMR (400 MHz, CHLOROFORM-d) δ ppm 7.98-7.94 (d, J=13.6 Hz, 1H), 7.62-7.58 (d, J=13.6 Hz, 1H), 6.71 (s, 2H), 3.94 (s, 6H), 2.92 (t, J=7.6 Hz, 2H), 1.53-1.37 (m, 4H), 0.88 (t, J=7.2 Hz, 3H).

### Step 2: Preparation of 2-(4-(butylthio)-3,5-dimethoxyphenyl)ethanamine

A solution of (E)-butyl(2,6-dimethoxy-4-(2-nitrovinyl)phenyl)sulfane (2 g, 6.73 mmol, 1 eq.) in THF (10 mL) was degassed and purged with N₂ 3 times. LiAlH₄ (1.02 g, 26.9 mmol, 4 eq.) was added at 0° C., and the mixture was warmed and stirred at 60° C. for 5 h under a N₂ atmosphere. Upon completion, the reaction mixture was quenched by dropwise addition of water (1 mL) at 0° C. followed by a solution of 30% aq. NaOH (1 mL) at 0° C. After stirring to a smooth dispersion, the mixture was filtered, and the filtrate was concentrated to give a residue. The residue was purified by column chromatography (SiO₂, Petroleum ether/Ethyl acetate=10/1-1/1) to afford 2-(4-(butylthio)-3,5-dimethoxyphenyl)ethanamine (0.8 g, 2.67 mmol, 40% yield, 90% purity) as a yellow oil. 1H NMR (400 MHz, CHLOROFORM-d) δ ppm 6.59 (s, 2H), 4.69 (s, 2H), 3.89 (s, 6H), 2.84-2.76 (m, 2H), 1.49-1.36 (m, 4H), 0.86 (t, J=7.2 Hz, 3H).

### Step 3: Preparation of 2-(((4-(butylthio)-3,5-dimethoxyphenethyl)amino)methyl)phenol (50)

A mixture of 2-(4-(butylthio)-3,5-dimethoxyphenyl)ethanamine (0.3 g, 1.11 mmol, 1 eq.), 2-hydroxybenzaldehyde (136 mg, 1.11 mmol, 118 uL, 1 eq.), and NaBH₃CN (105 mg, 1.67 mmol, 1.5 eq.) in MeOH (3 mL) was degassed and purged with N₂ 3 times then stirred at 25° C. for 12 h under a N₂ atmosphere. Upon completion, the reaction mixture was quenched by addition of water (5 mL) and extracted with DCM (10 mL×3). The combined organic layer was washed with brine, dried over Na₂SO₄, filtered, and concentrated to give a residue. The residue was purified by prep-HPLC (column: Welch Xtimate C18 100×25 mm×3 um; mobile phase: [water (0.05% HCl)-ACN]; B %: 25%-45%, 8 min) to afford 2-(((4-(butylthio)-3,5-dimethoxyphenethyl)amino)methyl)phenol (150 mg, 348 μmol, 31% yield, 96% purity, HCl) as an off-white solid. LCMS R_{T}=2.180 min, MS cal.: 375.52, [M+H]⁺=376.1; 1H NMR (400 MHz, DMSO-d6, HCl salt) δ ppm 10.25 (br s, 1H), 9.01 (br s, 2H), 7.40-7.38 (d, J=7.2 Hz, 1H), 7.24 (t, J=7.6 Hz, 1H), 6.96 (d, J=8.0 Hz, 1H), 6.85 (t, J=7.2 Hz, 1H), 6.56 (s, 2H), 4.11 (s, 2H), 3.83 (s, 6H), 3.17 (s, 2H), 3.02-2.91 (m, 2H), 2.72-2.65 (m, 2H), 1.37-1.28 (m, 4H), 0.84-0.77 (m, 3H); ¹³C NMR (101 MHz, DMSO-d6, HCl salt) δ ppm 161.00, 156.52, 139.52, 132.08, 130.95, 119.56, 118.60, 115.87, 108.43, 105.33, 56.47, 47.76, 45.60, 41.35, 41.31, 41.27, 41.09, 33.18, 32.29, 31.67, 21.53, 13.98.

### Example 49: Preparation of 2-(3,5-demethoxy-4-(pentylthio)phenyl)-N-(2-methoxybenzyl)ethanamine (51)

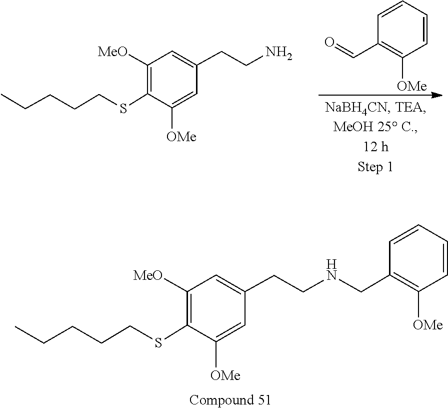 [SMILES: CCCCCSc1c(OC)cc(CCN)cc1OC ; COc1ccccc1C=O ; CCCCCSc1c(OC)cc(CCNCc2ccccc2OC)cc1OC]

### Step 1: Preparation of 2-(3,5-dimethoxy-4-(pentylthio)phenyl)-N-(2-methoxybenzyl)ethanamine (51)

To a solution of 2-(3,5-dimethoxy-4-(pentylthio)phenyl)ethanamine (300 mg, 1.06 mmol, 1 eq.) in MeOH (4 mL) was added Et₃N (1071 mg, 10.6 mmol, 10 eq.) followed by 2-methoxybenzaldehyde (130 mg, 953 μmol, 0.9 eq.). The mixture was stirred at 25° C. for 2 h. Then NaBH₃CN (67 mg, 1.06 μmol, 1 eq.) was added. The mixture was stirred at 25° C. for 12 h. Upon completion, the MeOH was removed and the reaction was diluted in H₂O (5 mL) and extracted with DCM (10 mL×2). The organic layer was washed with brine, dried over Na₂SO₄, filtered, and concentrated. The residue was purified by prep-HPLC ([water (0.05% HCl)-ACN]; B %: 20%-40%, 8 min) to afford 2-(3,5-dimethoxy-4-(pentylthio)phenyl)-N-(2-methoxybenzyl)ethanamine (231 mg, 572 μmol, 48% yield, HCl) as a white solid. LCMS R_{T}=2.340 min, MS cal.: 403.22, [M+H]⁺=404.1; 1H NMR (400 MHz, CHLOROFORM-d, HCl salt) δ=9.37 (ddd, J=0.8, 4.8, 7.2 Hz, 2H), 7.42-7.29 (m, 2H), 6.96 (br t, J=7.6 Hz, 1H), 6.83 (d, J=8.0 Hz, 1H), 6.36 (s, 2H), 4.14 (br s, 2H), 3.83 (s, 6H), 3.68 (s, 3H), 3.12 (br s, 4H), 2.78 (t, J=7.6 Hz, 2H), 1.60-1.45 (m, 2H), 1.42-1.17 (m, 4H), 0.86 (t, J=7.2 Hz, 3H); ¹³C NMR (101 MHz, CHLOROFORM-d, HCl salt) δ=161.31, 157.61, 137.88, 132.05, 131.48, 121.16, 118.00, 110.57, 109.59, 104.64, 56.38, 55.50, 47.36, 47.21, 34.07, 32.78, 30.95, 29.35, 22.31, 14.01.

### Example 50: Preparation of 1-(4-(butylthio)-3,5-dimethoxyphenyl)-N-(2-methoxybenzyl)propan-2-amine (52)

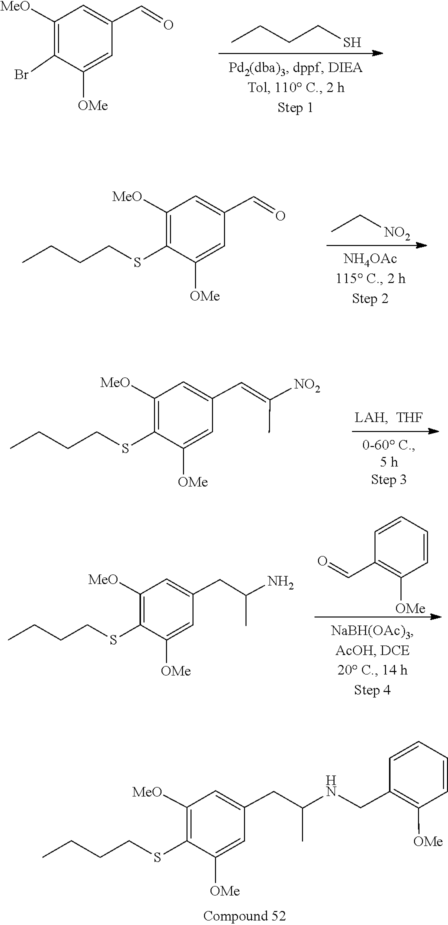

### Step 1: Preparation of 4-(butylthio)-3,5-dimethoxybenzaldehyde

To a mixture of 4-bromo-3,5-dimethoxybenzaldehyde (3.5 g, 14.3 mmol, 1 eq.) and butane-1-thiol (1.67 g, 18.6 mmol, 2.0 mL, 1.3 eq.) in toluene (30 mL) was added DIEA (5.54 g, 43 mmol, 7.46 mL, 3 eq.), DPPF (792 mg, 1.43 mmol, 0.1 eq.), and Pd₂(dba)₃ (1.31 g, 1.43 mmol, 0.1 eq.) in one portion at 20° C. under N₂. The mixture was warmed to 110° C. and stirred for 2 h. Upon completion, the reaction mixture was filtered, and the filtrate was concentrated. The residue was purified by column chromatography (SiO₂, Petroleum ether/Ethyl acetate=15/1-0/1) to give product 4-(butylthio)-3,5-dimethoxybenzaldehyde (2.5 g, 9.83 mmol, 69% yield) as a yellow oil. ¹H NMR (400 MHz, CHLOROFORM-d) δ=9.97-9.91 (m, 1H), 7.07 (s, 2H), 3.97 (s, 6H), 3.00-2.91 (m, 2H), 1.55-1.37 (m, 4H), 0.88 (t, J=7.2 Hz, 3H).

### Step 2: Preparation of (E)-butyl(2,6-dimethoxy-4-(2-nitroprop-1-en-1-yl)phenyl)sulfane

To a mixture of 4-(butylthio)-3,5-dimethoxybenzaldehyde (2.3 g, 9.04 mmol, 1 eq.) in nitroethane (16.97 g, 226 mmol, 16.16 mL, 25 eq.) was added NH₄OAc (1.39 g, 18.1 mmol, 2 eq.) in one portion at 20° C. under N₂. The mixture was warmed to 115° C. and stirred for 2 h. Upon completion, the solvent was removed and the residue purified by column chromatography (SiO₂, Petroleum ether/Ethyl acetate=15/1-0/1) to give product (E)-butyl(2,6-dimethoxy-4-(2-nitroprop-1-en-1-yl)phenyl)sulfane (2.1 g, 6.74 mmol, 75% yield) as a yellow solid. ¹H NMR (400 MHz, CHLOROFORM-d) δ=8.10-7.99 (m, 1H), 6.60 (s, 2H), 3.91 (s, 6H), 2.88 (t, J=7.6 Hz, 2H), 2.49 (d, J=1.2 Hz, 3H), 1.55-1.35 (m, 4H), 0.88 (t, J=7.2 Hz, 3H).

### Step 3: Preparation of 1-(4-butylsulfanyl-3,5-dimethoxy-phenyl)propan-2-amine

A solution of (E)-butyl(2,6-dimethoxy-4-(2-nitroprop-1-en-1-yl)phenyl)sulfane (1.0 g, 3.21 mmol, 1 eq.) in THF (30 mL) was stirred and cooled to 0° C. Then LiAlH₄ (488 mg, 12.9 mmol, 4 eq.) was added. The mixture was warmed to 60° C. and stirred for 5 h. Upon completion, the reaction mixture was quenched by the dropwise addition of H₂O (0.5 mL) at 0° C. followed by dropwise addition of 30% aq. NaOH (0.5 mL). The mixture was stirred to a smooth dispersion then filtered, and the filtrate was concentrated to give 1-(4-(butylthio)-3,5-dimethoxyphenyl)propan-2-amine (1.13 g, crude) as a yellow oil. This material was used as is in the next step.

### Step 4: Preparation of 1-(4-(butylthio)-3,5-dimethoxyphenyl)-N-(2-methoxybenzyl)propan-2-amine (52)

To a solution of 1-(4-(butylthio)-3,5-dimethoxyphenyl)propan-2-amine (540 mg, 1.91 mmol, 1 eq.) and 2-methoxybenzaldehyde (156 mg, 1.14 mmol, 0.6 eq.) in DCE (20 mL) was added AcOH (12 mg, 191 μmol, 10.9 uL, 0.1 eq.) at 20° C. under N₂. The mixture was stirred at 20° C. for 2 h. Then the mixture was treated with NaBH(OAc)₃ (1.21 g, 5.72 mmol, 3 eq.) in one portion at 20° C. under N₂. The mixture was stirred at 20° C. for 12 h. Upon completion, the reaction mixture was diluted with H₂O (20 mL) and extracted with DCM (20 mL×3). The combined organic layer was washed with brine (20 mL×2), dried over Na₂SO₄, filtered, and concentrated. The crude product was purified by Prep-HPLC (column: Phenomenex luna C18 250×50 mm×10 μm; mobile phase: [water (0.04% HCl)-ACN]; B %: 25%-55%, 10 min) to give 1-(4-(butylthio)-3,5-dimethoxyphenyl)-N-(2-methoxybenzyl)propan-2-amine (337 mg, 587 μmol, 43% yield, 100% purity, HCl) as an off-white oil. LCMS R_{T}=2.332 min, MS cal.: 403.22, [M+H]⁺=404.1; ¹H NMR (400 MHz, DMSO-d6, HCl salt) δ=9.16-8.92 (m, 2H), 7.54 (dd, J=1.2, 7.5 Hz, 1H), 7.50-7.40 (m, 1H), 7.11 (d, J=8.0 Hz, 1H), 7.02 (t, J=7.6 Hz, 1H), 6.55 (s, 2H), 4.28-4.12 (m, 2H), 3.85-3.78 (m, 9H), 3.49 (br d, J=4.4 Hz, 2H), 3.26 (br dd, J=4.4, 13.1 Hz, 1H), 2.83-2.64 (m, 3H), 1.37-1.30 (m, 4H), 1.23 (d, J=6.4 Hz, 3H), 0.86-0.78 (m, 3H); ¹³C NMR (101 MHz, DMSO-d6, HCl salt) δ=160.95, 158.00, 139.10, 132.01, 131.28, 120.88, 120.41, 111.57, 105.83, 56.50, 56.05, 55.01, 43.14, 39.19, 33.12, 31.67, 21.51, 16.11, 13.97.

### Example 51: Preparation of 1-(4-hexyl-2,5-dimethoxyphenyl)-N-(2-methoxybenzyl)propan-2-amine (53)

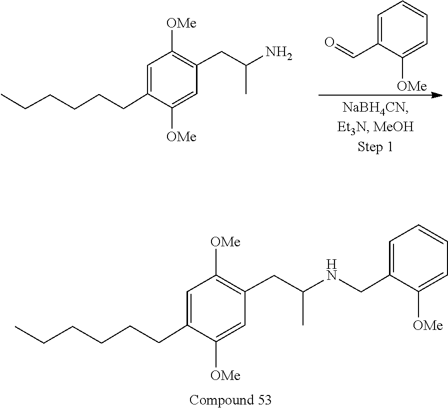 [SMILES: CCCCCCc1cc(OC)c(CC(C)N)cc1OC ; COc1ccccc1C=O ; CCCCCCc1cc(OC)c(CC(C)NCc2ccccc2OC)cc1OC]

### Step 1: Preparation of 1-(4-hexyl-2,5-dimethoxyphenyl)-N-(2-methoxybenzyl)propan-2-amine (53)

To a mixture of 1-(4-hexyl-2,5-dimethoxyphenyl)propan-2-amine (440 mg, 1.39 mmol, 1 eq, HCl) in MeOH (5 mL) was added Et₃N (1.41 g, 13.9 mmol, 1.94 mL, 10 eq.) until the pH of the reaction was ˜8. Then 2-methoxybenzaldehyde (171 mg, 1.25 mmol, 0.9 eq.) was added at 20° C. The mixture was stirred for 1 h then treated with NaBH₃CN (87.5 mg, 1.39 mmol, 1 eq.) and stirred at 20° C. for 12 h. The reaction was diluted in H₂O (5 mL) and concentrated to remove the MeOH. The residue was diluted in H₂O (10 mL) and extracted with DCM (10 mL×3). The combined organic layer was washed with brine and dried over Na₂SO₄ then filtered, and concentrated. The residue was purified by prep-HPLC (column: Phenomenex luna C18 250×50 mm×10 μm; mobile phase: [water (0.04% HCl)-ACN]; B %: 40%-70%, 10 min) to give 1-(4-hexyl-2,5-dimethoxyphenyl)-N-(2-methoxybenzyl)propan-2-amine (267 mg, 667 μmol, 48% yield) as a white solid. ¹H NMR (400 MHz, DMSO-d6, HCl salt) δ ppm 8.90-8.51 (m, 2H), 7.49-7.45 (m, 1H), 7.45-7.40 (m, 1H), 7.11 (d, J=8.2 Hz, 1H), 7.02 (t, J=7.2 Hz, 1H), 6.80 (s, 1H), 6.76 (s, 1H), 4.19 (s, 2H), 3.82 (s, 3H), 3.72 (s, 3H), 3.70 (s, 3H), 3.38 (br s, 1H), 3.10 (dd, J=4.4, 13.2 Hz, 1H), 2.70 (dd, J=10.4, 13.2 Hz, 1H), 1.49 d, J=7.6 Hz, 2H), 1.27 (d, J=2.4 Hz, 6H), 1.17 (d, J=6.4 Hz, 3H), 0.91-0.81 (m, 3H); ¹³C NMR (101 MHz, DMSO-d6, HCl salt) δ ppm 157.52, 150.84, 150.77, 131.39, 130.84, 129.96, 122.15, 120.42, 113.86, 113.09, 111.08, 55.90, 55.84, 55.57, 53.62, 33.13, 31.11, 29.65, 29.57, 28.63, 22.07, 15.67, 13.96.

### Example 52: Preparation of 1-(4-(butylthio)-2,5-dimethoxyphenyl)-N-(2-methoxybenzyl)propan-2-amine (54)

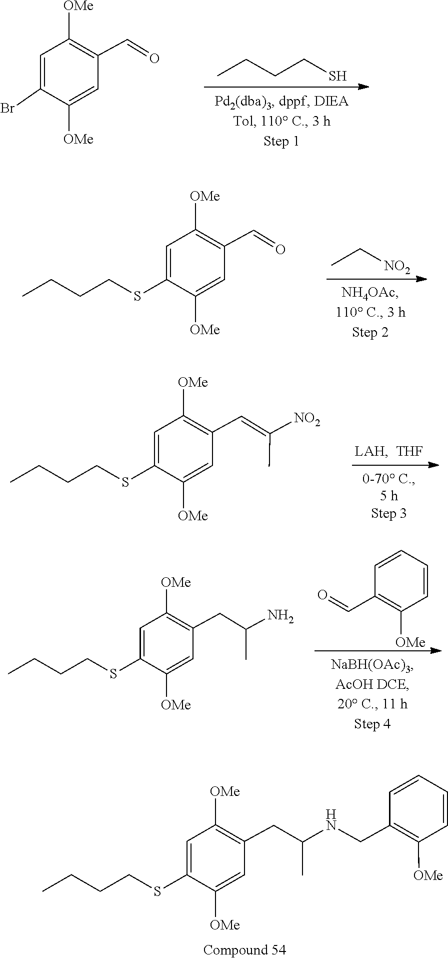

### Step 1: Preparation of 4-(butylthio)-2,5-dimethoxybenzaldehyde

A mixture of 4-bromo-2,5-dimethoxybenzaldehyde (3 g, 12.24 mmol, 1 eq.), butane-1-thiol (1.44 g, 15.9 mmol, 1.70 mL, 1.3 eq.), DPPF (679 mg, 1.22 mmol, 0.1 eq.), DIEA (4.75 g, 36.7 mmol, 6.40 mL, 3 eq.), and Pd₂(dba)₃ (1.12 g, 1.22 mmol, 0.1 eq.) in toluene (20 mL) was degassed and purged with N₂ 3 times. The resulting mixture was warmed to 110° C. and stirred for 2 h under a N₂ atmosphere. Upon completion, the reaction mixture was filtered, and the filtrate concentrated. To the residue was added water (100 mL) and the aqueous phase was extracted with ethyl acetate (50 mL×2). The combined organic phase was washed with brine (60 mL×1), dried over anhydrous Na₂SO₄, filtered, and concentrated. The residue was purified by column chromatography (SiO₂, PE/EA=20/1-0/1) to afford 4-(butylthio)-2,5-dimethoxybenzaldehyde (2.38 g, 9.36 mmol, 76% yield) as a yellow solid. ¹H NMR (400 MHz, CHLOROFORM-d) δ 10.37 (s, 1H), 7.25 (s, 1H), 6.78 (s, 1H), 3.93 (s, 3H), 3.90 (s, 3H), 2.97 (t, J=7.2 Hz, 2H), 1.80-1.70 (m, 2H), 1.59-1.48 (m, 3H), 0.98 (t, J=7.2 Hz, 3H).

### Step 2: Preparation of (E)-butyl(2,5-dimethoxy-4-(2-nitroprop-1-en-1-yl)phenyl)sulfane

To a solution of 4-(butylthio)-2,5-dimethoxybenzaldehyde (2.38 g, 9.36 mmol, 1 eq.) in nitroethane (20 mL) was added NH₄OAc (1.44 g, 18.7 mmol, 2 eq.). The mixture was warmed to 110° C. and stirred for 3 h. Upon completion, the solvent was removed to give a residue. The residue was purified by column chromatography (SiO₂, Petroleum ether/Ethyl acetate=20/1-0/1) to afford (E)-butyl(2,5-dimethoxy-4-(2-nitroprop-1-en-1-yl)phenyl)sulfane (1.68 g, 5.40 mmol, 58% yield) as a yellow solid. ¹H NMR (400 MHz, CHLOROFORM-d) δ 8.29 (s, 1H), 6.80 (d, J=8.0 Hz, 2H), 3.87 (d, J=1.2 Hz, 6H), 2.96 (t, J=7.6 Hz, 2H), 2.43 (s, 3H), 1.72 (quin, J=7.2 Hz, 2H), 1.57-1.46 (m, 2H), 0.97 (t, J=7.2 Hz, 3H).

### Step 3: Preparation of 1-(4-(butylthio)-2,5-dimethoxyphenyl)propan-2-amine

A solution of (E)-butyl(2,5-dimethoxy-4-(2-nitroprop-1-en-1-yl)phenyl)sulfane (1.58 g, 5.07 mmol, 1 eq.) in THF (20 mL) was stirred at 0° C. and treated with LiAlH₄ (770 mg, 20.3 mmol, 4 eq.). After 15 min. at 15° C. the mixture was warmed to 70° C. and stirred for 5 h. Upon completion, the mixture was cooled to 0° C. and stirred while water (1 mL) was added dropwise. After stirring for ˜5 min. 30% aq. NaOH (1 mL) was added dropwise and stirred to a smooth dispersion. The mixture was filtered, and the filtrate was concentrated in vacuo to afford 1-(4-(butylthio)-2,5-dimethoxyphenyl)propan-2-amine (1.56 g, crude) as a yellow oil. ¹H NMR (400 MHz, CHLOROFORM-d) δ 6.84 (s, 1H), 6.68 (s, 1H), 3.87-3.83 (m, 3H), 3.81-3.77 (m, 3H), 3.28-3.16 (m, 1H), 2.89 (t, J=7.2 Hz, 2H), 2.72 (dd, J=5.2, 13.0 Hz, 1H), 2.60-2.47 (m, 1H), 1.68-1.58 (m, 2H), 1.52-1.43 (m, 2H), 1.13 (d, J=6.4 Hz, 3H), 0.93 (t, J=7.2 Hz, 3H).

### Step 4: Preparation of 1-(4-(butylthio)-2,5-dimethoxyphenyl)-N-(2-methoxybenzyl)propan-2-amine (54)

The stirred solution of 1-(4-(butylthio)-2,5-dimethoxyphenyl)propan-2-amine (400 mg, 1.41 mmol, 1 eq.), 2-methoxybenzaldehyde (154 mg, 1.13 mmol, 0.8 eq.) in DCE (15 mL) was treated with AcOH (8.5 mg, 141 μmol, 8.1 uL, 0.1 eq.). The mixture was stirred at 20° C. for 1 h. Then NaBH(OAc)₃ (1.05 g, 4.94 mmol, 3.5 eq.) was added. The mixture was stirred at 20° C. for 10 h. Upon completion, the mixture was basified to pH=8 with sat. aq. NaHCO₃ soln. (10 mL) and extracted with DCM (50 mL×2). The organic layer was dried over over Na₂SO₄, filtered, and concentrated. The residue was purified by prep-HPLC (column: Kromasil C18 (250×50 mm×10 μm); mobile phase: [water (0.05% NH₃H₂O+10 mM NH₄HCO₃)-ACN]; B %: 45%-85%, 10 min) to obtain 1-(4-(butylthio)-2,5-dimethoxyphenyl)-N-(2-methoxybenzyl)propan-2-amine (260 mg) as a yellow oil. ¹H NMR (400 MHz, CHLOROFORM-d) δ 7.23-7.17 (m, 1H), 7.16-7.11 (m, 1H), 6.87 (t, J=7.2 Hz, 1H), 6.81-6.75 (m, 2H), 6.64 (s, 1H), 3.88-3.61 (m, 11H), 2.88 (d, J=7.2 Hz, 2H), 2.77-2.69 (m, 1H), 2.67-2.61 (m, 1H), 2.01 (s, 1H), 1.64 (quin, J=7.2 Hz, 2H), 1.47 (qd, J=7.2, 14.8 Hz, 2H), 1.14 (d, J=6.4 Hz, 3H), 0.93 (t, J=7.2 Hz, 3H); ¹³C NMR (101 MHz, CHLOROFORM-d) δ 157.63, 151.89, 151.66, 129.90, 128.16, 126.89, 122.50, 120.19, 114.12, 113.46, 110.00, 56.40, 56.09, 54.97, 51.59, 46.89, 38.04, 32.45, 31.24, 22.03, 20.15, 13.68.

### Example 53: Preparation of 2-(((1-(2,5-demethoxy-4-pentylphenyl)propan-2-yl)amino)methyl)phenol (55)

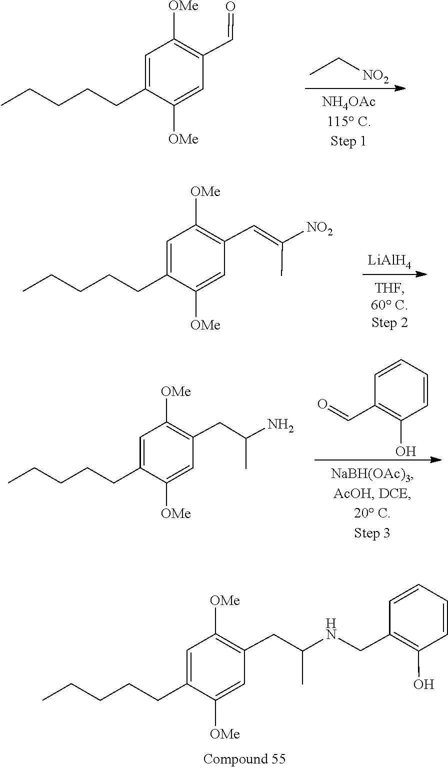

### Step 1: Preparation of 1,4-dimethoxy-2-[(E)-2-nitroprop-1-enyl]-5-pentyl-benzene

To a mixture of 2,5-dimethoxy-4-pentylbenzaldehyde (2 g, 8.46 mmol, 1 eq.) in nitroethane (15.75 g, 210 mmol, 15 mL, 25 eq.) was added NH₄OAc (1.30 g, 16.9 mmol, 2 eq.) at 20° C. Then the mixture was warmed to 115° C. and stirred for 1.5 h. Upon completion, the reaction was concentrated and the residue was purified by column chromatography (SiO₂, Petroleum ether:Ethyl acetate=80:1-50:1) to afford (E)-1,4-dimethoxy-2-(2-nitroprop-1-en-1-yl)-5-pentylbenzene (1.5 g, 5.11 mmol, 60% yield) as a yellow oil. 1H NMR (400 MHz, CHLOROFORM-d) δ ppm 8.29 (s, 1H), 6.78 (s, 1H), 6.76 (s, 1H), 3.85 (s, 3H), 3.81 (s, 3H), 2.70-2.56 (m, 2H), 2.43 (s, 4H), 1.69-1.55 (m, 3H), 1.45-1.30 (m, 5H), 0.92 (t, J=6.8 Hz, 3H).

### Step 2: Preparation of 1-(2,5-dimethoxy-4-pentylphenyl)propan-2-amine

A stirred solution of (E)-1,4-dimethoxy-2-(2-nitroprop-1-en-1-yl)-5-pentylbenzene (1.5 g, 5.11 mmol, 1 eq.) in THF (15 mL) was cooled to 0° C. then LiAlH₄ (776 mg, 20.5 mmol, 4 eq.) was added. The mixture was warmed to 60° C. and stirred for 5 h. Upon completion, the mixture was cooled to 0° C. Then H₂O (0.6 mL) was added dropwise followed by 30% aq. NaOH solution (0.6 mL), dropwise. After stirring to a smooth mixture, the solids were filtered, and the filtrate was concentrated to afford 1-(2,5-dimethoxy-4-pentylphenyl)propan-2-amine (800 mg, 3.01 mmol, 59% yield) as a yellow oil. ¹H NMR (400 MHz, CHLOROFORM-d) δ ppm 6.73 (s, 1H), 6.71 (s, 1H), 4.46 (s, 1H), 3.00 (m, 1H), 2.56-2.40 (m, 5H), 1.58-1.45 (m, 3H), 1.41-1.17 (m, 7H), 0.94 (d, J=6.4 Hz, 3H), 0.87 (t, J=6.8 Hz, 1H).

### Step 3: Preparation of 2-(((1-(2,5-dimethoxy-4-pentylphenyl)propan-2-yl)amino)methyl)phenol (55)

A solution of 1-(2,5-dimethoxy-4-pentylphenyl)propan-2-amine (522 mg, 1.97 mmol, 1 eq.), 2-hydroxybenzaldehyde (204 mg, 1.67 mmol, 178 uL, 0.85 eq.), and AcOH (11.8 mg, 197 μmol, 11.3 uL, 0.1 eq.) in DCE (8 mL) was stirred at 20° C. for 1 h. Then NaBH(OAc)₃ (1.04 g, 4.92 mmol, 2.5 eq.) was added. The mixture was stirred at 20° C. for 12 h. Upon completion, the mixture was basified to pH=8 with sat. aq. NaHCO₃ soln. and extracted with DCM (10 mL×2). The combined organic layer was washed with brine and dried over Na₂SO₄, filtered, and concentrated. The residue was purified by prep-HPLC (column: Phenomenex luna C18 250×50 mm×10 μm; mobile phase: [water (0.04% HCl)-ACN]; B %: 30%-60%, 10 min) to afford 2-(((1-(2,5-dimethoxy-4-pentylphenyl)propan-2-yl)amino)methyl)phenol (430 mg, 1.05 mmol, 54% yield, HCl) as a brown solid. ¹H NMR (400 MHz, DMSO-d₆, HCl salt) δ ppm 10.35 (s, 1H), 9.19 (d, J=4.8 Hz, 1H), 8.97 (d, J=4.8 Hz, 1H), 7.47 (d, J=6.4 Hz, 1H), 7.27-7.19 (m, 1H), 7.02 (d, J=8.0 Hz, 1H), 6.84 (t, J=7.2 Hz, 1H), 6.76 (d, J=7.6 Hz, 2H), 4.14 (s, 2H), 3.71 (s, 3H), 3.68 (s, 3H), 3.44-3.28 (m, 3H), 3.15 (dd, J=3.6, 12.8 Hz, 1H), 2.72 (dd, J=10.4, 12.8 Hz, 1H), 2.52-2.49 (m, 2H), 1.50 (m, 2H), 1.35-1.21 (m, 4H), 1.16 (d, J=6.4 Hz, 3H), 0.85 (t, J=6.8 Hz, 3H); ¹³C NMR (101 MHz, DMSO-d₆, HCl salt) δ ppm 156.11, 150.86, 150.76, 131.53, 130.23, 129.83, 122.42, 118.97, 118.31, 115.43, 113.81, 113.06, 55.89, 55.82, 53.26, 42.51, 33.06, 31.22, 29.64, 29.30, 21.98, 15.62, 13.94.

### Example 54: Preparation of 1-(2,5-demethoxy-4-pentylphenyl)-N-(2-methoxybenzyl)butan-2-amine (56)

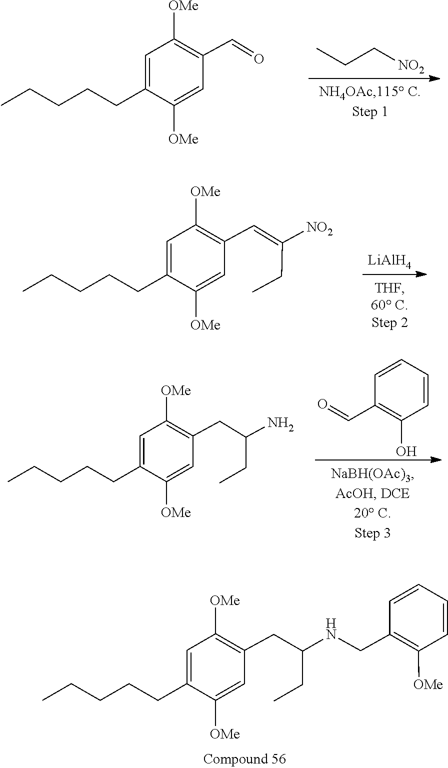

### Step 1: Preparation of (E)-1,4-dimethoxy-2-(2-nitrobut-1-en-1-yl)-5-pentylbenzene

To a mixture of 2,5-dimethoxy-4-pentylbenzaldehyde (2 g, 8.46 mmol, 1 eq.) in 1-nitropropane (10 mL) was added NH₄OAc (1.30 g, 16.9 mmol, 2 eq.) at 20° C. Then the mixture was warmed to 115° C. and stirred for 1.5 h. Upon completion, the reaction was concentrated and the residue purified by column chromatography (SiO₂, Petroleum ether:Ethyl acetate=100:1-50:1) to afford (E)-1,4-dimethoxy-2-(2-nitrobut-1-en-1-yl)-5-pentylbenzene (1.5 g, 4.88 mmol, 58% yield) as an orange oil. ¹H NMR (400 MHz, CHLOROFORM-d) δ ppm 10.41 (s, 1H), 8.25 (s, 1H), 6.80 (s, 1H), 6.76 (s, 1H), 3.84 (s, 4H), 3.81 (s, 4H), 2.87 (mz, 2H), 2.69-2.58 (m, 3H), 1.65-1.54 (m, 5H), 1.36 (td, J=3.6, 7.2 Hz, 6H), 1.33-1.25 (m, 1H), 0.98-0.88 (m, 5H).

### Step 2: Preparation of 1-(2,5-dimethoxy-4-pentylphenyl)butan-2-amine

A stirred solution of (E)-1,4-dimethoxy-2-(2-nitrobut-1-en-1-yl)-5-pentylbenzene (1.5 g, 4.88 mmol, 1 eq.) in THF (15 mL) was cooled to 0° C. then LiAlH₄ (741 mg, 20 mmol, 4 eq.) was added. The mixture was warmed to 60° C. and stirred for 5 h. Upon completion, the mixture was cooled to 0° C. Then H₂O (0.6 mL) was added dropwise followed by dropwise addition of 30% aq. NaOH (0.6 mL). The mixture was stirred until a smooth dispersion formed and the solids were filtered and concentrated to afford 1-(2,5-dimethoxy-4-pentylphenyl) butan-2-amine (1 g, 3.58 mmol, 73% yield) as a yellow oil. ¹H NMR (400 MHz, DMSO-d₆) δ ppm 7.05-6.94 (m, 1H), 6.78-6.67 (m, 1H), 4.45 (s, 1H), 3.77-3.62 (m, 3H), 3.65-3.55 (m, 1H), 2.82-2.68 (m, 1H), 2.60 (dd, J=5.6, 12.8 Hz, 1H), 2.36 (dd, J=7.6, 12.8 Hz, 1H), 1.81-1.71 (m, 1H), 1.60-1.44 (m, 1H), 1.42-1.23 (m, 4H), 1.23-1.10 (m, 1H), 0.93-0.81 (m, 3H).

### Step 3: Preparation of 1-(2,5-dimethoxy-4-pentylphenyl)-N-(2-methoxybenzyl)butan-2-amine (56)

A solution of 1-(2,5-dimethoxy-4-pentylphenyl)butan-2-amine (600 mg, 2.15 mmol, 1 eq.), 2-methoxybenzaldehyde (220 mg, 1.61 mmol, 0.75 eq.) and AcOH (13 mg, 215 μmol, 12.3 uL, 0.1 eq.) in DCE (3 mL) was stirred at 20° C. for 1 h. Then NaBH(OAc)₃ (910 mg, 4.29 mmol, 2 eq.) was added and the mixture was stirred at 20° C. for 12 h. Upon completion, the mixture was basified to pH=8 with sat. aq. NaHCO₃ soln. and extracted with DCM (10 mL×2). The combined organic layer was washed with brine, dried over Na₂SO₄, filtered, and concentrated. The residue was purified by prep-HPLC (column: Phenomenex luna C18 250×50 mm×10 μm; mobile phase: [water (0.04% HCl)-ACN]; B %: 40%-70%, 10 min) to afford 1-(2,5-dimethoxy-4-pentylphenyl)-N-(2-methoxybenzyl)butan-2-amine (410 mg, 940 μmol, 48% yield, HCl) as a colorless oil. ¹H NMR (400 MHz, DMSO-d₆, HCl salt) δ ppm 8.69 (br s, 2H), 7.49-7.39 (m, 2H), 7.08 (d, J=8.4 Hz, 1H), 7.01 (t, J=7.2 Hz, 1H), 6.78 (s, 2H), 4.16 (t, J=5.6 Hz, 2H), 3.79 (s, 3H), 3.71 (s, 3H), 3.68 (s, 3H), 3.25 (d, J=3.6 Hz, 1H), 3.03-2.95 (m, 1H), 2.92-2.82 (m, 1H), 2.53 (s, 2H), 1.67-1.57 (m, 2H), 1.51 (td, J=7.2, 14.8 Hz, 2H), 1.34-1.22 (m, 4H), 0.88 (td, J=7.2, 11.6 Hz, 6H); ¹³C NMR (101 MHz, DMSO-d₆, HCl salt) δ ppm 157.56, 150.89, 150.79, 131.61, 130.83, 129.97, 122.21, 120.38, 119.58, 113.84, 113.08, 110.98, 58.46, 55.92, 55.78, 55.48, 43.29, 31.15, 30.61, 29.56, 29.23, 22.51, 21.92, 13.89, 9.13.

### Example 55: Preparation of N-benzyl-2-(2,5-demethoxy-4-propylphenyl)ethanamine (57)

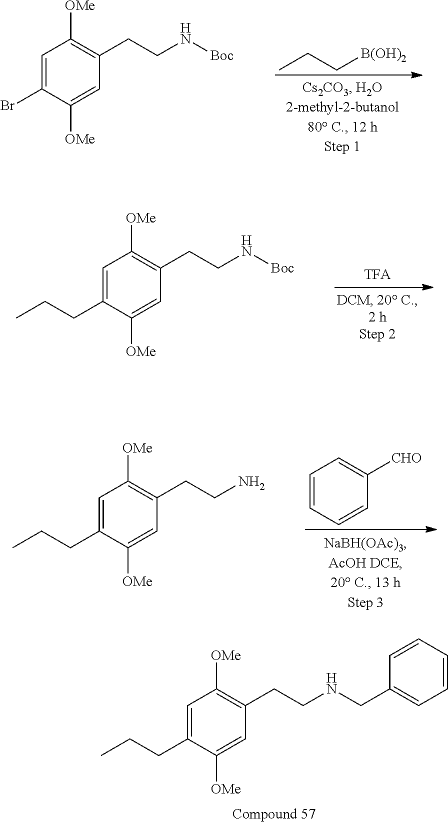

### Step 1: Preparation of tert-butyl (2,5-dimethoxy-4-propylphenethyl)carbamate

To a stirred mixture of tert-butyl (4-bromo-2,5-dimethoxyphenethyl)carbamate (3.8 g, 10.6 mmol, 1 eq.), propylboronic acid (2.78 g, 31.7 mmol, 3 eq.), and Cs₂CO₃ (10.31 g, 31.7 mmol, 3 eq.) in 2-methyl-2-butanol (20 mL) and H₂O (2 mL) was added [2-(2-aminophenyl)phenyl]-chloro-palladium; bis(1-adamantyl)-butyl-phosphane (705 mg, 1.05 mmol, 0.1 eq.) in one portion at 20° C. under N₂. The mixture was warmed and stirred at 80° C. for 12 h. Upon completion, the mixture was filtered, and concentrated. The residue was purified by column chromatography (SiO₂, Petroleum ether/Ethyl acetate=100/1-0/1) to afford tert-butyl (2,5-dimethoxy-4-propylphenethyl)carbamate (2.2 g, 6.80 mmol, 65% yield) as a yellow oil. 1H NMR (400 MHz, CHLOROFORM-d) δ=6.66 (d, J=7.6 Hz, 2H), 4.69 (br s, 1H), 3.84-3.72 (m, 6H), 3.40-3.28 (m, 2H), 2.82-2.74 (m, 2H), 2.60-2.50 (m, 2H), 1.60 (sxt, J=7.6 Hz, 2H), 1.48-1.40 (m, 9H), 0.97 (t, J=7.2 Hz, 3H).

### Step 2: Preparation of 2-(2,5-dimethoxy-4-propylphenyl)ethanamine

To a mixture of tert-butyl (2,5-dimethoxy-4-propylphenethyl)carbamate (1.2 g, 3.71 mmol, 1 eq.) in DCM (12 mL) was added TFA (2.5 mL) in one portion at 20° C. under N₂. The mixture was stirred at 20° C. for 2 h. Upon completion, the mixture was basified to pH=8 with sat. aq. Na₂CO₃ soln. and extracted with DCM (10 mL×2). The organic layer was dried over Na₂SO₄, filtered, and concentrated to afford 2-(2,5-dimethoxy-4-propylphenyl)ethanamine (800 mg, 87% yield) as a brown oil. 1H NMR (400 MHz, CHLOROFORM-d) δ=6.71-6.64 (m, 2H), 4.21 (br s, 2H), 3.78 (d, J=3.2 Hz, 6H), 3.13-2.98 (m, 2H), 2.87-2.79 (m, 2H), 2.60-2.50 (m, 2H), 1.67-1.54 (m, 2H), 1.01-0.90 (m, 3H).

### Step 3: Preparation of N-benzyl-2-(2,5-dimethoxy-4-propylphenyl)ethanamine (57)

To a stirred solution of 2-(2,5-dimethoxy-4-propylphenyl)ethanamine (450 mg, 2.02 mmol, 1 eq.) and benzaldehyde (160 mg, 1.51 mmol, 153 uL, 0.75 eq.) in DCE (5 mL) was added AcOH (0.05 mL). After stirring at 20° C. for 1 h, NaBH(OAc)₃ (1.28 g, 6.05 mmol, 3 eq.) was added and the mixture was stirred at 20° C. for 12 h. Upon completion, the mixture was basified to pH=8 with sat. aq. NaHCO₃ soln. and extracted with DCM (10 mL×2). The organic layer was dried over Na₂SO₄, filtered, and concentrated. The residue was purified by prep-HPLC (column: Phenomenex luna C18 80×40 mm×3 μm; mobile phase: [water (0.04% HCl)-ACN]; B %: 37%-60%, 7 min) to afford N-benzyl-2-(2,5-dimethoxy-4-propylphenyl)ethanamine (188 mg, 600 μmol, 30% yield, HCl) as a white solid. LCMS R_{T}=2.277 min, MS cal.: 313.20, [M+H]⁺=314.1; 1H NMR (400 MHz, CHLOROFORM-d, HCl salt) δ=9.98 (br s, 2H), 7.57 (br d, J=7.2 Hz, 2H), 7.41-7.28 (m, 3H), 6.71 (s, 1H), 6.60 (s, 1H), 4.06 (br s, 2H), 3.83-3.63 (m, 6H), 3.22-3.00 (m, 4H), 2.52 (br t, J=7.6 Hz, 2H), 1.56 (qd, J=7.2, 15.0 Hz, 2H), 0.93 (t, J=7.2 Hz, 3H); ¹³C NMR (101 MHz, CHLOROFORM-d, HCl salt) δ=151.38, 151.10, 130.95, 130.40, 130.27, 129.32, 129.03, 122.41, 113.75, 112.82, 56.21, 55.74, 50.46, 45.33, 32.35, 27.84, 23.20, 14.07.

### Example 56: Preparation of 2-(2,5-demethoxy-4-propylphenyl)-N-(2-fluorobenzyl)ethanamine (58)

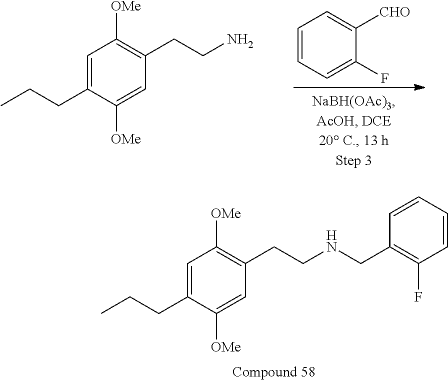 [SMILES: CCCc1cc(OC)c(CCN)cc1OC ; O=Cc1ccccc1F ; CCCc1cc(OC)c(CCNCc2ccccc2F)cc1OC]

### Step 1: Preparation of 2-(2,5-dimethoxy-4-propylphenyl)-N-(2-fluorobenzyl)ethanamine (58)

To a stirred solution of 2-(2,5-dimethoxy-4-propylphenyl)ethanamine (600 mg, 2.69 mmol, 1 eq.) and 2-fluorobenzaldehyde (267 mg, 2.15 mmol, 0.8 eq.) in DCE (5 mL) was added AcOH (0.05 mL). After stirring at 20° C. for 1 h, NaBH(OAc)₃ (1.71 g, 8.06 mmol, 3 eq.) was added and the mixture was stirred at 20° C. for 12 h. Upon completion, the mixture was basified to pH=8 with sat. aq. NaHCO₃ soln. and extracted with DCM (10 mL×2). The organic layer was dried over Na₂SO₄, filtered, and concentrated. The residue was purified by prep-HPLC (column: Welch Xtimate C18 100×25 mm×3 μm; mobile phase: [water (0.05% HCl)-ACN]; B %: 30%-50%, 8 min) to afford 2-(2,5-dimethoxy-4-propylphenyl)-N-(2-fluorobenzyl)ethanamine (198 mg, 597 μmol, 22% yield, HCl) as a white solid. LCMS R_{T}=2.296 min, MS cal.: 331.19, [M+H]⁺=332.1; 1H NMR (400 MHz, CHLOROFORM-d, HCl salt) δ=9.99 (br s, 2H), 7.86 (br s, 1H), 7.38-7.30 (m, 1H), 7.23-7.17 (m, 1H), 7.08 (br t, J=8.8 Hz, 1H), 6.78-6.52 (m, 2H), 4.37-4.09 (m, 2H), 3.72 (d, J=19.6 Hz, 6H), 3.29-3.01 (m, 4H), 2.64-2.44 (m, 2H), 1.57 (qd, J=7.6, 15.0 Hz, 2H), 0.94 (t, J=7.2 Hz, 3H); ¹³C NMR (101 MHz, CHLOROFORM-d, HCl salt) δ=162.59, 160.12, 151.39, 151.06, 132.72, 131.57, 131.49, 131.03, 125.05, 125.02, 122.23, 117.80, 117.66, 115.80, 115.59, 113.72, 112.86, 56.22, 55.80, 45.83, 43.22, 32.35, 27.99, 23.18, 14.09.

### Example 57: Preparation of 4-[2-(benzylamino)ethyl]-2,5-dimethoxybenzonitrile (59)

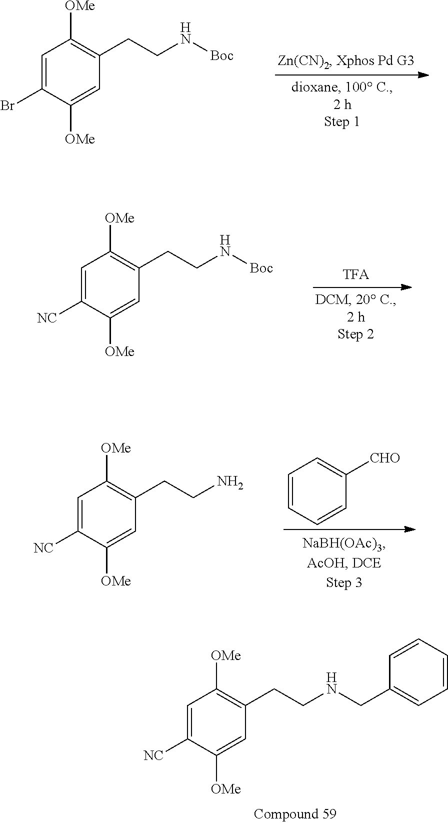 [SMILES: COc1cc(CCNC(=O)OC(C)(C)C)c(OC)cc1Br ; COc1cc(CCNC(=O)OC(C)(C)C)c(OC)cc1C#N ; COc1cc(CCN)c(OC)cc1C#N ; O=Cc1ccccc1 ; COc1cc(CCNCc2ccccc2)c(OC)cc1C#N]

### Step 1: Preparation of tert-butyl (4-cyano-2,5-dimethoxyphenethyl)carbamate

Under a N₂ atmosphere stirred at 20° C., tert-butyl (4-bromo-2,5-dimethoxyphenethyl)carbamate (1.5 g, 4.16 mmol, 1 eq.) and Zn(CN)₂ (342.3 mg, 2.91 mmol, 185 uL, 0.7 eq.) were dissolved in dioxane (10 mL) and treated with XPhos Pd G3 (529 mg, 625 μmol, 0.15 eq.). Then the mixture was warmed to 100° C. and stirred for 2 h. Upon completion, the reaction was concentrated and the residue was purified by column chromatography (SiO₂, Petroleum ether:Ethyl acetate=100:1-5:1) to give tert-butyl (4-cyano-2,5-dimethoxyphenethyl)carbamate (900 mg, 2.94 mmol, 71% yield) as a yellow solid. 1H NMR (400 MHz, CHLOROFORM-d) δ=6.97 (s, 1H), 6.80 (s, 1H), 4.62 (br s, 1H), 3.89 (s, 3H), 3.80 (s, 3H), 3.36 (q, J=6.4 Hz, 2H), 2.85 (br t, J=6.8 Hz, 2H), 1.43 (s, 9H).

### Step 2: Preparation of 4-(2-aminoethyl)-2,5-dimethoxybenzonitrile

tert-butyl (4-cyano-2,5-dimethoxyphenethyl)carbamate (0.9 g, 2.94 mmol, 1 eq.) was dissolved in DCM (10 mL) and treated with TFA (3.08 g, 27 mmol, 2 mL, 9.2 eq.) at 20° C. and stirred for 2 h. Upon completion, the reaction was carefully treated with sat. aq. Na₂CO₃ soln. until basic and extracted with DCM (5 mL×3). The organic layer was dried over Na₂SO₄, filtered, and concentrated to afford 4-(2-aminoethyl)-2,5-dimethoxybenzonitrile (410 mg, 2.0 mmol, 68% yield) as a yellow solid. 1H NMR (400 MHz, CHLOROFORM-d) δ ppm 7.01-6.93 (m, 1H), 6.81 (s, 1H), 3.94-3.85 (m, 3H), 3.83-3.74 (m, 3H), 3.00-2.92 (m, 2H), 2.87-2.76 (m, 2H), 1.94 (br s, 2H).

### Step 3: Preparation of 4-[2-(benzylamino)ethyl]-2,5-dimethoxybenzonitrile (59)

A solution of 4-(2-aminoethyl)-2,5-dimethoxybenzonitrile (410 mg, 2.0 mmol, 1 eq.), benzaldehyde (169 mg, 1.6 mmol, 161 uL, 0.8 eq.), and AcOH (12 mg, 199 μmol, 11.4 uL, 0.1 eq.) in DCE (10 mL) was stirred at 20° C. for 1 h. Then NaBH(OAc)₃ (1.26 g, 5.96 mmol, 3 eq.) was added and the mixture was stirred at 20° C. for 12 h. Upon completion, the mixture was basified to pH=8 with sat. aq. NaHCO₃ soln. and extracted with DCM (10 mL×2). The combined organic layer was washed with brine and dried over Na₂SO₄, filtered, and concentrated. The residue was purified by prep-HPLC (column: Kromasil C18 (250×50 mm×10 μm); mobile phase: [water (10 mM NH₄HCO₃)-ACN]; B %: 30%-60%, 10 min) to afford 4-[2-(benzylamino)ethyl]-2,5-dimethoxybenzonitrile (95 mg, 321 μmol, 16% yield) as a white solid. 1H NMR (400 MHz, DMSO-d₆) δ ppm 7.29 (d, J=4.4 Hz, 4H), 7.25 (s, 1H), 7.21 (td, J=4.0, 8.4 Hz, 1H), 7.08 (s, 1H), 7.10-7.07 (m, 1H), 3.83-3.82 (m, 1H), 3.83 (s, 2H), 3.75 (s, 3H), 3.70 (s, 2H), 2.82-2.74 (m, 2H), 2.73-2.65 (m, 2H); ¹³C NMR (400 MHz, DMSO-d₆) δ ppm 155.66, 151.47, 141.46, 137.39, 128.52, 128.38, 126.93, 114.92, 114.90, 97.82, 56.85, 56.61, 53.17, 48.63, 31.33.

### Example 58: Preparation of 4-(2-((benzo[d][1,3]dioxol-4-ylmethyl)amino)propyl)-2,5-dimethoxybenzonitrile (60)

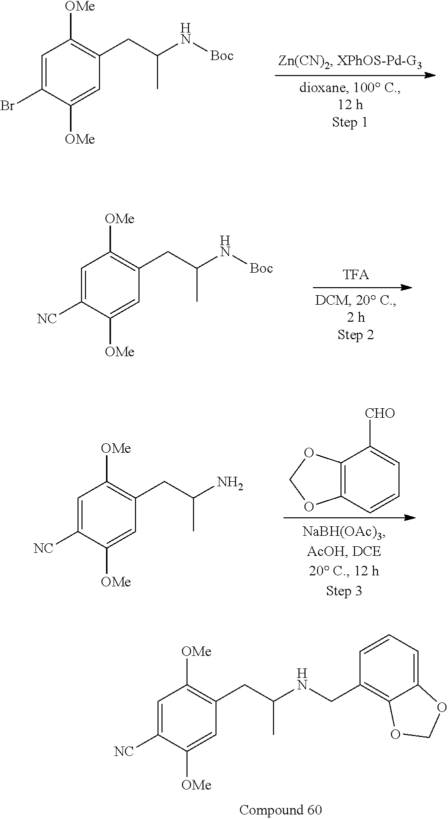 [SMILES: COc1cc(CC(C)NC(=O)OC(C)(C)C)c(OC)cc1Br ; COc1cc(CC(C)NC(=O)OC(C)(C)C)c(OC)cc1C#N ; COc1cc(CC(C)N)c(OC)cc1C#N ; O=Cc1cccc2c1OCO2 ; COc1cc(CC(C)NCc2cccc3c2OCO3)c(OC)cc1C#N]

### Step 1: Preparation of tert-butyl (1-(4-cyano-2,5-dimethoxyphenyl)propan-2-yl)carbamate

A mixture of Zn(CN)₂ (308 mg, 2.62 mmol, 166.2 uL, 0.7 eq.) and tert-butyl (1-(4-bromo-2,5-dimethoxyphenyl)propan-2-yl)carbamate (1.4 g, 3.74 mmol, 1 eq.) in dioxane (10 mL) was treated with XPhos Pd G₃ (475 mg, 562 μmol, 0.15 eq.). The mixture was stirred and warmed to 100° C. for 12 h. Upon completion, the reaction was concentrated and the residue purified by column chromatography (SiO₂, Petroleum ether:Ethyl acetate=100:1-5:1) to afford tert-butyl (1-(4-cyano-2,5-dimethoxyphenyl)propan-2-yl)carbamate (850 mg, 2.65 mmol, 70.93% yield) as a yell ow solid. ¹H NMR (400 MHz, CHLOROFORM-d) δ ppm 6.96 (s, 1H), 6.85-6.74 (m, 1H) 4.55 (s, 1H), 4.23-4.00 (m, 1H), 3.88 (s, 3H), 3.84-3.75 (m, 3H), 2.86-2.73 (m, 2H), 1.50-1.33 (m, 9H), 1.17-1.08 (m, 3H).

### Step 2: Preparation of 4-(2-aminopropyl)-2,5-dimethoxybenzonitrile

To a stirred solution of tert-butyl (1-(4-cyano-2,5-dimethoxyphenyl)propan-2-yl)carbamate (850 mg, 2.65 mmol, 1 eq.) in DCM (10 mL) was added TFA (3.08 g, 27 mmol, 2 mL, 10 eq.) at 20° C. and the mixture was stirred at 20° C. for 3 h. Upon completion, the reaction was carefully treated with sat. aq. Na₂CO₃ soln. (1.5 g Na₂CO₃) and extracted with DCM (10 mL×3). The combined organic layer was washed with brine and dried over Na₂SO₄, filtered, and concentrated to give 4-(2-aminopropyl)-2,5-dimethoxybenzonitrile (410 mg, 1.86 mmol, 70% yield) as a yellow oil. 1H NMR (400 MHz, CHLOROFORM-d) δ ppm 7.26 (d, J=4.4 Hz, 1H), 6.99 (d, J=3.2 Hz, 1H), 4.26-4.08 (m, 2H), 3.91-3.88 (m, 4H), 3.83-3.77 (m, 3H), 3.40-3.29 (m, 1H), 1.74 (s, 2H), 1.20-1.16 (m, 1H), 1.19-1.15 (m, 3H).

### Step 3: Preparation of 4-(2-((benzo[d][1,3]dioxol-4-ylmethyl)amino)propyl)-2,5-dimethoxybenzonitrile (60)

A solution of 4-(2-aminopropyl)-2,5-dimethoxybenzonitrile (230 mg, 1.04 mmol, 1 eq.), benzo[d][1,3]dioxole-4-carbaldehyde (62.7 mg, 418 μmol, 48 uL, 0.4 eq.), and AcOH (6.3 mg, 104 μmol, 6.0 uL, 0.1 eq.) in DCE (5 mL) was stirred at 20° C. for 1 h. To this solution was added NaBH(OAc)₃ (664 mg, 3.13 mmol, 3 eq.) and the mixture stirred at 20° C. for 12 h. Upon completion, the mixture was basified to pH=8 with sat. aq. NaHCO₃ soln. and extracted with DCM (5 mL×2). The combined organic layer was washed with brine and dried over Na₂SO₄, filtered, and concentrated. The residue was purified by prep-HPLC (neutral condition) to afford 4-(2-((benzo[d][1,3]dioxol-4-ylmethyl)amino)propyl)-2,5-dimethoxybenzonitrile (50 mg, 141 μmol, 14% yield) as a white solid. ¹H NMR (400 MHz, DMSO-d₆) δ ppm 7.25 (s, 1H), 7.02 (s, 1H), 6.82-6.73 (m, 3H), 5.93 (d, J=9.6 Hz, 2H), 3.81 (s, 3H), 3.70 (s, 3H), 3.70-3.61 (m, 2H), 2.91-2.74 (m, 2H), 2.54 (s, 1H), 1.82 (s, 1H), 0.95 (d, J=6.0 Hz, 3H); ¹³C NMR (101 MHz, DMSO-d₆) δ ppm 155.47, 151.64, 147.01, 145.29, 136.81, 123.00, 122.23, 121.59, 117.15, 115.48, 114.92, 107.25, 56.83, 56.55, 52.18, 44.49, 38.06, 20.61.

### Example 59: Preparation of 1-(2,5-demethoxy-4-propylphenyl)-N-(2-methoxybenzyl)propan-2-amine (61)

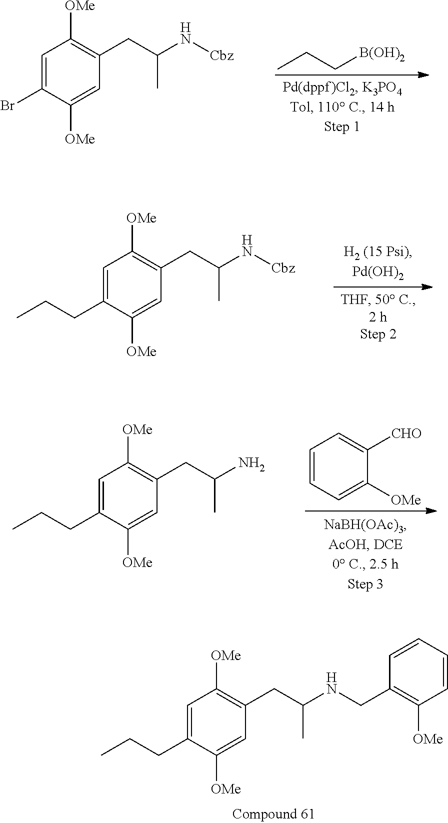

### Step 1: Preparation of benzyl (1-(2,5-dimethoxy-4-propylphenyl)propan-2-yl)carbamate

To a stirred solution of benzyl (1-(4-bromo-2,5-dimethoxyphenyl)propan-2-yl)carbamate (2 g, 4.90 mmol, 1 eq.) in toluene (20 mL) was added propylboronic acid (517 mg, 5.9 mmol, 1.2 eq.), K₃PO₄ (3.12 g, 14.7 mmol, 3 eq.), and Pd(dppf)Cl₂ (358 mg, 490 μmol, 0.1 eq.) under N₂. The mixture was warmed to 110° C. and stirred for 12 h. Upon completion, the mixture was cooled, filtered, and concentrated. The residue was purified by column chromatography (SiO₂, Petroleum ether/Ethyl acetate=100/1-1/1) to afford benzyl (1-(2,5-dimethoxy-4-propylphenyl)propan-2-yl)carbamate (1.3 g, 3.50 mmol, 71% yield) as a white solid. 1H NMR (400 MHz, CHLOROFORM-d) δ ppm 7.37-7.28 (m, 5H), 6.67 (s, 1H), 6.62 (s, 1H), 5.11 (s, 1H), 5.05 (s, 2H), 4.01-3.90 (m, 1H), 3.79-3.73 (m, 6H), 2.85-2.67 (m, 2H), 2.60-2.53 (m, 2H), 1.63-1.56 (m, 2H), 1.18 (d, J=6.5 Hz, 3H), 0.96 (t, J=7.4 Hz, 3H).

### Step 2: Preparation of 1-(2,5-dimethoxy-4-propylphenyl)propan-2-amine

To a solution benzyl (1-(2,5-dimethoxy-4-propylphenyl)propan-2-yl)carbamate (1.2 g, 3.23 mmol, 1 eq.) in THF (15 mL) was added Pd(OH)₂ (454 mg, 323 μmol, 10% purity, 0.1 eq.). The mixture was warmed to 50° C. and stirred for 2 h under H₂ (15 Psi). Upon completion, the mixture was filtered, and concentrated to afford 1-(2,5-dimethoxy-4-propylphenyl)propan-2-amine (0.75 g, 3.16 mmol, 98% yield) as a white solid. 1H NMR (400 MHz, CHLOROFORM-d) δ ppm 6.68 (s, 1H), 6.69-6.67 (m, 1H), 6.67-6.65 (m, 1H), 3.78 (d, J=1.7 Hz, 4H), 3.80-3.75 (m, 1H), 3.24-3.15 (m, 1H), 2.72 (dd, J=5.2, 13.0 Hz, 1H), 2.59-2.45 (m, 3H), 1.61 (m, 2H), 1.33 (s, 2H), 1.12 (d, J=6.2 Hz, 3H), 0.97 (t, J=7.3 Hz, 3H).

### Step 3: Preparation of 1-(2,5-dimethoxy-4-propylphenyl)-N-(2-methoxybenzyl)propan-2-amine (61

A mixture of 1-(2,5-dimethoxy-4-propylphenyl)propan-2-amine (0.75 g, 3.16 mmol, 1 eq.), 2-methoxybenzaldehyde (387.2 mg, 2.84 mmol, 0.9 eq.), and AcOH (380 mg, 6.32 mmol, 362 uL, 2 eq.) in DCE (20 mL) was stirred at 0° C. for 1.5 h. To this was added NaBH(OAc)₃ (2.01 g, 9.48 mmol, 3 eq.) and the mixture was stirred at 0° C. for 1 h. Upon completion, the mixture was basified to pH=9 with sat. aq. Na₂CO₃ soln. and extracted with DCM (10 mL×2). The organic layer was washed with brine, dried over Na₂SO₄, filtered, and concentrated. The residue was purified by prep-HPLC (column: Phenomenex luna C18 250×50 mm×10 μm; mobile phase: [water (0.04% HCl)-ACN]; B %: 30%-70%, 10 min) to afford 1-(2,5-dimethoxy-4-propylphenyl)-N-(2-methoxybenzyl)propan-2-amine (400 mg, 1.02 mmol, 32% yield, HCl) as a white solid. LCMS R_{T}=2.338 min, MS cal.: 357.49, [M+H]⁺=358.1; 1H NMR (400 MHz, DMSO-d₆, HCl salt) δ ppm 9.50-9.32 (m, 1H), 9.16-9.00 (m, 1H), 7.56 (dd, J=1.6, 7.6 Hz, 1H), 7.41 (t, J=7.6 Hz, 1H), 7.09 (d, J=8.0 Hz, 1H), 7.00 (t, J=7.2 Hz, 1H), 6.77 (d, J=4.0 Hz, 2H), 4.16 (t, J=4.8 Hz, 2H), 3.81 (s, 3H), 3.70 (d, J=14.0 Hz, 5H), 3.72 (d, J=10.8 Hz, 1H), 3.33 (d, J=4.4 Hz, 1H), 3.17 (dd, J=4.4, 13.0 Hz, 1H), 2.73 (dd, J=10.0, 12.8 Hz, 1H), 2.50-2.46 (m, 2H), 1.53 (m, 2H), 1.18 (d, J=6.4 Hz, 3H), 0.89 (t, J=7.2 Hz, 3H); ¹³C NMR (101 MHz, DMSO-d₆, HCl salt) δ ppm 157.51, 150.82, 150.78, 131.48, 130.60, 129.60, 122.46, 120.31, 119.91, 113.85, 113.16, 110.98, 55.86, 55.81, 55.52, 53.27, 42.43, 33.09, 31.72, 22.73, 15.55, 13.89.

### Example 60: Preparation of 1-(2,5-dimethoxy-4-propylphenyl)butan-2-amine (63)

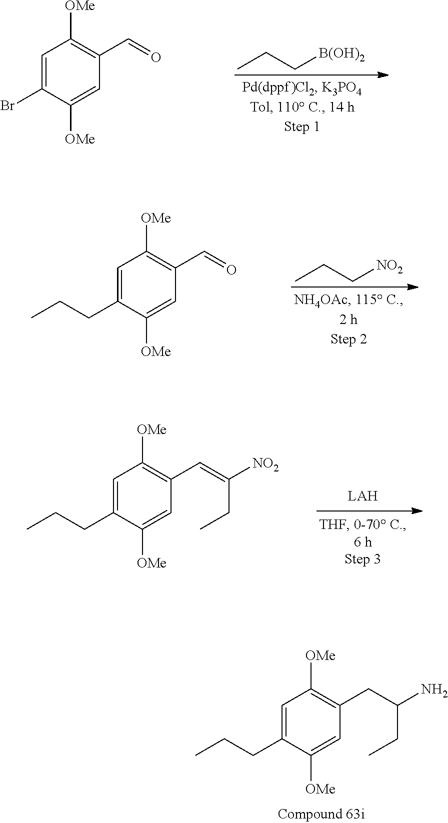

### Step 1: Preparation of 2,5-dimethoxy-4-propylbenzaldehyde

To a solution of 4-bromo-2,5-dimethoxybenzaldehyde (5 g, 20.4 mmol, 1 eq.) and propylboronic acid (2.33 g, 26.5 mmol, 1.3 eq.) in toluene (50 mL) was added K₃PO₄ (12.99 g, 61.21 mmol, 3 eq.) and Pd(dppf)Cl₂ (746.43 mg, 1.02 mmol, 0.05 eq.). The mixture was warmed to 110° C. and stirred for 12 h. Upon completion, the mixture was filtered, and concentrated. The residue was purified by column chromatography (SiO₂, Petroleum ether/Ethyl acetate=100/0 to 4/1) to afford 2,5-dimethoxy-4-propylbenzaldehyde (3.65 g, 17.5 mmol, 86% yield) as a yellow oil. 1H NMR (400 MHz, CHLOROFORM-d) δ ppm 10.88 (s, 1H), 7.74 (s, 1H), 7.27 (s, 1H), 4.37 (s, 3H), 4.30 (s, 3H), 3.14-3.07 (m, 2H), 2.10 (m, 2H), 1.45 (t, J=7.4 Hz, 3H).

### Step 2: Preparation of 1,4-dimethoxy-2-[(E)-2-nitrobut-1-en-1-yl]-5-propylbenzene

A solution of 2,5-dimethoxy-4-propylbenzaldehyde (3.65 g, 17.5 mmol, 1 eq.), 1-nitropropane (35.93 g, 403 mmol, 36 mL, 23 eq.), and NH₄OAc (2.70 g, 35 mmol, 2 eq.) was warmed to 115° C. and stirred for 2 h. Upon completion, the solvent was removed. The residue was purified by column chromatography (SiO₂, Petroleum ether/Ethyl acetate=100/0 to 4/1) to afford 1,4-dimethoxy-2-[(E)-2-nitrobut-1-en-1-yl]-5-propylbenzene (2.0 g, 7.16 mmol, 41% yield) as a yellow oil. 1H NMR (400 MHz, CHLOROFORM-d) δ ppm 8.26 (s, 1H), 6.80 (s, 1H), 6.76 (s, 1H), 3.84 (s, 3H), 3.81 (s, 3H), 2.87 (q, J=7.3 Hz, 2H), 2.66-2.58 (m, 2H), 1.69-1.58 (m, 2H), 1.30 (t, J=7.3 Hz, 3H), 0.98 (t, J=7.4 Hz, 3H).

### Step 3: Preparation of 1-(2,5-dimethoxy-4-propylphenyl)butan-2-amine (63i)

A solution of 1,4-dimethoxy-2-[(E)-2-nitrobut-1-en-1-yl]-5-propylbenzene (2 g, 7.16 mmol, 1 eq.) in THF (30 mL) was cooled to 0° C. and treated with LiAlH₄ (1.09 g, 28.6 mmol, 4 eq.). The mixture was warmed to 85° C. and stirred for 6 h. Upon completion, the mixture was cooled to 0° C. then stirred and treated dropwise with H₂O (1.09 mL). 30% aq. NaOH (1.09 mL) was added dropwise and stirring continued until a smooth dispersion formed. The mixture was filtered and concentrated. The residue was purified by prep-HPLC (column: Welch Xtimate C18 100×25 mm×3 μm; mobile phase: [water (0.04% HCl)-ACN]; B %: 20%-40%, 8 min) to afford 1-(2,5-dimethoxy-4-propylphenyl)butan-2-amine (1.08 g, 3.63 mmol, 51% yield, 97% purity, HCl) as a white solid. LCMS R_{T}=2.107 min, MS cal.: 251.36, [M+H]⁺=252.1; 1H NMR (400 MHz, CHLOROFORM-d, HCl salt) δ ppm 8.46-8.19 (m, 3H), 6.73 (s, 1H), 6.68 (s, 1H), 3.80 (s, 3H), 3.79 (s, 3H), 3.55-3.43 (m, 1H), 3.08-2.95 (m, 2H), 2.60-2.50 (m, 2H), 1.88-1.68 (m, 2H), 1.63-1.57 (m, 2H), 1.10 (t, J=7.4 Hz, 3H), 0.96 (t, J=7.3 Hz, 3H); ¹³C NMR (101 MHz, CHLOROFORM-d, HCl salt) δ ppm 151.06, 150.89, 130.86, 121.49, 114.09, 112.77, 76.89, 55.94, 55.68, 53.80, 33.96, 32.05, 25.14, 22.85, 13.80, 9.88.

### Example 61: Preparation of 1-(2,5-dimethoxy-4-propylphenyl)-N-(2-methoxybenzyl)butan-2-amine (63)

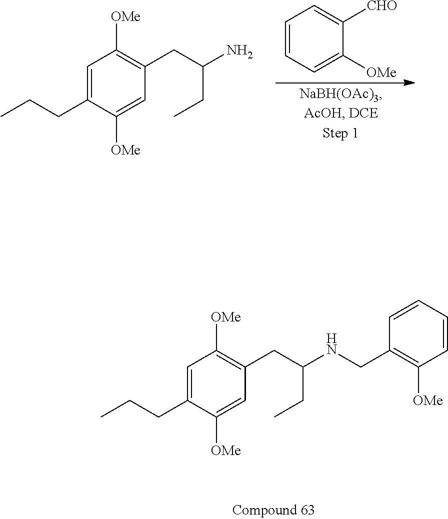 [SMILES: CCCc1cc(OC)c(CC(N)CC)cc1OC ; COc1ccccc1C=O ; CCCc1cc(OC)c(CC(CC)NCc2ccccc2OC)cc1OC]

### Step 1: Preparation of 1-(2,5-dimethoxy-4-propylphenyl)-N-(2-methoxybenzyl)butan-2-amine

A solution of 1-(2,5-dimethoxy-4-propylphenyl)butan-2-amine (1 g, 3.98 mmol, 1 eq.), 2-methoxybenzaldehyde (379 mg, 2.8 mmol, 0.7 eq.), and AcOH (24 mg, 398 μmol, 23 uL, 0.1 eq.) in DCE (20 mL) was stirred at 20° C. for 2.5 h. Then NaBH(OAc)₃ (2.11 g, 9.95 mmol, 2.5 eq.) was added and the mixture stirred at 20° C. for 10 h. Upon completion, the mixture was basified to pH=9 with sat. aq. NaHCO₃ soln. and extracted with DCM (10 mL×2). The organic layer was washed with brine, dried over Na₂SO₄, filtered, and concentrated. The residue was purified by prep-HPLC (column: Phenomenex luna C18 (250×70 mm, 15 μm); mobile phase: [water (0.04% HCl)-ACN]; B %: 19%-49%, 20 min) to afford 1-(2,5-dimethoxy-4-propylphenyl)-N-(2-methoxybenzyl)butan-2-amine (500 mg, 1.47 mmol, 37% yield, 100% purity, HCl) as a white solid. LCMS R_{T}=2.405 min, MS cal.: 371.51, [M+H]⁺=372.1; 1H NMR (400 MHz, CHLOROFORM-d, HCl salt) δ ppm 10.57-10.31 (m, 1H), 7.51-7.38 (m, 1H), 7.37-7.31 (m, 1H), 7.20 (d, J=7.2 Hz, 1H), 6.95 (t, J=7.2 Hz, 1H), 6.83 (s, 1H), 6.75 (d, J=8.4 Hz, 1H), 6.58 (s, 1H), 4.33 (d, J=13.6 Hz, 1H), 4.08-3.96 (m, 1H), 3.79 (s, 3H), 3.53 (s, 3H), 3.50 (s, 3H), 3.13-3.05 (m, 1H), 3.02-2.94 (m, 2H), 2.62-2.47 (m, 2H), 2.04-1.88 (m, 2H), 1.58 (m, 2H), 1.04 (t, J=7.2 Hz, 3H), 1.00-0.94 (m, 3H); ¹³C NMR (101 MHz, CHLOROFORM-d, HCl salt) δ ppm 157.28, 151.52, 150.91, 131.75, 131.21, 131.06, 121.32, 121.14, 118.30, 114.47, 112.84, 110.23, 57.49, 56.21, 55.53, 55.18, 45.40, 32.73, 32.40, 23.21, 23.18, 14.12, 10.09.

### Example 62: Preparation of 4-(2-((2-hydroxybenzyl)amino)propyl)-2,5-dimethoxybenzonitrile (64)

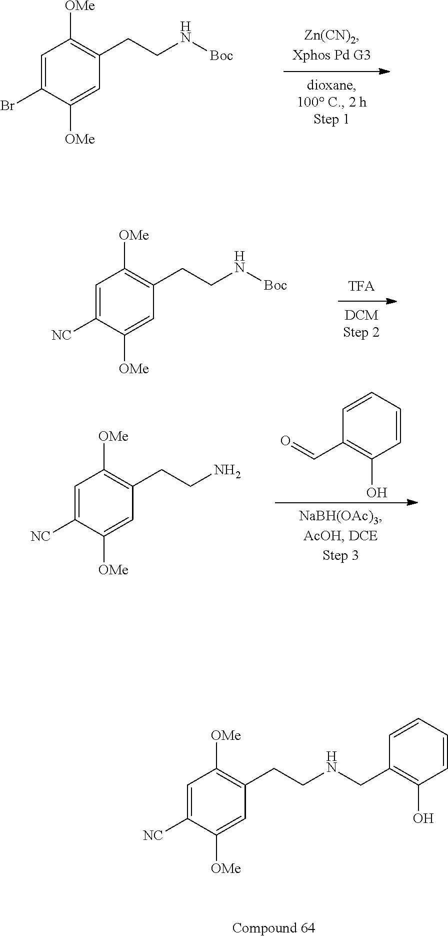 [SMILES: COc1cc(CCNC(=O)OC(C)(C)C)c(OC)cc1Br ; COc1cc(CCNC(=O)OC(C)(C)C)c(OC)cc1C#N ; COc1cc(CCN)c(OC)cc1C#N ; O=Cc1ccccc1O ; COc1cc(CCNCc2ccccc2O)c(OC)cc1C#N]

### Step 1: Preparation of tert-butyl (4-cyano-2,5-dimethoxyphenethyl)carbamate

A solution of tert-butyl (4-bromo-2,5-dimethoxyphenethyl)carbamate (2 g, 5.55 mmol, 1 eq.), Zn(CN)₂ (456 mg, 3.89 mmol, 247 uL, 0.7 eq.), and XPhos Pd G₃ (705 mg, 833 μmol, 0.15 eq.) in dioxane (30 mL) was warmed to 100° C. and stirred for 1 h. Upon completion, the mixture was filtered, and concentrated. The residue was purified on silica gel chromatography (PE:EA=10:1-3:1) to afford tert-butyl (4-cyano-2,5-dimethoxyphenethyl)carbamate (1.6 g, 5.22 mmol, 94% yield) as a white solid.

### Step 2: Preparation of 4-(2-aminoethyl)-2,5-dimethoxybenzonitrile

A solution of tert-butyl (4-cyano-2,5-dimethoxyphenethyl)carbamate (1 g, 3.26 mmol, 1 eq.) and TFA (4.62 g, 40.5 mmol, 3 mL, 12.4 eq.) in DCM (10 mL) was stirred at 20° C. for 2 h. Upon completion, the solvent was removed. The residue was dissolved with DCM (10 mL) and basified to pH=9 with sat. aq. Na₂CO₃ soln. The organic layer was washed with brine, dried over Na₂SO₄, filtered, and concentrated to afford 4-(2-aminoethyl)-2,5-dimethoxybenzonitrile (580 mg, 2.81 mmol, 86% yield) as a yellow oil.

### Step3: Preparation of 4-(2-((2-hydroxybenzyl)amino)propyl)-2,5-dimethoxybenzonitrile (64)

A solution of 4-(2-aminoethyl)-2,5-dimethoxybenzonitrile (570 mg, 2.76 mmol, 1 eq.), 2-hydroxybenzaldehyde (270.01 mg, 2.21 mmol, 234.79 uL, 0.8 eq.), and AcOH (105 mg, 1.75 mmol, 0.1 mL) in DCE (8 mL) was stirred at 15° C. for 2 h. Then NaBH(OAc)₃ (1.17 g, 5.53 mmol, 2 eq.) was added and the mixture was stirred at 15° C. for 12 h. Upon completion, the mixture was basified to pH=9 with sat. aq. NaHCO₃ soln. and extracted with DCM (5 mL×2). The organic layer was washed with brine, dried over Na₂SO₄, filtered, and concentrated. The residue was purified by prep-HPLC (column: Kromasil C18 (250×50 mm×10 μm); mobile phase: [water (10 mM NH₄HCO₃)-ACN]; B %: 30%-60%, 10 min) to afford 4-(2-((2-hydroxybenzyl)amino)propyl)-2,5-dimethoxybenzonitrile (217 mg, 615.3 μmol, 22% yield, 98.9% purity) as an off-white solid. LCMS R_{T}=1.879 min, MS cal.: 312.36, [M+H]⁺=313.1; 1H NMR (400 MHz, CHLOROFORM-d) δ ppm 7.19-7.15 (m, 1H), 6.99-6.97 (m, 2H), 6.83-6.76 (m, 3H), 4.00 (s, 2H), 3.89 (s, 3H), 3.79 (s, 3H), 2.95-2.87 (m, 4H); ¹³C NMR (101 MHz, CHLOROFORM-d) δ ppm 157.74, 155.65, 151.44, 136.65, 129.07, 128.35, 124.57, 118.90, 117.16, 115.78, 114.99, 114.94, 97.99, 56.83, 56.62, 50.72, 48.07, 30.73.

### Example 63: Preparation of 2-(4-(butylthio)-3,5-demethoxy-2-methylphenyl)ethanamine (67)

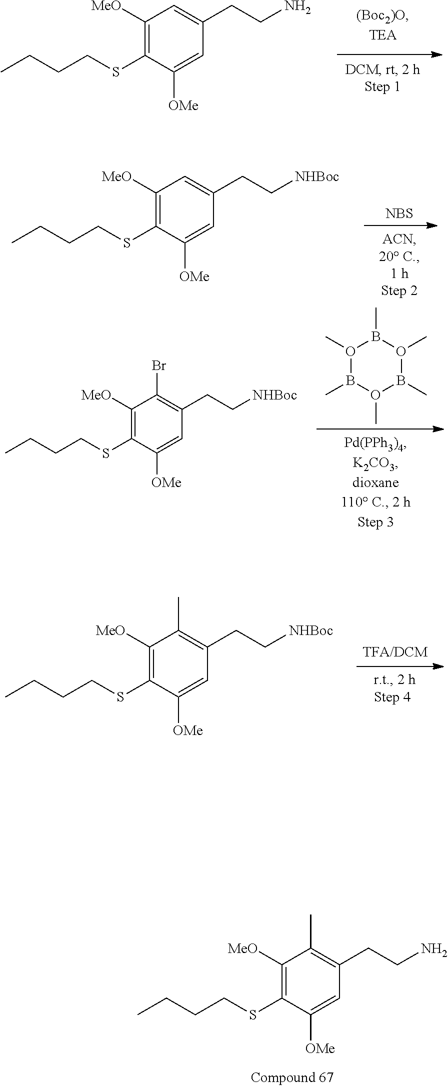

### Step 1: Preparation of tert-butyl (4-(butylhtio)-3,5-dimethoxyphenethyl)carbamate

A stirred solution of 2-(4-(butylthio)-3,5-dimethoxyphenyl)ethanamine (3 g, 11.1 mmol, 1 eq.), (Boc)₂O (4.86 g, 22.3 mmol, 5.12 mL, 2 eq.), and TEA (3.38 g, 33.4 mmol, 4.65 mL, 3 eq.) in DCM (30 mL) was degassed and purged with N₂ 3 times then warmed to 60° C. for 1 h under a N₂ atmosphere. Upon completion, the reaction mixture was concentrated. The crude product was purified by column chromatography (SiO₂, PE:EA=100:1-30:1) to afford tert-butyl (4-(butylthio)-3,5-dimethoxyphenethyl)carbamate (1.3 g, 3.52 mmol, 32% yield) as a yellow oil. ¹H NMR (400 MHz, CHLOROFORM-d) δ ppm 6.40 (s, 2H), 4.61 (s, 1H), 3.88 (s, 6H), 3.39 (s, 2H), 2.78 (t, J=7.6 Hz, 4H), 1.37-1.53 (m, 13H), 0.87 (t, J=7.2 Hz, 3H).

### Step 2: Preparation of tert-butyl (2-bromo-4-(butylthio)-3,5-dimethoxyphenethyl)carbamate

To a stirred solution of tert-butyl (4-(butylthio)-3,5-dimethoxyphenethyl)carbamate (800 mg, 2.16 mmol, 1 eq.) in ACN (8 mL) was added NBS (424 mg, 2.38 mmol, 1.1 eq.). The mixture was stirred at 20° C. for 1 h. Upon completion, the reaction mixture was quenched by addition of sat. aq. NaHCO₃ soln. (10 mL) at 20° C. The mixture was extracted with EA (4 mL×3) and the combined organic layers were dried over Na₂SO₄, filtered, and concentrated to give a residue. The residue was purified by column chromatography (SiO₂, Petroleum ether/Ethyl acetate=10/1-3/1) to give tert-butyl (2-bromo-4-(butylthio)-3,5-dimethoxyphenethyl)carbamate (800 mg, 1.78 mmol, 82% yield). ¹H NMR (400 MHz, CHLOROFORM-d) δ ppm 6.59 (s, 1H), 4.64 (s, 1H), 3.88 (d, J=3.6, 6H), 3.40 (m, 2H), 2.964 (t, J=7.6 Hz, 2H), 2.86 (t, J=7.6 Hz, 2H), 1.37-1.53 (m, 13H), 0.88 (t, J=7.2 Hz, 3H).

### Step 3: Preparation of tert-butyl (4-(butylthio)-3,5-dimethoxy-2-methylphenethyl)carbamate

A mixture of tert-butyl (2-bromo-4-(butylthio)-3,5-dimethoxyphenethyl)carbamate (700 mg, 1.56 mmol, 1 eq.), 2,4,6-trimethyl-1,3,5,2,4,6-trioxatriborinane (1.18 g, 4.68 mmol, 1.31 mL, 50% purity, 3 eq.), Pd(PPh₃)₄ (180.39 mg, 156.11 μmol, 0.1 eq.), and K₂CO₃ (647 mg, 4.7 mmol, 3 eq.) in dioxane (10 mL) was de-gassed and then warmed to 110° C. and stirred for 3 h under N₂. Upon completion, the reaction mixture was poured into H₂O (20 mL). The mixture was extracted with ethyl acetate (5 mL×3). The organic phase was dried over anhydrous Na₂SO₄, filtered, and concentrated to give a residue. The crude material was purified by column chromatography (SiO₂, Petroleum ether/Ethyl acetate=20:1-5:1) to afford tert-butyl (4-(butylthio)-3,5-dimethoxy-2-methylphenethyl)carbamate (480 mg, 1.25 mmol, 80% yield). ¹H NMR (400 MHz, CHLOROFORM-d) δ ppm 6.50 (s, 1H), 4.48-4.70 (m, 1H), 3.86 (s, 3H), 3.80 (s, 3H), 3.33 (t, J=4.0 Hz, 2H), 2.83-2.89 (m, 2H), 2.81 (t, J=7.2 Hz, 2H), 2.21 (s, 3H), 1.49-1.56 (m, 1H), 1.47-1.57 (m, 2H), 1.42-1.47 (m, 11H), 0.88 (t, J=7.2 Hz, 3H).

### Step 4: Preparation of 2-(4-(butylthio)-3,5-dimethoxy-2-methylphenyl)ethanamine (67)

To a solution of tert-butyl (4-(butylthio)-3,5-dimethoxy-2-methylphenethyl)carbamate (480 mg, 1.25 mmol, 1 eq.) in DCM (4 mL) was added TFA (2.19 g, 19.22 mmol, 1.42 mL, 15 eq.). The mixture was stirred at 15° C. for 4 h. Upon completion, the mixture was concentrated. The residue was purified by prep-HPLC (column: Phenomenex luna C18 80×40 mm×3 μm; mobile phase: [water (0.04% HCl)-ACN]; B %: 27%-32%, 7 min) to give 2-(4-(butylthio)-3,5-dimethoxy-2-methylphenyl)ethanamine (300 mg, 1.06 mmol, 85% yield, HCl) as a white solid. ¹H NMR (400 MHz, DMSO-d₆, HCl salt) δ ppm 8.43 (s, 3H), 6.60 (s, 1H), 3.88 (s, 3H), 3.79 (s, 3H), 3.13-3.21 (m, 4H), 2.08-2.19 (t, J=7.6 Hz, 2H), 2.24 (s, 3H), 1.34-1.42 (m, 4H), 0.833 (t, J=7.2 Hz, 3H); ¹³C NMR (101 MHz, DMSO-d₆, HCl salt) δ ppm 160.74, 158.72, 135.69, 122.72, 116.31, 108.64, 60.69, 56.37, 39.88, 34.01, 32.18, 31.88, 21.97, 13.73, 12.01.

### Example 64: Preparation of 2-(2-bromo-4-(butylthio)-3,5-dimethoxyphenyl)ethanamine (67i)

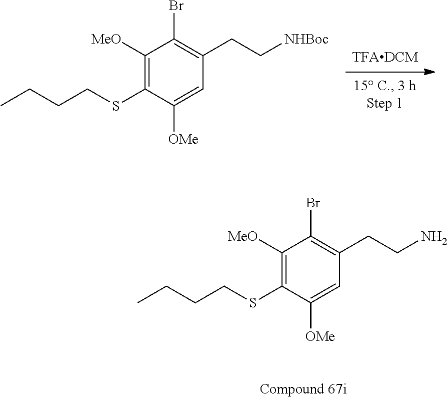 [SMILES: CCCCSc1c(OC)cc(CCNC(=O)OC(C)(C)C)c(Br)c1OC ; CCCCSc1c(OC)cc(CCN)c(Br)c1OC]

### Step 1: Preparation of 2-(2-bromo-4-(butylthio)-3,5-dimethoxyphenyl)ethanamine (67i)

To a solution of tert-butyl (2-bromo-4-(butylthio)-3,5-dimethoxyphenethyl)carbamate (100 mg, 223 μmol, 1 eq.) in DCM (2 mL) was added TFA (2.00 g, 17.5 mmol, 1.30 mL, 79 eq.). The mixture was stirred at 15° C. for 4 h. Upon completion, the mixture was concentrated and the residue purified by prep-HPLC (column: Welch Xtimate C18 100×25 mm×3 μm; mobile phase: [water (0.05% HCl)-ACN]; B %: 25%-55%, 8 min) to give 2-(2-bromo-4-(butylthio)-3,5-dimethoxyphenyl)ethanamine (38 mg, 109.1 μmol, 49% yield, HCl) as a white solid. ¹H NMR (400 MHz, DMSO-d₆, HCl salt) δ ppm 8.12 (s, 3H), 6.91 (s, 1H), 3.85 (s, 3H), 3.76 (s, 3H), 3.04 (s, 4H), 2.08-2.19 (t, J=6.8 Hz, 2H), 1.34-1.41 (m, 4H), 0.833 (t, J=6.8 Hz, 3H); ¹³C NMR (101 MHz, DMSO-d₆, HCl salt) δ ppm 159.57, 157.84, 137.91, 116.72, 110.38, 110.00, 60.31, 56.30, 38.06, 33.80, 32.94, 31.28, 21.12, 13.44.

### Example 65: Preparation of 1-(2,5-demethoxy-3-methyl-4-pentylphenyl)propan-2-amine (68)

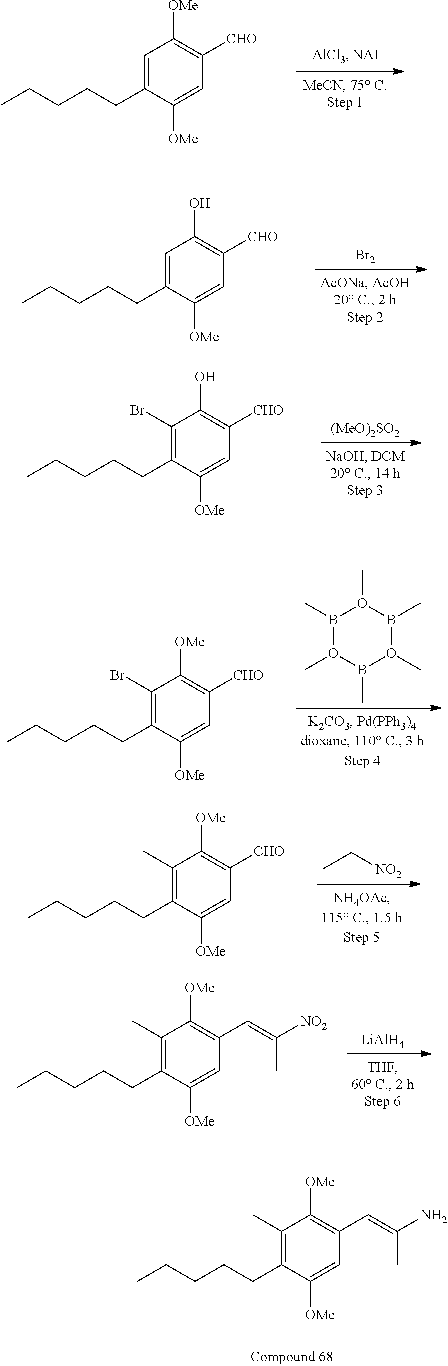

### Step 1: Preparation of 2-hydroxy-5-methoxy-4-pentylbenzaldehyde

A mixture of 2,5-dimethoxy-4-pentylbenzaldehyde (4 g, 16.9 mmol, 1 eq.) in MeCN (50 mL) was added dropwise to a solution of MeCN (50 mL) containing AlCl₃ (2.28 g, 17.1 mmol, 1.01 eq.). The resulting mixture was stirred and warmed to 45° C. NaI (3.81 g, 25.39 mmol, 1.5 eq.) was then added and the mixture was vigorously stirred at 80° C. for 2 h. Upon completion, the reaction was concentrated to give a residue. The residue was dissolved in 30 mL of EtOAc and treated with 30 mL of sat. aq. tartaric acid disodium soln. with vigorous stirring until two distinct layers formed (˜1 h). The aqueous phase was extracted with EtOAc (30 mL×2). The combined organic layer was washed first with sat. aq. Na₂S₂O₃ soln. (3 mL×2), then washed with brine (10 mL). The combined organic layer was dried over Na₂SO₄, filtered, and concentrated to give 2-hydroxy-5-methoxy-4-pentylbenzaldehyde (3.70 g, 16.7 mmol, 98% yield) as a yellow solid. 1H NMR (400 MHz, CHLOROFORM-d) δ=10.78 (s, 1H), 9.84-9.79 (m, 1H), 6.95-6.87 (m, 1H), 6.85-6.75 (m, 1H), 3.90-3.79 (m, 3H), 2.71-2.56 (m, 2H), 1.64-1.52 (m, 2H), 1.39-1.28 (m, 5H), 0.94-0.85 (m, 3H).

### Step 2: Preparation of 3-bromo-2-hydroxy-5-methoxy-4-pentylbenzaldehyde

A solution of the 2-hydroxy-5-methoxy-4-pentylbenzaldehyde (4.1 g, 18.5 mmol, 1 eq.) and anhydrous AcONa (2.27 g, 27.7 mmol, 1.5 eq.) in AcOH (20 mL) was stirred at 20° C. After the complete dissolution of all solids, a solution of Br₂ (3.10 g, 19.4 mmol, 999 uL, 1.05 eq.) dissolved in AcOH (10 mL) was added dropwise (˜30 min) to the phenol solution at 20° C. and the mixture stirred for 2 h. Upon completion, the reaction was diluted in H₂O (10 mL) and extracted with EtOAc (20 mL×3). The combined organic layer was washed with brine (30 mL), dried over Na₂SO₄, filtered, and concentrated to give 3-bromo-2-hydroxy-5-methoxy-4-pentylbenzaldehyde (4.8 g, 16 mmol, 86% yield) as a yellow solid. ¹H NMR (400 MHz, CHLOROFORM-d) δ=11.41 (s, 1H), 9.79 (s, 1H), 6.99-6.88 (m, 1H), 3.91-3.79 (m, 3H), 2.98-2.81 (m, 2H), 1.58-1.47 (m, 2H), 1.45-1.32 (m, 4H), 0.97-0.87 (m, 3H).

### Step 3: Preparation of 3-bromo-2,5-dimethoxy-4-pentylbenzaldehyde

To a solution of 3-bromo-2-hydroxy-5-methoxy-4-pentylbenzaldehyde (4.7 g, 15.61 mmol, 1 eq.) in DCM (100 mL) under a N₂ atmosphere stirred at 20° C. was added an aqueous solution of NaOH (1.04 g, 26.06 mmol, 1.67 eq in 35 mL H₂O). After stirring vigorously Me₂SO₄ (3.94 g, 31.21 mmol, 2.96 mL, 2 eq.) and methyl(trioctyl) ammonium chloride (315.35 mg, 780.28 μmol, 358.36 uL, 0.05 eq.) were added. The mixture was stirred at 20° C. for 4 h. Upon completion, the excess Me₂SO₄ was destroyed by adding 0.8 g pellets of NaOH into the vigorously stirred solution. Stirring was continued at 20° C. for 10 h. The layers were separated, and the aqueous layer extracted with DCM (2×20 mL). The organic layer was dried over Na₂SO₄, filtered, and concentrated. The residue was purified by column chromatography (SiO₂, Petroleum ether:Ethyl acetate=100:1-5:1) to give 3-bromo-2,5-dimethoxy-4-pentylbenzaldehyde. ¹H NMR (400 MHz, CHLOROFORM-d) δ=10.38-10.27 (m, 1H), 7.23 (s, 1H), 3.96-3.90 (m, 3H), 3.86 (s, 3H), 2.91-2.82 (m, 2H), 1.57-1.46 (m, 2H), 1.41-1.32 (m, 4H), 0.99-0.83 (m, 4H).

### Step 4: Preparation of 2,5-dimethoxy-3-methyl-4-pentylbenzaldehyde

To a stirred solution of 3-bromo-2,5-dimethoxy-4-pentylbenzaldehyde (3.9 g, 12.37 mmol, 1 eq.) in dioxane (20 mL) under N₂ was added 2,4,6-trimethyl-1,3,5,2,4,6-trioxatriborinane (9.32 g, 37.1 mmol, 10.4 mL, 50% purity, 3 eq.). K₂CO₃ (5.13 g, 37.1 mmol, 3 eq.) and Pd(PPh₃)₄ (1.43 g, 1.24 mmol, 0.1 eq.) were added at 20° C. and the reaction was warmed to 110° C. for 3 h. Upon completion, the reaction was diluted in H₂O (10 mL) and extracted with EtOAc (10 mL×3). The combined organic layer was washed with brine, dried over Na₂SO₄, filtered, and concentrated in vacuo. The residue was purified by column chromatography (SiO₂, Petroleum ether:Ethyl acetate=100:1-50:1) to give 2,5-dimethoxy-3-methyl-4-pentylbenzaldehyde (1.4 g, 5.59 mmol, 45% yield) as an orange solid. ¹H NMR (400 MHz, CHLOROFORM-d) δ=10.51-10.40 (m, 1H), 7.27 (s, 1H), 3.96 (s, 3H), 3.94 (s, 3H), 2.85-2.75 (m, 2H), 2.45-2.34 (m, 3H), 1.64-1.54 (m, 2H), 1.53-1.43 (m, 4H), 1.10-0.97 (m, 3H).

### Step 5: Preparation of 1,4-dimethoxy-3-methyl-5-[(E)-2-nitroprop-1-en-1-yl]-2-pentylbenzene

To a stirred solution of 2,5-dimethoxy-3-methyl-4-pentylbenzaldehyde (1.4 g, 5.59 mmol, 1 eq.) in nitroethane (21 g, 280 mmol, 20 mL, 50 eq.) was added NH₄OAc (862 mg, 11.2 mmol, 2 eq.). The mixture was warmed to 115° C. and stirred for 1.5 h. Upon completion, the reaction mixture was concentrated to give a residue that was purified by column chromatography to give 1,4-dimethoxy-3-methyl-5-[(E)-2-nitroprop-1-en-1-yl]-2-pentylbenzene (1.58 g, 5.14 mmol, 92% yield) as a yellow solid. 1H NMR (400 MHz, CHLOROFORM-d) δ=8.26 (s, 1H), 6.65 (s, 1H), 3.83-3.78 (m, 3H), 3.70-3.61 (m, 3H), 2.70-2.61 (m, 2H), 2.44 (d, J=0.8 Hz, 3H), 2.31-2.24 (m, 3H), 1.51-1.42 (m, 2H), 1.42-1.34 (m, 4H), 0.96-0.88 (m, 3H).

### Step 6: Preparation of 1-(2,5-dimethoxy-3-methyl-4-pentyl-phenyl)propan-2-amine (68)

A mixture of 1,4-dimethoxy-3-methyl-5-[(E)-2-nitroprop-1-en-1-yl]-2-pentyl-benzene (500 mg, 1.63 mmol, 1 eq.) in THF (7 mL) was cooled to 0° C. LiAlH₄ (247 mg, 6.5 mmol, 4 eq.) was added in several portions. The mixture was warmed to 60° C. and stirred for 2 h. Upon completion, the mixture was cooled to 0° C. To the reaction mixture was added water dropwise (0.25 mL) and it was stirred for 5 min. Then (0.25 mL) of 30% aq. NaOH soln. was added. The mixture was stirred to a smooth dispersion and filtered, and the filtrate was concentrated. The residue was purified by prep-HPLC (column: Phenomenex luna C18 (250×70 mm, 15 μm); mobile phase: [water (0.05% HCl)-ACN]; B %: 25%-55%, 20 min) to give 1-(2,5-dimethoxy-3-methyl-4-pentylphenyl)propan-2-amine (185 mg, 662 μmol, 41% yield, HCl) as an off-white solid. ¹H NMR (400 MHz, DMSO-d₆, HCl salt) δ=8.22 (br s, 3H), 6.66 (s, 1H), 3.77-3.68 (m, 3H), 3.63-3.56 (m, 3H), 2.98 (dd, J=5.2, 13.3 Hz, 1H), 2.70 (dd, J=9.2, 13.3 Hz, 1H), 2.56-2.53 (m, 1H), 2.53 (br s, 1H), 2.55-2.51 (m, 1H), 2.15 (s, 3H), 1.36 (br d, J=8.8 Hz, 2H), 1.33-1.27 (m, 4H), 1.11 (d, J=6.4 Hz, 3H), 0.90-0.83 (m, 3H); ¹³C NMR (101 MHz, DMSO-d₆, HCl salt) δ=153.63, 150.94, 130.21, 129.60, 127.26, 110.82, 60.96, 56.15, 47.74, 35.41, 32.02, 28.92, 26.47, 22.41, 18.30, 14.36, 12.48.

### Example 66: Preparation of 1-(2-methoxy-4-propylphenyl)propan-2-amine (69)

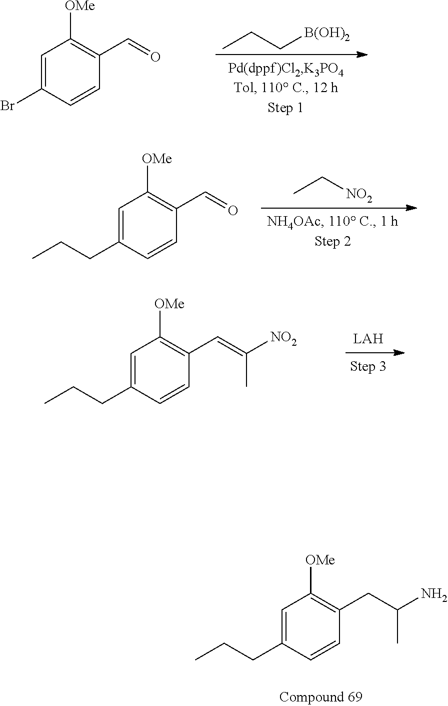

### Step 1: Preparation of 2-methoxy-4-propylbenzaldehyde

A stirred solution of 4-bromo-2-methoxybenzaldehyde (7 g, 32.55 mmol, 1 eq.) in toluene (10 mL) under N₂ was treated with K₃PO₄ (13.82 g, 65.1 mmol, 1 eq.), propylboronic acid (4.29 g, 49 mmol, 1.5 eq.), and Pd(dppf)Cl₂ (2.38 g, 3.26 mmol, 0.1 eq.). The mixture was stirred and warmed to 110° C. for 12 h. Upon completion, the mixture was filtered, and concentrated. The residue was purified by silica gel chromatography (PE:EA=100:1-10:1) to afford 2-methoxy-4-propylbenzaldehyde (3 g, 16.83 mmol, 52% yield) as a colorless oil. 1H NMR (400 MHz, CHLOROFORM-d) δ ppm 10.40 (s, 1H), 7.74-7.72 (d, J=8 Hz, 1H), 6.99-6.97 (d, J=8 Hz, 1H), 6.75 (s, 1H), 3.91 (s, 3H), 2.62-2.74 (m, 2H), 1.71-1.61 (m, 2H), 0.90 (t, J=6.8 Hz, 3H).

### Step 2: Preparation of 2-methoxy-1-[(E)-2-nitroprop-1-en-1-yl]-4-propylbenzene

A mixture of 2-methoxy-4-propylbenzaldehyde (1.4 g, 7.86 mmol, 1 eq.) and NH₄OAc (606 mg, 7.86 mmol, 1 eq.) in nitroethane (10 mL) was stirred and warmed to 100° C. for 1 h. Upon completion, the solvent was removed and the residue was purified by silica gel chromatography (PE:EA=50:1-0:1) to afford 2-methoxy-1-[(E)-2-nitroprop-1-en-1-yl]-4-propylbenzene (1 g, 4.25 mmol, 54% yield) as a yellow oil. 1H NMR (400 MHz, CHLOROFORM-d) δ ppm 8.30 (s, 1H), 7.23 (d, J=7.8 Hz, 1H), 6.88-6.80 (m, 1H), 6.80-6.73 (m, 1H), 3.88 (s, 3H), 2.68-2.58 (m, 2H), 2.41 (d, J=1.0 Hz, 3H), 1.74-1.63 (m, 2H), 0.98 (t, J=7.2 Hz, 3H).

### Step 3: Preparation of 1-(2-methoxy-4-propylphenyl)propan-2-amine (69)

To a solution of 2-methoxy-1-[(E)-2-nitroprop-1-en-1-yl]-4-propylbenzene (1 g, 4.25 mmol, 1 eq.) in THF (10 mL) was added in portions LiAlH₄ (645 mg, 17.0 mmol, 4 eq.) at 0° C. over 10 min. The resulting mixture was warmed to 70° C. and stirred for 5 h. Upon completion, the stirred mixture was cooled to 0° C. and treated dropwise with H₂O (0.65 mL). Then 30% aq. NaOH soln. (0.65 mL) was added dropwise After a smooth dispersion formed the mixture was filtered, and the filtrate concentrated. The residue was purified by prep-HPLC (column: Welch Xtimate C18 100×25 mm×3 μm; mobile phase: [water (0.05% HCl)-ACN]; B %: 5%-35%, 8 min) to afford 1-(2-methoxy-4-propylphenyl)propan-2-amine (292 mg, 1.20 mmol, 28% yield, 100% purity, HCl) as a white solid. LCMS R_{T}=0.649 min, MS cal.: 207.3, [M+H]⁺=208.1; 1H NMR (400 MHz, CHLOROFORM-d, HCl salt) δ ppm 8.35 (br s, 3H), 7.07 (d, J=7.6 Hz, 1H), 6.74-6.62 (m, 2H), 3.82 (s, 3H), 3.68 (s, 1H), 3.15 (dd, J=5.2, 13.2 Hz, 1H), 2.86 (dd, J=8.2, 13.2 Hz, 1H), 2.60-2.49 (m, 2H), 1.69-1.55 (m, 2H), 1.36 (d, J=6.4 Hz, 3H), 0.93 (t, J=7.2 Hz, 3H); ¹³C NMR (101 MHz, CHLOROFORM-d, HCl salt) δ ppm 157.35, 143.54, 131.21, 121.38, 120.50, 110.72, 55.19, 48.25, 38.12, 36.19, 24.50, 18.41, 13.89.

### Example 67: Preparation of 2-(2-methoxy-4-propylphenyl)ethanamine (70)

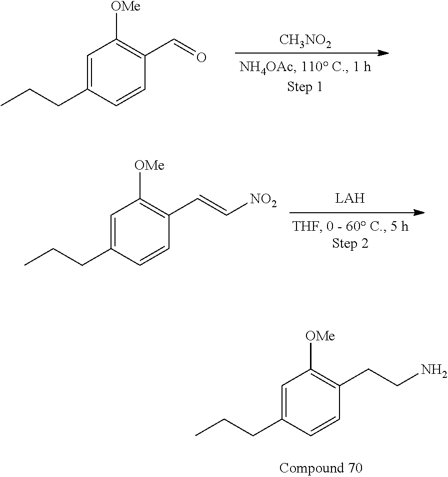 [SMILES: CCCc1ccc(C=O)c(OC)c1 ; "CH3NO2" ; CCCc1ccc(/C=C/[N+](=O)[O-])c(OC)c1 ; CCCc1ccc(CCN)c(OC)c1]

### Step 1: Preparation of 2-methoxy-1-[(E)-2-nitrovinyl]-4-propylbenzene

A solution of 2-methoxy-4-propylbenzaldehyde (1.4 g, 7.86 mmol, 1 eq.) in nitromethane (11.3 g, 185 mmol, 10 mL, 24 eq.) was treated with NH₄OAc (1.21 g, 15.71 mmol, 2 eq.) and the mixture was stirred at 110° C. for 1 h. Upon completion, the solvent was removed. The residue was purified by silica gel chromatography (PE:EA=20:1-5:1) to afford 2-methoxy-1-[(E)-2-nitrovinyl]-4-propylbenzene (1 g, 4.52 mmol, 58% yield) as a yellow solid. 1H NMR (400 MHz, CHLOROFORM-d) δ ppm 8.13 (d, J=4 Hz, 1H), 7.85 (d, J=13.6 Hz, 1H), 7.35 (d, J=3.6 Hz, 1H), 6.95 (d, J=3.6 Hz, 1H), 6.85 (s, 1H), 3.95 (s, 3H), 2.63-2.68 (m, 2H), 1.72-1.63 (m, 2H), 0.99-0.95 (m, 3H).

### Step 2: Preparation of 2-(2-methoxy-4-propylphenyl)ethanamine (70)

To a solution of 2-methoxy-1-[(E)-2-nitrovinyl]-4-propylbenzene (600 mg, 2.71 mmol, 1 eq.) in THF (15 mL) was added LiAlH₄ (411.7 mg, 10.9 mmol, 4 eq.) at 0° C. in portions over 10 min. The resulting mixture was warmed to 70° C. and stirred for 5 h. Upon completion, the mixture was cooled to 0° C. To the stirred mixture was added H₂O dropwise (0.4 mL). Then 30% aq. NaOH soln. (0.4 mL) was added dropwise. After stirring to a smooth dispersion, the mixture was filtered and concentrated. The residue was purified by prep-HPLC (column: Phenomenex luna C18 250×50 mm×10 μm; mobile phase: [water (0.04% HCl)-ACN]; B %: 10%-40%, 10 min) to afford 2-(2-methoxy-4-propylphenyl)ethanamine (170 mg, 740 μmol, 27% yield, HCl) as a white solid. LCMS R_{T}=1.845 min, MS cal.: 194.1, [M+H]⁺=195.1; 1H NMR (400 MHz, DMSO-d6, HCl salt) δ ppm 8.28 (br s, 3H), 7.10 (br d, J=7.3 Hz, 1H), 6.76-6.61 (m, 2H), 3.82 (s, 3H), 3.23 (br s, 2H), 3.05 (br s, 2H), 2.62-2.47 (m, 2H), 1.71-1.55 (m, 2H), 0.94 (t, J=7.3 Hz, 3H); ¹³C NMR (101 MHz, DMSO-d6, HCl salt) δ ppm 157.40, 143.64, 130.60, 121.75, 120.67, 110.83, 55.27, 39.86, 38.13, 29.09, 24.49, 13.85.

### Example 68: Preparation of 1-(4-(ethylthio)-2-methoxyphenyl)propan-2-amine (71)

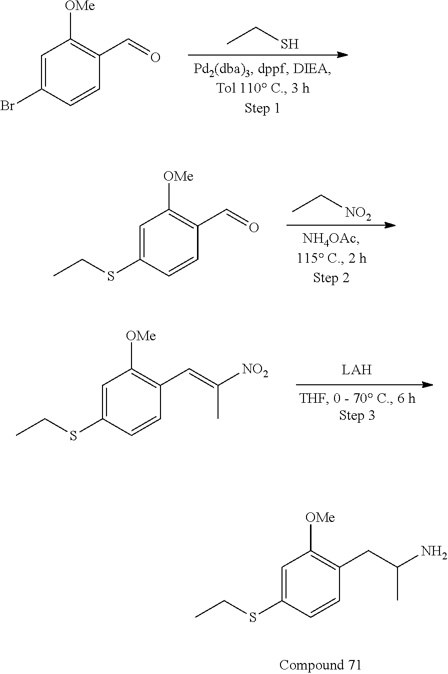

### Step 1: Preparation of 4-(ethylthio)-2-methoxybenzaldehyde

A mixture of 4-bromo-2-methoxybenzaldehyde (3 g, 13.95 mmol, 1 eq.), ethanethiol (2.6 g, 41.9 mmol, 3 eq.), Pd₂(dba)₃ (1.3 g, 1.39 mmol, 0.1 eq.), dppf (771 mg, 1.39 mmol, 0.1 eq.), and DIEA (5.4 g, 41.85 mmol, 3 eq.) in toluene (30 mL) was stirred under N₂ and warmed to 110° C. for 3 h. Upon completion, the mixture was filtered and concentrated. The residue was purified by column chromatography (SiO₂, Petroleum ether/Ethyl acetate=10/1-5/1) to afford 4-(ethylthio)-2-methoxybenzaldehyde (1.5 g, 7.64 mmol, 55% yield) as a yellow solid.

### Step 2: Preparation of (E)-ethyl(3-methoxy-4-(2-nitroprop-1-en-1-yl)phenyl)sulfane

To a solution of 4-(ethylthio)-2-methoxybenzaldehyde (1.3 g, 6.62 mmol, 1 eq.) in nitroethane (10 mL) was added NH₄OAc (1.02 g, 13.25 mmol, 2 eq.). The mixture was warmed to 110° C. and stirred for 1 h. Upon completion, the reaction mixture was concentrated to give a residue. The residue was purified by column chromatography (SiO₂, Petroleum ether/Ethyl acetate=10/1-5/1) to give (E)-ethyl(3-methoxy-4-(2-nitroprop-1-en-1-yl)phenyl)sulfane (0.8 g, 3.16 mmol, 48% yield) as a yellow solid. ¹H NMR (400 MHz, CHLOROFORM-d) δ ppm 8.18 (s, 1H), 7.19-7015 (m, 1H), 6.84-6.82 (m, 1H), 6.76 (s, 1H), 3.80 (s, 3H), 2.97-2.90 (m, 2H), 2.32 (s, 3H), 0.94-0.89 (m, 3H).

### Step 3: Preparation of 1-(4-(ethylthio)-2-methoxyphenyl)propan-2-amine (71)

A stirred solution of (E)-ethyl(3-methoxy-4-(2-nitroprop-1-en-1-yl)phenyl)sulfane (0.8 g, 3.16 mmol, 1 eq.) in THF (20 mL) was cooled to 0° C., then LiAlH₄ (719 mg, 19 mmol, 6 eq.) was added at 0° C. in portions. The mixture warmed to 70° C. and stirred for 6 h. Upon completion, the mixture was cooled to 0° C. The stirred mixture was treated dropwise with H₂O (1 mL). Then 30% aq. NaOH soln. (1 mL) was added dropwise. The mixture was stirred to a smooth dispersion then filtered, and the filtrate concentrated. The residue was purified by prep-HPLC (column: Phenomenex luna C18 (250×70 mm, 15 μm); mobile phase: [water (0.04% HCl)-ACN]; B %: 10%-40%, 20 min) to afford 1-(4-(ethylthio)-2-methoxyphenyl)propan-2-amine (350 mg, 1.31 mmol, 42% yield, 98% purity, HCl) as a white solid. LCMS R_{T}=1.881 min, MS cal.: 225.35, [M+H]⁺=226.1; 1H NMR (400 MHz, CHLOROFORM-d, HCl salt) δ ppm 8.37 (br s, 3H), 7.28-7.11 (m, 1H), 6.87-6.85 (m, 2H), 3.86 (s, 3H), 3.69 (m, 1H), 3.15-3.11 (m, 1H), 2.98-2.96 (m, 2H), 2.94-2.88 (m, 1H), 1.39 (t, J=9.2, 6.8 Hz, 3H), 1.34 (t, J=7.6, 7.2 Hz, 3H); ¹³C NMR (101 MHz, CHLOROFORM-d, HCl salt) δ ppm 157.62, 137.19, 131.75, 121.99, 120.87, 111.50, 55.44, 48.14, 36.17, 27.78, 18.45, 14.38.

### Example 69: Preparation of 1-(2-methoxy-4-pentylphenyl)propan-2-amine (72)

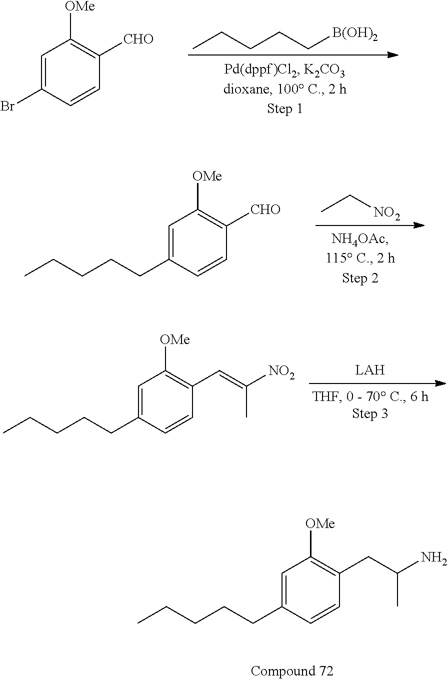

### Step 1: Preparation of 2-methoxy-4-pentylbenzaldehyde

To a solution of 4-bromo-2-methoxybenzaldehyde (5 g, 23.3 mmol, 1 eq.) in Tol (50 mL) was added pentylboronic acid (4.04 g, 34.9 mmol, 1.5 eq.), K₃PO₄ (9.87 g, 46.5 mmol, 2 eq.), and Pd(dppf)Cl₂ (1.70 g, 2.33 mmol, 0.1 eq.) under N₂. The mixture was stirred and warmed to 110° C. for 12 h. Upon completion, the mixture was filtered and concentrated. The residue was purified by column chromatography (SiO₂, Petroleum ether/Ethyl acetate=100/1-10/1) to afford 2-methoxy-4-pentylbenzaldehyde (3.8 g, 18.42 mmol, 79% yield) as a yellow oil. 1H NMR (400 MHz, CHLOROFORM-d) δ ppm 10.41 (s, 1H), 7.75 (d, J=8.0 Hz, 1H), 6.86 (d, J=8.0 Hz, 1H), 6.79 (s, 1H), 3.93 (s, 3H), 2.68-2.62 (m, 2H), 1.69-1.61 (m, 2H), 1.38-1.31 (m, 4H), 0.94-0.89 (m, 3H).

### Step 2: Preparation of 2-methoxy-1-[(E)-2-nitroprop-1-en-1-yl]-4-pentylbenzene

To a solution of 2-methoxy-4-pentylbenzaldehyde (3.8 g, 18.42 mmol, 1 eq.) in nitroethane (42 g, 560 mmol, 40 mL, 30.4 eq.) was added NH₄OAc (2.84 g, 36.84 mmol, 2 eq.). The mixture was stirred and warmed to 100° C. for 1 h. Upon completion, the mixture was concentrated, and the residue diluted with H₂O (40 mL) and extracted with EtOAc (30 mL×2), washed with brine, dried over Na₂SO₄, filtered, and concentrated. The residue was purified by column chromatography (SiO₂, Petroleum ether/Ethyl acetate=100/1-10/1) to afford 2-methoxy-1-[(E)-2-nitroprop-1-en-1-yl]-4-pentylbenzene (3.8 g, 14.43 mmol, 78% yield) as a yellow oil. 1H NMR (400 MHz, CHLOROFORM-d) δ ppm 8.31 (s, 1H), 7.23 (d, J=8.0 Hz, 1H), 6.84 (dd, J=1.2, 7.6 Hz, 1H), 6.77 (s, 1H), 3.89 (s, 3H), 2.67-2.62 (m, 2H), 2.41 (d, J=1.2 Hz, 3H), 1.69-1.61 (m, 2H), 1.39-1.32 (m, 4H), 0.94-0.89 (m, 3H).

### Step 3: Preparation of 1-(2-methoxy-4-pentylphenyl)propan-2-amine (72)

A solution of 2-methoxy-1-[(E)-2-nitroprop-1-en-1-yl]-4-pentyl-benzene (1.5 g, 5.70 mmol, 1 eq.) in THF (15 mL) was cooled to 0° C. Then LiAlH₄ (865 mg, 23 mmol, 4 eq.) was added in portions. The stirred mixture was warmed to 70° C. and stirred for 5 h. Upon completion, the mixture was cooled to 0° C. The stirred mixture was treated dropwise with H₂O (0.9 mL) then 30% aq. NaOH soln. (0.9 mL). The mixture was stirred at 0° C. for 10 min., then the solids were filtered, and the filtrate concentrated. The residue was purified by prep-HPLC (column: Phenomenex luna C18 (250×70 mm, 15 μm); mobile phase: [water (0.05% HCl)-ACN]; B %: 15%-45%, 25 min) to afford 1-(2-methoxy-4-pentylphenyl)propan-2-amine (401 mg, 1.48 mmol, 26% yield, HCl) as a white solid. LCMS R_{T}=2.181 min, MS cal.: 235.37, [M+H]⁺=236.1; 1H NMR (400 MHz, DMSO-d₆, HCl salt) δ ppm 8.28 (br s, 3H), 7.02 (d, J=7.6 Hz, 1H), 6.79 (s, 1H), 6.69 (d, J=7.6 Hz, 1H), 3.76 (s, 3H), 3.34 (td, J=6.0, 8.7 Hz, 1H), 2.96 (dd, J=4.8, 13.2 Hz, 1H), 2.65 (dd, J=9.6, 12.8 Hz, 1H), 2.56-2.50 (m, 2H), 1.55 (quin, J=7.2 Hz, 2H), 1.34-1.20 (m, 4H), 1.08 (d, J=6.4 Hz, 3H), 0.84 (t, J=6.8 Hz, 3H); ¹³C NMR (101 MHz, DMSO-d₆, HCl salt) δ ppm 157.15, 142.75, 130.62, 121.81, 120.11, 110.93, 55.24, 46.73, 35.24, 34.64, 31.00, 30.62, 21.97, 17.62, 13.92.

### Example 70: Preparation of 1-(4-(6-fluorohexyl)-2,5-dimethoxyphenyl)propan-2-amine (73)

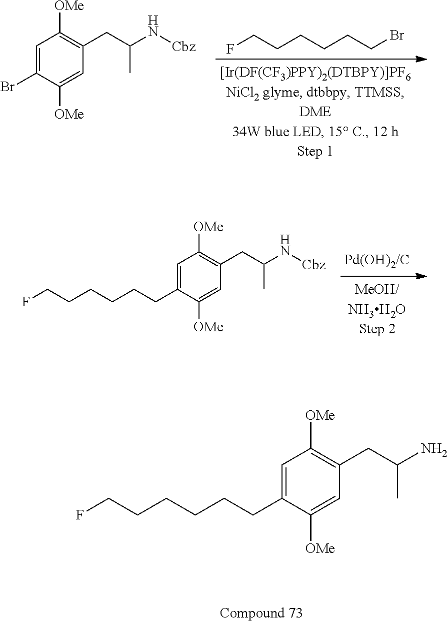 [SMILES: COc1cc(CC(C)NC(=O)OCc2ccccc2)c(OC)cc1Br ; FCCCCCCBr ; COc1cc(CC(C)NC(=O)OCc2ccccc2)c(OC)cc1CCCCCCF ; COc1cc(CC(C)N)c(OC)cc1CCCCCCF]

### Step 1: Preparation of benzyl (1-(4-(6-fluorohexyl)-2,5-dimethoxyphenyl)propan-2-yl)carbamate

A mixture of benzyl (1-(4-bromo-2,5-dimethoxyphenyl)propan-2-yl)carbamate (400 mg, 980 umol, 1 eq, 4 batches), 1-bromo-6-fluorohexane (717 mg, 3.92 mmol, 4 eq.), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (11 mg, 9.8 umol, 0.01 eq.), NiCl₂.glyme (1.08 mg, 4.90 umol, 0.005 eq.), Na₂CO₃ (207.68 mg, 1.96 mmol, 2 eq.), dtbbpy (1.31 mg, 4.90 umol, 0.005 eq.), and TTMSS (244 mg, 980 umol, 302 uL, 1 eq.) in DME (4 mL) was degassed and purged with Ar 3 times. The mixture was then stirred at 15° C. for 12 h under an Ar atmosphere exposed to a 34 W blue LED light. Upon completion, the reaction mixture was filtered, and the filtrate concentrated. The residue was purified by prep-HPLC (column: Phenomenex luna C18 (250×70 mm, 15 um); mobile phase: [water (0.05% HCl)-ACN]; B %: 60%-85%, 30 min) to afford benzyl (1-(4-(6-fluorohexyl)-2,5-dimethoxyphenyl)propan-2-yl)carbamate (667 mg, 1.54 mmol, 39% yield) as a white solid. ¹H NMR (400 MHz, CHLOROFORM-d) δ ppm 7.38-7.27 (m, 5H), 6.62 (s, 2H), 5.08-5.02 (m, 2H), 4.51 (t, J=6.0 Hz, 1H), 4.39 (t, J=6.0 Hz, 1H), 4.01-3.90 (m, 1H), 3.84-3.69 (m, 5H), 2.86-2.74 (m, 1H), 2.74-2.65 (m, 1H), 2.62-2.53 (m, 2H), 1.79-1.57 (m, 4H), 1.50-1.37 (m, 4H), 1.23-1.13 (m, 3H).

### Step 2: Preparation of 1-(4-(6-fluorohexyl)-2,5-dimethoxyphenyl)propan-2-amine (73)

To a solution of benzyl (1-(4-(6-fluorohexyl)-2,5-dimethoxyphenyl)propan-2-yl)carbamate (667 mg, 1.54 mmol, 1 eq.) in MeOH (30 mL) and NH₃·H₂O (3 mL) was added Pd(OH)₂ (1 g, 7.1 mmol, 5.4 eq.). The mixture was stirred at 15° C. for 1 h under H₂ (15 psi). Upon completion, the reaction mixture was filtered, and the filtrate was concentrated to afford 1-(4-(6-fluorohexyl)-2,5-dimethoxyphenyl)propan-2-amine (430 mg, 1.44 mmol, 94% yield) as a white solid. LCMS R_{T}=2.140 min, MS cal.: 297.21 [M+H]⁺=298.1; ¹H NMR (400 MHz, DMSO-d6) δ ppm 6.79-6.67 (m, 2H), 4.47 (t, J=6.0 Hz, 1H), 4.35 (t, J=6.0 Hz, 1H), 3.69 (d, J=3.2 Hz, 6H), 3.02-2.98, (m, 1H), 2.69-2.51 (m, 2H), 2.48-2.39 (m, 2H), 1.65-1.31, (m, 8H), 0.99-0.89 (m, 3H); ¹³C NMR (101 MHz, DMSO-d6) δ ppm 151.45, 150.99, 128.87, 126.44, 114.36, 113.34, 85.07, 83.47, 56.28, 47.44, 41.01, 30.36, 30.16, 30.09, 30.00, 29.02, 25.03, 24.98, 23.95.

### Example 71: Preparation of 1-(2,5-dimethoxy-4-(5,5,5-trifluoropentyl)phenyl)propan-2-amine (74)

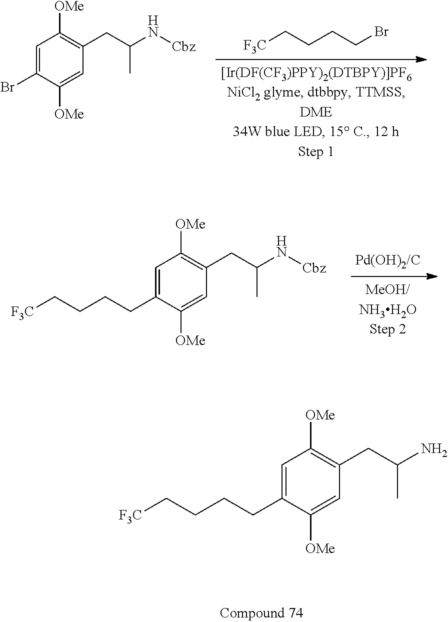 [SMILES: COc1cc(CC(C)NC(=O)OCc2ccccc2)c(OC)cc1Br ; FC(F)(F)CCCCBr ; COc1cc(CC(C)NC(=O)OCc2ccccc2)c(OC)cc1CCCCC(F)(F)F ; COc1cc(CC(C)N)c(OC)cc1CCCCC(F)(F)F]

### Step 1: Preparation of benzyl (1-(2,5-dimethoxy-4-(5,5,5-trifluoropentyl)phenyl)propan-2-yl)carbamate

A mixture of benzyl (1-(4-bromo-2,5-dimethoxyphenyl)propan-2-yl)carbamate (350 mg, 857 umol, 1 eq, 3 batches), 5-bromo-1,1,1-trifluoropentane (703 mg, 3.43 mmol, 4 eq.), NiCl₂.glyme (942 ug, 4.29 umol, 0.005 eq.), Na₂CO₃ (182 mg, 1.71 mmol, 2 eq.), dtbbpy (1.15 mg, 4.29 umol, 0.005 eq.), TTMSS (213 mg, 857 umol, 1 eq.), and bis[3,5-difluoro-2-[5-(trifluoromethyl)-2-pyridyl]phenyl]iridium(1⁺) 4-tert-butyl-2-(4-tert-butyl-2-pyridyl)pyridine hexafluorophosphate (9.62 mg, 8.57 umol, 0.01 eq.) in DME (3 mL) was degassed and purged with Ar 3 times. Then the mixture was stirred at 15° C. for 12 h under Ar atmosphere exposed to a 34 W blue LED. Upon completion, the reaction mixture was filtered and the filtrate was concentrated. The residue was purified by prep-HPLC (column: Phenomenex luna C18 (250×70 mm, 15 um, [water (0.04% HCl)-ACN]; B %: 50%-80%, 20 min) to afford benzyl (1-(2,5-dimethoxy-4-(5,5,5-trifluoropentyl)phenyl)propan-2-yl)carbamate (500 mg, 964 umol, 38% yield, 94.5% purity) as an off-white solid. ¹H NMR (400 MHz, DMSO-d6) δ=7.39-7.27 (m, 5H), 6.68-6.59 (m, 2H), 5.13-4.98 (m, 3H), 4.03-3.87 (m, 1H), 3.81-3.68 (m, 6H), 2.85-2.74 (m, 1H), 2.73-2.66 (m, 1H), 2.63-2.55 (m, 2H), 2.23-2.02 (m, 2H), 1.72-1.53 (m, 5H), 1.18 (d, J=6.4 Hz, 3H).

### Step 2: Preparation of 1-(2,5-dimethoxy-4-(5,5,5-trifluoropentyl)phenyl)propan-2-amine (74)

To a solution of benzyl (1-(2,5-dimethoxy-4-(5,5,5-trifluoropentyl)phenyl)propan-2-yl)carbamate (500 mg, 1.11 mmol, 1 eq.) in MeOH (30 mL) and NH₃·H₂O (3 mL) was added Pd(OH)₂ (1.25 g, 8.90 mmol, 8.1 eq.). The mixture was stirred at 20° C. for 1 h under H₂ (15 psi). Upon completion, the reaction mixture was filtered, and the filtrate was concentrated to afford 1-(2,5-dimethoxy-4-(5,5,5-trifluoropentyl)phenyl)propan-2-amine_(371 mg, 1.10 umol, 99% yield, 95.5% purity) as a white solid. LCMS R_{T}=2.165 min, MS cal.: 319.18 [M+H]⁺=320.1; ¹H NMR (400 MHz, DMSO-d₆) δ=6.74 (s, 1H), 6.71 (s, 1H), 3.69 (d, J=4.8 Hz, 6H), 2.98 (q, J=6.4 Hz, 1H), 2.57-2.51 (m, 2H), 2.47-2.41 (m, 2H), 2.35-2.15 (m, 2H), 1.63-1.53 (m, 2H), 1.52-1.42 (m, 2H), 0.92 (d, J=6.4 Hz, 3H); ¹³C NMR (101 MHz, DMSO-d₆) δ=151.47, 151.00, 128.19, 126.75, 114.37, 113.46, 56.29, 56.25, 47.42, 32.55, 29.50, 28.99, 24.04, 21.63.

### Example 72: Preparation of 1-(4-((5-fluoropentyl)thio)-2,5-dimethoxyphenyl)butan-2-amine (75)

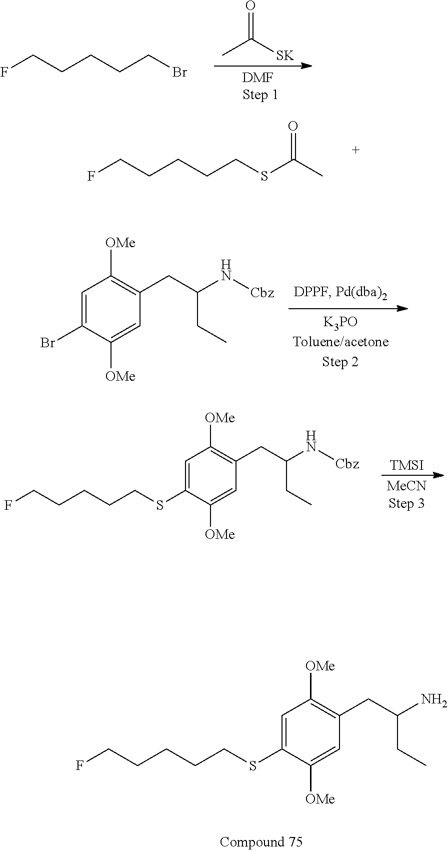

### Step 1: Preparation of S-(5-fluoropentyl) ethanethioate

To a mixture of 1-bromo-5-fluoropentane (10 g, 59.2 mmol, 1 eq.) in DMF (100 mL) was added potassium ethanethioate (10.13 g, 89 mmol, 1.5 eq.) in one portion at 20° C. under N₂. The mixture was stirred at 20° C. for 4 h. Upon completion, the reaction mixture was quenched by the addition of H₂O (25 mL) at 20° C., and then the product was extracted with EtOAc (3×10 mL). The combined organic layers were washed with brine (3×10 mL), dried over Na₂SO₄, filtered, and concentrated to give S-(5-fluoropentyl) ethanethioate (9 g, 55 mmol, 93% yield) as a colorless oil. ¹H NMR (400 MHz, CHLOROFORM-d) δ=4.50 (t, J=6.0 Hz, 1H), 4.39 (t, J=6.0 Hz, 1H), 2.89 (t, J=7.2 Hz, 2H), 2.34 (s, 3H), 1.81-1.58 (m, 4H), 1.54-1.44 (m, 2H).

### Step 2: Preparation of benzyl (1-(4-((5-fluoropentyl)thio)-2,5-dimethoxyphenyl)butan-2-yl)carbamate

To a mixture of benzyl (1-(4-bromo-2,5-dimethoxyphenyl)butan-2-yl)carbamate (330 mg, 781 umol, 1 eq, 3 batches), S-(5-fluoropentyl) ethanethioate (578 mg, 3.52 mmol, 4.5 eq.), DPPF (43.3 mg, 78 umol, 0.1 eq.), and K₃PO₄ (166 mg, 781 umol, 1 eq.) in toluene (2 mL) and acetone (1 mL) was added Pd(dba)₂ (44.93 mg, 78.14 umol, 0.1 eq.) in one portion at 20° C. under N₂. The mixture was warmed to 115° C. and stirred for 12 h. Upon completion, the reaction mixture was cooled then quenched by the addition of aq. NH₄Cl (10 mL) at 20° C., and then extracted with EtOAc (3×10 mL). The combined organic layers were washed with brine (30 mL), dried over Na₂SO₄, filtered, and concentrated to give a residue. The residue was purified by prep-HPLC (column: Phenomenex luna C18 (250×70 mm, 15 um); [water (0.04% HCl)-ACN]; B %: 50%-80%, 20 min) to afford benzyl (1-(4-((5-fluoropentyl)thio)-2,5-dimethoxyphenyl)butan-2-yl)carbamate (700 mg, 1.40 mmol, 60% yield) as an off-white solid. ¹H NMR (400 MHz, CHLOROFORM-d) δ=7.28 (m, 5H), 6.82 (s, 1H), 6.71-6.61 (m, 1H), 5.04 (s, 2H), 4.90-4.78 (m, 1H), 4.49 (t, J=6.0 Hz, 1H), 4.38 (t, J=6.0 Hz, 1H), 3.90-3.82 (m, 1H), 3.82-3.68 (m, 6H), 2.97-2.86 (m, 2H), 2.81-2.72 (m, 2H), 1.81-1.64 (m, 4H), 1.63-1.55 (m, 3H), 1.50-1.39 (m, 1H), 1.00-0.90 (m, 3H).

### Step3: Preparation of 1-(4-((5-fluoropentyl)thio)-2,5-dimethoxyphenyl)butan-2-amine (75)

To a mixture of benzyl (1-(4-((5-fluoropentyl)thio)-2,5-dimethoxyphenyl)butan-2-yl)carbamate (600 mg, 1.29 mmol, 1 eq.) in MeCN (10 mL) was added TMSI (777 mg, 3.88 mmol, 3 eq.) in one portion at 0° C. under N₂. The mixture was stirred at 20° C. for 12 h. Upon completion, the reaction mixture was quenched by the addition of saturated aq. NaHCO₃ solution (5 ml) at 20° C. The mixture was extracted with EtOAc (3×5 mL) and the organic layer was dried over Na₂SO₄, filtered, and concentrated to give a residue. The residue was purified by prep-HPLC (column: Phenomenex luna C18 (250×70 mm, 15 um); [water (0.04% HCl)-ACN]; B %: 17%-43%, 7 min) to afford 1-(4-((5-fluoropentyl)thio)-2,5-dimethoxyphenyl)butan-2-amine (157 mg, 429.04 umol, 33.15% yield, HCl) as a yellow solid. The residue was further purified by prep-HPLC (column: Phenomenex luna C18 (250×70 mm, 15 um); [water (NH₄HCO₃)-ACN]; B %: 25%-60%, 8 min) to afford 1-(4-((5-fluoropentyl)thio)-2,5-dimethoxyphenyl)butan-2-amine (90 mg, 273 umol, 21% yield) as an off-white solid. LCMS R_{T}=2.114 min, MS cal.: 329.47 [M+H]⁺=330.1; ¹H NMR (400 MHz, CHLOROFORM-d) δ=6.85 (s, 1H), 6.70 (s, 1H), 4.50 (t, J=6.0 Hz, 1H), 4.41-4.32 (m, 1H), 3.85 (s, 3H), 3.79 (s, 3H), 3.03-2.93 (m, 1H), 2.93-2.86 (m, 2H), 2.81 (dd, J=4.8, 13.2 Hz, 1H), 2.47 (dd, J=8.8, 13.2 Hz, 1H), 1.79-1.66 (m, 4H), 1.60-1.49 (m, 3H), 1.46-1.30 (m, 1H), 0.99 (t, J=7.2 Hz, 3H); ¹³C NMR (101 MHz, CHLOROFORM-d) δ=151.93, 151.84, 127.43, 122.06, 114.23, 114.00, 84.71, 83.07, 56.47, 56.15, 52.96, 38.58, 32.74, 30.37, 30.09, 29.90, 28.76, 24.54, 24.48, 10.62.

### Example 73: Preparation of 1-(2,5-dimethoxy-4-((5,5,5-trifluoropentyl)thio)phenyl)butan-2-amine (76)

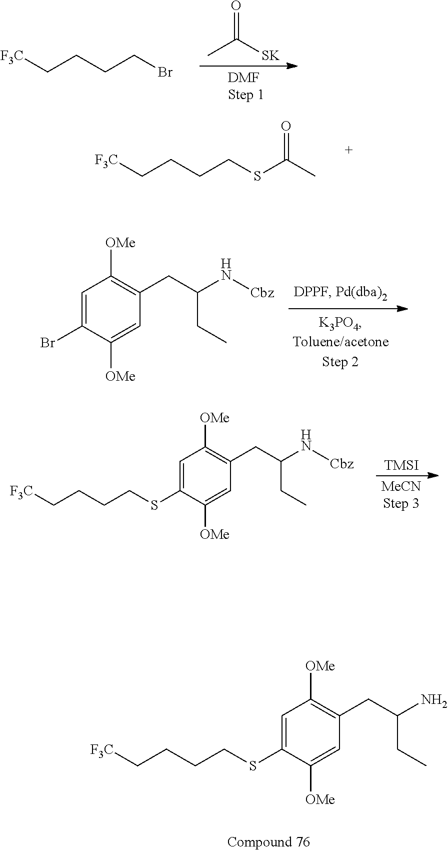

### Step1: Preparation of S-(5,5,5-trifluoropentyl) ethanethioate

To a mixture of potassium ethanethioate (836 mg, 7.32 mmol, 1.5 eq.) in DMF (10 mL) was added 5-bromo-1,1,1-trifluoropentane (1 g, 4.88 mmol, 1 eq.) in one portion at 20° C. under N₂. The mixture was stirred at 20° C. for 12 h. Upon completion, the reaction mixture was quenched by the addition of H₂O 25 mL at 20° C. It was extracted with EtOAc (3×10 mL) and the combined organic layers were washed with brine (3×10 mL), dried over Na₂SO₄, filtered, and concentrated to give S-(5,5,5-trifluoropentyl) ethanethioate (900 mg, 4.50 mmol, 92% yield) as a colorless oil. ¹H NMR (400 MHz, CHLOROFORM-d) δ=2.89 (t, J=6.4 Hz, 2H), 2.34 (s, 3H), 2.10 (td, J=7.2, 10.4 Hz, 2H), 1.77-1.57 (m, 4H).

### Step 2: Preparation of benzyl (1-(2,5-dimethoxy-4-((5,5,5-trifluoropentyl)thio)phenyl)butan-2-yl)carbamate

To a mixture of benzyl (1-(4-bromo-2,5-dimethoxyphenyl)butan-2-yl)carbamate (422 mg, 999 umol, 1 eq.), S-(5,5,5-trifluoropentyl) ethanethioate (800 mg, 4.00 mmol, 4 eq.), DPPF (55.4 mg, 99.9 umol, 0.1 eq.), and K₃PO₄ (212 mg, 999 umol, 1 eq.) in Tol. (4 mL) and acetone (2 mL) was added Pd(dba)₂ (57.44 mg, 99.9 umol, 0.1 eq.) in one portion at 20° C. under N₂. The mixture was stirred at 115° C. for 12 h. Upon completion, the reaction mixture was quenched by addition of aq. NH₄Cl 10 mL at 20° C., and then extracted with EtOAc (3×10 mL). The combined organic layers were washed with brine (3×10 mL), dried over Na₂SO₄, filtered, and concentrated to give a residue. The residue was purified by prep-HPLC (column: Phenomenex luna C18 (250×70 mm, 15 um); [water (0.04% HCl)-ACN]; B %: 60%-90%, 10 min) to afford benzyl (1-(2,5-dimethoxy-4-((5,5,5-trifluoropentyl)thio)phenyl)butan-2-yl)carbamate (280 mg, 522 umol, 52% yield) as an off-white solid. ¹H NMR (400 MHz, CHLOROFORM-d) δ=7.51-7.27 (m, 5H), 6.84 (s, 1H), 6.67 (s, 1H), 5.04 (s, 2H), 4.88-4.79 (m, 1H), 3.78 (br d, J=9.2 Hz, 6H), 2.89 (br t, J=6.4 Hz, 2H), 2.76 (br d, J=6.8 Hz, 2H), 2.17-1.98 (m, 2H), 1.78-1.65 (m, 4H), 1.64-1.53 (m, 4H), 1.51-1.38 (m, 2H), 1.26 (s, 4H), 1.12 (s, 2H), 1.03-0.91 (m, 3H).

### Step 3: Preparation of 1-(2,5-dimethoxy-4-((5,5,5-trifluoropentyl)thio)phenyl)butan-2-amine

To a mixture of benzyl (1-(2,5-dimethoxy-4-((5,5,5-trifluoropentyl)thio)phenyl)butan-2-yl)carbamate (230 mg, 460.38 umol, 1 eq.) in MeCN (20 mL) was added TMSI (276 mg, 1.38 mmol, 188 uL, 3 eq.) in one portion at 0° C. under N₂. The mixture was stirred at 20° C. for 12 h. Upon completion, the reaction mixture was quenched by addition of aq. NaHCO₃ (5 mL) at 20° C., and then filtered and concentrated to give a residue. The residue was purified by prep-HPLC (column: Phenomenex luna C18 (250×70 mm, 15 um); [water (0.04% HCl)-ACN]; B %: 15%-40%, 8 min) to afford 1-(2,5-dimethoxy-4-((5,5,5-trifluoropentyl)thio)phenyl)butan-2-amine (105 mg, 261 umol, 57% yield, HCl) as a yellow solid. The residue was further purified by prep-HPLC (column: Phenomenex luna C18 (250×50 mm, 15 um); [water (NH₄HCO₃)-ACN]; B %: 30%-70%, 8 min) to afford 1-(2,5-dimethoxy-4-((5,5,5-trifluoropentyl)thio)phenyl)butan-2-amine (60 mg, 164.18 umol, 35.66% yield) as an off-white solid. LCMS R_{T}=2.277 min, MS cal.: 365.46 [M+H]⁺=366.1; 1H NMR (400 MHz, CHLOROFORM-d) δ=6.87 (s, 1H), 6.71 (s, 1H), 3.89 (s, 3H), 3.75 (s, 3H), 3.02-2.86 (m, 3H), 2.81 (dd, J=4.8, 13.2 Hz, 1H), 2.45 (dd, J=8.4, 13.2 Hz, 1H), 2.09 (td, J=7.6, 10.8 Hz, 2H), 1.79-1.63 (m, 4H), 1.53 (m, 1H), 1.42-1.31 (m, 1H), 0.99 (t, J=7.6 Hz, 3H); ¹³C NMR (101 MHz, CHLOROFORM-d) δ=152.25, 151.81, 128.22, 121.17, 114.72, 114.28, 56.46, 56.15, 52.91, 38.83, 33.52, 33.24, 32.67, 30.61, 28.24, 21.13, 21.10, 10.67.

### Example 74: Preparation of 1-(4-(6-fluorohexyl)-2,5-dimethoxyphenyl)butan-2-amine (77)

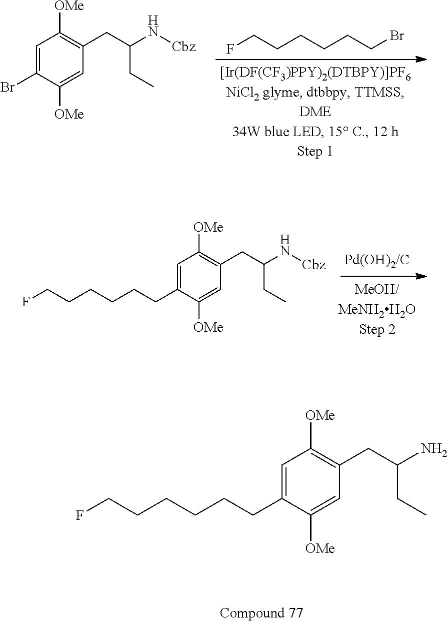 [SMILES: CCC(Cc1cc(OC)c(Br)cc1OC)NC(=O)OCc1ccccc1 ; FCCCCCCBr ; CCC(Cc1cc(OC)c(CCCCCCF)cc1OC)NC(=O)OCc1ccccc1 ; CCC(N)Cc1cc(OC)c(CCCCCCF)cc1OC]

### Step 1: Preparation of benzyl (1-(4-(6-fluorohexyl)-2,5-dimethoxyphenyl)butan-2-yl)carbamate

A mixture of benzyl (1-(4-bromo-2,5-dimethoxyphenyl)butan-2-yl)carbamate (400 mg, 947 umol, 1 eq, 3 batches), 1-bromo-6-fluorohexane (694 mg, 3.79 mmol, 4 eq.), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (10.6 mg, 9.47 umol, 0.01 eq.), NiCl₂.glyme (1.04 mg, 4.74 umol, 0.005 eq.), Na₂CO₃ (201 mg, 1.89 mmol, 2 eq.), dtbbpy (1.3 mg, 4.74 umol, 0.005 eq.), and TTMSS (236 mg, 947 umol, 292.21 uL, 1 eq.) in DME (4 mL) was degassed and purged with Ar 3 times. Then the mixture was stirred at 15° C. for 6 h under an Ar atmosphere exposed to a 34 W blue LED. Upon completion, the reaction mixture was filtered and the filtrate was concentrated. The residue was purified by prep-HPLC (column: Phenomenex luna C18 (250×70 mm, 15 um); mobile phase: [water (0.04% HCl)-ACN]; B %: 55%-85%, 20 min) to afford benzyl (1-(4-(6-fluorohexyl)-2,5-dimethoxyphenyl)butan-2-yl)carbamate (559 mg, 1.25 mmol, 44% yield) as a white solid. ¹H NMR (400 MHz, DMSO-d6) δ ppm 7.35-7.23 (m, 4H), 7.12-7.02 (m, 1H), 6.75-6.65 (m, 2H), 5.02-4.86 (m, 2H), 4.51-4.42 (m, 1H), 4.40-4.31 (m, 1H), 3.76-3.61 (m, 6H), 3.61-3.53 (m, 1H), 2.75-2.64 (m, 1H), 2.57-2.51 (m, 3H), 1.68-1.10 (m, 10H), 0.84 (t, J=7.2 Hz, 3H).

### Step 2: Preparation of 1-(4-(6-fluorohexyl)-2,5-dimethoxyphenyl)butan-2-amine (77)

To a solution of benzyl (1-(4-(6-fluorohexyl)-2,5-dimethoxyphenyl)butan-2-yl)carbamate (559 mg, 1.25 mmol, 1 eq.) in MeOH (30 mL) and MeNH₂·H₂O (3 mL) was added Pd(OH)₂/C (1 g, 7.12 mmol, 8.8 eq.). The mixture was stirred at 15° C. for 1 h under H₂ (15 psi). Upon completion, the reaction mixture was filtered and the filtrate was concentrated to afford 1-(4-(6-fluorohexyl)-2,5-dimethoxyphenyl)butan-2-amine_(361.6 mg, 1.16 mmol, 92.8% yield) as a white solid. LCMS R_{T}=2.215 min, MS cal.: 311.23 [M+H]⁺=312.1; ¹H NMR (400 MHz, DMSO-d₆) δ ppm 6.72 (d, J=3.6 Hz, 2H), 4.50-4.45 (m, 1H), 4.38-4.33 (m, 1H), 3.71-3.67 (m, 6H), 2.77-2.56 (m, 2H), 2.49-2.45 (m, 2H), 2.40-2.31 (m, 1H), 1.70-1.45 (m, 4H), 1.41-1.12 (m, 8H), 0.91-0.81 (m, 3H); ¹³C NMR (101 MHz, DMSO-d₆) δ ppm 151.51, 151.01, 128.83, 126.54, 114.42, 113.37, 85.09, 83.48, 56.33, 56.30, 53.11, 38.92, 30.43, 30.37, 30.17, 30.11, 30.01, 29.04, 25.04, 24.99, 11.00.

### Example 75: Preparation of 1-(2,5-demethoxy-4-(4,4,4-trifluorobutyl)phenyl)butan-2-amine (78)

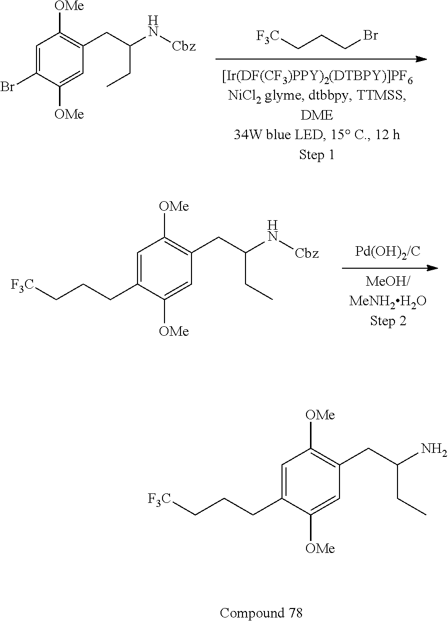 [SMILES: CCC(Cc1cc(OC)c(Br)cc1OC)NC(=O)OCc1ccccc1 ; FC(F)(F)CCCBr ; CCC(Cc1cc(OC)c(CCCC(F)(F)F)cc1OC)NC(=O)OCc1ccccc1 ; CCC(N)Cc1cc(OC)c(CCCC(F)(F)F)cc1OC]

### Step 1: Preparation of benzyl (1-(2,5-dimethoxy-4-(4,4,4-trifluorobutyl)phenyl)butan-2-yl)carbamate

A mixture of benzyl (1-(4-bromo-2,5-dimethoxyphenyl)butan-2-yl)carbamate (400 mg, 947 umol, 1 eq, 4 batches), 4-bromo-1,1,1-trifluorobutane (724 mg, 3.79 mmol, 4 eq.), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (10.6 mg, 9.47 umol, 0.01 eq.), NiCl₂.glyme (1.04 mg, 4.74 umol, 0.005 eq.), Na₂CO₃ (201 mg, 1.89 mmol, 2 eq.), dtbbpy (1.27 mg, 4.74 umol, 0.005 eq.), and TTMSS (236 mg, 947.2 umol, 292 uL, 1 eq.) in DME (4 mL) was degassed and purged with Ar 3 times. Then the mixture was stirred at 15° C. for 6 h under an Ar atmosphere exposed to a 34 W blue LED. Upon completion, the reaction mixture was filtered, and the filtrate was concentrated. The residue was purified by prep-HPLC (column: Phenomenex luna C18 (250×70 mm, 15 um); mobile phase: [water (0.04% HCl)-ACN]; B %: 55%-85%, 20 min) to afford benzyl (1-(2,5-dimethoxy-4-(4,4,4-trifluorobutyl)phenyl)butan-2-yl)carbamate (776 mg, 1.71 mmol, 46% yield) as a white solid. ¹H NMR (400 MHz, DMSO-d6) δ ppm 7.40-7.05 (m, 5H), 6.72 (s, 2H), 4.99-4.83 (m, 2H), 3.74-3.62 (m, 5H), 3.62-3.55 (m, 1H), 2.53 (s, 4H), 2.30-2.15 (m, 2H), 1.79-1.65 (m, 2H), 1.50-1.32 (m, 2H), 0.89-0.78 (m, 3H).

### Step 2: Preparation of 1-(2,5-dimethoxy-4-(4,4,4-trifluorobutyl)phenyl)butan-2-amine (78)

To a solution of benzyl (1-(2,5-dimethoxy-4-(4,4,4-trifluorobutyl)phenyl)butan-2-yl)carbamate (776 mg, 1.71 mmol, 1 eq.) in MeOH (30 mL) and MeNH₂·H₂O (3 mL) was added Pd(OH)₂/C (1 g). The mixture was stirred at 15° C. for 1 h under H₂ (15 psi). Upon completion, the reaction mixture was filtered, and the filtrate was concentrated to afford 1-(2,5-dimethoxy-4-(4,4,4-trifluorobutyl)phenyl)butan-2-amine (501 mg, 1.56 mmol, 92% yield) as a white solid. LCMS R_{T}=2.149 min, MS cal.: 319.18 [M+H]⁺=320.1; 1H NMR (400 MHz, DMSO-d₆) δ ppm 6.78-6.74 (m, 2H), 3.74-3.68 (m, 6H), 2.80-2.70 (m, 1H), 2.65-2.55 (m, 3H), 2.42-2.29 (m, 1H), 2.29-2.16 (m, 2H), 1.74-1.70 (m, 2H), 1.41-1.28 (m, 1H), 1.27-1.12 (m, 3H), 0.87 (t, J=7.2 Hz, 3H); ¹³C NMR (101 MHz, DMSO-d₆) δ ppm 151.56, 151.01, 127.30, 127.15, 114.52, 113.44, 56.33, 56.29, 53.08, 38.95, 32.90, 32.63, 30.47, 29.03, 22.50, 11.00.

### Example 76: Preparation of 1-(2,5-dimethoxy-4-(5,5,5-trifluoropentyl)phenyl)butan-2-amine (79)

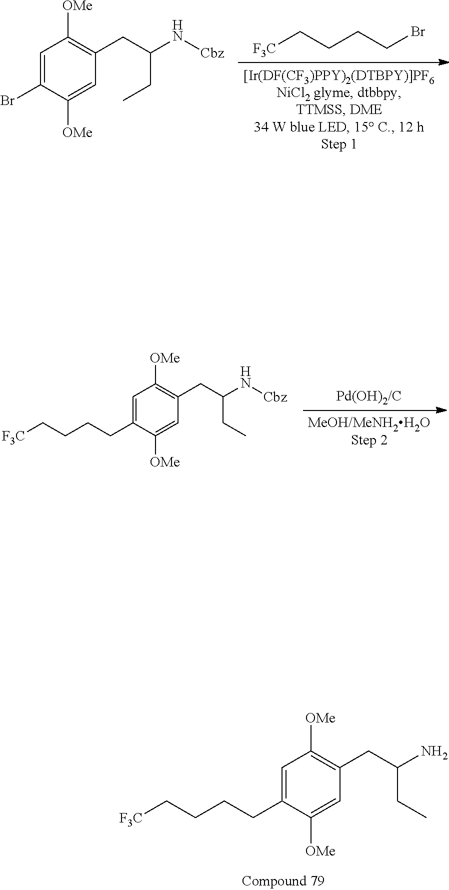 [SMILES: CCC(Cc1cc(OC)c(Br)cc1OC)NC(=O)OCc1ccccc1 ; FC(F)(F)CCCCBr ; CCC(Cc1cc(OC)c(CCCCC(F)(F)F)cc1OC)NC(=O)OCc1ccccc1 ; CCC(N)Cc1cc(OC)c(CCCCC(F)(F)F)cc1OC]

### Step 1: Preparation of benzyl (1-(2,5-dimethoxy-4-(5,5,5-trifluoropentyl)phenyl)butan-2-yl)carbamate

A mixture of 5-bromo-1,1,1-trifluoropentane (641 mg, 3.13 mmol, 4 eq.), benzyl (1-(4-bromo-2,5-dimethoxyphenyl)butan-2-yl)carbamate (330 mg, 781 umol, 1 eq, 3 batches), NiCl₂.glyme (859 ug, 3.91 umol, 0.005 eq.), Na₂CO₃ (166 mg, 1.56 mmol, 2 eq.), dtbbpy (1.05 mg, 3.91 umol, 0.005 eq.), TTMSS (194 mg, 781.41 umol, 241 uL, 1 eq.), and bis[3,5-difluoro-2-[5-(trifluoromethyl)-2-pyridyl]phenyl]iridium(1⁺) 4-tert-butyl-2-(4-tert-butyl-2-pyridyl)pyridine hexafluorophosphate (8.77 mg, 7.81 umol, 0.01 eq.) in DME (3 mL) was degassed and purged with Ar 3 times. Then the mixture was stirred at 15° C. for 12 h under an Ar atmosphere exposed to a 34 W blue LED. Upon completion, the reaction mixture was filtered, and the filtrate was concentrated. The residue was purified by prep-HPLC (column: Phenomenex luna C18 (250×70 mm, 15 um); [water (0.04% HCl)-ACN]; B %: 50%-80%, 20 min) to afford benzyl (1-(2,5-dimethoxy-4-(5,5,5-trifluoropentyl)phenyl)butan-2-yl)carbamate (500 mg, 973 umol, 42% yield) as a white solid. ¹H NMR (400 MHz, DMSO-d6) δ=7.52-7.27 (m, 5H), 6.75-6.51 (m, 2H), 5.04 (s, 2H), 4.92 (d, J=8.4 Hz, 1H), 3.80 (d, J=6.8 Hz, 1H), 3.75 (d, J=13.2 Hz, 6H), 2.76 (d, J=6.4 Hz, 2H), 2.60 (t, J=7.0 Hz, 2H), 2.21-2.02 (m, 2H), 1.77-1.54 (m, 6H), 1.51-1.39 (m, 1H), 0.96 (t, J=7.4 Hz, 3H).

### Step 2: Preparation of 1-(2,5-dimethoxy-4-(5,5,5-trifluoropentyl)phenyl)butan-2-amine (79)

To a solution of benzyl (1-(2,5-dimethoxy-4-(5,5,5-trifluoropentyl)phenyl)butan-2-yl)carbamate (400 mg, 856 umol, 1 eq.) in MeOH (30 mL) and MeNH₂·H₂O (3 mL) was added Pd(OH)₂/C (1 g, 7.1 mmol, 8.3 eq.). The mixture was stirred at 20° C. for 1 h under H₂ (15 psi). Upon completion, the reaction mixture was filtered and the filtrate concentrated to afford 1-(2,5-dimethoxy-4-(5,5,5-trifluoropentyl)phenyl)butan-2-amine (230 mg, 690 umol, 81% yield) as an off-white solid. LCMS R_{T}=2.237 min, MS cal.: 333.39 [M+H]⁺=334.1; 1H NMR (400 MHz, DMSO-d₆) δ=6.74 (d, J=7.2 Hz, 2H), 3.70 (d, J=5.4 Hz, 6H), 2.74 (dt, J=2.4, 5.2 Hz, 1H), 2.63-2.52 (m, 3H), 2.37 (dd, J=7.6, 13.2 Hz, 1H), 2.31-2.16 (m, 2H), 1.63-1.53 (m, 2H), 1.53-1.43 (m, 2H), 1.41-1.28 (m, 1H), 1.23-1.10 (m, 1H), 0.91-0.77 (m, 3H); ¹³C NMR (101 MHz, DMSO-d₆) δ ppm 151.51, 151.01, 128.15, 126.79, 114.40, 113.46, 56.31, 56.24, 53.08, 38.92, 30.44, 29.50, 29.00, 21.63, 21.60, 10.97.

### Example 77: Preparation of 2-(4-((5-fluoropentyl)thio)-3,5-dimethoxyphenyl)ethanamine (80)

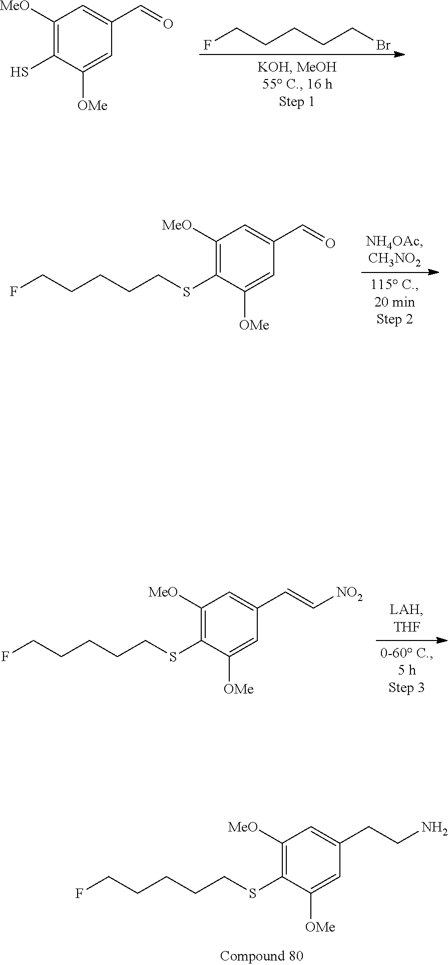

### Step 1: Preparation of 4-((5-fluoropentyl)thio)-3,5-dimethoxybenzaldehyde

To a mixture of 4-mercapto-3,5-dimethoxybenzaldehyde (1.47 g, 7.42 mmol, 1 eq.) dissolved in a solution of KOH (1.58 g, 28.2 mmol, 3.8 eq.) in MeOH (20 mL) was added 1-bromo-5-fluoropentane (1.88 g, 11.1 mmol, 1.5 eq.). The mixture was stirred at 55° C. for 16 h. Upon completion, the reaction mixture was quenched by addition of aq. HCl (1M) to pH=6-7. The mixture was diluted with H₂O (10 mL) and extracted with EtOAc (2×20 mL). The combined organic layer was washed with brine (10 mL), dried over Na₂SO₄, and filtered. The residue was purified by column chromatography (SiO₂, Petroleum ether/Ethyl acetate=100/1 to 0/1) to give 4-((5-fluoropentyl)thio)-3,5-dimethoxybenzaldehyde (1.5 g, 5.24 mmol, 71% yield) as a white solid. ¹HNMR (400 MHz, CHLOROFORM-d) δ=9.94 (s, 1H), 7.08 (s, 2H), 4.53-4.42 (m, 1H), 4.35 (t, J=6.0 Hz, 1H), 3.97 (s, 6H), 2.96 (t, J=6.4 Hz, 2H), 1.74-1.46 (m, 7H).

### Step 2: Preparation of (E)-(2,6-dimethoxy-4-(2-nitrovinyl)phenyl)(5-fluoropentyl)sulfane

To a mixture of 4-((5-fluoropentyl)thio)-3,5-dimethoxybenzaldehyde (1.5 g, 5.24 mmol, 1 eq.) dissolved in nitromethane (17.0 g, 278 mmol, 15 mL, 53 eq.) was added NH₄OAc (808 mg, 10.5 mmol, 2 eq.). The mixture was stirred at 115° C. for 20 min. Upon completion, the residue was treated with H₂O (10 mL). The aqueous phase was extracted with DCM (3×5 mL). The combined organic phase was dried over anhydrous Na₂SO₄, filtered, and concentrated. The residue was purified by column chromatography (SiO₂, Petroleum ether/Ethyl acetate=3/1 to 1/1) to give (E)-(2,6-dimethoxy-4-(2-nitrovinyl)phenyl)(5-fluoropentyl)sulfane (670 mg, 2.03 mmol, 39% yield) as a yellow solid. ¹HNMR (400 MHz, CHLOROFORM-d) δ=7.96 (d, J=13.6 Hz, 1H), 7.60 (d, J=13.6 Hz, 1H), 6.71 (s, 2H), 4.47 (t, J=6.0 Hz, 1H), 4.35 (t, J=6.0 Hz, 1H), 3.94 (s, 6H), 2.93 (t, J=6.8 Hz, 2H), 1.75-1.50 (m, 6H).

### Step 3: Preparation of 2-(4-((5-fluoropentyl)thio)-3,5-dimethoxyphenyl)ethanamine (80)

A solution of (E)-(2,6-dimethoxy-4-(2-nitrovinyl)phenyl)(5-fluoropentyl)sulfane (620 mg, 1.88 mmol, 1 eq.) in THF (10 mL) was cooled to 0° C. To this stirred solution, LiAlH₄ (572 mg, 15.1 mmol, 8 eq.) was added portionwise. The mixture was stirred at 60° C. for 5 h. Upon completion, the mixture was cooled to 0° C. and treated with water dropwise (0.6 mL) and stirred for 5 min. Then 0.6 mL 30% aq. NaOH solution was added dropwise. The mixture was stirred until a smooth dispersion formed then filtered and the filtrate was concentrated. The residue was purified by prep-HPLC (column: Phenomenex luna C18 (250×70 mm, 15 um); [water (0.04% HCl)-ACN]; B %: 5%-20%, 8 min) to give 2-(4-((5-fluoropentyl)thio)-3,5-dimethoxyphenyl)ethanamine (274 mg, 754 umol, 40% yield, HCl) as a white solid. LCMS (ESI+): m/z [M+H]⁺302.1; ¹H NMR (400 MHz, CHLOROFORM-d, HCl salt) δ=8.74-7.94 (m, 3H), 6.49 (s, 2H), 4.45 (t, J=6.0 Hz, 1H), 4.33 (t, J=6.0 Hz, 1H), 3.88 (s, 6H), 3.28 (s, 2H), 3.09 (s, 2H), 2.86-2.69 (m, 2H), 1.75-1.56 (m, 2H), 1.50 (d, J=3.2 Hz, 4H); ¹³CNMR (400 MHz, CHLOROFORM-d, HCl salt) δ=161.26, 137.73, 109.28, 104.95, 84.77, 83.14, 77.25, 56.43, 34.30, 33.80, 30.07, 29.87, 29.16, 24.27, 24.22.

### Example 79: Preparation of 2-(3,5-dimethoxy-4-((4,4,4-trifluorobutyl)thio)phenyl)ethanamine (81)

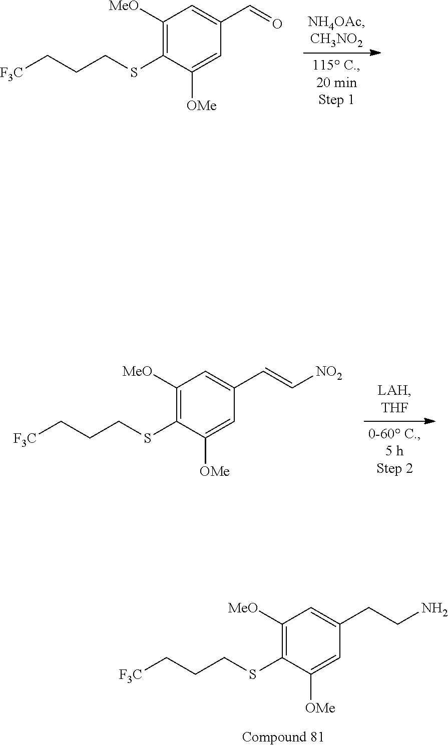 [SMILES: COc1cc(C=O)cc(OC)c1SCCCC(F)(F)F ; COc1cc(/C=C/[N+](=O)[O-])cc(OC)c1SCCCC(F)(F)F ; COc1cc(CCN)cc(OC)c1SCCCC(F)(F)F]

### Step 1: Preparation of (E)-(2,6-dimethoxy-4-(2-nitrovinyl)phenyl)(4,4,4-trifluorobutyl)sulfane

The mixture of 3,5-dimethoxy-4-((4,4,4-trifluorobutyl)thio)benzaldehyde (1.5 g, 4.87 mmol, 1 eq.) in nitromethane (27.2 g, 445 mmol, 24 mL, 91 eq.) was added NH₄OAc (750 mg, 9.73 mmol, 2 eq.). The mixture was stirred at 115° C. for 2 h. Upon completion, the mixture was concentrated and the residue purified by column chromatography (SiO₂, Petroleum ether/Ethyl acetate=100/1 to 10/1) to give (E)-(2,6-dimethoxy-4-(2-nitrovinyl)phenyl)(4,4,4-trifluorobutyl)sulfane (1 g, 2.64 mmol, 54% yield) as a yellow solid. ¹HNMR (400 MHz, CHLOROFORM-d) δ=7.96 (d, J=13.6 Hz, 1H), 7.61 (d, J=13.6 Hz, 1H), 6.72 (s, 2H), 4.00-3.88 (m, 6H), 2.94 (t, J=6.8 Hz, 2H), 2.39-2.21 (m, 2H), 1.77-1.63 (m, 2H).

### Step 2: Preparation of 2-(3,5-dimethoxy-4-((4,4,4-trifluorobutyl)thio)phenyl)ethanamine (81)

A stirred solution of (E)-(2,6-dimethoxy-4-(2-nitrovinyl)phenyl)(4,4,4-trifluorobutyl)sulfane (900 mg, 2.56 mmol, 1 eq.) in THF (15 mL) was cooled to 0° C. Then LiAlH₄ (583 mg, 15.4 mmol, 6 eq.) was added portionwise. The mixture was warmed to 60° C. and stirred for 5 h. Upon completion, the mixture was cooled to 0° C. and treated dropwise with H₂O (0.4 mL) with stirring followed by 30% aq. NaOH (0.4 mL). After stirring to a smooth dispersion, the mixture was filtered, and the filtrate concentrated. The residue was purified by prep-HPLC (column: Phenomenex luna C18 250×50 mm×10 um; mobile phase: [water (0.04% HCl)-ACN]; B %: 10%-40%, 10 min) to provide 2-(3,5-dimethoxy-4-((4,4,4-trifluorobutyl)thio)phenyl)ethanamine (270 mg, 835 umol, 33% yield, HCl) as a white solid. LCMS (ESI+): m/z [M+H]⁺ 324.0; ¹HNMR (400 MHz, DMSO-d₆, HCl salt) δ=8.16 (br s, 3H), 6.60 (s, 2H), 3.81-3.79 (m, 6H), 3.06 (s, 2H), 2.91-2.87 (m, 1H), 2.75-2.72 (m, 1H), 2.39-2.34 (m, 2H), 1.53-1.46 (m, 2H);

¹³CNMR (400 MHz, DMSO-d₆) δ=161.30, 140.44, 105.33, 56.46, 33.93, 32.33, 21.84, 21.82.

### Example 79: Preparation of 2-(3,5-dimethoxy-4-((5,5,5-trifluoropentyl)thio)phenyl)ethanamine (82)

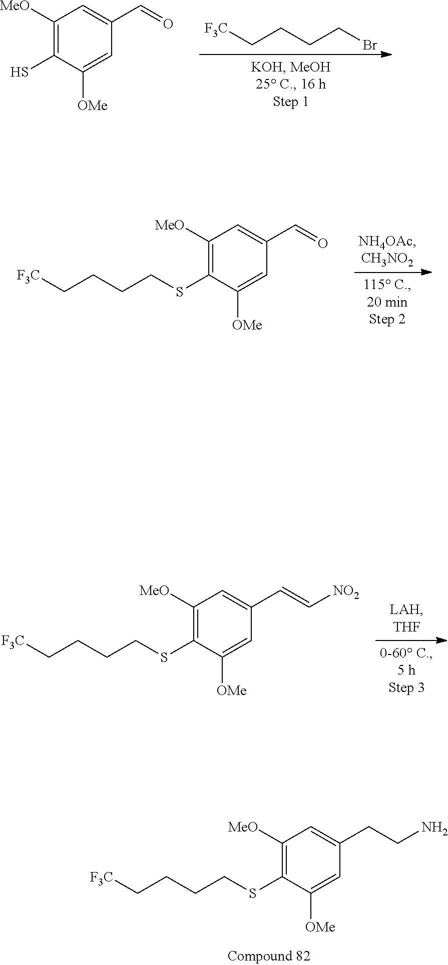 [SMILES: COc1cc(C=O)cc(OC)c1S ; FC(F)(F)CCCCBr ; COc1cc(C=O)cc(OC)c1SCCCCC(F)(F)F ; COc1cc(/C=C/[N+](=O)[O-])cc(OC)c1SCCCCC(F)(F)F ; COc1cc(CCN)cc(OC)c1SCCCCC(F)(F)F]

### Step 1: Preparation of 3,5-dimethoxy-4-((5,5,5-trifluoropentyl)thio)benzaldehyde

To a mixture of 4-mercapto-3,5-dimethoxybenzaldehyde (736 mg, 3.71 mmol, 1 eq.) and KOH (3.40 g, 60.6 mmol, 16.3 eq.) in MeOH (20 mL) was added 5-bromo-1,1,1-trifluoropentane (1.14 g, 5.57 mmol, 1.5 eq.). The mixture was stirred at 25° C. for 16 h. Upon completion, the reaction mixture was quenched by addition of aq. HCl (1M) to pH=6-7. After dilution with H₂O (10 mL), the mixture was extracted with EtOAc (2×20 mL). The combined organic layer was washed with brine (10 mL), dried over Na₂SO₄, and filtered. After concentration, the residue was purified by column chromatography (SiO₂, Petroleum ether/Ethyl acetate=100/1 to 0/1) to give 3,5-dimethoxy-4-((5,5,5-trifluoropentyl)thio)benzaldehyde (840 mg, 2.61 mmol, 70.19% yield) as a white solid. ¹HNMR (400 MHz, CHLOROFORM-d) δ=9.95 (s, 1H), 7.08 (s, 2H), 3.97 (s, 6H), 2.96 (t, J=7.0 Hz, 2H), 2.13-1.97 (m, 2H), 1.76-1.64 (m, 2H), 1.63-1.52 (m, 2H).

### Step 2: Preparation of (E)-(2,6-dimethoxy-4-(2-nitrovinyl)phenyl)(5,5,5-trifluoropentyl)sulfane

A stirred mixture of 3,5-dimethoxy-4-((5,5,5-trifluoropentyl)thio)benzaldehyde (840 mg, 2.61 mmol, 1 eq.) and NH₄OAc (402 mg, 5.22 mmol, 2 eq.) dissolved in CH₃NO₂ (17 g, 278 mmol, 15 mL) was purged with N₂ 3 times, then warmed and stirred at 115° C. for 15 min under a N₂ atmosphere. Upon completion, the reaction mixture was filtered and concentrated to give a residue. The residue was purified by column chromatography (SiO₂, Petroleum ether/Ethyl acetate=10/1) to afford (E)-(2,6-dimethoxy-4-(2-nitrovinyl)phenyl)(5,5,5-trifluoropentyl)sulfane (730 mg, 2.00 mmol, 77% yield) as a yellow solid. ¹H NMR (400 MHz, CHLOROFORM-d) δ ppm 1.51-1.62 (m, 11H), 1.63-1.75 (m, 2H), 2.00-2.12 (m, 2H), 2.92 (t, J=7.03 Hz, 2H), 3.94 (s, 6H), 6.72 (s, 2H), 7.60 (d, J=13.57 Hz, 1H), 7.97 (d, J=13.57 Hz, 1H).

### Step 3: Preparation of 2-(3,5-dimethoxy-4-((5,5,5-trifluoropentyl)thio)phenyl)ethanamine (82)

A solution of LiAlH₄ (155.82 mg, 4.11 mmol, 6 eq.) in THF (50 mL) was warmed to 60° C. Then a solution of (E)-(2,6-dimethoxy-4-(2-nitrovinyl)phenyl)(5,5,5-trifluoropentyl)sulfane (250 mg, 684 umol, 1 eq.) in THF (4 ml) was added dropwise. The mixture was stirred at 60° C. for 1 h under a N₂ atmosphere. Upon completion, the mixture was cooled to 0° C. and quenched with H₂O (2 mL) dropwise with stirring. Then 30% aq. NaOH (2 mL) was added dropwise with stirring until a smooth dispersion formed. The reaction mixture was filtered, and the filtrate concentrated to give a residue. The residue was purified by prep-HPLC (column: Phenomenex luna C18 250×50 mm×10 um; mobile phase: [water (HCl)-ACN]; B %: 15%-45%, 10 min) to give 2-(3,5-dimethoxy-4-((5,5,5-trifluoropentyl)thio)phenyl)ethanamine_(200 mg, 593 umol, 43% yield, HCl) as a white solid. ¹HNMR (400 MHz, CHLOROFORM-d, HCl salt) δ ppm 1.36-1.47 (m, 2H), 1.51-1.64 (m, 2H), 2.10-2.25 (m, 2H), 2.71 (t, J=6.91 Hz, 2H), 2.85-2.93 (m, 2H), 3.06 (s, 2H), 3.76-3.83 (m, 6H), 6.59 (s, 2H), 8.15 (br s, 3H); ¹³CNMR (400 MHz, CHLOROFORM-d, HCl salt) δ ppm 20.67, 20.70, 28.22, 32.49, 33.03, 33.91, 56.45, 105.38, 107.72, 126.79, 129.54, 139.93, 160.98, 161.10.

### Example 80: Preparation of 1-(4-((4-fluorobutyl)thio)-3,5-dimethoxyphenyl)butan-2-amine (83)

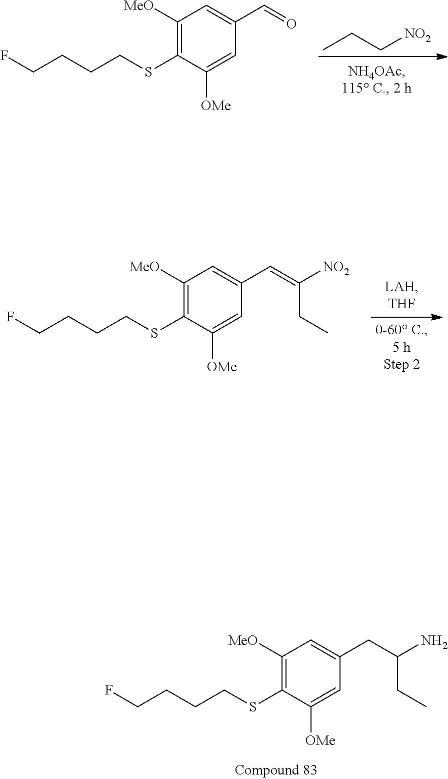

### Step1: Preparation of (E)-(2,6-dimethoxy-4-(2-nitrobut-1-en-1-yl)phenyl)(4-fluorobutyl)sulfane

A stirred mixture of 4-((4-fluorobutyl)thio)-3,5-dimethoxybenzaldehyde (900 mg, 3.30 mmol, 1 eq.) and NH₄OAc (510 mg, 6.61 mmol, 2 eq.) in 1-nitropropane (8.98 g, 101 mmol, 9.00 mL, 30.5 eq.) was warmed to 115° C. for 2 h. Upon completion, the mixture was cooled and concentrated. The residue was purified by column chromatography (SiO₂, Petroleum ether/Ethyl acetate=100/1 to 3/1) to give (E)-(2,6-dimethoxy-4-(2-nitrobut-1-en-1-yl)phenyl)(4-fluorobutyl)sulfane (600 mg, 1.75 mmol, 53% yield) as a yellow solid. ¹HNMR (400 MHz, CHLOROFORM-d) δ=7.98 (s, 1H), 6.61 (s, 2H), 4.54-4.32 (m, 2H), 3.96-3.87 (m, 6H), 2.97-2.83 (m, 4H), 1.91-1.74 (m, 2H), 1.63 (m, 2H), 1.35-1.27 (m, 3H).

### Step 2: Preparation of 1-(4-((4-fluorobutyl)thio)-3,5-dimethoxyphenyl)butan-2-amine (83)

A solution of (E)-(2,6-dimethoxy-4-(2-nitrobut-1-en-1-yl)phenyl)(4-fluorobutyl)sulfane (500 mg, 1.46 mmol, 1 eq.) in THF (10 mL) was cooled to 0° C. To this was added LiAlH₄ (332 mg, 8.74 mmol, 6 eq.) portionwise. The mixture was stirred at 60° C. for 5 h. Upon completion, the mixture was cooled and quenched with H₂O (0.5 mL) dropwise with stirring followed by 30% aq. NaOH (0.5 mL). The dispersion was stirred then filtered and the filtrate concentrated. The residue was purified by prep-HPLC (column: Phenomenex luna C18 250×50 mm×10 um; mobile phase: [water (0.04% HCl)-ACN]; B %: 10%-40%, 10 min) to give 1-(4-((4-fluorobutyl)thio)-3,5-dimethoxyphenyl)butan-2-amine_(160 mg, 507 umol, 35% yield, HCl) as a white solid. LCMS (ESI+): m/z [M+H]⁺ 316.1; ¹HNMR (400 MHz, DMSO-d₆, HCl salt) δ=8.14 (br s, 3H), 6.62 (s, 2H), 4.45 (t, J=6.1 Hz, 1H), 4.33 (t, J=6.1 Hz, 1H), 3.80 (s, 6H), 2.95-2.87 (m, 1H), 2.84-2.77 (m, 1H), 2.73 (t, J=7.1 Hz, 2H), 2.61-2.52 (m, 1H), 1.79-1.64 (m, 2H), 1.56 (quin, J=7.1 Hz, 2H), 1.43 (m, 2H), 0.94 (t, J=7.5 Hz, 3H); ¹³CNMR (400 MHz, DMSO-d₆, HCl salt) δ=160.98, 139.09, 107.93, 106.08, 84.75, 83.14, 56.48, 53.34, 38.66, 33.10, 29.27, 29.07, 25.37, 25.32, 9.90.

### Example 81: Preparation of 1-(3,5-demethoxy-4-((4,4,4-trifluorobutyl)thio)phenyl)butan-2-amine (84)

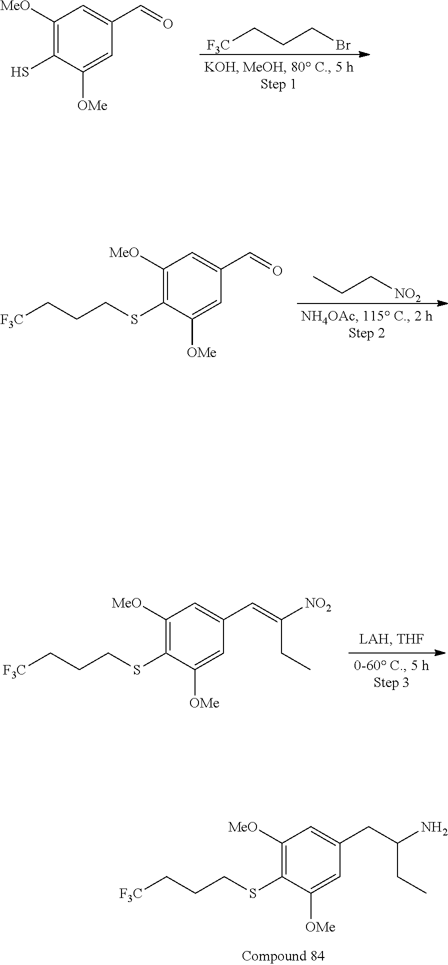

### Step 1: Preparation of 3,5-dimethoxy-4-((4,4,4-trifluorobutyl)thio)benzaldehyde

4-Mercapto-3,5-dimethoxybenzaldehyde (3.7 g, 18.7 mmol, 1 eq.) and KOH (1.05 g, 18.7 mmol, 1 eq.) were dissolved in MeOH (15 mL). To this was added 4-bromo-1,1,1-trifluorobutane (5.35 g, 28.0 mmol, 1.5 eq.). The mixture was stirred and warmed to 55° C. for 5 h. Upon completion, the reaction mixture was cooled and quenched by addition of aq. HCl (1M) to pH=6-7. The mixture was then diluted with H₂O (10 mL) and extracted with EtOAc (2×20 mL). The combined organic layer was washed with brine (10 mL), dried over Na₂SO₄, filtered and concentrated. The residue was purified by column chromatography (SiO₂, Petroleum ether/Ethyl acetate=100/1 to 5/1) to give 3,5-dimethoxy-4-((4,4,4-trifluorobutyl)thio)benzaldehyde (4.7 g, 17.3 mmol, 93% yield) as a yellow solid. ¹HNMR (400 MHz, CHLOROFORM-d) δ=9.92 (s, 1H), 7.07 (s, 2H), 4.00-3.90 (m, 6H), 2.96 (t, J=6.9 Hz, 2H), 2.36-2.16 (m, 2H), 1.77-1.62 (m, 2H).

### Step 2: Preparation of (E)-(2,6-dimethoxy-4-(2-nitrobut-1-en-1-yl)phenyl)(4,4,4-trifluorobutyl)sulfane

A solution of 3,5-dimethoxy-4-((4,4,4-trifluorobutyl)thio)benzaldehyde benzaldehyde (1.5 g, 4.87 mmol, 1 eq.) in 1-nitropropane (24 g, 269 mmol, 24 mL, 55.3 eq.) was treated with NH₄OAc (750 mg, 9.7 mmol, 2 eq.). The mixture was warmed and stirred at 115° C. for 2 h. Upon completion, the mixture was concentrated. The residue was purified by column chromatography (SiO₂, Petroleum ether/Ethyl acetate=100/1 to 10/1) to give (E)-(2,6-dimethoxy-4-(2-nitrobut-1-en-1-yl)phenyl)(4,4,4-trifluorobutyl)sulfane (1 g, 2.64 mmol, 54% yield) as a yellow oil. 1HNMR (400 MHz, CHLOROFORM-d) δ=7.98 (s, 1H), 6.61 (s, 2H), 3.98-3.86 (m, 6H), 2.97-2.84 (m, 4H), 2.41-2.20 (m, 2H), 1.77-1.63 (m, 2H), 1.36-1.27 (m, 3H).

### Step 3: Preparation of 1-(3,5-dimethoxy-4-((4,4,4-trifluorobutyl)thio)phenyl)butan-2-amine (84)

A stirred solution of (E)-(2,6-dimethoxy-4-(2-nitrobut-1-en-1-yl)phenyl)(4,4,4-trifluorobutyl)sulfane (900 mg, 2.37 mmol, 1 eq.) in THF (10 mL) was cooled to 0° C. and treated with LiAlH₄ (540.15 mg, 14.2 mmol, 6 eq.) portionwise. The mixture was warmed and stirred at 60° C. for 5 h. Upon completion, the mixture was cooled and then quenched dropwise with H₂O (0.5 mL) followed by 30% aq. NaOH (0.5 mL). After stirring to a smooth dispersion, the mixture was filtered and the filtrate was concentrated. The residue was purified by prep-HPLC (column: Phenomenex luna C18 250×50 mm×10 um; mobile phase: [water (0.04% HCl)-ACN]; B %: 10%-40%, 10 min) to give 1-(3,5-dimethoxy-4-((4,4,4-trifluorobutyl)thio)phenyl)butan-2-amine (280 mg, 797 umol, 34% yield, HCl) as a white solid. LCMS (ESI+): m/z [M+H]⁺ 352.1; ¹HNMR (400 MHz, DMSO-d₆, HCl salt) δ=8.00 (br s, 3H), 6.62 (s, 2H), 3.83-3.77 (m, 6H), 3.35 (s, 1H), 2.92-2.79 (m, 2H), 2.78-2.72 (m, 2H), 2.43-2.31 (m, 3H), 1.59-1.47 (m, 4H), 0.94 (t, J=7.5 Hz, 3H); ¹³CNMR (400 MHz, DMSO-d₆, HCl salt) δ=161.23, 139.58, 106.84, 106.00, 56.47, 53.31, 32.26, 31.43, 25.36, 21.83, 21.81, 9.86.

### Example 82: Preparation of 1-(4-((5-fluoropentyl)thio)-2,5-dimethoxyphenyl)propan-2-amine (85)

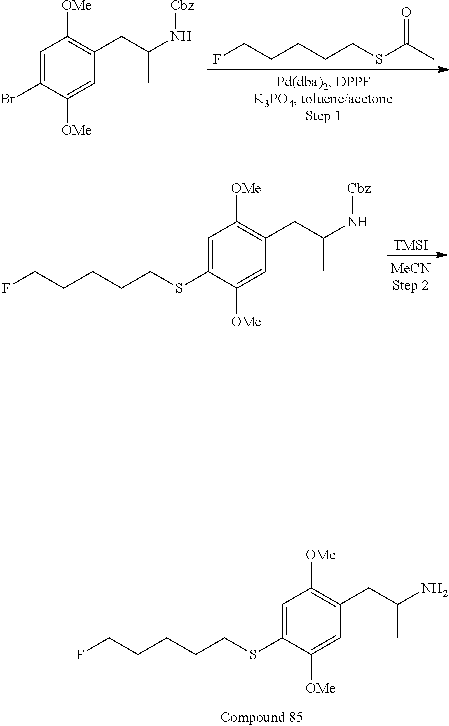

### Step 1: Preparation of benzyl (1-(4-((5-fluoropentyl)thio)-2,5-dimethoxyphenyl)propan-2-yl)carbamate

To a mixture of benzyl (1-(4-bromo-2,5-dimethoxyphenyl)propan-2-yl)carbamate (400 mg, 980 umol, 1 eq, 2 batches), S-(5-fluoropentyl) ethanethioate (483 mg, 2.94 mmol, 3 eq.), DPPF (54.3 mg, 98 umol, 0.1 eq.), and K₃PO₄ (208 mg, 980 umol, 1 eq.) in toluene (3 mL) and acetone (1.5 mL), was added Pd(dba)₂ (56.3 mg, 98 umol, 0.1 eq.) in one portion at 20° C. under N₂. The mixture was stirred and warmed to 115° C. for 12 h. Upon completion, the reaction mixture was cooled and quenched by the addition of aq. NH₄Cl (10 mL) at 20° C., and then extracted with EtOAc (3×10 mL). The combined organic layer was washed with brine (3×10 mL), dried over Na₂SO₄, filtered, and concentrated to give a residue. The residue was purified by prep-HPLC (column: Phenomenex luna C18 (250×70 mm, 15 um); [water (0.04% HCl)-ACN]; B %: 50%-80%, 20 min) to afford benzyl (1-(4-((5-fluoropentyl)thio)-2,5-dimethoxyphenyl)propan-2-yl)carbamate (700 mg, 1.43 mmol, 73% yield, 99.6% purity) as an off-white solid. ¹H NMR (400 MHz, CHLOROFORM-d) δ=7.44-7.28 (m, 5H), 6.83 (s, 1H), 6.65 (s, 1H), 5.05 (s, 2H), 4.99 (s, 1H), 4.57-4.44 (m, 1H), 4.44-4.33 (m, 1H), 4.07-3.88 (m, 1H), 3.88-3.62 (m, 6H), 2.90 (t, J=7.2 Hz, 2H), 2.84-2.66 (m, 2H), 1.80-1.63 (m, 4H), 1.62-1.56 (m, 2H), 1.18 (d, J=6.4 Hz, 3H).

### Step 2: Preparation of 1-(4-((5-fluoropentyl)thio)-2,5-dimethoxyphenyl)propan-2-amine (85)

To a stirred solution of benzyl (1-(4-((5-fluoropentyl)thio)-2,5-dimethoxyphenyl)propan-2-yl) carbamate (450 mg, 1.00 mmol, 1 eq.) in MeCN (5 mL) was added TMSI (601 mg, 3.00 mmol, 409 uL, 3 eq.) in one portion at 0° C. under N₂. The mixture was stirred at 20° C. for 12 h. Upon completion, the reaction mixture was quenched by the addition of sat. aq. NaHCO₃ solution (5 mL) at 20° C., and the mixture was filtered and concentrated to give a residue. The residue was purified by prep-HPLC (column: Phenomenex luna C18 (250×70 mm, 15 um); [water (HCl)-ACN]; B %: 20%-50%, 10 min) to afford 1-[4-(5-fluoropentylsulfanyl)-2,5-dimethoxy-phenyl]propan-2-amine (270 mg, 760 umol, 76% yield, 99% purity, HCl) as a yellow solid. The residue was further purified by prep-HPLC (column: Phenomenex luna [water (NH₄HCO₃)-ACN]; B %: 15%-45%, 8 min) to afford 1-(4-((5-fluoropentyl)thio)-2,5-dimethoxyphenyl)propan-2-amine (202 mg, 634 umol, 83% yield) as an off-white solid. LCMS R_{T}=2.041 min, MS cal.: 315.45 [M+H]⁺=316.1; 1H NMR (400 MHz, CHLOROFORM-d) δ=6.84 (s, 1H), 6.68 (s, 1H), 4.49 (t, J=6.0 Hz, 1H), 4.37 (t, J=6.0 Hz, 1H), 3.88 (s, 3H), 3.78 (s, 3H), 3.20 (d, J=5.6 Hz, 1H), 2.90 (t, J=7.2 Hz, 2H), 2.72 (dd, J=5.2, 12.9 Hz, 1H), 2.52 (dd, J=8.0, 13.2 Hz, 1H), 1.79-1.63 (m, 4H), 1.62-1.56 (m, 2H), 1.12 (d, J=6.4 Hz, 3H); ¹³C NMR (101 MHz, CHLOROFORM-d δ=151.91, 151.81, 127.42, 124.83, 122.10, 114.25, 113.98, 84.69, 83.05, 56.47, 56.12, 47.18, 41.02, 32.74, 30.09, 29.89, 28.76, 24.53, 24.48, 23.60.

### Example 83: Preparation of 2-(4-((5-fluoropentyl)thio)-2,5-dimethoxyphenyl)ethanamine (86)

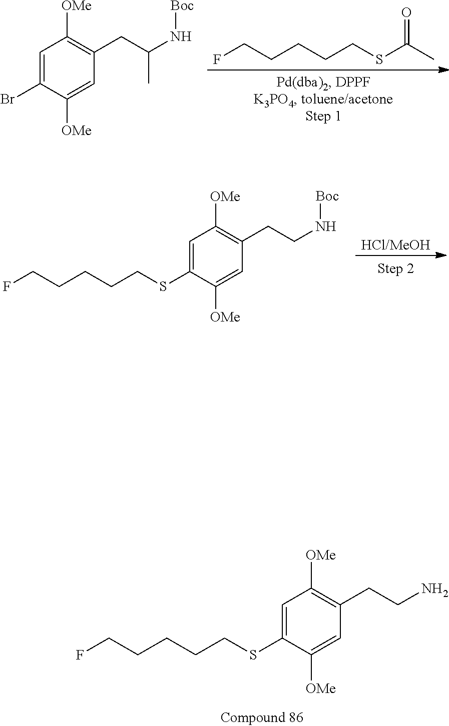

### Step 1: Preparation of tert-butyl (4-((5-fluoropentyl)thio)-2,5-dimethoxyphenethyl)carbamate

To a mixture of S-(5-fluoropentyl) ethanethioate (547.10 mg, 3.33 mmol, 4 eq.), tert-butyl (4-bromo-2,5-dimethoxyphenethyl)carbamate (300 mg, 833 umol, 1 eq, 3 batches), DPPF (46.2 mg, 83.3 umol, 0.1 eq.), and K₃PO₄ (177 mg, 833 umol, 1 eq.) in toluene (3 mL) and acetone (1.5 mL), was added Pd(dba)₂ (47.9 mg, 83.2 umol, 0.1 eq.) in one portion at 20° C. under N₂. The mixture was stirred at 115° C. for 12 h. Upon completion, the reaction mixture was filtered, and the filtrate was concentrated. The residue was purified by prep-HPLC (column: Phenomenex luna C18 (250×70 mm, 15 um); [water (0.04% HCl)-ACN]; B %: 50%-80%, 20 min) to afford tert-butyl (4-((5-fluoropentyl)thio)-2,5-dimethoxyphenethyl)carbamate (800 mg, 1.83 mmol, 73% yield) as a white solid. ¹H NMR (400 MHz, CHLOROFORM-d) δ=6.85 (s, 1H), 6.68 (s, 1H), 4.63 (d, J=2.8 Hz, 1H), 4.50 (t, J=6.0 Hz, 1H), 4.38 (t, J=6.0 Hz, 1H), 3.85 (s, 3H), 3.80 (s, 3H), 3.34 (d, J=5.4 Hz, 2H), 2.90 (t, J=7.2 Hz, 2H), 2.79 (t, J=6.8 Hz, 2H), 1.79-1.65 (m, 4H), 1.62-1.57 (m, 2H), 1.44 (s, 9H).

### Step 2: Preparation of 2-(4-((5-fluoropentyl)thio)-2,5-dimethoxyphenyl)ethanamine (86)

To a solution of tert-butyl (4-((5-fluoropentyl)thio)-2,5-dimethoxyphenethyl)carbamate (750 mg, 1.87 mmol, 1 eq.) in MeOH (15 mL) was added HCl/MeOH (4 M, 5.36 mL, 11.5 eq.) at 0° C. The mixture was stirred at 20° C. for 12 h. Upon completion, the reaction mixture was concentrated to afford 2-(4-((5-fluoropentyl)thio)-2,5-dimethoxyphenyl)ethanamine_(690 mg, 1.84 mmol, 98% yield, HCl) as a white solid. LCMS R_{T}=2.043 min, MS cal.: 301.42 [M+H]⁺=302.1; 1H NMR (400 MHz, DMSO-d₆, HCl salt) δ=8.09 (br s, 3H), 6.85 (s, 1H), 6.82 (s, 1H), 4.48 (t, J=6.0 Hz, 1H), 4.36 (t, J=6.0 Hz, 1H), 3.77 (d, J=6.8 Hz, 6H), 3.01-2.88 (m, 4H), 2.87-2.79 (m, 2H), 1.75-1.54 (m, 4H), 1.53-1.42 (m, 2H); ¹³C NMR (101 MHz, DMSO-d₆, HCl salt) δ=151.83, 150.87, 124.00, 123.29, 114.14, 111.71, 85.00, 83.39, 56.68, 56.54, 30.98, 29.92, 29.73, 28.44, 28.25, 24.52, 24.47.

### Example 84: Preparation of 2-(4-(6-fluorohexyl)-2,5-dimethoxyphenyl)ethanamine (87)

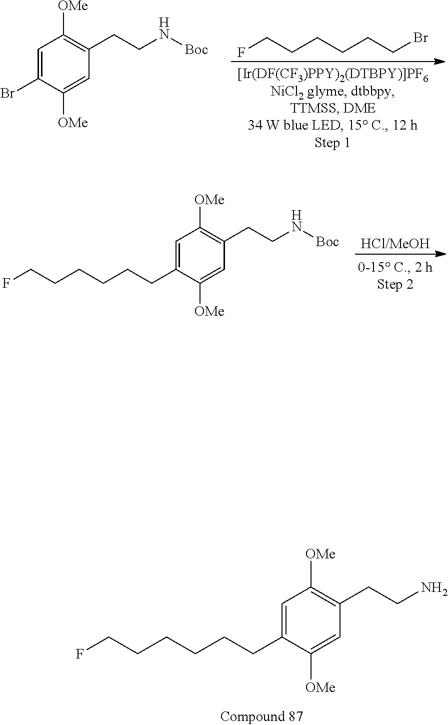 [SMILES: COc1cc(CCNC(=O)OC(C)(C)C)c(OC)cc1Br ; FCCCCCCBr ; COc1cc(CCNC(=O)OC(C)(C)C)c(OC)cc1CCCCCCF ; "HCl/MeOH" ; COc1cc(CCCCCCF)c(OC)cc1CCN]

### Step 1: Preparation of tert-butyl (4-(6-fluorohexyl)-2,5-dimethoxyphenethyl)carbamate

A mixture of tert-butyl (4-bromo-2,5-dimethoxyphenethyl)carbamate (354 mg, 983 umol, 1 eq, 2 batches), 1-bromo-6-fluorohexane (720 mg, 3.93 mmol, 4 eq.), bis[3,5-difluoro-2-[5-(trifluoromethyl)-2-pyridyl]phenyl]iridium(1⁺) 4-tert-butyl-2-(4-tert-butyl-2-pyridyl)pyridine hexafluorophosphate (11 mg, 9.83 umol, 0.01 eq.), NiCl₂.glyme (1.08 mg, 4.92 umol, 0.005 eq.), Na₂CO₃ (208 mg, 1.97 mmol, 2 eq.), dtbbpy (1.32 mg, 4.92 umol, 0.005 eq.), and TTMSS (245 mg, 983 umol, 303 uL, 1 eq.) in DME (4 mL) was degassed and purged with Ar 3 times. Then the mixture was stirred at 15° C. for 12 h under Ar atmosphere exposed to a 34 W blue LED. Upon completion, the reaction mixture was filtered, and the filtrate was concentrated. The residue was purified by prep-HPLC (column: Phenomenex luna C18 (250×70 mm, 15 um); mobile phase: [water (0.04% HCl)-ACN]; B %: 55%-85%, 20 min) to afford tert-butyl (4-(6-fluorohexyl)-2,5-dimethoxyphenethyl)carbamate (535 mg, 1.40 mmol, 71% yield) as a white solid. ¹H NMR (400 MHz, CHLOROFORM-d) δ ppm 6.68-6.61 (m, 2H), 4.77-4.64 (m, 1H), 4.51 (t, J=6.0 Hz, 1H), 4.42-4.36 (m, 1H), 3.78 (d, J=4.8 Hz, 6H), 3.40-3.28 (m, 2H), 2.78 (t, J=6.8 Hz, 2H), 2.63-2.54 (m, 2H), 1.78-1.52 (m, 6H), 1.44 (s, 9H), 1.43-1.39 (m, 2H).

### Step 2: Preparation of 2-(4-(6-fluorohexyl)-2,5-dimethoxyphenyl)ethanamine (87)

To a solution of tert-butyl (4-(6-fluorohexyl)-2,5-dimethoxyphenethyl)carbamate (530 mg, 1.39 mmol, 1 eq.) in MeOH (10 mL) was added HCl/MeOH (4 M, 30.8 mL) at 0° C. The mixture was stirred at 15° C. for 2 h. Upon completion, the reaction mixture was concentrated to afford 2-(4-(6-fluorohexyl)-2,5-dimethoxyphenyl)ethanamine (409 mg, 1.28 mmol, 92% yield, HCl) as a white solid. LCMS R_{T}=2.095 min, MS cal.: 283.19 [M+H]⁺=284.1; 1H NMR (400 MHz, DMSO-d₆, HCl salt) 6 ppm 8.02-7.92 (m, 3H), 6.79 (d, J=4.0 Hz, 2H), 4.48 (t, J=6.0 Hz, 1H), 4.39-4.33 (m, 1H), 3.73 (d, J=3.2 Hz, 6H), 2.99-2.90 (m, 2H), 2.85-2.77 (m, 2H), 2.55-2.51 (m, 2H), 1.68-1.47 (m, 4H), 1.41-1.26 (m, 4H); ¹³C NMR (101 MHz, DMSO-d₆, HCl salt) δ ppm 151.32, 151.27, 130.11, 123.29, 113.84, 113.53, 85.09, 83.48, 56.36, 39.23, 30.37, 30.18, 30.03, 29.02, 28.47, 25.04, 24.99.

### Example 85: Preparation of 2-(2,5-dimethoxy-4-(5,5,5-trifluoropentyl)phenyl)ethanamine (88)

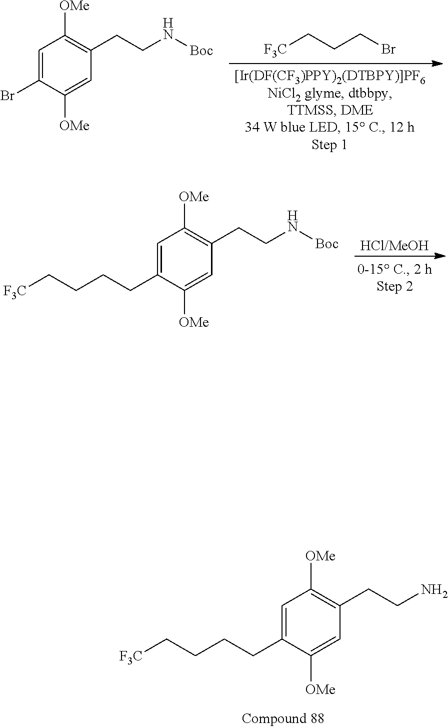 [SMILES: COc1cc(CCNC(=O)OC(C)(C)C)c(OC)cc1Br ; FC(F)(F)CCCBr ; COc1cc(CCNC(=O)OC(C)(C)C)c(OC)cc1CCCCC(F)(F)F ; "HCl/MeOH" ; COc1cc(CCCCC(F)(F)F)c(OC)cc1CCN]

### Step 1: Preparation of tert-butyl (2,5-dimethoxy-4-(5,5,5-trifluoropentyl)phenethyl)carbamate

A mixture of tert-butyl (4-bromo-2,5-dimethoxyphenethyl)carbamate (350 mg, 972 umol, 1 eq, 3 batches), 5-bromo-1,1,1-trifluoropentane (797 mg, 3.89 mmol, 4 eq.), NiCl₂.glyme (1.07 mg, 4.86 umol, 0.005 eq.), Na₂CO₃ (206 mg, 1.94 mmol, 2 eq.), dtbbpy (1.30 mg, 4.86 umol, 0.005 eq.), TTMSS (242 mg, 972 umol, 300 uL, 1 eq.) and bis[3,5-difluoro-2-[5-(trifluoromethyl)-2-pyridyl]phenyl]iridium(1⁺) 4-tert-butyl-2-(4-tert-butyl-2pyridyl)pyridine hexafluorophosphate (10.9 mg, 9.72 umol, 0.01 eq.) in DME (4 mL) was degassed and purged with Ar 3 times. Then the mixture was stirred at 15° C. for 12 h under an Ar atmosphere exposed to a 34 W blue LED. Upon completion, the reaction mixture was filtered, and the filtrate was concentrated. The residue was purified by prep-HPLC (column: Phenomenex luna C18 (250×70 mm, 15 um); [water (0.04% HCl)-ACN]; B %: 50%-80%, 20 min) to afford tert-butyl (2,5-dimethoxy-4-(5,5,5-trifluoropentyl)phenethyl)carbamate (1 g, 2.22 mmol, 76% yield) as a white solid. ¹H NMR (400 MHz, CHLOROFORM-d) δ=6.65-6.64 (m, 2H), 4.71-4.65 (m, 1H), 3.78 (d, J=4.0 Hz, 6H), 3.34 (d, J=5.6 Hz, 2H), 2.78 (t, J=6.8 Hz, 2H), 2.61 (t, J=7.2 Hz, 2H), 2.13-2.11 (m, 2H), 1.67-1.60 (m, 4H), 1.44 (s, 9H).

### Step 2: Preparation of 2-(2,5-dimethoxy-4-(5,5,5-trifluoropentyl)phenyl)ethanamine (88)

To a solution of tert-butyl (2,5-dimethoxy-4-(5,5,5-trifluoropentyl)phenethyl)carbamate (950 mg, 2.34 mmol, 1 eq.) in MeOH (30 mL) was added HCl/MeOH (4 M, 10 mL, 17 eq.) at 0° C. The mixture was stirred at 20° C. for 10 h. Upon completion, the reaction mixture was concentrated to afford 2-(2,5-dimethoxy-4-(5,5,5-trifluoropentyl)phenyl)ethanamine (700 mg, 2.05 mmol, 87% yield, HCl) as an off-white solid. LCMS R_{T}=2.124 min, MS cal.: 305.34 [M+H]⁺=306.1; 1H NMR (400 MHz, CHLOROFORM-d, HCl salt) δ=8.32 (br s, 3H), 6.73 (s, 1H), 6.66 (s, 1H), 3.81 (s, 3H), 3.78 (s, 3H), 3.30-3.17 (m, 2H), 3.08-2.99 (m, 2H), 2.60 (t, J=7.2 Hz, 2H), 2.20-2.01 (m, 2H), 1.67-1.60 (m, 4H); ¹³C NMR (101 MHz, CHLOROFORM-d, HCl salt) δ=151.31, 151.22, 130.21, 122.34, 113.82, 112.90, 56.08, 55.89, 39.82, 33.43, 29.86, 29.59, 29.14, 21.67, 21.64.

### Example 86: Preparation of 1-(4-(5-fluoropentyl)-2,5-dimethoxyphenyl)butan-2-amine (89)

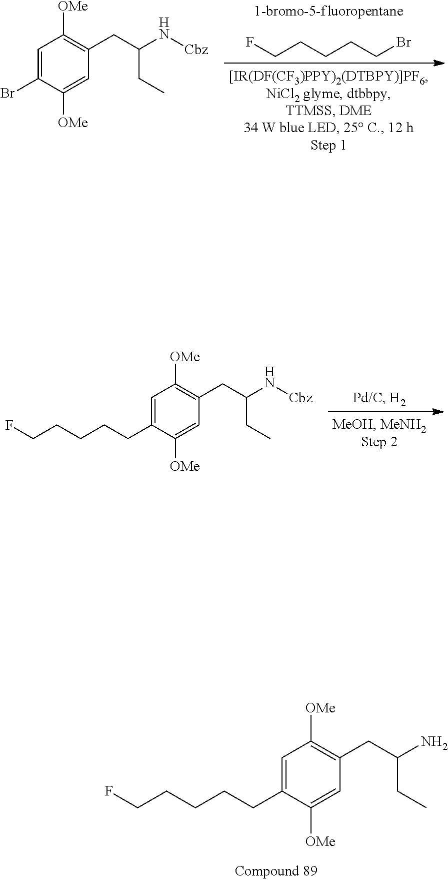 [SMILES: CCC(Cc1cc(OC)c(Br)cc1OC)NC(=O)OCc1ccccc1 ; FCCCCCBr ; CCC(Cc1cc(OC)c(CCCCCF)cc1OC)NC(=O)OCc1ccccc1 ; CCC(N)Cc1cc(OC)c(CCCCCF)cc1OC]

### Step 1: Preparation of benzyl (1-(4-(5-fluoropentyl)-2,5-dimethoxyphenyl)butan-2-yl)carbamate

A mixture of benzyl (1-(4-bromo-2,5-dimethoxyphenyl)butan-2-yl)carbamate (100 mg, 237 umol, 1 eq, 6 batches), 1-bromo-5-fluoropentane (320 mg, 1.89 mmol, 8 eq.), Na₂CO₃ (50 mg, 474 umol, 2 eq.), NiCl₂.glyme (260 ug, 1.18 umol, 73 uL, 0.005 eq.), TTMSS (59 mg, 237 umol, 73 uL, 1 eq.), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (2.66 mg, 2.37 umol, 0.01 eq.), and dtbbpy (318 ug, 1.18 umol, 0.005 eq.) in DME (6 mL) was degassed and purged with Ar 3 times. The mixture was stirred at 25° C. for 12 h under Ar atmosphere while exposed to a 34 W blue LED. Upon completion, the reaction mixture was filtered. The filtrate was concentrated. The residue was purified by prep-HPLC (column: Phenomenex Luna C18 100×30 mm×5 um; mobile phase: [water (HCl)-ACN]; B %: 60%-90%, 10 min) to afford benzyl (1-(4-(5-fluoropentyl)-2,5-dimethoxyphenyl)butan-2-yl)carbamate (200 mg, 458 umol, 32% yield, 98.8% purity) as a white solid. ¹H NMR (400 MHz, CHLOROFORM-d) δ ppm 7.37-7.29 (m, 4H), 6.64 (d, J=4.1 Hz, 2H), 5.04 (s, 2H), 4.94 (d, J=8.4 Hz, 1H), 4.51 (t, J=6.2 Hz, 1H), 4.39 (t, J=6.2 Hz, 1H), 3.79 (s, 1H), 3.76 (s, 3H), 3.73 (s, 3H), 2.75 (d, J=6.4 Hz, 2H), 2.64-2.55 (m, 2H), 1.81-1.69 (m, 2H), 1.64-1.57 (m, 4H), 1.53-1.42 (m, 3H), 0.96 (t, J=7.4 Hz, 3H).

### Step 2: Preparation of 1-(4-(5-fluoropentyl)-2,5-dimethoxyphenyl)butan-2-amine (89)

A mixture of benzyl (1-(4-(5-fluoropentyl)-2,5-dimethoxyphenyl)butan-2-yl)carbamate (200 mg, 464 umol, 1 eq.) and Pd/C (50 mg, 46 umol, 10% purity, 0.1 eq.) in MeOH (4 mL) was stirred for 30 min at 30° C. under a hydrogen balloon. Then MeNH₂ (0.8 mL) was added to the reaction mixture. The mixture was then stirred at 25° C. for 2 h. Upon completion, the reaction mixture was filtered, and the filtrate concentrated to give a residue. The residue was purified by prep-HPLC (column: Phenomenex Luna 80×30 mm×3 um; mobile phase: [water (HCl)-ACN]; B %: 20%-50%, 8 min) to afford 1-(4-(5-fluoropentyl)-2,5-dimethoxyphenyl)butan-2-amine (130 mg, 389.38 umol, 84.02% yield, 100% purity, HCl) as a white solid. LCMS R_{T}=2.167 min, MS cal.: 297.41 [M+H]⁺=298.1; ¹H NMR (400 MHz, CHLOROFORM-d, HCl salt) δ ppm 8.40-8.32 (m, 3H), 6.75 (s, 1H), 6.63 (s, 1H), 4.55-4.46 (m, 1H), 4.44-4.33 (m, 1H), 3.83 (s, 3H), 3.76 (s, 3H), 3.57-3.43 (m, 1H), 3.13-3.04 (m, 1H), 3.03-2.92 (m, 1H), 2.64-2.53 (m, 2H), 1.83-1.67 (m, 4H), 1.65-1.56 (m, 2H), 1.50-1.41 (m, 2H), 1.13-1.04 (m, 3H); ¹³C NMR (101 MHz, CHLOROFORM-d, HCl salt) δ ppm 151.354, 151.236, 130.750, 122.151, 114.410, 112.928, 85.009, 83.377, 56.221, 55.937, 53.965, 34.309, 30.389, 30.203, 29.678, 25.319, 25.121, 10.105.

### Example 87: Preparation of 1-(4-(2-ethoxyethyl)-2,5-dimethoxyphenyl)butan-2-amine (90)

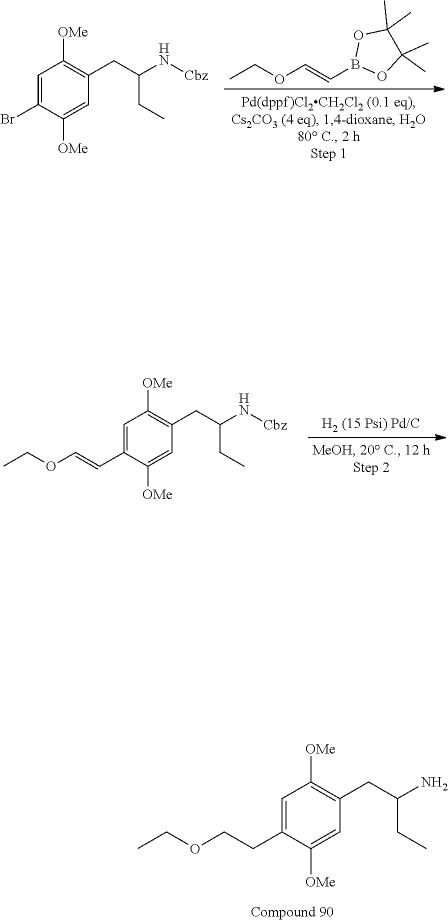

### Step 1: Preparation of benzyl (E)-(1-(4-(2-ethoxyvinyl)-2,5-dimethoxyphenyl)butan-2-yl)carbamate

A mixture of benzyl (1-(4-bromo-2,5-dimethoxyphenyl)butan-2-yl)carbamate (2 g, 4.74 mmol, 1 eq), (E)-2-(2-ethoxyvinyl)-4,4,5,5-tetramethyl-1,3,2-dioxaborolane (1.41 g, 7.10 mmol, 1.5 eq), Cs₂CO₃ (6.17 g, 18.94 mmol, 2.37 mL, 4 eq), and Pd(dppf)Cl₂·CH₂Cl₂ (386.75 mg, 473.58 umol, 0.1 eq) in 1,4-dioxane (15 mL) and H₂O (5 mL) was degassed and purged with N₂ three times, then the mixture was stirred at 80° C. for 2 h under a N₂ atmosphere. On completion, the mixture was diluted with H₂O (20 mL) and extracted with EtOAc (3×20 mL). The combined organic layers were washed with brine, dried over Na₂SO₄, filtered, and concentrated under reduced pressure to give a residue. The residue was purified by column chromatography (SiO₂, Petroleum ether/Ethyl acetate=20/1 to 0/1) to give benzyl (E)-(1-(4-(2-ethoxyvinyl)-2,5-dimethoxyphenyl)butan-2-yl)carbamate (1.27 g, 3.07 mmol, 65% yield) as a white solid. ¹H NMR (400 MHz, CHLOROFORM-d) δ ppm 0.92-1.04 (m, 3H) 1.23-1.31 (m, 1H) 1.35 (t, J=7.00 Hz, 3H) 1.42-1.66 (m, 4H) 2.71-2.83 (m, 2H) 3.69-3.82 (m, 8H) 3.93 (q, J=7.00 Hz, 2H) 4.83-4.96 (m, 1H) 5.00-5.09 (m, 2H) 6.02 (d, J=13.01 Hz, 1H) 6.61-6.75 (m, 2H) 7.08 (d, J=13.01 Hz, 1H) 7.28-7.36 (m, 4H).

### Step 2: Preparation of 1-(4-(2-ethoxyethyl)-2,5-dimethoxyphenyl)butan-2-amine (90)

To a solution of benzyl (E)-(1-(4-(2-ethoxyvinyl)-2,5-dimethoxyphenyl)butan-2-yl)carbamate (1 g, 2.42 mmol, 1 eq) in MeOH (10 mL) was added Pd/C (300 mg, 10% purity) under N₂ atmosphere. The suspension was degassed and purged with H₂ 3 times. The mixture was stirred under H₂ (15 Psi) at 20° C. for 1 h. Upon completion, the reaction mixture was filtered, washed with methanol (3×20 mL), and concentrated under reduced pressure to give a residue. The residue was purified by prep-HPLC (column: Phenomenex luna C18 80×40 mm×3 um; mobile phase: [water (HCl)-ACN]; B %: 1%-30%, 7 min) to give 1-(4-(2-ethoxyethyl)-2,5-dimethoxyphenyl)butan-2-amine (322.60 mg, 1.01 mmol, 42% yield, HCl) obtained as a white solid. LCMS R_{T}=1.893 min, MS cal.: 281.20 [M+H]⁺=282.1; 1H NMR (400 MHz, DMSO-d₆, HCl salt) δ ppm 0.91 (t, J=7.52 Hz, 3H), 1.07-1.13 (m, 3H), 1.46-1.56 (m, 2H), 2.72-2.85 (m, 4H), 3.19-3.30 (m, 1H), 3.43 (q, J=7.01 Hz, 2H), 3.50 (t, J=7.34 Hz, 2H), 3.73 (d, J=3.67 Hz, 6H), 6.85 (d, J=6.36 Hz, 2H), 8.03 (br s, 3H); ¹³C NMR (101 MHz, DMSO-d₆, HCl salt) δ ppm 151.488, 151.416, 126.472, 123.323, 114.592, 114.187, 69.874, 65.592, 56.420, 56.316, 52.650, 40,683, 33.101, 30.616, 25.243, 15.638, 9.926.

### Example 88: Preparation of 1-(2,5-demethoxy-4-(propoxymethyl)phenyl)butan-2-amine (91)

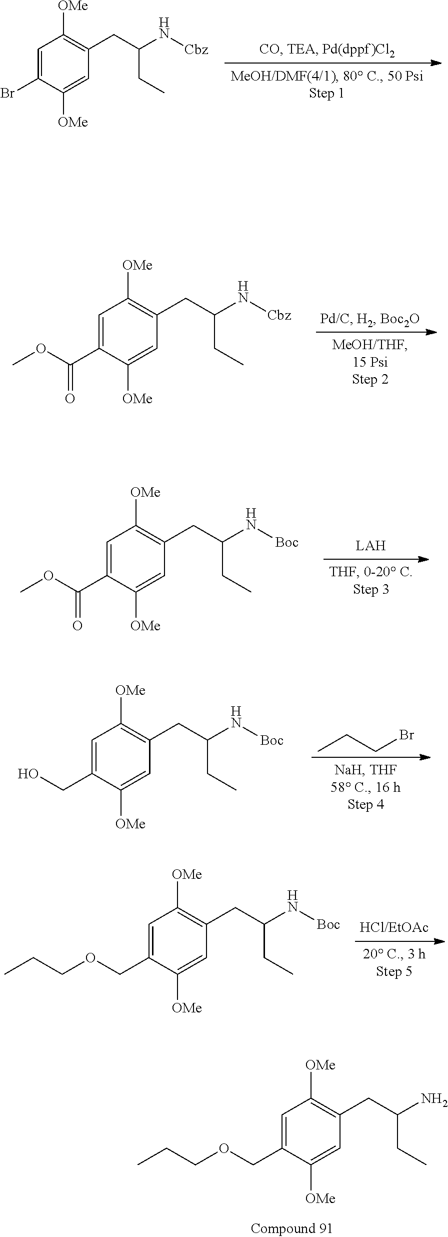

### Step 1: Preparation of methyl 4-(2-(((benzyloxy)carbonyl)amino)butyl)-2,5-dimethoxybenzoate

To a solution of benzyl (1-(4-bromo-2,5-dimethoxyphenyl)butan-2-yl)carbamate (2 g, 4.74 mmol, 1 eq.) in MeOH (80 mL) and DMF (20 mL) was added TEA (1.44 g, 14.21 mmol, 1.98 mL, 3 eq.) and Pd(dppf)Cl₂·CH₂Cl₂ (580 mg, 710 umol, 0.15 eq.). The mixture was then stirred at 80° C. for 40 h under CO (50 Psi). Upon completion, the reaction mixture was filtered, then the filtrate was extracted with EtOAc (3×50 mL). The combined organic layer was washed with brine (50 mL), dried over Na₂SO₄, filtered, and concentrated to give a residue. The residue was purified by silica gel chromatography (PE:EA=100:1-8:1) to afford methyl 4-(2-(((benzyloxy)carbonyl)amino)butyl)-2,5-dimethoxybenzoate (1.6 g, 3.69 mmol, 77% yield, 92% purity) as a white solid. ¹H NMR (400 MHz, CHLOROFORM-d) δ ppm 7.31-7.21 (m, 5H), 6.74 (s, 1H), 4.97 (s, 2H), 4.74 (d, J=8.8 Hz, 1H), 3.85 (s, 3H), 3.75 (d, J=5.0 Hz, 6H), 2.76 (d, J=6.8 Hz, 2H), 1.56-1.36 (m, 2H), 0.91 (t, J=7.3 Hz, 3H).

### Step 2: Preparation of methyl 4-(2-((tert-butoxycarbonyl)amino)butyl)-2,5-dimethoxybenzoate

To a solution of methyl 4-(2-(((benzyloxy)carbonyl)amino)butyl)-2,5-dimethoxybenzoate (1.3 g, 3.24 mmol, 1 eq.) in MeOH (13 mL) and THF (5 mL) was added Pd/C (323.82 umol, 0.1 eq.). The mixture was then stirred at 25° C. for 1 h under H₂ (15 Psi). Then Boc₂O (706.74 mg, 3.24 mmol, 743.93 uL, 1 eq.) was added and the mixture was stirred at 25° C. for 1 h. Upon completion, the mixture was filtered, and the filtrate concentrated. The residue was purified by silica gel chromatography (PE:EA=100:0-8:1) to afford methyl 4-(2-((tert-butoxycarbonyl)amino)butyl)-2,5-dimethoxybenzoate (0.7 g, 1.91 mmol, 59% yield) as a white solid. ¹H NMR (400 MHz, CHLOROFORM-d) δ ppm 7.31 (s, 1H), 6.83 (s, 1H), 3.90 (s, 3H), 3.88 (s, 3H), 3.83 (s, 3H), 3.81-3.68 (m, 1H), 2.79 (d, J=5.9 Hz, 2H), 1.58 (s, 3H), 1.37 (s, 9H), 0.96 (t, J=7.5 Hz, 3H).

### Step 3: Preparation of tert-butyl (1-(4-(hydroxymethyl)-2,5-dimethoxyphenyl)butan-2-yl)carbamate

To a solution of methyl 4-(2-((tert-butoxycarbonyl)amino)butyl)-2,5-dimethoxybenzoate (700 mg, 1.9 mmol, 1 eq.) in THF (10 mL) was added LiAlH₄ (145 mg, 3.81 mmol, 2 eq.) under a N₂ atmosphere at 0° C. The mixture was stirred and warmed to 20° C. for 3 h. Upon completion, the reaction mixture was quenched by dropwise addition of ethyl acetate (5 ml) and MeOH (5 ml) and then filtered and concentrated to give a residue. The residue was purified by silica gel chromatography (PE:EA=100:0-8:1) to afford tert-butyl (1-(4-(hydroxymethyl)-2,5-dimethoxyphenyl)butan-2-yl)carbamate (150 mg, 433 umol, 22% yield, 98% purity) as a white solid. ¹H NMR (400 MHz, CHLOROFORM-d) δ ppm 6.83 (s, 1H), 6.72 (s, 1H), 4.66 (s, 2H), 3.83 (s, 3H), 3.81 (s, 3H), 3.77-3.66 (m, 1H), 2.76 (d, J=6.3 Hz, 2H), 1.55 (m, 2H), 1.38 (s, 11H), 0.95 (t, J=7.4 Hz, 3H).

### Step 4: tert-butyl (1-(2,5-dimethoxy-4-(propoxymethyl)phenyl)butan-2-yl)carbamate

To a solution of tert-butyl (1-(4-(hydroxymethyl)-2,5-dimethoxyphenyl)butan-2-yl)carbamate (150 mg, 442 umol, 1 eq.) in THF (3 mL) was added NaH (40 mg, 663 umol, 40% purity, 1.5 eq.) and 1-bromopropane (82 mg, 663 umol, 60 uL, 1.5 eq.) under N₂ at 20° C. Then the mixture was stirred at 58° C. for 16 h. Upon completion, the reaction mixture was quenched by careful addition of saturated ammonium chloride solution (5 mL). The mixture was filtered and concentrated to give a residue. The residue was purified by prep-TLC (Petroleum ether:Ethyl acetate=5:1, R_{f}=0.2) to afford tert-butyl (1-(2,5-dimethoxy-4-(propoxymethyl)phenyl)butan-2-yl)carbamate (60 mg, 154 umol, 35% yield, 98% purity) as a white solid. ¹H NMR (400 MHz, CHLOROFORM-d) δ ppm 6.95 (s, 1H), 6.69 (s, 1H), 4.69-4.58 (m, 1H), 4.52 (s, 2H), 3.81 (s, 3H), 3.79 (s, 3H), 3.48 (t, J=6.7 Hz, 2H), 2.75 (d, J=6.4 Hz, 2H), 1.72-1.62 (m, 2H), 1.52 (s, 2H), 1.38 (s, 10H), 0.96 (q, J=7.3 Hz, 6H).

### Step 5: Preparation of 1-(2,5-dimethoxy-4-(propoxymethyl)phenyl)butan-2-amine (91)

To a solution of tert-butyl (1-(2,5-dimethoxy-4-(propoxymethyl)phenyl)butan-2-yl)carbamate (40 mg, 105 umol, 1 eq.) in EtOAc (0.5 mL) was added HCl/EtOAc (4 M, 0.5 mL, 19.1 eq.). The mixture was stirred at 20° C. for 3 h. Upon completion, the mixture was concentrated to give a residue. The residue was purified by prep-HPLC (column: Phenomenex luna C18 80×40 mm×3 um; mobile phase: [water (HCl)-ACN]; B %: 1%-35%, 7 min) to afford 1-(2,5-dimethoxy-4-(propoxymethyl)phenyl)butan-2-amine (2.2 mg, 7.2 umol, 7% yield, 91.8% purity, HCl) as a white solid. A second purification was carried out. by prep-HPLC (column: Waters Xbridge BEH C18 100×30 mm×10 um; mobile phase: [water (NH₄HCO₃)-ACN]; B %: 25%-55%, 8 min) to afford 1-(2,5-dimethoxy-4-(propoxymethyl)phenyl)butan-2-amine (0.67 mg, 2.38 umol, 31% yield, 100% purity) as a white solid. LCMS R_{T}=2.039 min, MS cal.: 281.39 [M+H]⁺=282.1; ¹H NMR (400 MHz, CD₃OD) δ ppm 7.01 (s, 1H), 6.81 (s, 1H), 4.52 (s, 2H), 3.81 (d, J=2.9 Hz, 6H), 3.50 (t, J=6.6 Hz, 2H), 3.20-3.07 (m, 1H), 2.90 (dd, J=5.8, 13.1 Hz, 1H), 2.73-2.60 (m, 1H), 1.69-1.61 (m, 2H), 1.61-1.44 (m, 2H), 1.02 (t, J=7.5 Hz, 3H), 0.97 (t, J=7.4 Hz, 3H).

### Example 89: Preparation of 1-(2,5-demethoxy-4-(pentylthio)phenyl)butan-2-amine (94)

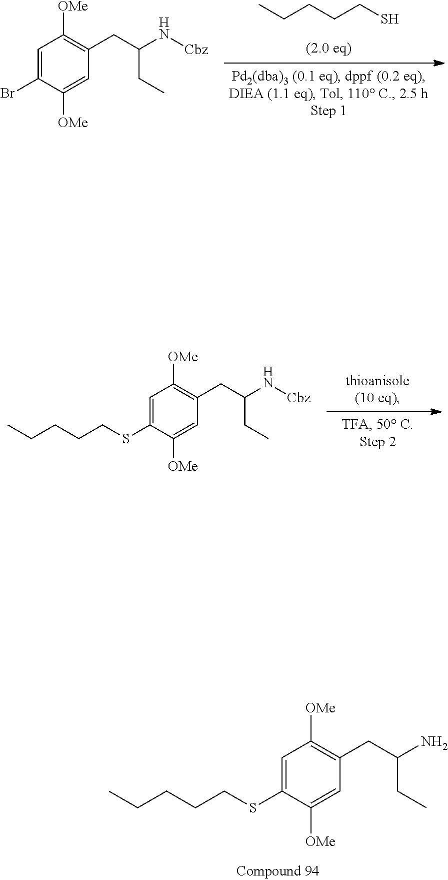

### Step 1: Preparation of benzyl (1-(2,5-dimethoxy-4-(pentylthio)phenyl)butan-2-yl)carbamate

To a solution of benzyl (1-(4-bromo-2,5-dimethoxyphenyl)butan-2-yl)carbamate (2 g, 4.74 mmol, 1 eq.) and pentane-1-thiol (987 mg, 9.47 mmol, 2 eq.) in toluene (14 mL) was added DIEA (673 mg, 5.21 mmol, 907 uL, 1.1 eq.), DPPF (525 mg, 947.2 umol, 0.2 eq.), and Pd₂(dba)₃ (434 mg, 474 umol, 0.1 eq.) under a N₂ atmosphere. The mixture was stirred at 110° C. for 2.5 h. Upon completion, the reaction mixture was partitioned between ethyl acetate (3×10 mL) and water (10 mL). The combined organic phase was washed with brine (20 mL), dried over anhydrous Na₂SO₄, filtered, and concentrated. The residue was purified by silica gel chromatography (PE:EA=100:1-50:1) to afford benzyl (1-(2,5-dimethoxy-4-(pentylthio)phenyl)butan-2-yl)carbamate (1.04 g, 2.20 mmol, 46% yield, 94.2% purity) as a white solid. ¹H NMR (400 MHz, CHLOROFORM-d) δ ppm 7.38-7.28 (m, 5H), 6.81 (s, 1H), 6.65 (s, 1H), 5.04 (s, 2H), 4.85 (d, J=8.4 Hz, 1H), 3.79 (s, 3H), 3.77 (s, 3H), 3.74 (s, 1H), 2.88 (t, J=7.4 Hz, 2H), 2.76 (d, J=6.8 Hz, 2H), 1.70-1.59 (m, 4H), 1.45-1.33 (m, 4H), 0.96 (t, J=7.4 Hz, 3H), 0.92-0.88 (m, 3H).

### Step 2: Preparation of 1-(2,5-dimethoxy-4-(pentylthio)phenyl)butan-2-amine (94)

A solution of benzyl (1-(2,5-dimethoxy-4-(pentylthio)phenyl)butan-2-yl)carbamate (600 mg, 1.35 mmol, 1 eq.) and thioanisole (1.67 g, 13.46 mmol, 1.59 mL, 10 eq.) in TFA (6 mL) was heated to 50° C. for 2 h. Upon completion, the reaction mixture was concentrated to remove TFA, then water (8 mL) was added and the mixture was extracted with PE (2×10 mL) to remove the thioanisole, the water phase was lyophized to give the crude product. The crude was purified by prep-HPLC (column: Phenomenex luna C18 80×40 mm×3 um; mobile phase: [water (HCl)-ACN]; B %: 30%-50%, 7 min) to afford 1-(2,5-dimethoxy-4-(pentylthio)phenyl)butan-2-amine (194 mg, 558 umol, 41% yield, 100% purity, HCl) as a white solid. LCMS R_{T}=2.314 min, MS cal.: 311.48 [M+H]⁺=312.1; ¹H NMR (400 MHz, CD₃OD, HCl salt) δ ppm 6.94 (s, 1H), 6.84 (d, J=4.0 Hz, 1H), 3.85 (s, 3H), 3.84 (s, 3H), 3.46-3.36 (m, 1H), 3.04-2.95 (m, 1H), 2.91 (t, J=7.3 Hz, 3H), 1.75-1.60 (m, 4H), 1.50-1.32 (m, 4H), 1.07 (t, J=7.5 Hz, 3H), 0.93 (t, J=7.2 Hz, 3H); ¹³C NMR (101 MHz, CD₃OD, HCl salt) δ ppm 150.95 150.76, 129.51, 122.38, 113.98, 113.11, 55.84, 46.93, 34.78, 31.78, 22.77, 17.84, 13.96.

### Example 90: 5-HT2A Receptor Binding

The binding affinities of disclosed compounds at the ketanserin binding site of the 5-HT2A receptor were determined in radioligand binding experiments, with the results summarized in Table 1. Disclosed compounds exhibited substantial binding affinity for the 5-HT2A receptor.

TABLE 1
5-HT2A receptor binding affinities of disclosed compounds.

| | Kᵢ at 5-HT2A |
|---|---|
| | Receptor (nM) |
| Compound | ([³H]ketanserin) |
| DOI | 14.5 |
| 25D-NBOMe | 2.52 |
| 2C-TFM | 15.8 |
| 25CN-NBOH | 5.79 |
| LSD | 0.89 |
| Mescaline | 17,400 |
| DMT | 416 |
| 2C-B | 8.25 |
| 2C-E | 16.7 |
| Psilocin | 58.6 |
| 5-MeO-DMT | 39 |
| 2 | 101 |
| 3 | 9.54 |
| 4 | 2.03 |
| 4ent1 | 1.79 |
| 4ent2 | 3.05 |
| 5 | 3.4 |
| 6 | 7.1 |
| 7 | 26.3 |
| 8 | 211 |
| 9 | 602 |
| 10 | 1.79 |
| 11 | 185 |
| 12 | 249 |
| 13 | 0.1 |
| 14 | 1.74 |
| 15 | 9.42 |
| 16 | 10.8 |
| 17 | 45.8 |
| 18 | 120 |
| 19 | 99.4 |
| 20 | 128 |
| 21 | 306 |
| 22 | 17.6 |
| 23 | 5.41 |
| 24 | 4,090 |
| 25 | 1,470 |
| 26 | 22.9 |
| 27 | 10.8 |
| 28 | 1.58 |
| 29 | 27.5 |
| 30 | 19.4 |
| 31 | 38.8 |
| 33 | 651 |
| 35 | 51.9 |
| 36 | 0.7 |
| 37 | 57.4 |
| 38 | 7.79 |
| 39 | 4.46 |
| 40 | 9.06 |
| 41 | 50.3 |
| 42 | 223 |
| 43i | 51.6 |
| 44 | 28 |
| 45 | 35 |
| 46 | 978 |
| 47 | 761 |
| 47i | >1,230 |
| 48 | 293 |
| 49 | 1.66 |
| 50 | 1.11 |
| 51 | 0.04 |
| 52 | 14.5 |
| 53 | 0.94 |
| 54 | 0.76 |
| 55 | 2.71 |
| 56 | 1.53 |
| 57 | 0.97 |
| 58 | 1.35 |
| 59 | 31.5 |
| 60 | 126 |
| 61 | 1.99 |
| 63 | 3.82 |
| 63i | 58.6 |
| 64 | 2.71 |
| 67 | 86.4 |
| 67i | 130 |
| 68 | 57 |
| 69 | 338 |
| 70 | 196 |
| 71 | 402 |
| 72 | 75.7 |
| 73 | 23.6 |
| 74 | 15.9 |
| 75 | 5.56 |
| 76 | 24.8 |
| 77 | 132 |
| 78 | >1,225 |
| 79 | 51.1 |
| 80 | 156 |
| 81 | >1,225 |
| 82 | 388 |
| 83 | >1,225 |
| 84 | >1,225 |
| 85 | 3.39 |
| 86 | 2.13 |
| 87 | 1.53 |
| 88 | 6.31 |
| 89 | 56.8 |
| 90 | 215 |
| 91 | >1,225 |
| 94 | 5.66 |

### Methods

5-HT2A Receptor Radioligand Binding. Affinity of the test compounds for the 5-HT2A receptor was determined in radioligand binding experiments with [³H]ketanserin by WuXi AppTec (Hong Kong) Limited, using methods adapted from the literature and under conditions described in Table 2.

TABLE 2
Assay conditions for 5-HT2A receptor radioligand binding.

| Receptor Source | HEK293 stable cell line | |
|---|---|---|
| Vehicle | 1.0% DMSO | |
| Incubation Time | 1 | h |
| Incubation Temperature | 25° | C. |

| | |
|---|---|
| Incubation Buffer | 50 mM Tris-HCl, pH 7.4 |
| Ligand | 1 nM [³H]ketanserin |
| Non-Specific Ligand | 1 μM ketanserin |

### Example 91: Functional Activity at 5-HT2A, 5-HT2B, 5-HT2C, and 5-HT1A Receptors

The functional activity of disclosed compounds at several 5-HT receptor subtypes (5-HT2A, 5-HT2B, 5-HT2C, and 5-HT1A) was determined in Ca²⁺ flux assays, with the results summarized in Table 3. All compounds tested exhibited high efficacy agonist activity at the 5-HT2A receptor, but the potency of that activity varied over a ˜1000-fold range depending on the specific compound. In contrast, none of the disclosed compounds exhibited an EC₅₀ below 5 μM at the 5-HT1A receptor, demonstrating the high selectivity of this molecular scaffold for 5-HT2A over 5-HT1A. Selectivity for the 5-HT2A receptor over the 5-HT2B and 5-HT2C receptors was typically more modest and depended on the specific compound in question, but several interesting observations were made. The nature of the substituent at position 4 of the arene (R¹ in Formula (I)) was important for controlling selectivity across the 5-HT2 receptor subtypes. For instance, in the sub-series consisting of Compounds 23, 4, and 5, selectivity for 5-HT2A over 5-HT2B and 5-HT2C increased as the alkyl chain was extended from n-pentyl to n-heptyl. In contrast, installation of a 4-fluorobutyl group at this position, as in Compound 2, dramatically shifted selectivity in favor of 5-HT2C (e.g., compare Compounds 23 and 2). The nature of the substituent alpha to the amine (R⁵ in Formula (I)) was also found to be an important modulator of 5-HT2 receptor subtype selectivity. For example, compounds with an ethyl group at this position were often found to be substantially more selective for 5-HT2A compared to their counterparts bearing a methyl or hydrogen at this position (e.g., compare Compounds 7 vs. 23, 40 vs. 4, and 41 vs. 3). Further, the stereochemistry of this substituent also had a substantial effect on 5-HT2 subtype selectivity. For example, 4ent2 was much more selective for 5-HT2A over 5-HT2B than 4ent1, since 4ent2 lacked almost all agonist activity at 5-HT2B, while 4ent1 was a potent and efficacious agonist.

TABLE 3
Functional activity at 5-HT receptor subtypes.

| | 5-HT2A | % Act | 5-HT2B | % Act | 5-HT2C | % Act | 5-HT1A | % Act |
|---|---|---|---|---|---|---|---|---|
| | EC₅₀ | @ Max | EC₅₀ | @ Max | EC₅₀ | @ Max | EC₅₀ | @ Max |
| Compound | (nM) | Conc. | (nM) | Conc. | (nM) | Conc. | (nM) | Conc. |
| DOI | 0.19 | 106 | 4.88 | 86.3 | 0.818 | 92.6 | >10,000 | −2.47 |
| 25D-NBOMe | 0.251 | 97.5 | 1.27 | 43.3 | <0.508 | 91.5 | >10,000 | −4.43 |
| 2C-TFM | 1.05 | 81.5 | 16.3 | 82 | 0.593 | 88.2 | >10,000 | −0.647 |
| 25CN-NBOH | 0.408 | 95.3 | NT | NT | NT | NT | NT | NT |
| LSD | 4.33 | 108 | 59.4 | 19.3 | 88.3 | 92.1 | 25.1 | 91.4 |
| Mescaline | 114 | 102 | 853 | 45.4 | 536 | 91.1 | 12,500 | −5.02 |
| DMT | 22.2 | 93.4 | >100,000 | 42.8 | 31.3 | 92 | >100,000 | 70.2 |
| 2C-B | 0.478 | 99.7 | 20 | 73.3 | 1.14 | 89.4 | 33,100 | 47.8 |
| 2C-E | 0.747 | 105 | 29.7 | 66.2 | 1.82 | 89.5 | 43,700 | 17.8 |
| Psilocin | 6.5 | 95.6 | 4,290 | 1.44 | 30.3 | 95.1 | >3,160 | 0.72 |
| 5-MeO-DMT | 1.76 | 106 | 36.6 | 17.6 | 10.1 | 89.8 | 279 | 100 |
| 2 | 4.22 | 95.6 | 66.3 | 44.2 | 0.756 | 91.2 | >10,000 | −1.28 |
| 3 | 1.19 | 102 | 39 | 68.7 | 1.03 | 89.7 | >10,000 | 7.68 |
| 4 | 2.65 | 120 | 294 | 39.8 | 6.34 | 99.8 | >10,000 | 30.4 |
| 4ent1 | 2.4 | 93 | 154 | 84.2 | 6.97 | 94.2 | 19,200 | 201 |
| 4ent2 | 5.39 | 86.3 | >10,000 | 3.22 | 26.7 | 107 | 14,500 | 225 |
| 5 | 2.79 | 124 | >10,000 | 9.38 | 14.3 | 101 | >10,000 | 23.4 |
| 6 | 3.34 | 105 | 259 | 62.2 | 1.65 | 85.6 | >10,000 | 18.9 |
| 7 | 36.6 | 71.5 | NS | 0.211 | 1,820 | 115 | >10,000 | 33.1 |
| 8 | 51.3 | 124 | 436 | 61.1 | 6.98 | 87.9 | >10,000 | 36.8 |
| 9 | 3.86 | 114 | 89.6 | 55.7 | 8.86 | 95.9 | >10,000 | 8.39 |
| 10 | 2.39 | 120 | >10,000 | 5.05 | 2.35 | 96.4 | >10,000 | 16.2 |
| 11 | 44.1 | 117 | 323 | 65.6 | 11.7 | 102 | >10,000 | 27.2 |
| 12 | 3.5 | 135 | 20.6 | 67.1 | 5.99 | 92.9 | >10,000 | 8.71 |
| 13 | 8.05 | 112 | 627 | 26.8 | 0.766 | 89.7 | >10,000 | 19.7 |
| 14 | 7.61 | 112 | NS | −0.00088 | 70.8 | 96.2 | >10,000 | 25 |
| 15 | 2.18 | 82.8 | NS | −0.878 | 2.28 | 87.4 | >10,000 | −2.75 |
| 16 | 3.99 | 82.5 | NS | −0.0459 | 3.61 | 88 | >10,000 | −3.45 |
| 17 | 4.25 | 88.8 | 54.6 | 20.1 | 9.96 | 87.6 | >10,000 | −4.49 |
| 18 | 16.1 | 90.2 | NS | −1.09 | 32.2 | 92.9 | >10,000 | −3.45 |
| 19 | 6.04 | 116 | 663 | 24.7 | 15.6 | 90.3 | >10,000 | 9.14 |
| 20 | 7.78 | 90.8 | 1070 | 11.3 | 145 | 90.2 | >10,000 | −2.68 |
| 21 | 23 | 82.5 | NS | −0.472 | 495 | 88.3 | >10,000 | −0.927 |
| 22 | 0.123 | 103 | 13.7 | 94.5 | 0.271 | 94.4 | >10,000 | 7.71 |
| 23 | 1.95 | 100 | 42 | 20 | 3.75 | 90.1 | >10,000 | 15 |
| 24 | 17 | 101 | 159 | 78.2 | 125 | 95.8 | >10,000 | −1.35 |
| 25 | 48.9 | 113 | 1,390 | 39.2 | 288 | 99.3 | >10,000 | 8.71 |
| 26 | 1.76 | 92.6 | 59.6 | 63.9 | 2.25 | 101 | >10,000 | 13.1 |
| 27 | 2.23 | 103 | 35.3 | 36.5 | 1.96 | 95.6 | 62,100 | 75.5 |
| 28 | 0.806 | 105 | 63.4 | 49.4 | 3.19 | 89.5 | 38,600 | 207 |
| 29 | 1.2 | 104 | >10,000 | 0.37 | 2.94 | 92 | 26,200 | 244 |
| 30 | 20.6 | 95.3 | 416 | 39 | 33.4 | 101 | 13,900 | 253 |
| 31 | 2.85 | 86.5 | >10,000 | −6.3 | 2.92 | 91.6 | 47,600 | 109 |
| 33 | 26.9 | 114 | >10,000 | 0.73 | 57.4 | 93 | >100,000 | −0.67 |
| 35 | 2.66 | 109 | >10,000 | 4.96 | 0.638 | 97.1 | >10,000 | 2.45 |
| 36 | 2.09 | 90.5 | 716 | 46.8 | 5.51 | 82.6 | 30,500 | 183 |
| 37 | 1.4 | 95 | 473 | 54.1 | .662 | 99.3 | >10,000 | 9.36 |
| 38 | 1.7 | 86.1 | 325 | 40.1 | 1.87 | 89.8 | >100,000 | 40.8 |
| 39 | 1.46 | 107 | 178 | 60.6 | 1.91 | 83.6 | 44,000 | 118 |
| 40 | 38.9 | 71.1 | >10,000 | 34.2 | 3,340 | 77.9 | 16,800 | 287 |
| 41 | 18.6 | 83.7 | >10,000 | 0.94 | 174 | 70.8 | 54,100 | 121 |
| 42 | 31.6 | 86.3 | >10,000 | 0.78 | 28.5 | 84.3 | >100,000 | 20 |
| 43i | 71.4 | 75.9 | >1,000 | 0.22 | 44 | 83.9 | 36,700 | 166 |
| 44 | 5.8 | 101 | 110 | 38.6 | 13 | 88.1 | >100,000 | 47.6 |
| 45 | 22.8 | 95.7 | 40.9 | 62.4 | 27.9 | 91.2 | 36,100 | 92.1 |
| 46 | 61.4 | 89 | 327 | 33.4 | 34.1 | 89 | >100,000 | 30.1 |
| 47 | 26.8 | 98.7 | 140 | 57.8 | 8.89 | 97.4 | >10,000 | −0.95 |
| 47i | 74.4 | 89 | 979 | 58.6 | 575 | 87.6 | >100,000 | 0.3 |
| 48 | 79.9 | 98.6 | >10,000 | −0.54 | 683 | 58 | >100,000 | 33.8 |
| 49 | 75.9 | 75.9 | >1,000 | 0.72 | 7.43 | 93.6 | 27,100 | 155 |
| 50 | 6.71 | 104 | >10,000 | 0.57 | 9.04 | 89.9 | 41,300 | 73.8 |
| 51 | 7.96 | 86.5 | >10,000 | 9.79 | 3.14 | 90.9 | 34,000 | 129 |
| 52 | 29.6 | 101 | >10,000 | −0.15 | 140 | 87.6 | 84,700 | 58.4 |
| 53 | 50.2 | 99.3 | >10,000 | −0.013 | 79.1 | 83.8 | 22,300 | 182 |
| 54 | 8.76 | 100 | >10,000 | 3.12 | 37 | 91.5 | NT | 49.3 |
| 55 | 27.8 | 102 | >10,000 | −0.084 | 176 | 81.5 | 27,200 | 139 |
| 56 | 23.4 | 97.3 | >10,000 | −1.92 | 66.5 | 82.4 | 25,700 | 166 |
| 57 | 4.43 | 109 | >10,000 | 0.57 | 1.19 | 79 | 25,800 | 96.7 |
| 58 | 8.82 | 95.2 | >10,000 | 3.12 | 2.17 | 79.2 | 40,400 | 99.2 |
| 59 | 13.7 | 107 | >10,000 | −0.74 | 28.7 | 95.1 | NT | 18.6 |
| 60 | 111 | 95.3 | >10,000 | −0.27 | >10,000 | 13.4 | >10,000 | 7.14 |
| 61 | 5.82 | 98.9 | 309 | 19.9 | 2.72 | 91.1 | 31,500 | 168 |
| 63 | 10.3 | 93.7 | 385 | 31.2 | 9.84 | 95.2 | 27,000 | 93.5 |
| 63i | 10.9 | 84.2 | >10,000 | −0.18 | 19.2 | 93.6 | 63,500 | 96.4 |
| 64 | 7.7 | 104 | >10,000 | 0.475 | 4.88 | 82.2 | >100,000 | −5.78 |
| 67 | 23.6 | 112 | 284 | 34.4 | 3.89 | 90.3 | 59,600 | 66.7 |
| 67i | 51.2 | 95.3 | 590 | 51.6 | 4.53 | 91.5 | 23,700 | 86.8 |
| 68 | 86.9 | 97.9 | >10,000 | 20.6 | 15.7 | 84.2 | 22,900 | 121 |
| 69 | 7.82 | 89.3 | 179 | 68.4 | 4.67 | 94.9 | >100,000 | 39.4 |
| 70 | 15.3 | 102 | 174 | 51.8 | 4.07 | 84.5 | >100,000 | 18.6 |
| 71 | 5.73 | 103 | 64.5 | 89.3 | 3.47 | 94.2 | >100,000 | 31.4 |
| 72 | 127 | 92.2 | >10,000 | 0.9 | 35.7 | 86.7 | 28,500 | 87 |
| 73 | 1.12 | 94.6 | 22.4 | 56.4 | 1.63 | 91.1 | NT | 76 |
| 74 | 6.26 | 87.9 | 15 | 72.2 | 18.1 | 87 | 42,000 | 118 |
| 75 | 16.2 | 62.4 | >10,000 | 0.63 | 53.6 | 102 | 95,600 | 52.3 |
| 76 | 40 | 66 | >10,000 | 1.01 | 565 | 74.8 | 34,000 | 101 |
| 77 | 22.1 | 64.2 | >10,000 | 0.13 | 186 | 64.6 | NT | 66.3 |
| 78 | 128 | 62.3 | >10,000 | −0.13 | 679 | 47.2 | NT | 60.4 |
| 79 | 97.7 | 47.7 | >10,000 | −0.63 | 4520 | 52.4 | 91,900 | 128 |
| 80 | 7.43 | 98.3 | 55.5 | 65.7 | 15.2 | 92.4 | 48,500 | 73.7 |
| 81 | 74.3 | 97.2 | 15.4 | 79.8 | 56.8 | 90.7 | 59,500 | 72.3 |
| 82 | 22.5 | 100 | 71.6 | 77.4 | NT | NT | 60,100 | 67 |
| 83 | 218 | 82.4 | >10,000 | 5.56 | 1,430 | 49.4 | >100,000 | 36.3 |
| 84 | 383 | 69.2 | 302 | 31.2 | 3,430 | 38.3 | 24,800 | 107 |
| 85 | 1.54 | 111 | 14 | 72.4 | 1.42 | 97.4 | >100,000 | 38.6 |
| 86 | 2.73 | 98.6 | 49.6 | 74.6 | 0.689 | 87.2 | 18,500 | 77.5 |
| 87 | 2.29 | 93.7 | 33.6 | 76.8 | 0.578 | 90.6 | | 66.9 |
| 88 | 12.5 | 80.8 | 19.6 | 77.6 | 6.41 | 82.1 | 30,500 | 79 |
| 89 | 44.2 | 63.2 | >10,000 | 0.66 | NT | NT | 22,500 | 108 |
| 90 | 138 | 65.8 | >10,000 | 0.74 | NT | NT | >10,000 | 3.45 |
| 91 | >3,160 | 38.2 | >10,000 | −0.25 | NT | NT | >100,000 | 6.43 |
| 94 | 14 | 68.8 | >10,000 | −0.7 | NT | NT | 9,070 | 196 |

NT = not tested;
NS = EC₅₀ not determined since no measurable agonist signal

### Methods

Functional Assays at 5-HT2A, 5-HT2B, and 5-HT1A Receptors. Agonist activity at 5-HT2A, 5-HT2B, and 5-HT1A receptors was determined using a FLIPR Ca²⁺ flux assay at WuXi AppTec (Hong Kong) Limited according to their standard protocols. Briefly, stably transfected cells expressing the receptor of interest (HEK293 for 5-HT2A and 5-HT2B; CHO cells for 5-HT1A) were grown and plated in a 384 well plate and incubated at 37° C. and 5% CO₂ overnight. A solution of 250 mM probenecid in 1 mL FLIPR assay buffer was prepared fresh. This was combined with a fluorescent dye (Fluo-4 Direct™) to make a final assay concentration of 2.5 mM. Compounds were diluted 1:3.16 for 10 points and 750 nL was added to a 384 well compound plate using ECHO along with 30 μL assay buffer. The fluorescent dye was then added to the assay plate along with assay buffer to a final volume of 40 μL. The cell plate was incubated for 50 min at 37° C. and 5% CO₂ and placed into the FLIPR Tetra along with the compound plate. 10 μL of references and compounds were then transferred from the compound plate into the cell plate and the fluorescent signal was read.

Functional Assays at 5-HT2C Receptors. Agonist activity at 5-HT2C receptors was determined using a FLIPR Ca²⁺ flux assay at Eurofins DiscoverX (Fremont, CA) according to their standard protocols. Briefly, stably transfected cells expressing the human 5-HT2C receptor were grown and plated in a 384 well plate and incubated at 37° C. and 5% CO₂ overnight. Assays were performed in 1× Dye Loading Buffer consisting of 1× Dye, 1× Additive A, and 2.5 mM Probenecid in HBSS/20 mM Hepes. Probenecid was prepared fresh. Cells were loaded with dye prior to testing and incubated at 37° C. for 30-60 minutes. After dye loading, cells were removed from the incubator and 10 μL HBSS/20 mM Hepes was added. 3× vehicle was included in the assay buffer. Cells were incubated for 30 mins at room temperature in the dark to equilibrate plate temperature. Intermediate dilution of sample stocks was performed to generate 4× sample in assay buffer. Compound agonist activity was measured on a FLIPR Tetra (MDS). Calcium mobilization was monitored for 2 minutes and 10 μL 4× sample in HBSS/20 mM Hepes was added to the cells 5 seconds into the assay.

### Example 92: Effects on the Head Twitch Response (HTR) in Mice

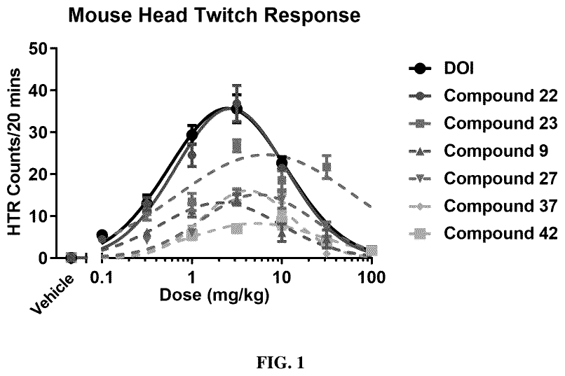

Compounds were tested for their ability to induce a head twitch response (HTR) in mice, with the results summarized in Table 4. Dose response curves for select compounds are shown in FIG. 1. Agonists of the 5-HT2A receptor are well known to induce this effect in rodents and the potency of this HTR is correlated with hallucinogenic potency in humans. All compounds tested induced a HTR. However, Eₘₐₓ values (and HTR counts/20 min at most efficacious dose) varied substantially over the 20-min observation period, suggesting that the tested compounds have a variable magnitude of hallucinogenic effect. Both Compound 22 and DOI exhibited a high Eₘₐₓ in this assay, consistent with the strong hallucinogenic effects of these two compounds reported in humans. In contrast, Compounds 9 and 23 showed an attenuated Eₘₐₓ in the HTR, consistent with the decreased hallucinogenic effects of these compounds reported in humans. Many of the disclosed compounds also showed an attenuated HTR Eₘₐₓ (e.g., <˜20 HTR/20 min), suggestive of an attenuated hallucinogenic effect.

### Methods

Animals. Adult male C57BL/6 mice, aged 8-10 weeks (body weight 20-25 g) were used in these experiments. Animals were housed under controlled temperatures and 12-hour light/dark cycles (lights on between 07:00-19:00 h), with ad libitum food and water. The protocol was approved by the Eurofins Advinus Institutional Animal Care and Use Committee. This study was carried out in strict accordance with the recommendations in the Guide for the Care and Use of Laboratory Animals of the National Institutes of Health. All efforts were made to minimize suffering.

Drugs and Drug administration. DOI, 25D-NBOMe and 2C-TFM were purchased from Cayman Chemical. All other compounds were synthesized as described above. All drugs were administered subcutaneously (SC) dissolved in saline vehicle (or saline acidified with 1-2 molar equivalents HCl to form the salt in situ for freebase compounds) and at a volume of 10 mL/kg. Drugs were administered at 5 doses per compound (in the range of 0.316 to 100 mg/kg, depending on the compound), using n=6 animals/group. Doses were calculated on the basis of the freebase, except for Compounds 22 and 23, which were calculated on the basis of their HCl salts.

Procedure. Mice were administered one dose of the drug (or vehicle) SC and immediately placed into a small open field for behavioral observation. Animals were observed continuously for 20 mins and the number of head twitches (HTs) were counted by an observer blind to the treatment condition.

Statistical Analysis. The data points shown are the mean±standard error of the mean (SEM). Analysis was performed using GraphPad Prism 9. Dose-response curves were fit via non-linear regression using the Gaussian 2020 function in Prism. Comparisons between groups were performed using the one-way analysis of variance (ANOVA), followed by the post-hoc Tukey test. P-values less than 0.05 were considered statistically significant.

### Example 93: Antagonist Blockade of the Mouse HTR

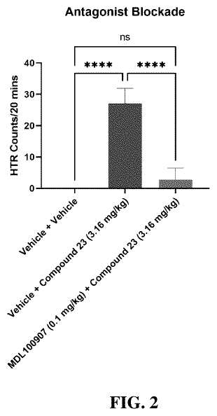

The ability of the selective 5-HT2A receptor antagonist MDL100907 to block the HTR of Compound 23 was tested, with the results shown in FIG. 2. Pre-treatment with MDL100907 significantly reduced the number of HTs counted during the 20-minute observation period F(2,25)=137.8, p<0.0001, suggesting that the HTR of Compound 23, although attenuated compared to more hallucinogenic compounds (e.g. Compound 22), remains dependent on activation of 5-HT2A receptors.

### Methods

Animals. Adult male C57BL/6 mice, aged 8-10 weeks (body weight 20-25 g) were used in these experiments. Animals were housed under controlled temperatures and 12-hour light/dark cycles (lights on between 07:00-19:00 h), with ad libitum food and water. The protocol was approved by the Eurofins Advinus Institutional Animal Care and Use Committee. This study was carried out in strict accordance with the recommendations in the Guide for the Care and Use of Laboratory Animals of the National Institutes of Health. All efforts were made to minimize suffering.

Drugs and Drug administration. Compound 23 was synthesized as described above. MDL100907 was purchased from Cayman Chemical. All drugs were administered SC at a volume of 10 mL/kg. Compound 23 was dissolved in a vehicle consisting of saline and MDL100907 was dissolved in a vehicle consisting of 0.1% DMSO in saline (DMSO added first to dissolve compound, followed by saline). Compound 23 was administered at 3.16 mg/kg and MDL100907 at 0.1 mg/kg. The dose of Compound 23 was calculated on the basis of the HCl salt and that of MDL100907 on the basis of the freebase. Group size was n=10 per treatment.

Procedure. Mice administered first administered MDL100907 or vehicle SC. Twenty minutes later, Compound 23 was administered SC and animals were immediately placed into a small open field for behavioral observation. Animals were observed continuously for 20 mins and the number of HTs were counted by an observer blind to the treatment condition.

Statistical Analysis. The bars shown are the mean±standard error of the mean (SEM). Analysis was performed using GraphPad Prism 9. Comparisons between groups were performed using the one-way analysis of variance (ANOVA), followed by the post-hoc Tukey test. P-values less than 0.05 were considered statistically significant.

### Example 94: Ex Vivo Binding in Mice

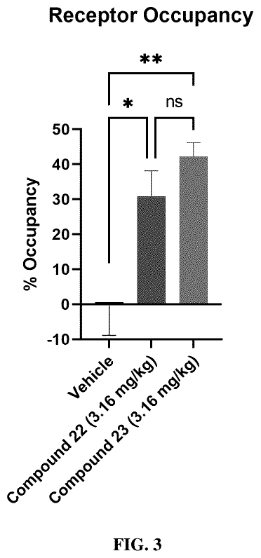

The 5-HT2A receptor occupancy of Compounds 22 and 23 in mouse brain after peripheral administration was determined using ex vivo labelling of 5-HT2A receptor binding sites with the selective radioligand [³H]MDL100907. At the dose resulting in maximal HTR for both compounds (3.16 mg/kg), 22 and 23 showed 310% and 42% receptor occupancy, respectively, 15 min after drug administration (FIG. 3). These findings suggest that the difference between these compounds observed in HTR Eₘₐₓ values cannot be explained by differences in receptor occupancy, as both drugs showed very similar levels of occupancy at 5-HT2A receptors at the same time at which different HTR response was observed. Thus, differences in receptor occupancy are likely not sufficient to explain the differences in the hallucinogenic properties of these two compounds.

### Methods

Animals. Adult male C57BL/6 mice, aged 8 weeks (body weight 20-25 g) were used in these experiments. Animals were housed under controlled temperatures and 12-hour light/dark cycles (lights on between 07:00-19:00 h), with ad libitum food and water. The protocol was approved by the RenaSci Institutional Animal Care and Use Committee. This study was carried out in strict accordance with the recommendations in the Guide for the Care and Use of Laboratory Animals of the National Institutes of Health. All efforts were made to minimize suffering.

Drugs and Drug administration. Compounds were synthesized as described above. All drugs were administered SC dissolved in saline vehicle and at a volume of 10 mL/kg. Compounds 22 and 23 were administered at 3.16 mg/kg (calculated on the basis of the HCl salt of each compound), using n=5 animals/group.

Procedure. Mice were administered drug or vehicle SC and 15 minutes later, brains were extracted, frozen on dry ice, and sectioned into 40 μm coronal sections. Sections containing the prefrontal cortex were then incubated with [³H]MDL100907. Data was collected by counting the number of β-particles emerging from the sections using a BetaIMAGER® (Biospace Lab). Values for specific binding were generated by subtracting the mean non-specific binding from the mean total binding. Specific binding as a percent of vehicle was then calculated, and subtracted from 100 to determine the receptor occupancy.

Statistical Analysis. The bars shown are the mean±standard error of the mean (SEM). Analysis was performed using GraphPad Prism 9. Comparisons between groups were performed using the one-way analysis of variance (ANOVA), followed by the post-hoc Tukey test. P-values less than 0.05 were considered statistically significant.

### Example 95: Forced Swim Test in Rats

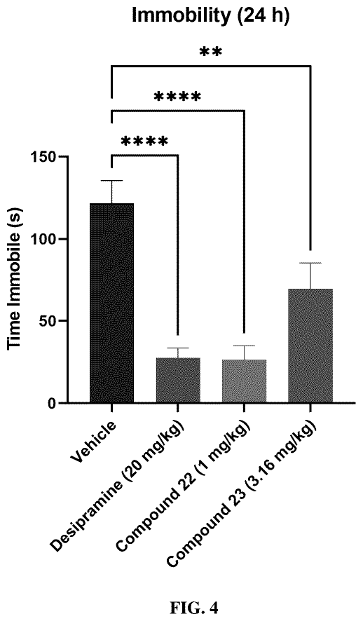

Disclosed compounds induced antidepressant-like effects in the forced swim test (FST) in rats with a 23.5-h pre-treatment time (FIG. 4). Specifically, the compounds reduced immobility time relative to vehicle control, indicative of an antidepressant-like effect. These effects on immobility were observed 23.5 hours after a single compound administration, a time point at which most or all of the drug has been cleared from the systemic circulation. An antidepressant-like effect was observed for both the hallucinogenic Compound 22 and non/less-hallucinogenic Compound 23, suggesting that the hallucinogenic effects and therapeutic effects of 5-HT2A receptor agonists may be separable.

### Methods

Animals. Male Sprague Dawley rats, aged 8-10 weeks, were used in the experiments. Animals were housed in groups of 2 under controlled temperature (22±3° C.) and relative humidity (30-70%) conditions, with 12-hour light/dark cycles, and with ad libitum food and water. These studies were carried out in strict accordance with the requirements of the Committee for the Purpose of Control and Supervision of Experiments on Animals (CPCSEA), India. All efforts were made to minimize suffering.

Drugs and Drug Administration. Compounds were synthesized as described above. Desipramine HCl was commercially obtained. Test compounds, saline vehicle, and the positive control desipramine were administered subcutaneously (SC), with doses calculated based on the freebase for desipramine and based on the HCl salts for Compounds 22 and 23. Normal saline was used as the vehicle. All compounds were administered at a volume of 5 mL/kg. Test compounds and vehicle were administered 0.5 h after the start of the training swim (Swim 1) and 23.5 h before the test swim (Swim 2). Desipramine was administered 3 times, at 23.5 h, 5 h, and 1 h before the test swim (Swim 2), each time at a dose of 20 mg/kg. Group size was n=10 per treatment.

Forced Swim Test (FST). Animals were randomized based on body weight, and it was ensured that inter-group variations were minimal and did not exceed ±20% of the mean body weight across the groups. Rats were handled for about 2 min daily for the 5 days prior to the beginning of the experimental procedure. On the first day of the experiment (i.e., Day 0), post randomization, training swim sessions (Swim 1) were conducted between 12:00 and 18:00 h with all animals by placing rats in individual glass cylinders (46 cm tall×20 cm in diameter) containing 23-25° C. water 30 cm deep for 15 minutes. At the conclusion of Swim 1, animals were dried with paper towels, placed in heated drying cages for 15 minutes, and then returned to their home cages. Animals were then administered the appropriate drug or vehicle treatment(s), as described above. For clarity, a compound administration time of 23.5 h before Swim 2 means 0.5 h after the start of Swim 1 and 0.25 h after the completion of Swim 1 (i.e., immediately after return to the home cage). On Day 1 (i.e., 24 h after start of Swim 1), animals performed the test swim (Swim 2) for a period of 5 min but otherwise under the same conditions as Swim 1. During all swim sessions, the water was changed between each animal.

Behavioral scoring was conducted by observers who were blind to the treatment groups. Animals were continuously observed during Swim 2 and the total time spent engaging in the following behaviors was recorded: immobile, swimming, and climbing. A rat was judged to be immobile when it remained floating in the water without struggling and was making only those movements necessary to keep its head above water. A rat was judged to be swimming when it made active swimming motions, more than necessary to merely maintain its head above water (e.g., moving around in the cylinder). A rat was judged to be climbing when it made active movements with its forepaws in and out of the water, usually directed against the walls.

Statistical Analysis. The data points shown represent the mean±standard error of the mean (SEM). Analysis was performed using GraphPad Prism 9. Comparisons between groups were performed using the one-way analysis of variance (ANOVA), followed by Dunnett's test for comparisons to vehicle.

### Example 96: Marble Burying in Mice

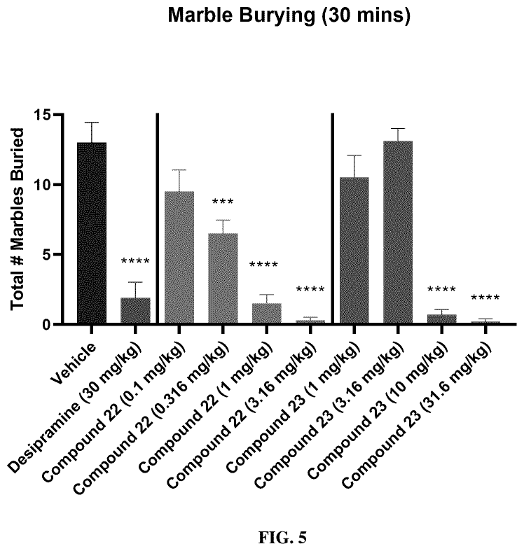

Disclosed compounds produced an anxiolytic-like effect in the marble burying test (MBT) in C57BL/6 mice (FIG. 5). Specifically, both the hallucinogenic Compound 22 and non/less-hallucinogenic Compound 23 reduced the number of marbles buried in a 30-minute period compared to vehicle, suggesting that the hallucinogenic effects and therapeutic effects of 5-HT2A receptor agonists may be separable.

### Methods

Animals. Adult male C57BL/6 mice, aged 8-10 weeks (body weight 20-25 g) were used in these experiments. Animals were housed under controlled temperatures and 12-hour light/dark cycles (lights on between 07:00-19:00 h), with ad libitum food and water. The protocol was approved by the Eurofins Advinus Institutional Animal Care and Use Committee. This study was carried out in strict accordance with the recommendations in the Guide for the Care and Use of Laboratory Animals of the National Institutes of Health. All efforts were made to minimize suffering.

Drugs and Drug Administration. Compounds were synthesized as described above. Desipramine HCl was commercially obtained. Test compounds, vehicle, and the positive control desipramine were administered subcutaneously (SC), with doses calculated based on the freebase for desipramine and based on the HCl salts for Compounds 22 and 23. Normal saline was used as the vehicle. All compounds were administered at a volume of 10 mL/kg. All compounds were administered 30 minutes prior to the start of behavioral testing. Group size was n=10 per treatment. Marble Burying Test (MBT). Animals were randomized based on body weight, and it was ensured that inter-group variations were minimal and did not exceed ±20% of the mean body weight across the groups. Mice were handled for about 2 min daily for the 3 days prior to the beginning of the experimental procedure. Twenty glass marbles (16 mm diameter) were placed with equal distances in a 5×4 pattern on a 5-cm layer of corn-cob bedding, with marbles at least 2 cm from the borders of the cage. The total number of marbles buried were counted in three 10-minute time bins (total 30 minutes). A marble was considered buried when it was >2/3 covered by bedding material.

Statistical Analysis. The data points shown are the mean±standard error of the mean (SEM). Analysis was performed using GraphPad Prism 9. Comparisons between groups were performed using the one-way analysis of variance (ANOVA), followed by Dunnett's test for comparisons to vehicle.

### Example 97: SmartCube® Testing

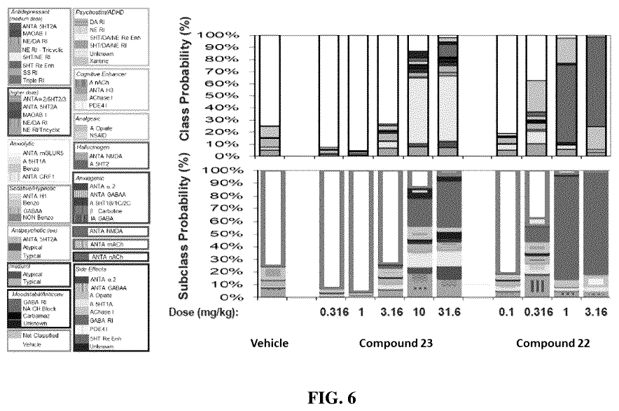

The behavioral class and subclass of disclosed compounds was determined in mice using the SmartCube® (Psychogenics, Inc.), a rodent behavioral phenotyping system, with the results shown in FIG. 6. Compound 22 displayed a profile consistent with its known hallucinogenic activity. In contrast, the non/less-hallucinogenic Compound 23 exhibited a predominantly anxiolytic-like profile and an attenuated hallucinogen-like signature, suggesting that the SmartCube® can distinguish between different types of 5-HT2A receptor agonists.

### Methods

Animals. Male C57BL/6 mice from Taconic Laboratories were used. All animals were examined, handled, and weighed prior to initiation of testing to assure adequate health and suitability and to minimize non-specific stress associated with manipulation. During the course of the study, mice were group-housed in OPTI ventilated mouse cages with 4 mice/cage. 12/12 light/dark cycles were maintained. The room temperature was maintained between 20 and 23° C. with a relative humidity maintained between 30 and 70%. Chow and water were provided ad libitum for the duration of the study. Animals were acclimated to the vivarium for at least two weeks prior to commencing testing and tested at 8-9 weeks of age. Body weight was measured prior to testing. All mouse husbandry and experimental procedures were conducted with the approval of the appropriate Animal Care and Use Committee.

Drugs and Drug Administration. Compounds were synthesized as described above. Test compounds and vehicle were administered subcutaneously (SC), with doses calculated based on their HCl salts. Normal saline was used as the vehicle. All compounds were administered at a volume of 10 mL/kg. All compounds were administered 30 minutes prior to the start of SmartCube® testing. Group size was n=12 per treatment.

SmartCube® Testing in Mice. The SmartCube® (Psychogenics, Inc.) is a proprietary mouse behavioral phenotyping system designed to measure numerous spontaneous behaviors and responses to challenges in the same testing environment (Alexandrov et al., 2015). The hardware includes force sensors and a number of aversive stimuli to elicit behavior. Mice are administered vehicle or test compound and placed in the SmartCube® following the appropriate pretreatment time. Three high-resolution video cameras provide a constant 3D view of the mouse in the SmartCube® apparatus throughout the entire 45-minute testing period.

Several analytical methods including Bayesian probabilistic density models are utilized alongside data mining algorithms to classify the behavioral phenotype of the mouse. The algorithms consider more than 2,000 measures including frequency and duration of behavioral states such as grooming, rearing, mobility, behavioral transitions, and many other features obtained during the test session. Two major types of analyses are routinely conducted, class and subclass, which classify the behavioral phenotype induced by a test drug compared to a reference database of over 300 clinically validated psychoactive compounds. The class consists of drugs that are currently on the market or which have been clinically validated for the indication specified (e.g., antidepressant class, anxiolytic class, etc.). The sub-class consists of both marketed drugs and other compounds that have been mechanistically validated and is a larger set than the class with more mechanistic specificity (e.g., SSRIs, benzodiazepines, etc.).

Data from the screening of test compounds is processed using proprietary computer vision and data mining algorithms and the results are compared to the signatures of the reference compounds in the class/sub-class database. The results for the class and subclass analyses are presented as standardized bar charts with percentages that sum to 100 for each dose. Importantly, SmartCube® has been demonstrated to be clinically predictive for SEP-363856, a novel treatment for schizophrenia that does not work via D2 receptor blockade, showing predictive power for both positive and negative clinical benchmarks (Leahy, 2019). In the present experiments, animals were treated with test compounds at 4 doses, as described above, and tested in the SmartCube®.

### Example 98: Metabolic Stability in Human Liver Microsomes

Disclosed compounds were tested for stability in human liver microsomes (HLM), with the results summarized in Table 5. Disclosed compounds exhibited variable stability in this model. Many compounds exhibited high stability in HLM, suggestive of potential oral bioavailability. Further, compounds bearing a benzyl substituent on the amine (at R⁶ in Formula (I)) were generally much less stable than those compounds containing a primary amine (where R⁶ is H), suggesting that they might be useful as short-acting drugs.

### Methods

HLM Stability. Pooled HLM from adult male and female donors (Corning 452117) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}), and intrinsic clearance (CLᵢₙₜ) were determined in a plot of ln(AUC) versus time, using linear regression analysis.

TABLE 5
Intrinsic clearance (Clᵢₙₜ) and half-life
(t_{1/2}) of compounds in the presence of HLM.

| Compound | Clint (μL/min/mg) | t_{1/2} (min) |
|---|---|---|
| DOI | <9.60 | >145 |
| 25D-NBOMe | 83.6 | 16.6 |
| 2C-TFM | <9.6 | >145 |
| LSD | 19.4 | 71.6 |
| Mescaline | <9.60 | >145 |
| DMT | 199 | 6.98 |
| 2C-B | 17.2 | 80.5 |
| 2C-E | <9.60 | >145 |
| Psilocin | 12.6 | 110 |
| 5-MeO-DMT | 102 | 13.6 |
| 2 | <9.60 | >145 |
| 3 | 13.4 | 104 |
| 4 | 16.8 | 82.6 |
| 4ent1 | 13.2 | 105 |
| 4ent2 | 14.1 | 98.2 |
| 5 | <9.60 | >145 |
| 6 | 15.9 | 87.4 |
| 7 | 67.2 | 20.6 |
| 8 | 10.1 | 138 |
| 9 | <9.60 | >145 |
| 10 | 374 | 3.7 |
| 11 | <9.60 | >145 |
| 12 | <9.60 | >145 |
| 13 | 106 | 13.1 |
| 14 | 112 | 12.4 |
| 15 | 132 | 10.5 |
| 16 | 164 | 8.43 |
| 17 | 209 | 6.64 |
| 18 | 257 | 5.4 |
| 19 | 352 | 3.93 |
| 20 | 136 | 10.2 |
| 21 | 110 | 12.6 |
| 22 | <9.60 | >145 |
| 23 | 15.8 | 87.6 |
| 24 | <9.60 | >145 |
| 25 | 13.9 | 99.9 |
| 26 | 12.2 | 114 |
| 27 | <9.60 | >145 |
| 28 | <9.60 | >145 |
| 29 | <9.60 | >145 |
| 30 | <9.60 | >145 |
| 31 | <9.60 | >145 |
| 33 | <9.60 | >145 |
| 35 | <9.60 | >145 |
| 36 | 16.2 | 85.4 |
| 37 | <9.60 | >145 |
| 38 | 13.7 | 102 |
| 39 | 47.5 | 29.2 |
| 40 | 26.9 | 51.5 |
| 41 | 54.6 | 25.4 |
| 42 | 17.2 | 80.5 |
| 43i | <9.60 | >145 |
| 44 | <9.60 | >145 |
| 45 | <9.60 | >145 |
| 46 | <9.60 | >145 |
| 47 | <9.60 | >145 |
| 47i | 20.4 | 68.0 |
| 48 | <9.60 | >145 |
| 49 | 38.4 | 36.1 |
| 50 | 178 | 7.81 |
| 51 | 223 | 6.2 |
| 52 | 450 | 3.08 |
| 53 | 41.3 | 33.6 |
| 54 | 285 | 4.87 |
| 55 | 75.6 | 18.3 |
| 56 | 83.3 | 16.6 |
| 57 | 117.0 | 11.8 |
| 58 | 184 | 7.51 |
| 59 | 50.8 | 27.3 |
| 60 | 204 | 6.8 |
| 61 | 134 | 10.3 |
| 63 | 220 | 6.3 |
| 63i | 22.9 | 60.4 |
| 64 | 20.3 | 68.2 |
| 67 | <9.60 | >145 |
| 67i | <9.60 | >145 |
| 68 | 9.93 | 140 |
| 69 | 118 | 11.8 |
| 70 | 449 | 3.08 |
| 71 | 314 | 4.42 |
| 72 | 38.2 | 36.3 |
| 73 | 9.9 | 140 |
| 74 | <9.60 | >145 |
| 75 | 20 | 69.2 |
| 76 | 13.3 | 104 |
| 77 | 37.9 | 36.6 |
| 78 | <9.60 | >145 |
| 79 | <9.60 | >145 |
| 80 | 16.1 | 86.3 |
| 81 | <9.60 | >145 |
| 82 | <9.60 | >145 |
| 83 | 13 | 106 |
| 84 | <9.60 | >145 |
| 85 | <9.60 | >145 |
| 86 | 102.8 | 13.5 |
| 87 | 45.1 | 30.7 |
| 88 | 44.3 | 31.1 |
| 89 | 32.5 | 42.7 |
| 90 | 27 | 51.4 |
| 91 | 39.6 | 35 |
| 94 | 53.4 | 26 |

### Example 99: Metabolic Stability in Mouse Liver Microsomes

Disclosed compounds were tested for stability in mouse liver microsomes (MLM), with the results summarized in Table 6. Disclosed compounds exhibited variable stability in this model. Many compounds exhibited high stability in MLM, suggestive of potential oral bioavailability. Further, compounds bearing a benzyl substituent on the amine (at R⁶ in Formula (I)) were generally much less stable than those compounds containing a primary amine (where R⁶ is H), suggesting that they might be useful as short-acting drugs.

### Methods

MLM Stability Pooled MLM from CD-1 mice (BIOIVT M00501) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus time, using linear regression analysis.

TABLE 6
Intrinsic clearance (Clᵢₙₜ) and half-life
(t_{1/2}) of compounds in the presence of MLM.

| Compound Number | Clᵢₙₜ (μL/min/mg) | t_{1/2} (min) |
|---|---|---|
| DOI | 13.3 | 104 |
| 25D-NBOMe | 589 | 2.35 |
| 2C-TFM | <9.60 | >145 |
| 2 | 96.9 | 14.3 |
| 3 | 35.5 | 39.0 |
| 4 | 20.1 | 69.0 |
| 4ent1 | 33.2 | 41.7 |
| 4ent2 | 12.4 | 112.1 |
| 5 | 19.2 | 72.3 |
| 6 | 15.5 | 89.4 |
| 7 | 27.2 | 50.9 |
| 8 | 14.6 | 95 |
| 9 | 13.9 | 99.9 |
| 10 | 733 | 1.89 |
| 11 | 17 | 81.5 |
| 12 | 14.8 | 93.5 |
| 13 | 163 | 8.5 |
| 14 | 271 | 5.12 |
| 15 | 414 | 3.35 |
| 16 | 1350 | 1.03 |
| 17 | 543 | 2.55 |
| 18 | 909 | 1.52 |
| 19 | 1270 | 1.09 |
| 20 | 407 | 3.41 |
| 21 | 786 | 1.76 |
| 22 | 14.8 | 93.4 |
| 23 | 22.5 | 61.7 |
| 24 | <9.60 | >145 |
| 25 | 13.0 | 107.0 |
| 26 | 20.5 | 67.6 |
| 27 | 20.4 | 68 |
| 28 | 42.6 | 32.5 |
| 29 | 21.7 | 64 |
| 30 | 17.8 | 77.8 |
| 31 | <9.60 | >145 |
| 33 | <9.60 | >145 |
| 35 | <9.60 | >145 |
| 36 | 12.2 | 114 |
| 37 | 125 | 11.1 |
| 38 | 17.5 | 79.1 |
| 39 | 22.1 | 62.6 |
| 40 | 26.8 | 51.8 |
| 41 | 48.2 | 28.8 |
| 42 | 22.6 | 61.4 |
| 43i | 31.4 | 44.1 |
| 44 | 12.8 | 108 |
| 45 | 23.2 | 59.7 |
| 46 | 14.6 | 94.6 |
| 47 | 9.8 | 141 |
| 47i | 19.9 | 69.7 |
| 48 | 26.7 | 52 |
| 49 | 196 | 7.08 |
| 50 | 207 | 6.68 |
| 51 | 672 | 2.1 |
| 52 | 643 | 2.15 |
| 53 | 165 | 8.41 |
| 54 | 838 | 1.65 |
| 55 | 173 | 8.03 |
| 56 | 356 | 3.89 |
| 57 | 763 | 1.82 |
| 58 | 1700 | 0.817 |
| 59 | 166 | 8.34 |
| 60 | 958 | 1.4 |
| 61 | 1170 | 1.2 |
| 63 | 815 | 1.7 |
| 63i | 42.5 | 32.6 |
| 64 | 666 | 2.08 |
| 67i | 34.3 | 40.4 |
| 67 | 17.7 | 78.3 |
| 68 | 17.3 | 79.9 |
| 69 | 60.4 | 23 |
| 70 | 113 | 12.2 |
| 71 | 88.8 | 15.6 |
| 72 | 37.8 | 36.7 |
| 73 | 40.5 | 34.2 |
| 74 | 11.7 | 118 |
| 75 | 82.1 | 16.9 |
| 76 | 29.9 | 46.4 |
| 77 | 57.4 | 24.1 |
| 78 | 11.6 | 120 |
| 79 | 13.3 | 104 |
| 80 | 19.4 | 71.5 |
| 81 | 12.8 | 108 |
| 82 | <9.60 | >145 |
| 83 | 27.7 | 50.1 |
| 84 | 20.8 | 66.8 |
| 85 | 41.2 | 33.6 |
| 86 | 24.4 | 56.9 |
| 87 | 15.9 | 87.1 |
| 88 | 10.4 | 133.5 |
| 89 | 32.4 | 42.8 |
| 90 | 17.4 | 79.7 |
| 91 | 20.7 | 66.9 |
| 94 | 67.8 | 20.4 |

### Example 100: Metabolic Stability in Rat Liver Microsomes

Disclosed compounds were tested for stability in rat liver microsomes (RLM), with the results summarized in Table 7. Disclosed compounds exhibited variable stability in this model. Some compounds exhibited high stability in RLM, suggestive of potential oral bioavailability. Further, compounds bearing a benzyl substituent on the amine (at R⁶ in Formula (I)) were generally much less stable than those compounds containing a primary amine (where R⁶ is H), suggesting that they might be useful as short-acting drugs.

### Methods

RLM Stability Pooled RLM from adult male and female donors (Xenotech R1000) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus time, using linear regression analysis.

TABLE 7
Intrinsic clearance (Clᵢₙₜ) and half-life
(t_{1/2}) of compounds in the presence of RLM.

| Compound | Clᵢₙₜ (μL/min/mg) | t_{1/2} (min) |
|---|---|---|
| DOI | 123 | 11.3 |
| 25D-NBOMe | 639 | 2.17 |
| 2C-TFM | 19.1 | 72.7 |
| 2 | <9.60 | >145 |
| 3 | 96.8 | 14.3 |
| 4 | 29.7 | 46.7 |
| 4ent1 | 19.1 | 72.4 |
| 4ent2 | 21.6 | 64.3 |
| 5 | 19.4 | 71.3 |
| 6 | 39.8 | 34.9 |
| 7 | 219 | 6.34 |
| 8 | 29.1 | 47.6 |
| 9 | 19.3 | 71.7 |
| 10 | 920 | 1.51 |
| 11 | 61.2 | 22.6 |
| 12 | 35.4 | 39.2 |
| 13 | 1180 | 1.18 |
| 14 | 548 | 2.53 |
| 15 | 1640 | 0.845 |
| 16 | 1970 | 0.705 |
| 17 | 622 | 2.23 |
| 18 | 1450 | 0.957 |
| 19 | 753 | 1.84 |
| 20 | 734 | 1.89 |
| 21 | 747 | 1.86 |
| 22 | 245 | 5.65 |
| 23 | 76.4 | 18.1 |
| 24 | 15.6 | 88.9 |
| 25 | 132 | 10.5 |
| 26 | 103 | 13.4 |
| 27 | 65 | 21.3 |
| 28 | 36.1 | 38.4 |
| 29 | 72.9 | 19.0 |
| 30 | 34.4 | 40.3 |
| 31 | 15.3 | 90.5 |
| 33 | 20.1 | 69.1 |
| 35 | 15.3 | 90.6 |
| 36 | 16.5 | 84.0 |
| 37 | 75.4 | 18.4 |
| 38 | 25.3 | 54.8 |
| 39 | 20.5 | 67.4 |
| 40 | 36.0 | 38.5 |
| 41 | 125 | 11.1 |
| 42 | 165 | 8.4 |
| 43i | 27.2 | 51.0 |
| 44 | <9.60 | >145 |
| 45 | 11.4 | 121 |
| 46 | 10.6 | 131 |
| 47 | <9.60 | >145 |
| 47i | 20.9 | 66.2 |
| 48 | 58.6 | 23.7 |
| 49 | 347 | 3.99 |
| 50 | 196 | 7.07 |
| 51 | 654 | 2.1 |
| 53 | 148 | 9.39 |
| 54 | 908 | 1.53 |
| 55 | 212 | 6.55 |
| 56 | 491 | 2.82 |
| 57 | 437 | 3.17 |
| 58 | 684 | 2.03 |
| 59 | 431 | 3.22 |
| 60 | 967 | 1.4 |
| 61 | 756 | 1.83 |
| 63 | 936 | 1.5 |
| 63i | 909 | 1.5 |
| 64 | 364 | 3.81 |
| 67 | 27.9 | 49.7 |
| 67i | 61.6 | 22.5 |
| 68 | 41.2 | 33.7 |
| 69 | 142 | 9.75 |
| 70 | 319 | 4.35 |
| 71 | 280 | 4.96 |
| 72 | 295 | 4.69 |
| 73 | 32.2 | 43.1 |
| 74 | 18 | 77 |
| 75 | 78.1 | 17.8 |
| 76 | 55.6 | 24.9 |
| 77 | 174 | 8 |
| 78 | 36.4 | 38.1 |
| 79 | 48.3 | 28.7 |
| 80 | 13.4 | 104 |
| 81 | <9.60 | >145 |
| 82 | <9.60 | >145 |
| 83 | 39.1 | 35.4 |
| 84 | 31.2 | 44.4 |
| 85 | 42.2 | 32.9 |
| 86 | 28.6 | 48.5 |
| 87 | 21.2 | 65.4 |
| 88 | 22.4 | 61.8 |
| 89 | 187 | 7.4 |
| 90 | 73.7 | 18.8 |
| 91 | 132.4 | 10.5 |
| 94 | 87.2 | 15.9 |

### Example 101: Pharmacokinetics in Mice

The pharmacokinetics (PK) of disclosed compounds were studied in the plasma (Table 8) and brains (Table 9) of mice after subcutaneous (SC) administration. Compounds 22 and 23 exhibited similar pharmacokinetics in terms of both time course (Tₘₐₓ and t_{1/2}) and exposure (Cₘₐₓ and AUC), suggesting that PK differences are not sufficient to explain the behavioral differences between these compounds.

### Methods

Animals. Male C57BL/6 mice, aged 8-12 weeks, were used in these studies. Four mice were housed in each cage. Temperature and humidity were maintained at 22±3° C. and 30-70%, respectively, and illumination was controlled to give a 12 h light and 12 h dark cycle. Temperature and humidity were recorded by an auto-controlled data logger system. All animals were provided laboratory rodent diet. Reverse osmosis water treated with ultraviolet light was provided ad libitum. Animals were randomly assigned to treatment groups.

Drugs and Drug Administration. Compounds were synthesized as described above. Test compounds and vehicle were administered SC at a dose of 3.16 mg/kg, calculated based on their HCl salts. Normal saline was used as the vehicle. All compounds were administered at a volume of 10 mL/kg.

Sample Collection and Bioanalysis. Blood samples (approximately 60 μL) were collected under light isoflurane anesthesia (Surgivet®) from the retro orbital plexus at 0.08, 0.25, 0.5, 1, 2, 4, 8, and 24 h (4 animals per time point). Immediately after blood collection, plasma was harvested by centrifugation at 4000 rpm for 10 min at 4° C. and samples were stored at −70±10° C. until bioanalysis. Following blood collection, animals were immediately sacrificed, the abdominal vena-cava was cut open, and the whole body was perfused from the heart using 10 mL of normal saline, and brain samples were collected from all animals. After isolation, brain samples were rinsed three times in ice-cold normal saline (for 5-10 seconds/rinse using ˜5-10 mL normal saline in disposable petri dish for each rinse) and dried on blotting paper. Brain samples were homogenized using ice-cold phosphate-buffered saline (pH 7.4). Total homogenate volume was three times the tissue weight. All homogenates were stored at −70±10° C. until bioanalysis. For bioanalysis, 25 μL aliquots of plasma/brain study samples or spiked plasma/brain calibration standards were added to individual pre-labeled micro-centrifuge tubes followed by 100 μL of an internal standard solution (glipizide, 500 ng/mL in acetonitrile) except for blanks, where 100 μL of acetonitrile was added. Samples were vortexed for 5 minutes and then centrifuged for 10 minutes at 4000 rpm at 4° C. Following centrifugation, 100 μL of each clear supernatant was transferred to a 96 well plate and analyzed with a fit-for-purpose LC-MS/MS method, with authentic samples of each analyte used for calibration and identification.

Data Analysis. Pharmacokinetic parameters were estimated using the non-compartmental analysis tool of Phoenix® WinNonlin software (Ver 8.0).

TABLE 8
Selected pharmacokinetic parameters of compounds in plasma of C57BL/6 mice.

| | | Tₘₐₓ | Cₘₐₓ | AUC_{0-inf} | t_{1/2} |
|---|---|---|---|---|---|
| Compound | Structure | (h) | (ng/mL) | (h*ng/mL) | (h) |
| 22 | 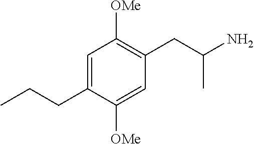 [SMILES: CCCc1cc(OC)c(CC(C)N)cc1OC] | 0.5 | 253 | 780 | 1.47 |
| 23 | 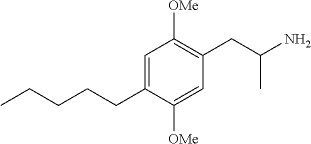 [SMILES: CCCCCc1cc(OC)c(CC(C)N)cc1OC] | 0.5 | 173 | 398 | 1.93 |

TABLE 9
Selected pharmacokinetic parameters of compounds in brains of C57BL/6 mice.

| | | Tₘₐₓ | Cₘₐₓ | AUC_{0-inf} | t_{1/2} |
|---|---|---|---|---|---|
| Compound | Structure | (h) | (ng/g) | (h*ng/g) | (h) |
| 22 | 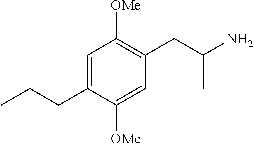 [SMILES: CCCc1cc(OC)c(CC(C)N)cc1OC] | 1.0 | 1,750 | 6,440 | 1.6 |
| 23 | 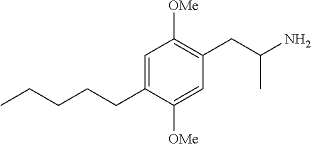 [SMILES: CCCCCc1cc(OC)c(CC(C)N)cc1OC] | 1.0 | 1,510 | 4,610 | 2.9 |

### Example 102: CYP Inhibition in Human Liver Microsomes

Inhibition of five major cytochrome P450 (CYP) enzymes (1A2, 2C9, 2C19, 2D6, and 3A4) by the disclosed compounds was determined in human liver microsomes (HLM) by using LC-MS/MS to monitor the metabolic conversion of a cocktail of reference CYP substrates in the presence and absence of the test compounds (Tables 10 and 11). Many of the disclosed compounds showed substantial CYP inhibition, especially those compounds bearing long-chain lipophilic substituents at position 4 of the arene (R¹ in Formula (I)). Compounds where the 4-position substituent was substituted with one or more fluorine atoms tended to show decreased CYP inhibition compared to their non-fluorinated counterparts, suggestive of a lower probability of drug-drug interactions (Table 12).

HLM Incubations. Pooled HLM from adult male and female donors (Corning 452117) were used. Microsomal incubations were carried out in multi-well plates. Liver microsomal incubation aliquots contained 1) PBS (100 mM, pH 7.4), MgCl₂ (3.3 mM), and NADPH (1 mM); 2) liver microsomal protein (0.2 mg/mL); 3) the reference CYP substrates: phenacetin for CYP1A2 (10 μM), diclofenac for CYP2C9 (5 μM), (S)-mephenytoin for CYP2C19 (30 μM), dextromethorphan for CYP2D6 (5 μM), and midazolam for CYP3A4 (2 μM); and 4) test compounds (10 μM), control inhibitors (3 μM α-naphthoflavone for CYP1A2, 3 μM sulfaphenazole for CYP2C9, 1 μM (+)-N-3-benzylnirvanol for 2C19, 3 μM quinidine for CYP2D6, or 3 μM ketoconazole for CYP3A4), or solvent (for uninhibited condition). Incubations were carried out at 37° C. with constant shaking for 10 minutes. The reaction aliquots were stopped by adding 400 μL of cold (4° C.) acetonitrile containing 200 ng/mL tolbutamide and 200 ng/mL labetalol as internal standards (IS), followed by protein sedimentation by centrifugation at 4000 rpm for 20 minutes at 4° C.

Sample Analysis. Supernatant samples (200 μL) were diluted with water (100 μL) and the reference metabolites of each reference CYP substrate were quantified using a fit-for-purpose liquid chromatography-tandem mass spectrometry (LC-MS/MS) method. Percent inhibition by test compounds or control inhibitors was calculated at each test concentration by comparing the metabolite formation in the presence of the inhibitor compared to the metabolite formation in the absence of the inhibitor. For compounds where multiple concentrations were evaluated, IC₅₀S were calculated.

TABLE 10
Percent inhibition of indicated CYPs by test compounds at 10 μM.

CYP (% inhibition at 10 μM)

| Compound | 3A4 | 2D6 | 1A2 | 2C19 | 2C9 |
|---|---|---|---|---|---|
| DOI | 0 | 64 | 24 | 0 | 0 |
| 25D-NBOMe | 55.7 | 95.7 | 10.2 | 77.2 | 8.6 |
| 2C-TFM | 0 | 17.4 | 26.1 | 0.254 | 0 |
| LSD | 2.3 | 78.2 | 0 | 0 | 0 |
| Mescaline | 2.4 | 0 | 17.3 | 0 | 0 |
| DMT | 5.5 | 5.8 | 7.3 | 0 | 2.7 |
| 2C-B | 8 | 19.7 | 14.7 | 0 | 18.5 |
| 2C-E | 0 | 15 | 24.4 | 0 | 26.1 |
| Psilocin | 0 | 13.7 | 22.5 | 0 | 0 |
| 5-MeO-DMT | 0 | 4.4 | 4 | 0 | 1 |
| 2 | 0 | 21.5 | 8.8 | 2.26 | 0 |
| 3 | 43.1 | 66.1 | 78.9 | 87.5 | 2.02 |
| 4 | 35.4 | 71.4 | 68.8 | 79.2 | 0 |
| 5 | 19 | 43.5 | 47.8 | 64.6 | 0 |
| 6 | 45.9 | 32.1 | 93.1 | 89.8 | 30.8 |
| 7 | 54.7 | 85.4 | 66.7 | 37.1 | 0 |
| 8 | 25.9 | 20.3 | 93.2 | 86.6 | 26 |
| 9 | 61.6 | 0 | 87.2 | 76.5 | 14.7 |
| 10 | 84.1 | 92.4 | 42.7 | 49.6 | 4.37 |
| 11 | 18 | 39.2 | 75.9 | 81.5 | 0 |
| 12 | 30.6 | 15.5 | 5.6 | 21.8 | 0.763 |
| 13 | 67.5 | 91.8 | 42.8 | 52.2 | 9.5 |
| 14 | 71.2 | 93.5 | 12.3 | 48.8 | 14.8 |
| 15 | 19.1 | 94.1 | 26.3 | 96.2 | 0 |
| 16 | 53.4 | 95.3 | 36.7 | NA | 13.2 |
| 17 | 67.3 | 94.1 | 21.7 | 86 | 1.85 |
| 18 | 30.4 | 95 | 35.8 | 95.5 | 4.06 |
| 19 | 79.3 | 93.2 | 27.3 | 77.4 | 1.35 |
| 20 | 62.2 | 91.5 | 11.0 | 55.8 | 7.21 |
| 21 | 39.8 | 95.1 | 22 | 81.9 | 30 |
| 22 | 13.7 | 45.5 | 46.7 | 12.3 | 0 |
| 23 | 26.2 | 64.2 | 76.3 | 41.5 | 0 |
| 24 | 5.75 | 7.26 | 0 | 6.37 | 0 |
| 25 | 5.7 | 26.4 | 1.23 | 5.68 | 0 |
| 26 | 20.7 | 33.3 | 0 | 49.3 | 0 |
| 64 | 50 | 0.51 | 50 | 4.06 | 22.9 |

TABLE 11
IC₅₀s for inhibition of indicated CYPs by test compounds.

| | CYP (IC₅₀, μM) | | | | |
|---|---|---|---|---|---|
| Compound | 3A4 | 2D6 | 1A2 | 2C19 | 2C9 |
| 4ent1 | 31.9 | 7.22 | 23.3 | 44.1 | >50.0 |
| 4ent2 | 25.6 | 8.47 | 14.7 | 26.1 | >35.8 |
| 27 | >50.0 | 11.3 | >50.0 | >50.0 | >50.0 |
| 28 | 13.1 | 6.38 | 6.53 | 9.48 | >50.0 |
| 29 | 43.6 | 11 | 12.3 | 19.1 | >50.0 |
| 30 | 31.2 | 13.6 | 21 | 43.4 | >50.0 |
| 31 | >50.0 | 7.78 | 27.4 | >50.0 | >50.0 |
| 33 | >50.0 | >50.0 | 31.1 | >50.0 | >50.0 |
| 35 | >50.0 | >50.0 | 21 | >50.0 | >50.0 |
| 36 | 17.7 | 19.5 | 0.483 | 0.555 | 11.8 |
| 37 | >50.0 | 22.7 | 0.88 | 8.96 | 18.6 |
| 38 | 11 | 20.2 | 0.302 | 0.916 | 26.9 |
| 39 | 8.03 | 18.3 | 0.735 | 0.254 | 9.78 |
| 40 | 14.2 | 1.57 | 14.2 | 30.9 | >50.0 |
| 41 | 7.38 | 1.06 | 6.4 | 27.8 | >50.0 |
| 42 | 15.2 | 4.78 | 25.8 | >50.0 | >50.0 |
| 43i | 39.9 | 11.3 | 18.1 | >50.0 | >50.0 |
| 44 | 9.88 | >50.0 | 0.729 | 0.318 | 22.3 |
| 45 | 22.4 | >50.0 | 2.94 | 0.386 | 21 |
| 46 | 22.4 | >50.0 | 0.58 | 0.66 | 46 |
| 47 | 30.4 | >50.0 | 3.25 | 7.92 | >50.0 |
| 47i | >50.0 | >50.0 | 24.4 | >50.0 | >50.0 |
| 48 | 18.4 | 11.3 | 45.4 | 48.7 | >50.0 |
| 49 | 6.05 | 0.193 | 6.9 | 40.3 | 9.01 |
| 50 | 9.95 | 0.951 | 17.4 | 15.0 | 44.9 |
| 51 | 1.51 | 0.95 | 13.7 | 10.8 | 38.7 |
| 52 | 4.57 | 0.261 | >50.0 | >50.0 | >50.0 |
| 53 | 7.13 | 0.986 | 27.4 | 17.6 | 27.3 |
| 54 | 1.05 | 0.308 | 38.8 | 14.4 | >50.0 |
| 55 | 22.2 | 0.61 | 30.7 | 18 | 41.8 |
| 56 | 1.9 | 0.267 | >50.0 | 18.1 | 35 |
| 57 | 21.1 | 0.161 | 16.7 | 0.586 | >50.0 |
| 58 | 5.65 | 0.102 | 14.0 | 0.303 | 32.3 |
| 59 | 33.0 | 1.93 | >50.0 | 7.66 | >50.0 |
| 60 | 1.53 | 0.372 | 8.4 | 4.4 | 15.8 |
| 61 | 3.42 | 0.0718 | >50.0 | >50.0 | 4.02 |
| 63 | 2.02 | 0.0634 | 11.4 | 11.3 | 42.5 |
| 63i | 46 | 4.15 | >50.0 | >50.0 | >50.0 |
| 64 | >50.0 | 0.51 | >50.0 | 4.06 | 22.9 |
| 65 | 19.6 | 15.2 | 1.3 | 17.2 | 9.88 |
| 67i | 6.53 | 13.3 | 0.160 | 2.33 | 14.5 |
| 67 | 7.63 | 49.2 | 0.233 | 3.87 | 27.5 |
| 68 | 41.8 | 7.20 | 7.54 | 6.78 | >47.6 |
| 69 | >50.0 | 0.891 | 5.8 | >50.0 | 44.4 |
| 70 | >50.0 | 13.1 | 0.347 | 9.71 | 11.6 |
| 71 | >50.0 | 0.719 | 3.52 | >50.0 | 40.8 |
| 72 | 48.8 | 2.42 | 5.48 | 8.17 | >50.0 |
| 73 | 46.3 | 7.33 | 49.8 | >50.0 | >50.0 |
| 74 | 35.5 | 12.3 | 23.7 | 28.4 | >50.0 |
| 75 | 5.71 | 3.09 | 14.2 | 8.41 | >50.0 |
| 76 | 2.6 | 4.49 | 14.4 | 4.14 | >50.0 |
| 77 | 6.57 | 3.54 | >50.0 | >50.0 | >50.0 |
| 78 | 21.1 | 2.52 | 26.8 | >50.0 | >50.0 |
| 79 | 7.97 | 1.92 | 7.24 | 18.7 | >50.0 |
| 80 | 35.5 | >50.0 | 4.34 | 2.75 | >50.0 |
| 81 | 29.9 | >50.0 | 1.52 | 1.45 | >50.0 |
| 82 | 21.4 | 31.1 | 31 | >50.0 | >50.0 |
| 83 | >50.0 | >50.0 | 50 | 23.1 | 13.3 |
| 84 | 9.8 | 19.6 | >50.0 | 25.8 | >50.0 |
| 85 | 32 | 11.8 | 26.3 | 20.9 | >50.0 |
| 86 | 12.8 | 46.7 | 1.06 | 0.669 | 14.6 |
| 87 | 9.99 | 18.8 | 0.726 | 0.452 | 5.27 |
| 88 | 29.5 | 43.5 | 0.809 | 0.703 | 19.7 |
| 89 | 3.78 | 8.19 | 14.4 | 34 | >50.0 |
| 90 | 35.5 | 36.6 | >50.0 | >50.0 | >50.0 |
| 91 | >50.0 | >50.0 | 25.7 | >50.0 | >50.0 |
| 94 | 4.83 | 10.8 | 12.9 | 14.7 | >50.0 |

TABLE 12
Inhibition of CYPs by fluorinated compounds compared to non-fluorinated analogs.

| | | CYP % inhibition at 10 uM or (IC₅₀, μM) | | | | |
|---|---|---|---|---|---|---|
| Compound | Structure | 3A4 | 2D6 | 1A2 | 2C19 | 2C9 |
| 23 | 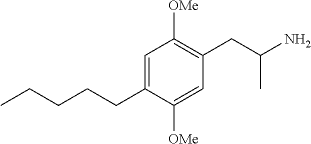 [SMILES: CCCCCc1cc(OC)c(CC(C)N)cc1OC] | 26.2 | 64.2 | 76.3 | 41.5 | 0 |
| 4 | 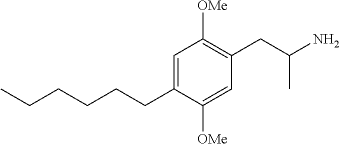 [SMILES: CCCCCCc1cc(OC)c(CC(C)N)cc1OC] | 35.4 | 71.4 | 68.8 | 79.2 | 0 |
| 6 | 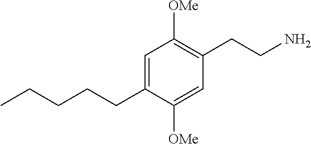 [SMILES: CCCCCc1cc(OC)c(CCN)cc1OC] | 45.9 | 32.1 | 93.1 | 89.8 | 30.8 |
| 7 | 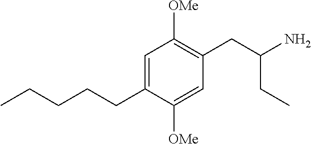 [SMILES: CCCCCc1cc(OC)c(CC(N)CC)cc1OC] | 54.7 | 85.4 | 66.7 | 37.1 | 0 |
| 9 | 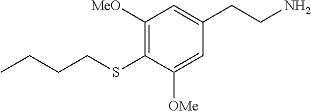 [SMILES: CCCCSc1c(OC)cc(CCN)cc1OC] | 61.6 | 0 | 87.2 | 76.5 | 14.7 |
| 2 | 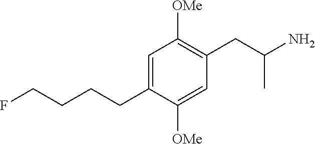 [SMILES: COc1cc(CC(C)N)c(OC)cc1CCCCF] | 0.0 | 21.5 | 8.8 | 2.26 | 0 |
| 27 | 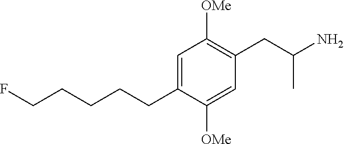 [SMILES: COc1cc(CC(C)N)c(OC)cc1CCCCCF] | (>50.0) | (11.3) | (>50.0) | (>50.0) | (>50.0) |
| 31 | 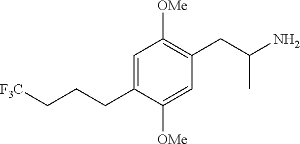 [SMILES: COc1cc(CC(C)N)c(OC)cc1CCCC(F)(F)F] | (>50.0) | (7.78) | (27.4) | (>50.0) | (>50.0) |
| 37 | 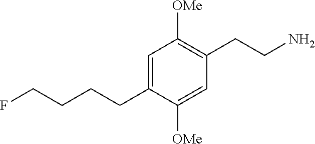 [SMILES: COc1cc(CCCCF)c(OC)cc1CCN] | (>50.0) | (22.7) | (0.88) | (8.96) | (18.6) |
| 42 | 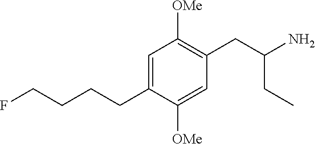 [SMILES: CCC(N)Cc1cc(OC)c(CCCCF)cc1OC] | (15.2) | (4.78) | (25.8) | (>50.0) | (>50.0) |
| 47 | 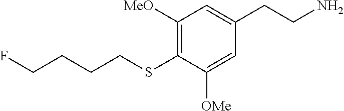 [SMILES: COc1cc(CCN)cc(OC)c1SCCCCF] | (30.4) | (>50.0) | (3.25) | (7.92) | (>50.0) |

### Example 103: Bi-Directional Permeability and Efflux in MDCK-MDR1 Cells

Disclosed compounds were tested for their ability to bi-directionally permeate an MDCK cell monolayer expressing P-glycoprotein (P-gp, MDR1) and for their extent of efflux by P-gp (efflux ratio in the presence and absence of a P-gp inhibitor), with the results presented in Table 13. The tested compounds exhibited substantial and unpredictable variability in their permeability (Papp) and efflux ratios, suggesting variability in gastrointestinal absorption and blood-brain barrier penetration depending on the specific structure.

Cell Culture. NIH-MDR1 cells were seeded onto polyethylene membranes (PET) in 96-well Corning insert systems at 2.5×10⁵ cells/mL for 4-7 days until confluent cell monolayer formation.

Bi-Directional Permeability Assay. The transport buffer in the study was HBSS with 10.0 mM HEPES at pH 7.40±0.05. Test compounds were tested at 2.00 μM in the presence and absence of 10.0 μM GF120918 (P-gp inhibitor) bi-directionally in duplicate. Final DMSO concentration was adjusted to less than 1%. The plate was incubated for 1.5 hours in a CO₂ incubator at 37±1° C., with 5% CO₂ at saturated humidity without shaking. Test and reference compounds were quantified in each compartment (apical and basolateral) by LC-MS/MS analysis based on the peak area ratio of analyte/IS.

After the transport assay, a Lucifer yellow rejection assay was applied to determine the cell monolayer integrity. Buffers were removed from both the apical and basolateral chambers, followed by the addition of 75 μL of 100 μM lucifer yellow in transport buffer and 250 μL transport buffer in apical and basolateral chambers, respectively. The plate was incubated for 30 minutes at 37° C. with 5% CO₂ and 95% relative humidity without shaking. After 30 minutes incubation, 20 μL of lucifer yellow samples were taken from the apical sides, followed by the addition of 60 μL of Transport Buffer. And then 80 μL of lucifer yellow samples were taken from the basolateral sides. The relative fluorescence unit (RFU) of lucifer yellow is measured at 425/528 nm (excitation/emission) with a microplate reader.

Data Analysis. The apparent permeability coefficient Papp (cm/s) was calculated using the equation: Papp=(dCr/dt)×Vr/(A×C0); where dCr/dt is the cumulative concentration of compound in the receiver chamber as a function of time (μM/s); Vr is the solution volume in the receiver chamber (0.075 mL on the apical side, 0.25 mL on the basolateral side); A is the surface area for the transport, i.e. 0.0804 cm² for the area of the monolayer; C0 is the initial concentration in the donor chamber (μM). The efflux ratio was calculated using the equation: Efflux Ratio=Papp (BA)/Papp (AB). Percent recovery was calculated using the equation: % Solution Recovery=100×[(Vr×Cr)+(Vd×Cd)]/(Vd×C0); where Vd is the volume in the donor chambers (0.075 mL on the apical side, 0.25 mL on the basolateral side); Cd and Cr are the final concentrations of transport compound in donor and receiver chambers, respectively. Percent of lucifer yellow in basolateral well is calculated using the equation: % Lucifer Yellow=((Vbasolateral×RFU basolateral)/(Vapical×RFUapical+Vbasolateral×RFU basolateral))×100; where RFUapical and RFUbasolateral are the relative fluorescence unit values of lucifer yellow in the apical and basolateral wells, respectively; Vapical and Vbasolateral are the volume of apical and basolateral wells (0.075 mL and 0.25 mL), respectively. The % Lucifer Yellow should be less than 1.0.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth, used in the specification and claims, are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A compound having the structure: 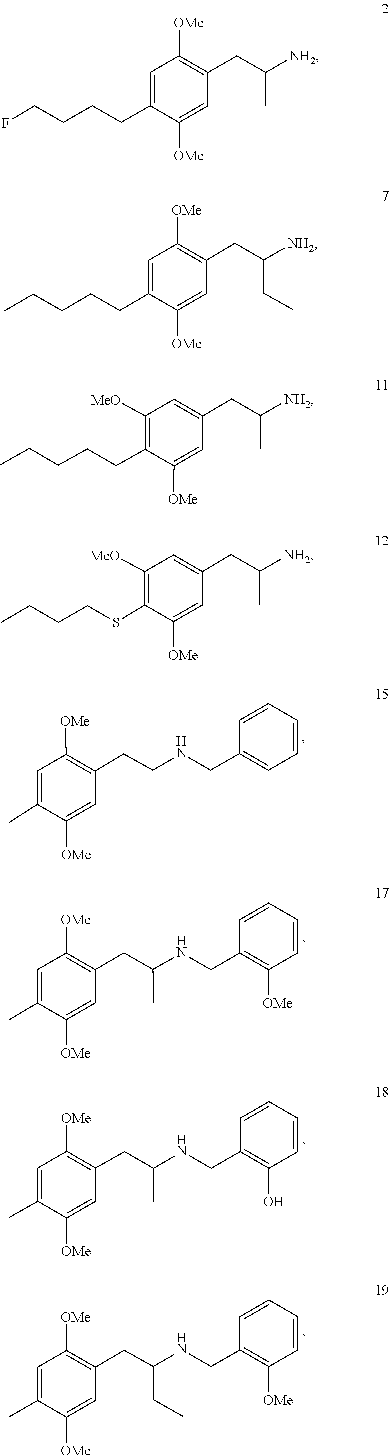 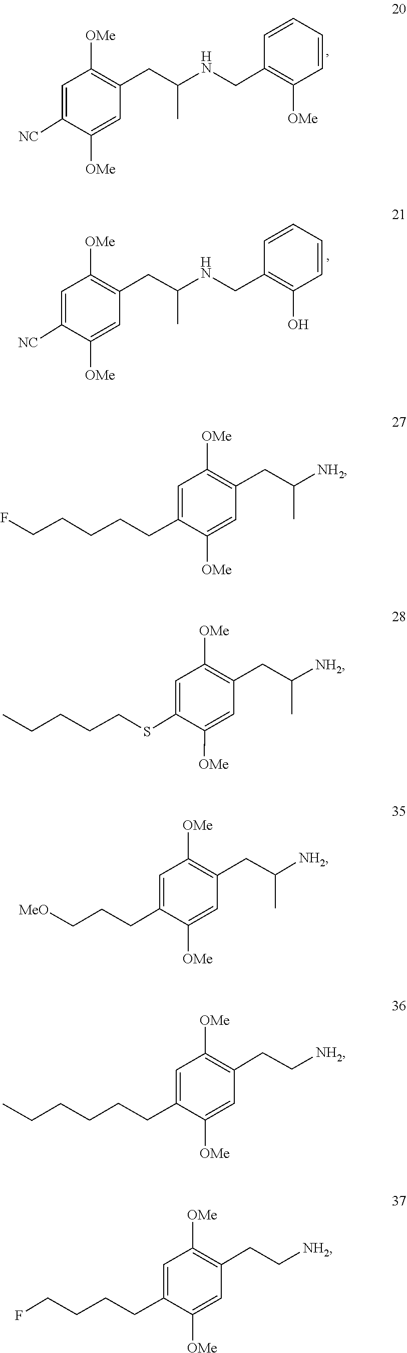 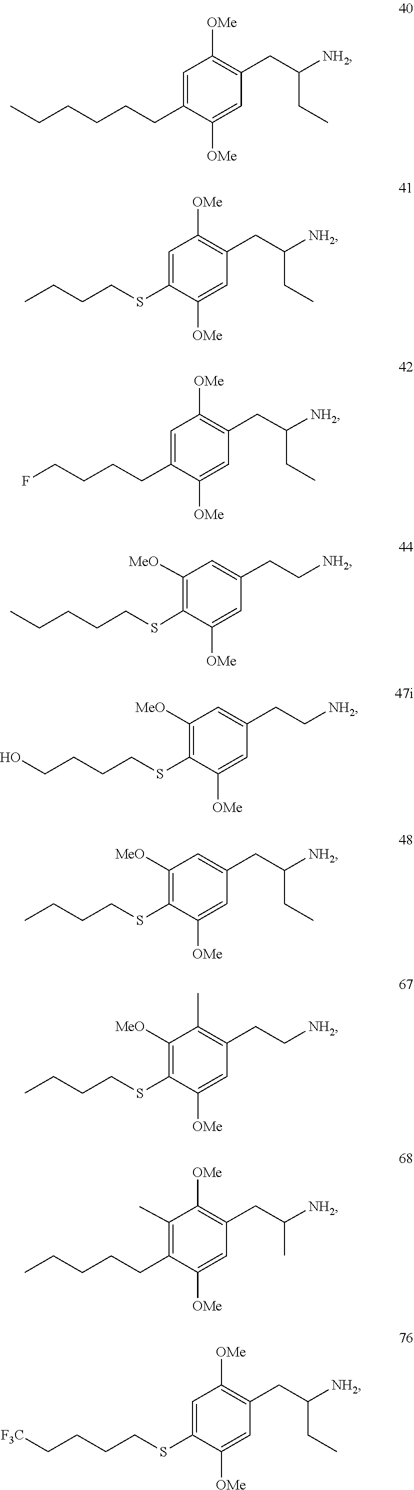 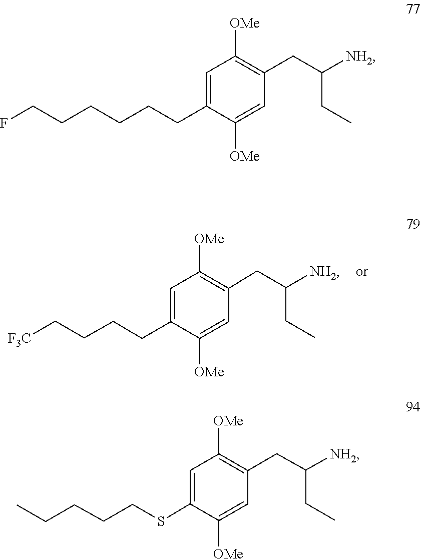
or a pharmaceutically acceptable salt thereof.

2. The compound of claim 1, having the structure: 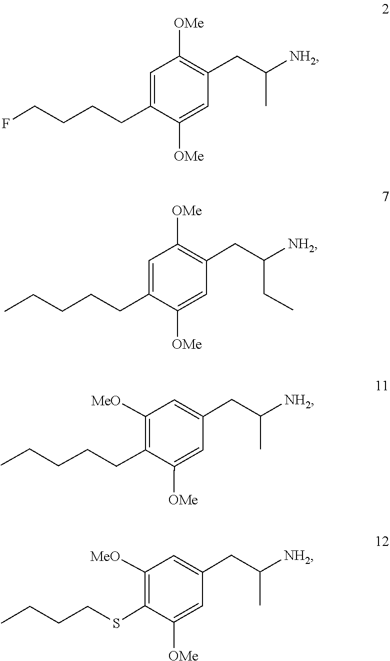 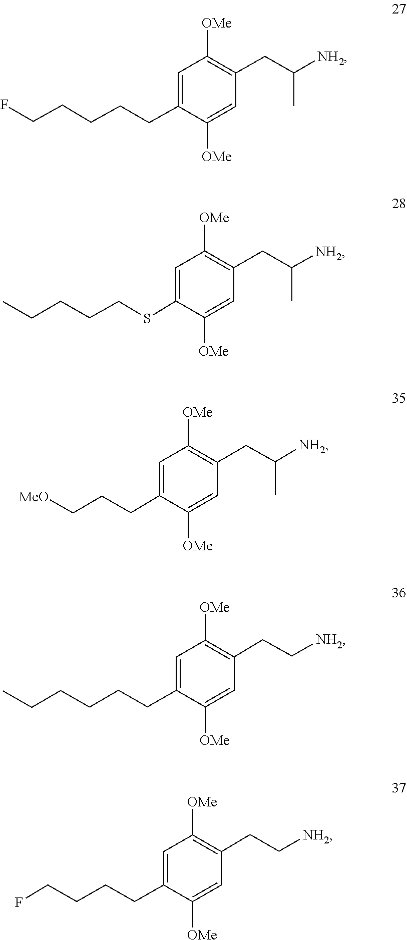 [SMILES: COc1cc(CC(C)N)c(OC)cc1CCCCCF ; CCCCCSc1cc(OC)c(CC(C)N)cc1OC ; COCCCc1cc(OC)c(CC(C)N)cc1OC ; CCCCCCc1cc(OC)c(CCN)cc1OC ; COc1cc(CCCCF)c(OC)cc1CCN] 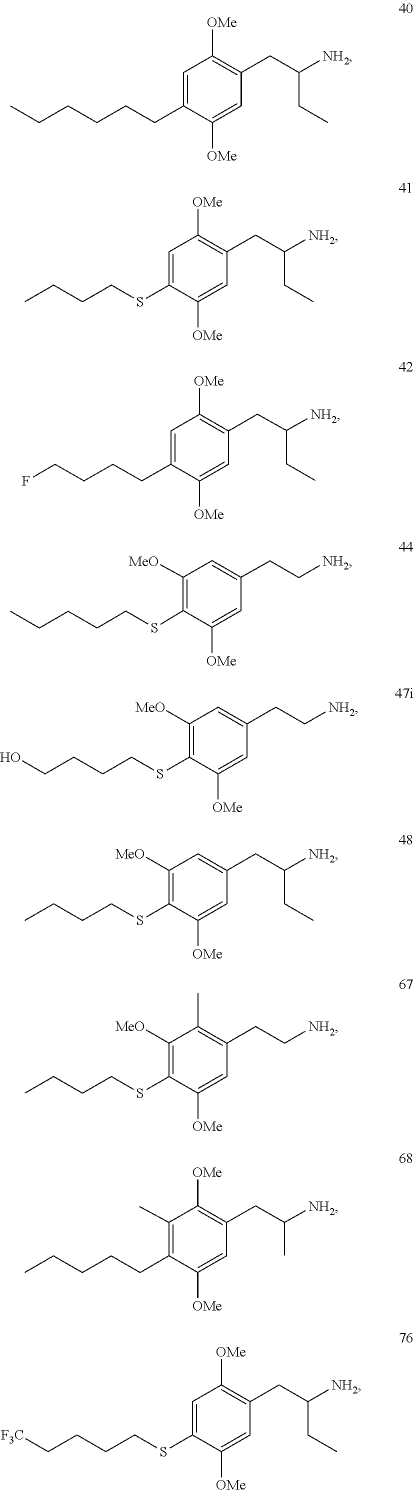 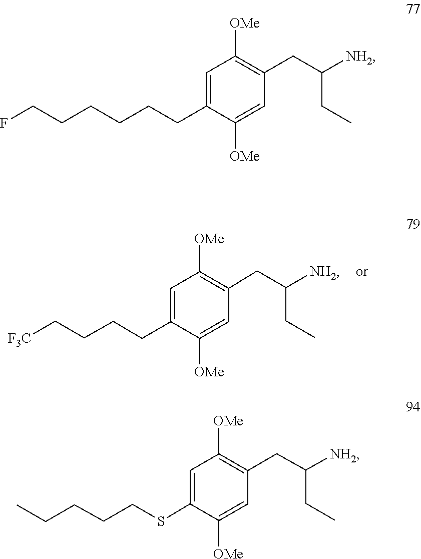
or a pharmaceutically acceptable salt thereof.

3. The compound of claim 1, having the structure: 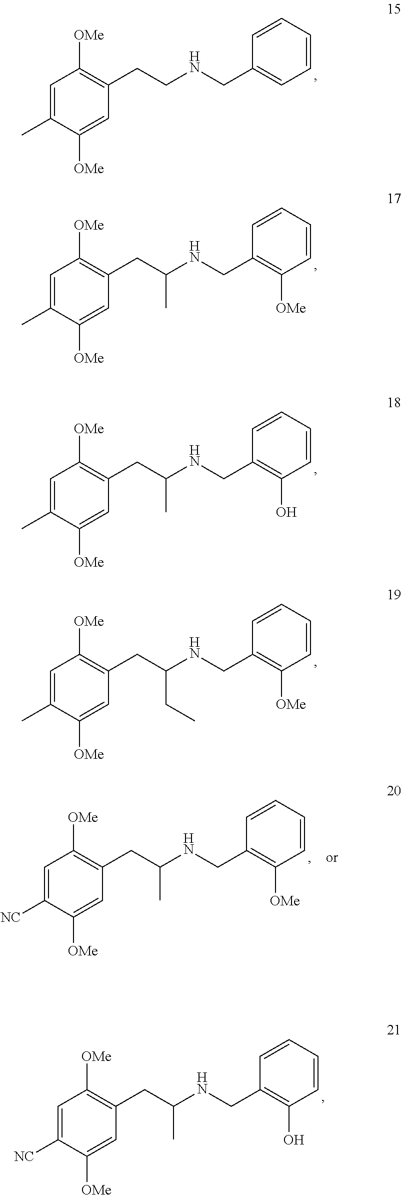 [SMILES: COc1cc(CCNCc2ccccc2)c(OC)cc1C ; COc1cc(CC(C)NCc2ccccc2OC)c(OC)cc1C ; COc1cc(CC(C)NCc2ccccc2O)c(OC)cc1C ; CCC(Cc1cc(OC)c(C)cc1OC)NCc1ccccc1OC ; COc1cc(CC(C)NCc2ccccc2OC)c(OC)cc1C#N ; COc1cc(CC(C)NCc2ccccc2O)c(OC)cc1C#N]
or a pharmaceutically acceptable salt thereof.

4. The compound of claim 1, having the structure: 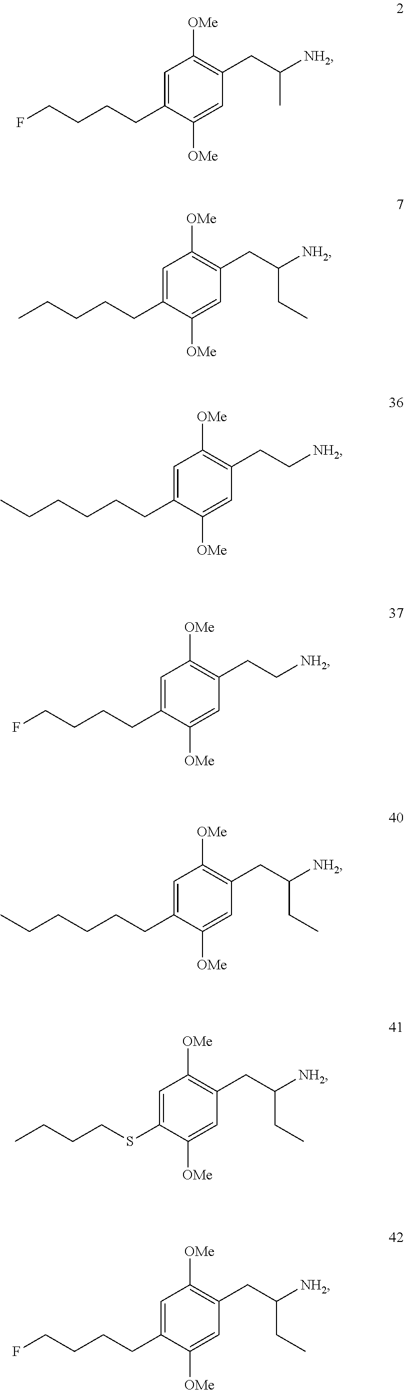 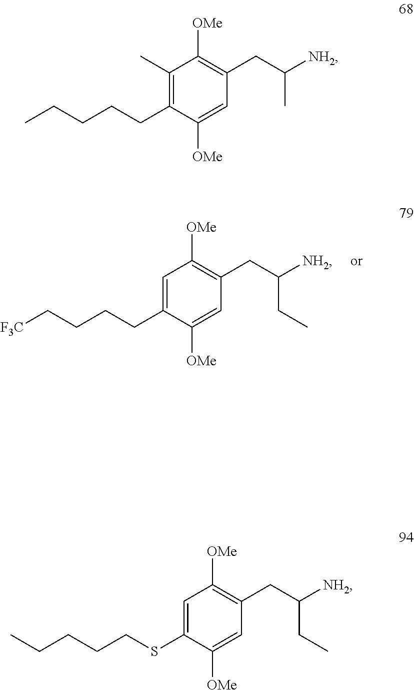
or a pharmaceutically acceptable salt thereof.

5. The compound of claim 1, having the structure: 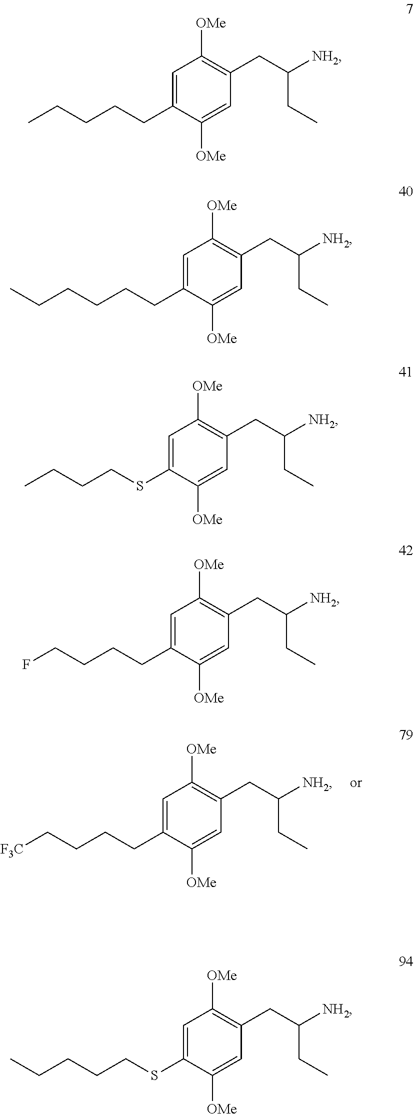
or a pharmaceutically acceptable salt thereof.

6. A compound of Formula (I) 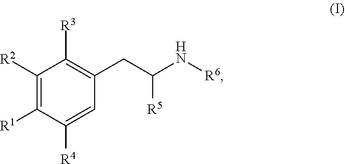
or a pharmaceutically acceptable salt thereof,
wherein
R¹ is C₄-C₈ alkyl, —S(C₄-C₈ alkyl), C₄-C₈ cycloalkylalkyl, —S(C₄-C₈ cycloalkylalkyl) or C₄-C₈ alkoxy,
wherein R¹ is substituted with one or more substituents, wherein each substituent is fluoro;
R² is hydrogen, hydroxyl, C₁-C₃ alkyl, halo, —CF₃, —OCF₃, C₁-C₃ alkoxy, or —S(C₁-C₃ alkyl);
R³ is hydrogen, hydroxyl, C₁-C₃ alkyl, halo, —CF₃, —OCF₃, C₁-C₃ alkoxy, or —S(C₁-C₃ alkyl);
wherein R² and R³ are not both hydrogen;
R⁴ is hydrogen, hydroxyl, C₁-C₃ alkyl, halo, —CF₃, —OCF₃, C₁-C₃ alkoxy, or —S(C₁-C₃ alkyl);
R⁵ is hydrogen or C₁-C₃ alkyl;
R⁶ is hydrogen or benzyl, wherein the phenyl ring of benzyl is optionally substituted with 1-5 instances of R^{6a};
each R^{6a} is independently selected for each occurrence from the group consisting of hydroxyl, C₁-C₆ alkoxy, C₁-C₆ alkyl, and halogen, or
wherein any two adjacent R^{6a} can be taken together with the atoms on which they are attached to form an optionally substituted C₅-C₇ cycloalkyl or optionally substituted 3-7 membered heterocyclyl; and
wherein when R¹ is —SCH₂CH₂CH₂CH₂F and R², R⁵, and R⁶ are each hydrogen, then at least one of R³ or R⁴ is other than —OMe.

7. The compound of claim 6, wherein:
R¹ is C₄-C₈ alkyl, —S(C₄-C₈ alkyl), or C₄-C₈ alkoxy,
wherein R¹ is substituted with one or more substituents, wherein each substituent is fluoro;
R² is hydrogen, C₁-C₃ alkoxy, or —S(C₁-C₃ alkyl);
R³ is hydrogen, C₁-C₃ alkoxy, or —S(C₁-C₃ alkyl);
wherein R² and R³ are not both hydrogen;
R⁴ is hydrogen, C₁-C₃ alkoxy, or —S(C₁-C₃ alkyl);
R⁵ is hydrogen or C₁-C₂ alkyl;
R⁶ is hydrogen or benzyl, wherein the phenyl ring of benzyl is optionally substituted with 1-5 instances of R^{6a};
each R^{6a} is independently selected for each occurrence from the group consisting of hydroxyl, C₁-C₆ alkoxy, and halogen, or
wherein any two adjacent R^{6a} can be taken together with the atoms on which they are attached to form an optionally substituted 3-7 membered heterocyclyl ring; and
wherein when R¹ is —SCH₂CH₂CH₂CH₂F and R², R⁵, and R⁶ are each hydrogen, then at least one of R³ or R⁴ is other than —OMe.

8. The compound of claim 6, wherein:
R¹ is C₄-C₈ alkyl or —S(C₄-C₈ alkyl),
wherein R¹ is substituted with one or more substituents, wherein each substituent is fluoro;
R² is hydrogen or C₁-C₃ alkoxy;
R³ is hydrogen or C₁-C₃ alkoxy;
wherein R² and R³ are not both hydrogen;
R⁴ is hydrogen or C₁-C₃ alkoxy;
R⁵ is hydrogen or C₁-C₂ alkyl;
R⁶ is hydrogen; and
wherein when R¹ is —SCH₂CH₂CH₂CH₂F and R², R⁵, and R⁶ are each hydrogen, then at least one of R³ or R⁴ is other than —OMe.

9. The compound of claim 6, wherein:
R¹ is C₄-C₈ alkyl or —S(C₄-C₈ alkyl),
wherein R¹ is substituted with one or more substituents, wherein each substituent is fluoro;
R² is hydrogen, —OMe, or —OEt;
R³ is hydrogen, —OMe, or —OEt;
wherein R² and R³ are not both hydrogen;
R⁴ is —OMe or —OEt;
R⁵ is hydrogen, -Me, or -Et;
R⁶ is hydrogen; and
wherein when R¹ is —SCH₂CH₂CH₂CH₂F and R², R⁵, and R⁶ are each hydrogen, then at least one of R³ or R⁴ is other than —OMe.

10. The compound of any one of claims 6-9, wherein the compound is a compound of Formula (I-a): 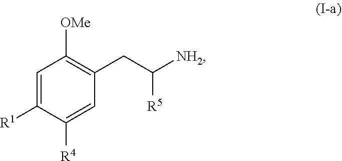
or a pharmaceutically acceptable salt thereof.

11. The compound of any one of claims 6-9, wherein the compound is a compound of Formula (I-b): 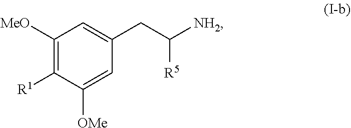
or a pharmaceutically acceptable salt thereof.

12. The compound of any one of claims 6-9, wherein the compound is a compound of Formula (I-c): 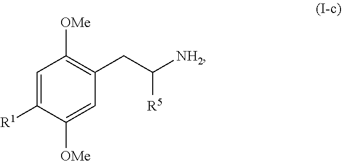
or a pharmaceutically acceptable salt thereof.

13. The compound of any one of claims 6-12, wherein R¹ is —S(C₄-C₈ alkyl) substituted with one or more substituents, wherein each substituent is fluoro.

14. The compound of any one of claims 6-12, wherein R¹ is C₄-C₈ alkyl substituted with one or more substituents, wherein each substituent is fluoro.

15. The compound of any one of claims 6-14, wherein R¹ is substituted with one, two, or three substituents, wherein each substituent is fluoro.

16. A compound selected from the group consisting of: 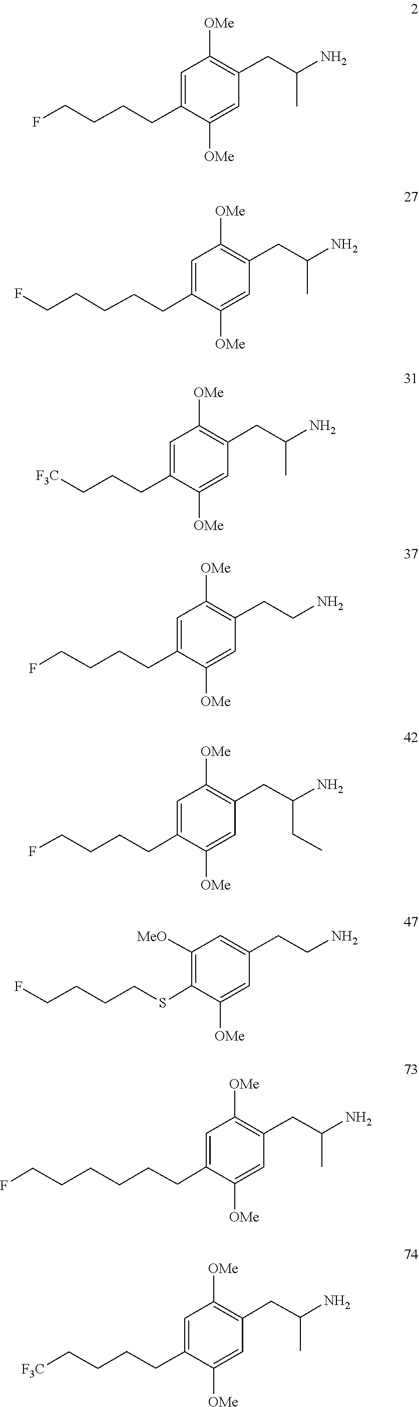 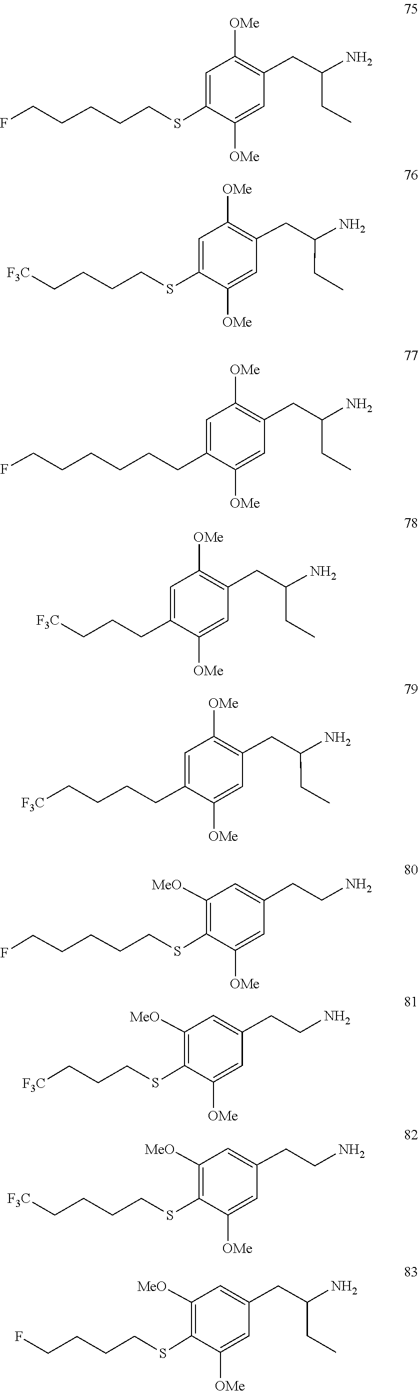 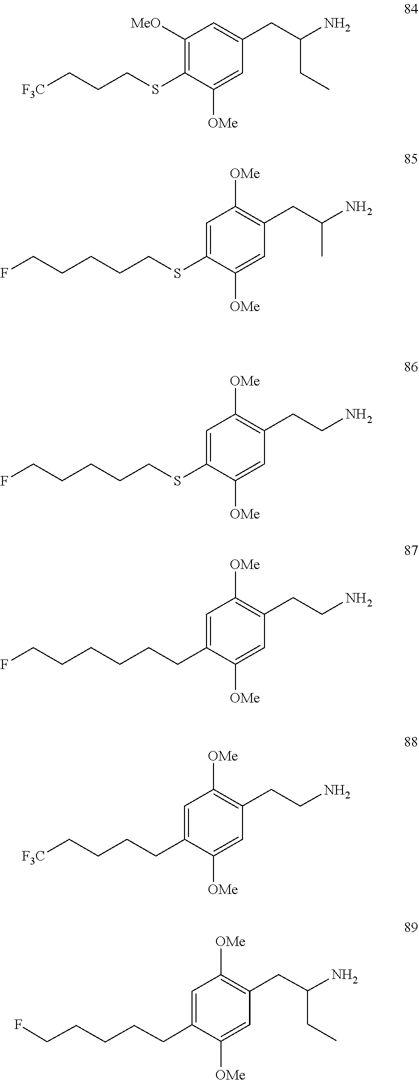 [SMILES: CCC(N)Cc1cc(OC)c(SCCCC(F)(F)F)c(OC)c1 ; COc1cc(SCCCCCF)c(OC)cc1CC(C)N ; COc1cc(SCCCCCF)c(OC)cc1CCN ; COc1cc(CCCCCCF)c(OC)cc1CCN ; COc1cc(CCCCC(F)(F)F)c(OC)cc1CCN ; CCC(N)Cc1cc(OC)c(CCCCCF)cc1OC]
or a pharmaceutically acceptable salt thereof.

17. A compound of Formula (II): 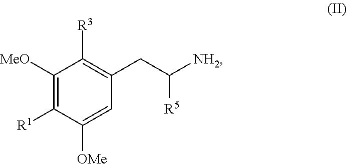
or a pharmaceutically acceptable salt thereof,
wherein
R¹ is C₄-C₈ alkyl or —S(C₄-C₈ alkyl),
wherein C₄-C₈ alkyl may be optionally substituted by one or more substituents, each independently selected from the group consisting of hydroxyl, oxo, —CN, —NR⁷R⁸, C₁-C₆ alkoxy, C₁-C₃ alkyl, phenyl, 5-6 membered heteroaryl, C₃-C₆ cycloalkyl, and 3-6-membered heterocyclyl;
R³ is selected from the group consisting of hydrogen, halogen, and C₁-C₃ alkyl;
R⁵ is hydrogen or C₁-C₃ alkyl;
R⁷ and R⁸ are independently selected, for each occurrence, from the group consisting of hydrogen and C₁-C₃ alkyl; wherein C₁-C₃ alkyl may optionally be substituted by one or more substituents selected from the group consisting of fluorine, cyano, oxo, and hydroxyl;
or R⁷ and R⁸, together with the nitrogen to which they are attached, may form a 4-6 membered heterocyclic ring, which may have an additional heteroatom selected from O, S, or N;
wherein the 4-6 membered heterocyclic ring may optionally be substituted by one or more substituents selected from the group consisting of fluorine, cyano, oxo, and hydroxyl; and
wherein when R¹ is —SCH₂CH₂CH₂CH₃, R³ and R⁵ are not both hydrogen.

18. The compound of claim 17, wherein:
R¹ is C₄-C₈ alkyl or —S(C₄-C₈ alkyl),
wherein C₄-C₈ alkyl may be optionally substituted by one or more substituents, each independently selected from the group consisting of hydroxyl, C₁-C₆ alkoxy, and C₃-C₆ cycloalkyl;
R³ is selected from the group consisting of hydrogen, halogen, and C₁-C₃ alkyl;
R⁵ is hydrogen, Me, or Et; and
wherein when R¹ is —SCH₂CH₂CH₂CH₃, R³ and R⁵ are not both hydrogen.

19. The compound of claim 17, wherein:
R¹ is C₄-C₈ alkyl or —S(C₄-C₈ alkyl),
wherein C₄-C₈ alkyl is unsubstituted;
R³ is selected from the group consisting of hydrogen, halogen, and Me;
R⁵ is hydrogen, Me, or Et; and
wherein when R¹ is —SCH₂CH₂CH₂CH₃, R³ and R⁵ are not both hydrogen.

20. The compound of any one of claims 6-19, wherein R⁵ is hydrogen.

21. The compound of any one of claims 6-19, wherein R⁵ is C₁-C₂ alkyl.

22. The compound of any one of claims 6-19, wherein R⁵ is Me.

23. The compound of any one of claims 6-19, wherein R⁵ is Et.

24. A compound selected from the group consisting of: 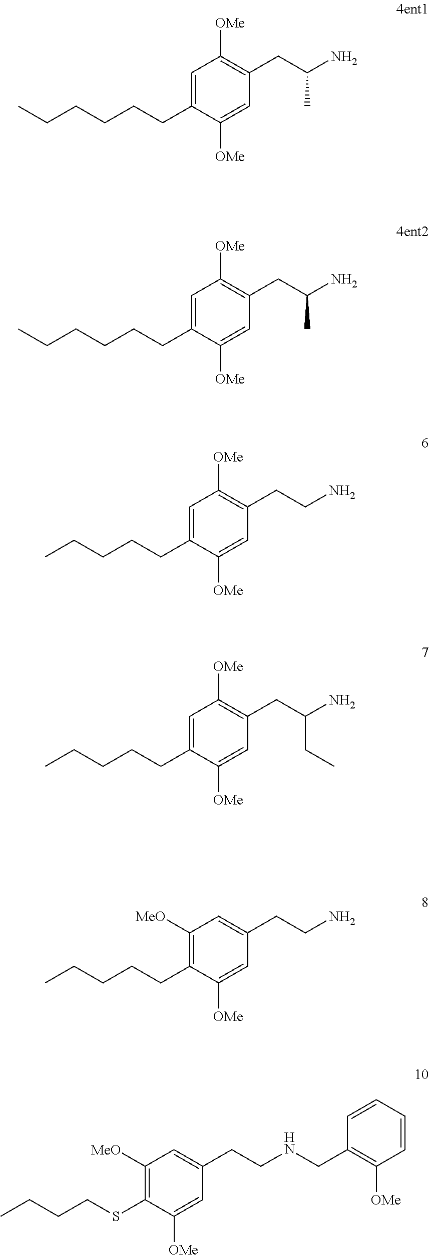 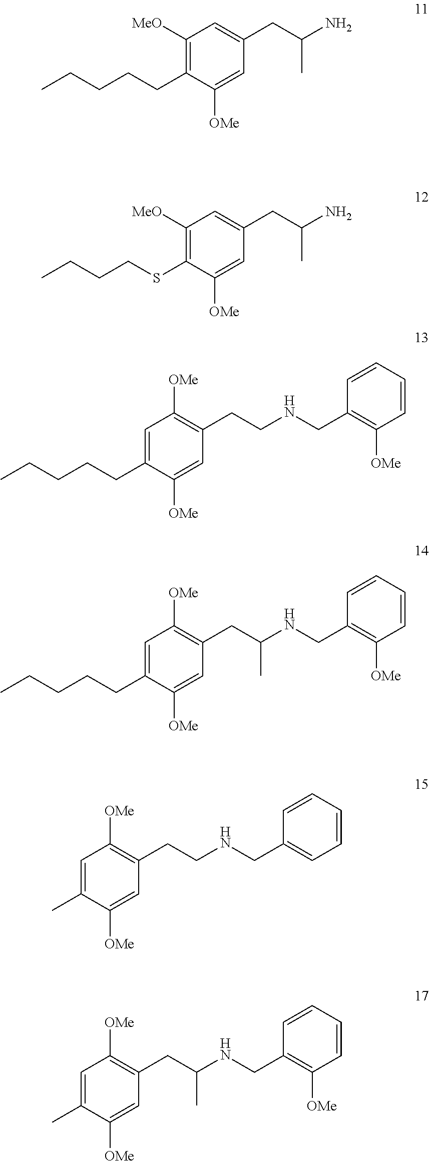 [SMILES: CCCCCc1c(OC)cc(CC(C)N)cc1OC ; CCCCSc1c(OC)cc(CC(C)N)cc1OC ; CCCCCc1cc(OC)c(CCNCc2ccccc2OC)cc1OC ; CCCCCc1cc(OC)c(CC(C)NCc2ccccc2OC)cc1OC ; COc1cc(CCNCc2ccccc2)c(OC)cc1C ; COc1cc(CC(C)NCc2ccccc2OC)c(OC)cc1C] 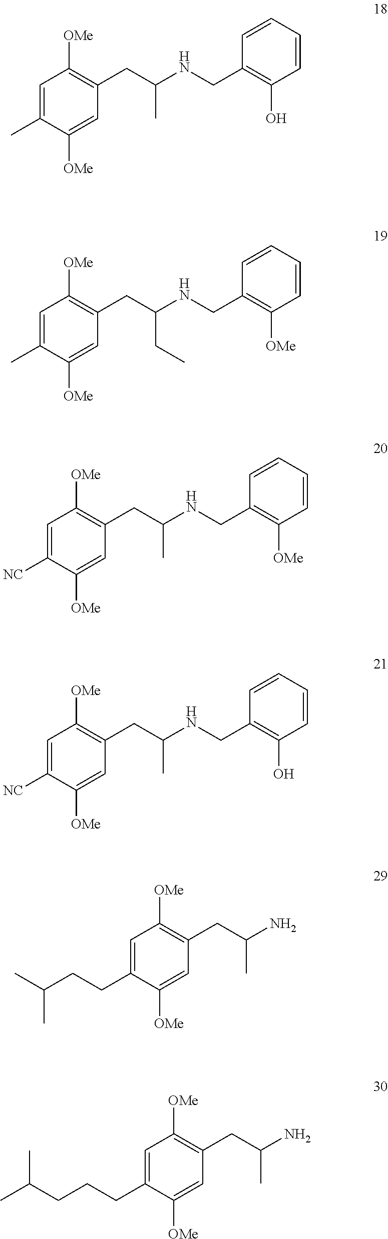 [SMILES: COc1cc(CC(C)NCc2ccccc2O)c(OC)cc1C ; CCC(Cc1cc(OC)c(C)cc1OC)NCc1ccccc1OC ; COc1cc(CC(C)NCc2ccccc2OC)c(OC)cc1C#N ; COc1cc(CC(C)NCc2ccccc2O)c(OC)cc1C#N ; COc1cc(CC(C)N)c(OC)cc1CCC(C)C ; COc1cc(CC(C)N)c(OC)cc1CCCC(C)C] 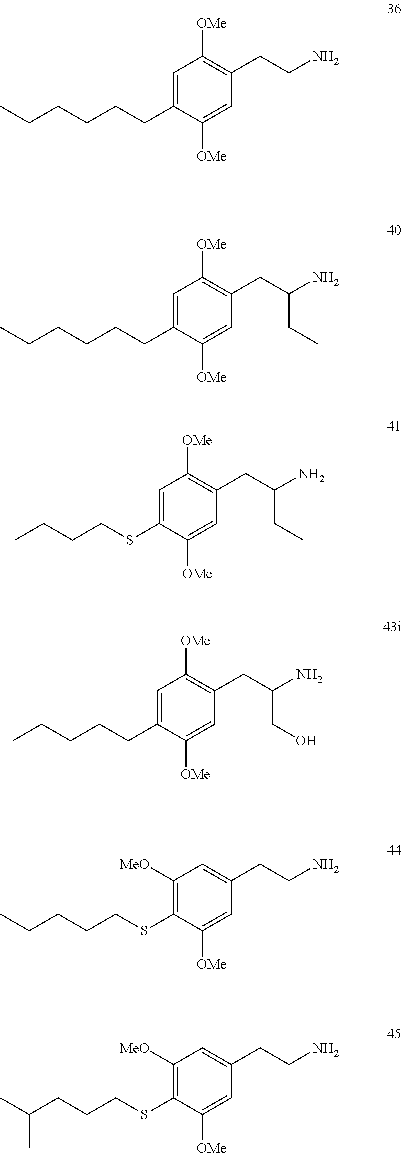 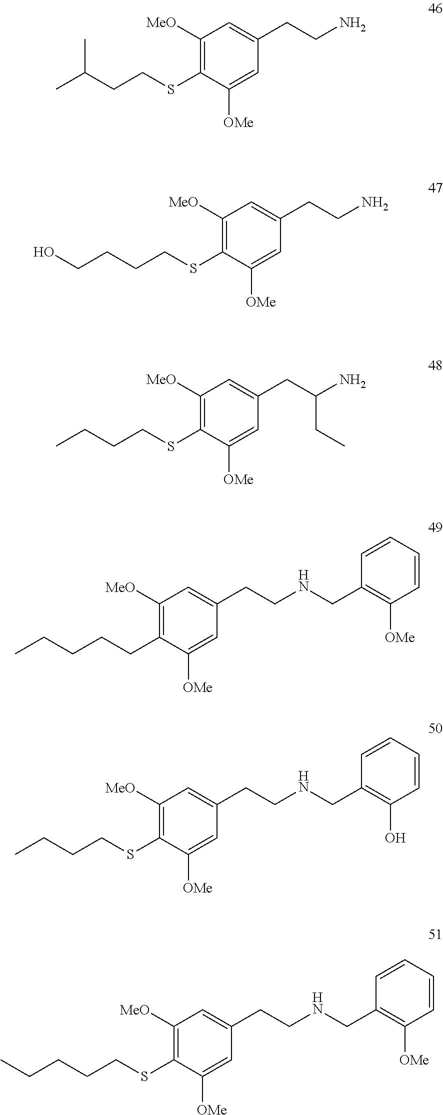 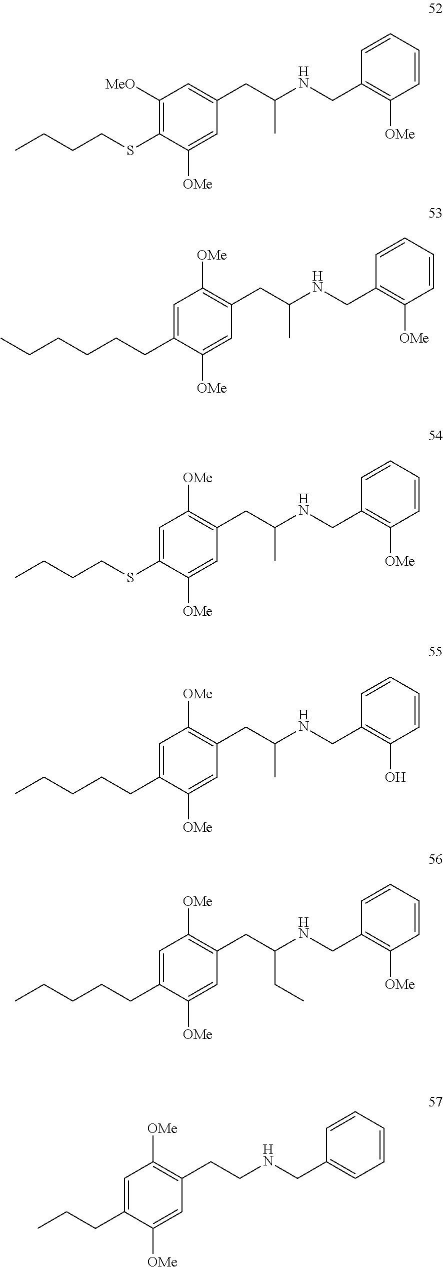 [SMILES: CCCCSc1c(OC)cc(CC(C)NCc2ccccc2OC)cc1OC ; CCCCCCc1cc(OC)c(CC(C)NCc2ccccc2OC)cc1OC ; CCCCSc1cc(OC)c(CC(C)NCc2ccccc2OC)cc1OC ; CCCCCc1cc(OC)c(CC(C)NCc2ccccc2O)cc1OC ; CCCCCc1cc(OC)c(CC(CC)NCc2ccccc2OC)cc1OC ; CCCc1cc(OC)c(CCNCc2ccccc2)cc1OC] 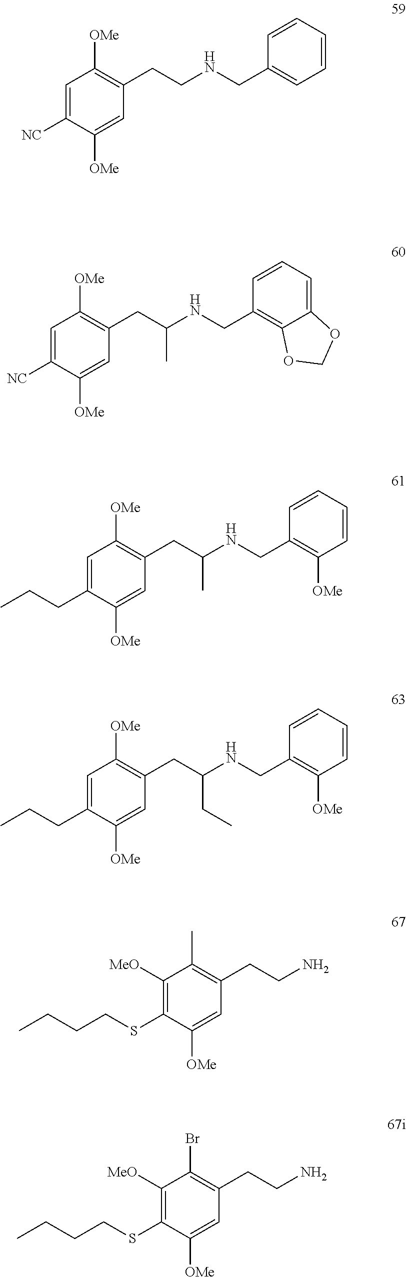 [SMILES: COc1cc(CCNCc2ccccc2)c(OC)cc1C#N ; COc1cc(CC(C)NCc2cccc3c2OCO3)c(OC)cc1C#N ; CCCc1cc(OC)c(CC(C)NCc2ccccc2OC)cc1OC ; CCCc1cc(OC)c(CC(CC)NCc2ccccc2OC)cc1OC ; CCCCSc1c(OC)cc(CCN)c(C)c1OC ; CCCCSc1c(OC)cc(CCN)c(Br)c1OC] 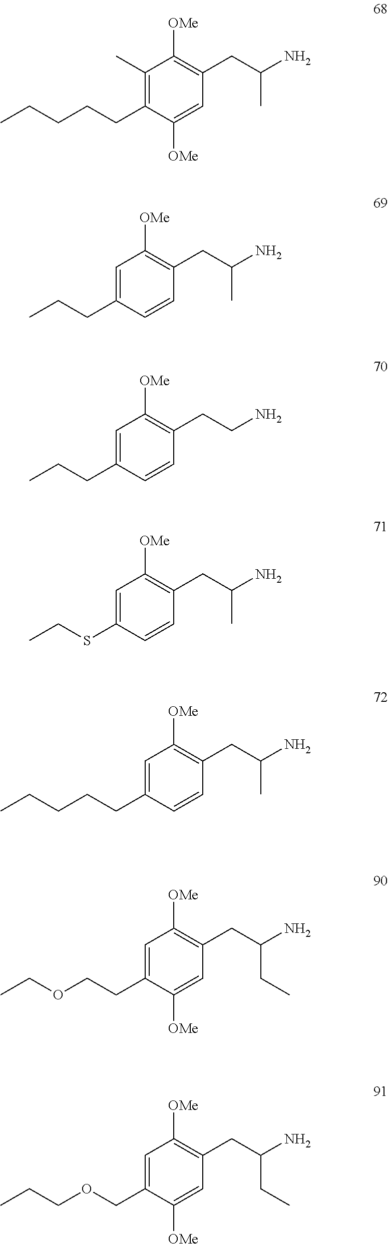 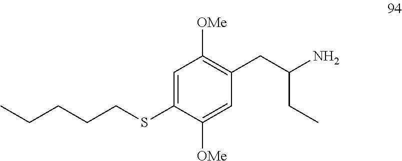 [SMILES: CCCCCSc1cc(OC)c(CC(N)CC)cc1OC]
or a pharmaceutically acceptable salt thereof.

25. A pharmaceutical composition comprising the compound of any one of claims 1-24 and a pharmaceutically acceptable carrier.

26. A method of treating a psychiatric disease or disorder, comprising administering to a patient in need thereof a therapeutically effective amount of a compound of any one of claims 1-24.

27. A method of treating headache or headache disorder, comprising administering to a patient in need thereof a therapeutically effective amount of a compound of any one of claims 1-24.

28. A method of treating an inflammatory disease or disorder, comprising administering to a patient in need thereof a therapeutically effective amount of a compound of any one of claims 1-24.

29. A method of treating high intraocular pressure, comprising administering to a patient in need thereof a therapeutically effective amount of a compound of any one of claims 1-24.

30. A method of treating a psychiatric disease or disorder, a headache or headache disorder, an inflammatory disease or disorder, or high intraocular pressure, comprising administering to a patient in need thereof a therapeutically effective amount of a compound selected from the group consisting of: 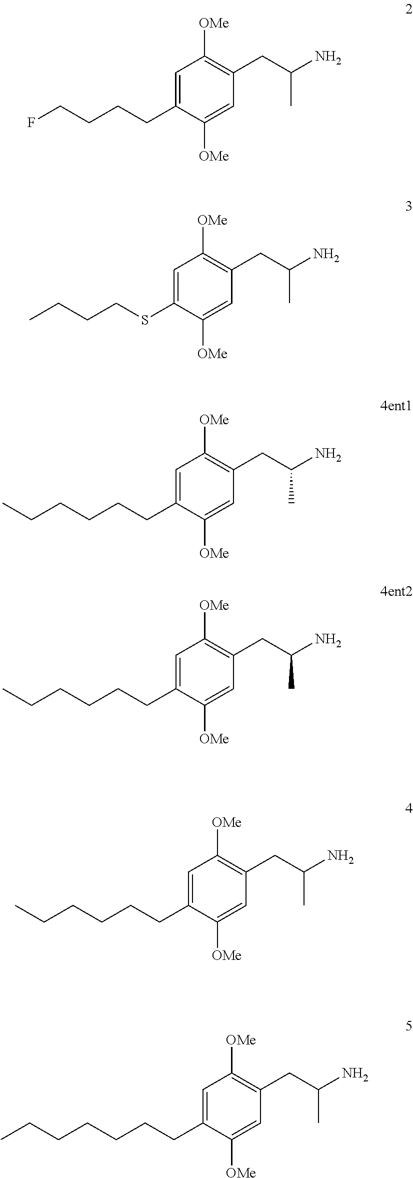 [SMILES: COc1cc(CC(C)N)c(OC)cc1CCCCF ; CCCCSc1cc(OC)c(CC(C)N)cc1OC ; CCCCCCc1cc(OC)c(C[C@@H](C)N)cc1OC ; CCCCCCc1cc(OC)c(C[C@H](C)N)cc1OC ; CCCCCCc1cc(OC)c(CC(C)N)cc1OC ; CCCCCCCc1cc(OC)c(CC(C)N)cc1OC] 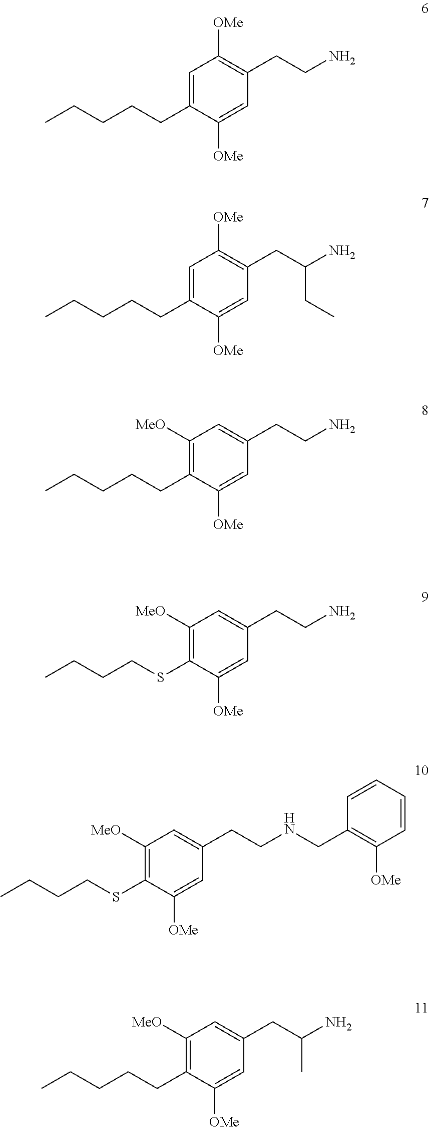 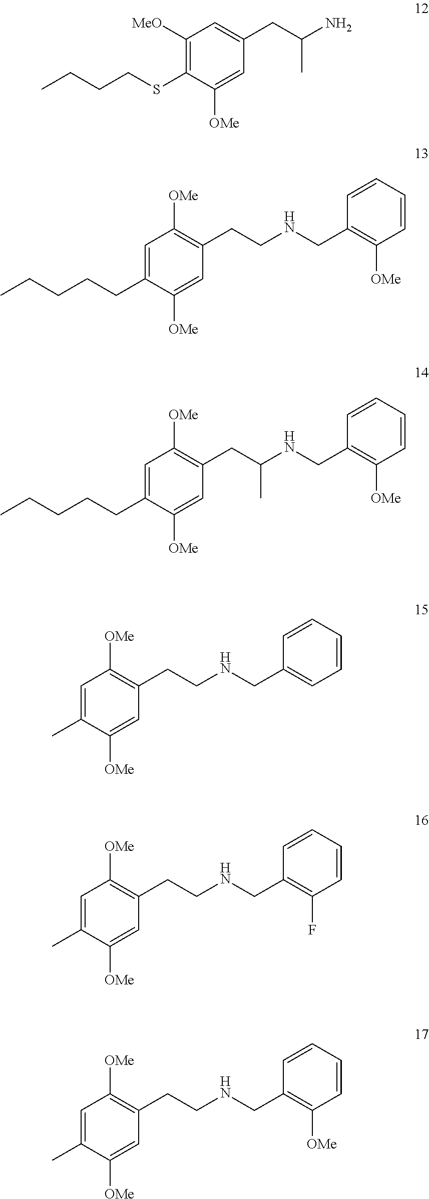 [SMILES: CCCCSc1c(OC)cc(CC(C)N)cc1OC ; CCCCCc1cc(OC)c(CCNCc2ccccc2OC)cc1OC ; CCCCCc1cc(OC)c(CC(C)NCc2ccccc2OC)cc1OC ; COc1cc(CCNCc2ccccc2)c(OC)cc1C ; COc1cc(CCNCc2ccccc2F)c(OC)cc1C ; COc1cc(CCNCc2ccccc2OC)c(OC)cc1C] 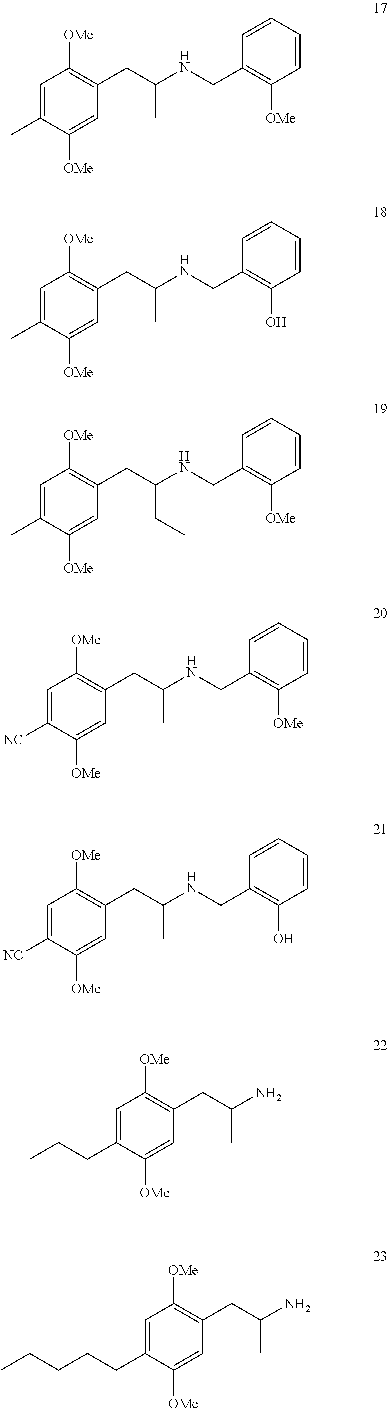 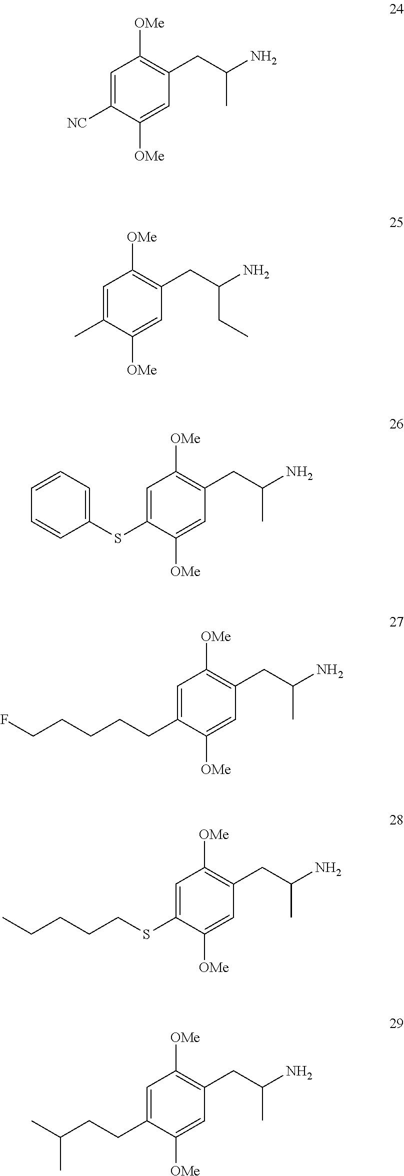 [SMILES: COc1cc(CC(C)N)c(OC)cc1C#N ; CCC(N)Cc1cc(OC)c(C)cc1OC ; COc1cc(Sc2ccccc2)c(OC)cc1CC(C)N ; COc1cc(CC(C)N)c(OC)cc1CCCCCF ; CCCCCSc1cc(OC)c(CC(C)N)cc1OC ; COc1cc(CC(C)N)c(OC)cc1CCC(C)C] 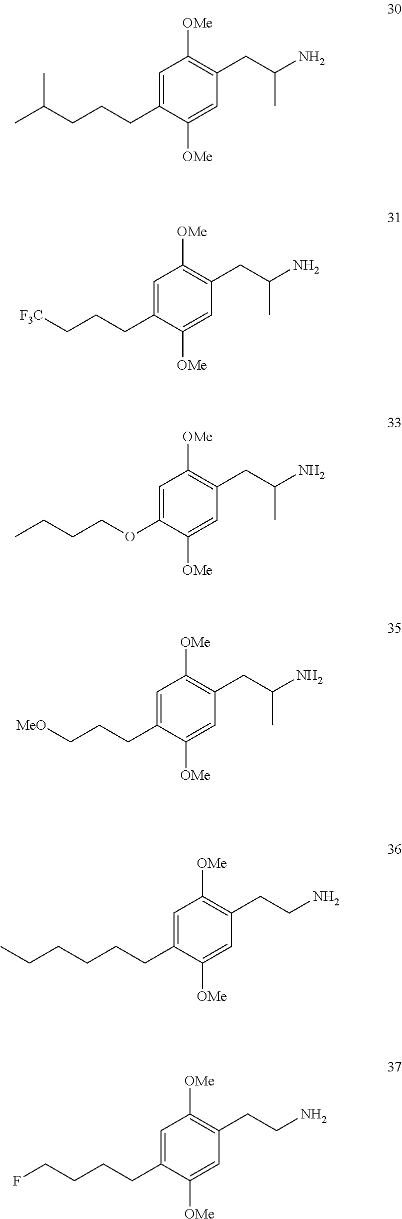 [SMILES: COc1cc(CC(C)N)c(OC)cc1CCCC(C)C ; COc1cc(CC(C)N)c(OC)cc1CCCC(F)(F)F ; CCCCOc1cc(OC)c(CC(C)N)cc1OC ; COCCCc1cc(OC)c(CC(C)N)cc1OC ; CCCCCCc1cc(OC)c(CCN)cc1OC ; COc1cc(CCCCF)c(OC)cc1CCN] 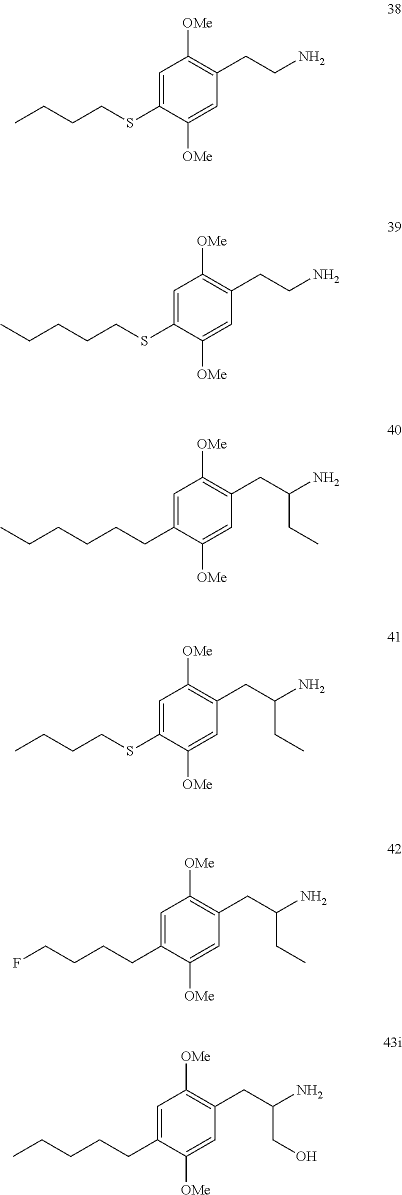 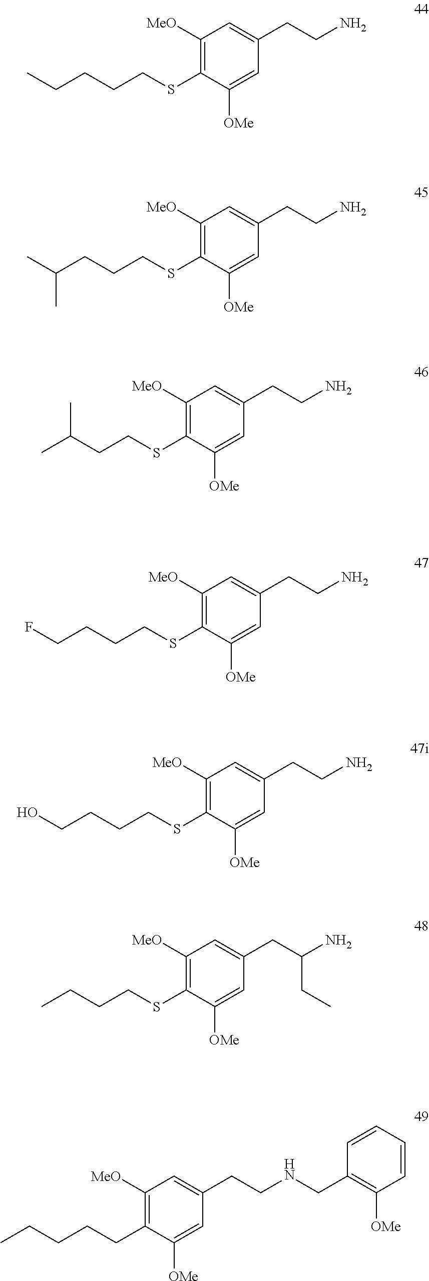 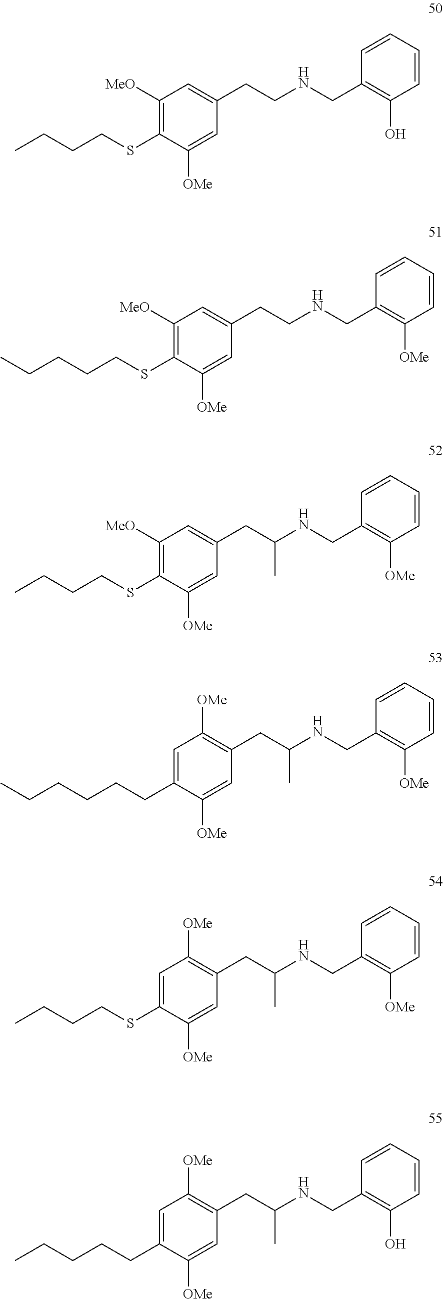 [SMILES: CCCCSc1c(OC)cc(CCNCc2ccccc2O)cc1OC ; CCCCCSc1c(OC)cc(CCNCc2ccccc2OC)cc1OC ; CCCCSc1c(OC)cc(CC(C)NCc2ccccc2OC)cc1OC ; CCCCCCc1cc(OC)c(CC(C)NCc2ccccc2OC)cc1OC ; CCCCSc1cc(OC)c(CC(C)NCc2ccccc2OC)cc1OC ; CCCCCc1cc(OC)c(CC(C)NCc2ccccc2O)cc1OC] 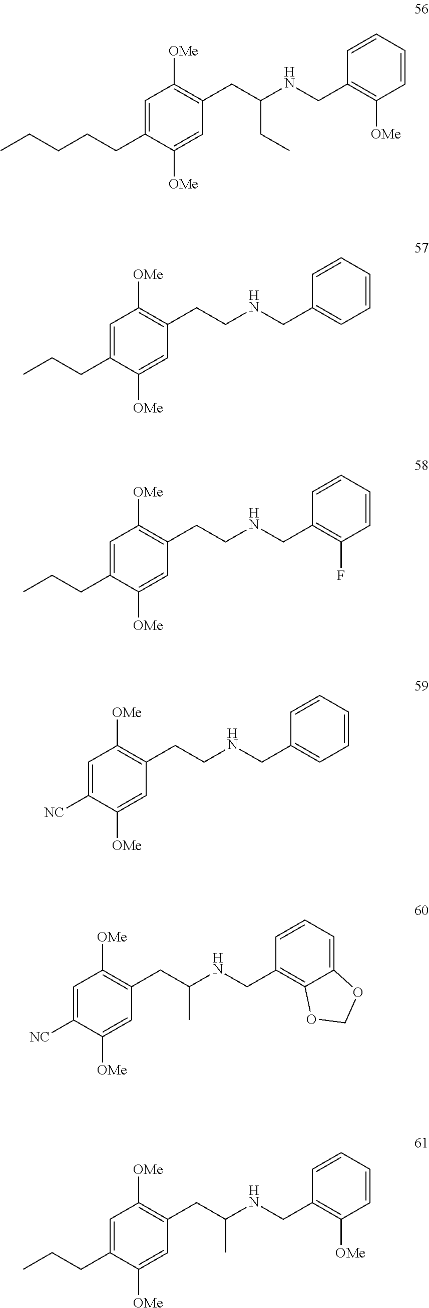 [SMILES: CCCCCc1cc(OC)c(CC(CC)NCc2ccccc2OC)cc1OC ; CCCc1cc(OC)c(CCNCc2ccccc2)cc1OC ; CCCc1cc(OC)c(CCNCc2ccccc2F)cc1OC ; COc1cc(CCNCc2ccccc2)c(OC)cc1C#N ; COc1cc(CC(C)NCc2cccc3c2OCO3)c(OC)cc1C#N ; CCCc1cc(OC)c(CC(C)NCc2ccccc2OC)cc1OC] 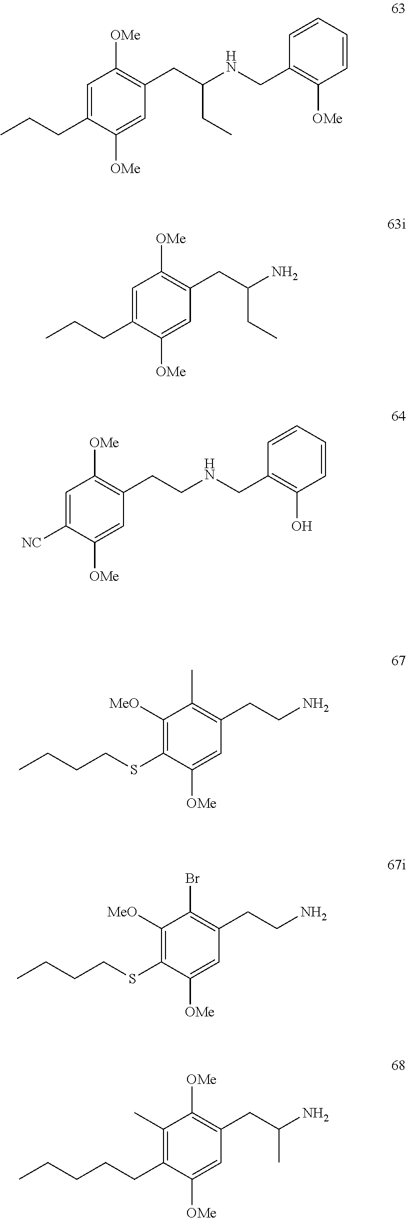 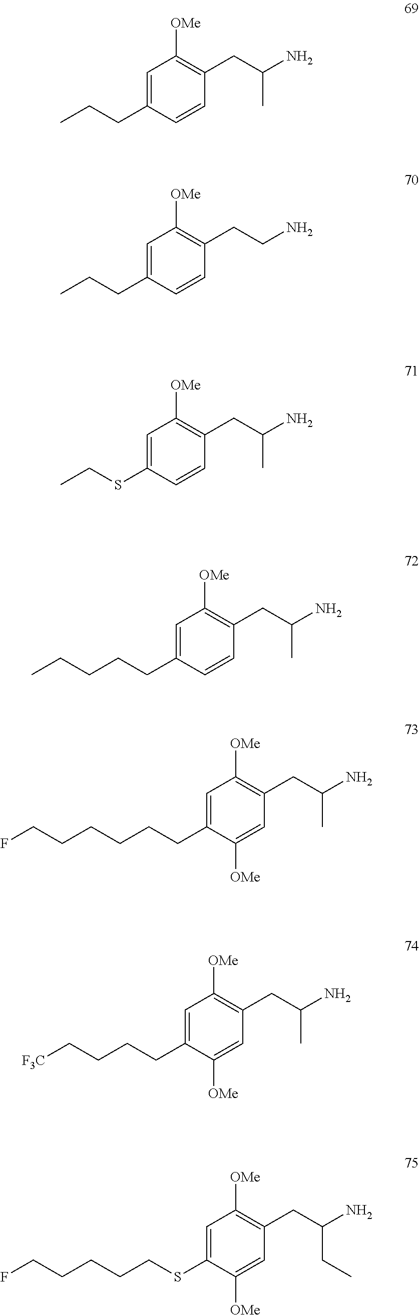 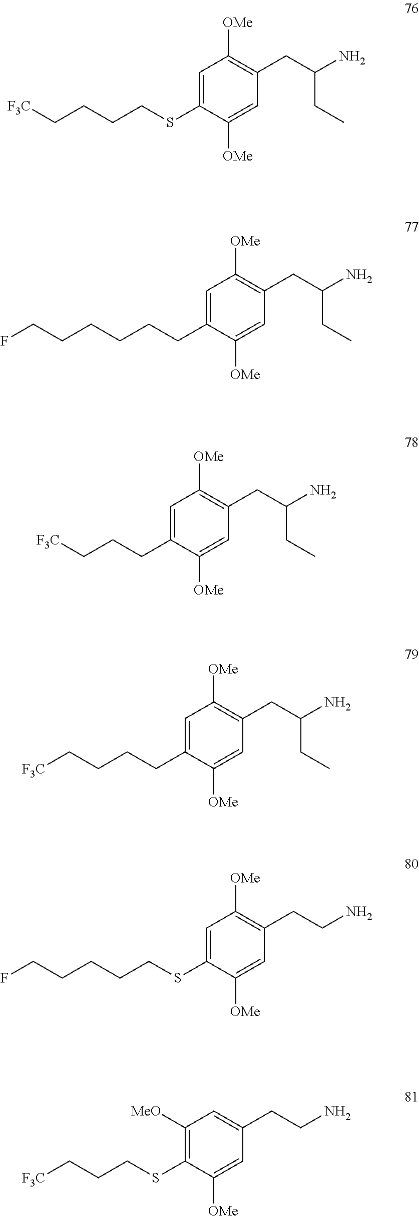 [SMILES: CCC(N)Cc1cc(OC)c(SCCCCC(F)(F)F)cc1OC ; CCC(N)Cc1cc(OC)c(CCCCCCF)cc1OC ; CCC(N)Cc1cc(OC)c(CCCC(F)(F)F)cc1OC ; CCC(N)Cc1cc(OC)c(CCCCC(F)(F)F)cc1OC ; COc1cc(SCCCCCF)c(OC)cc1CCN ; COc1cc(CCN)cc(OC)c1SCCCC(F)(F)F] 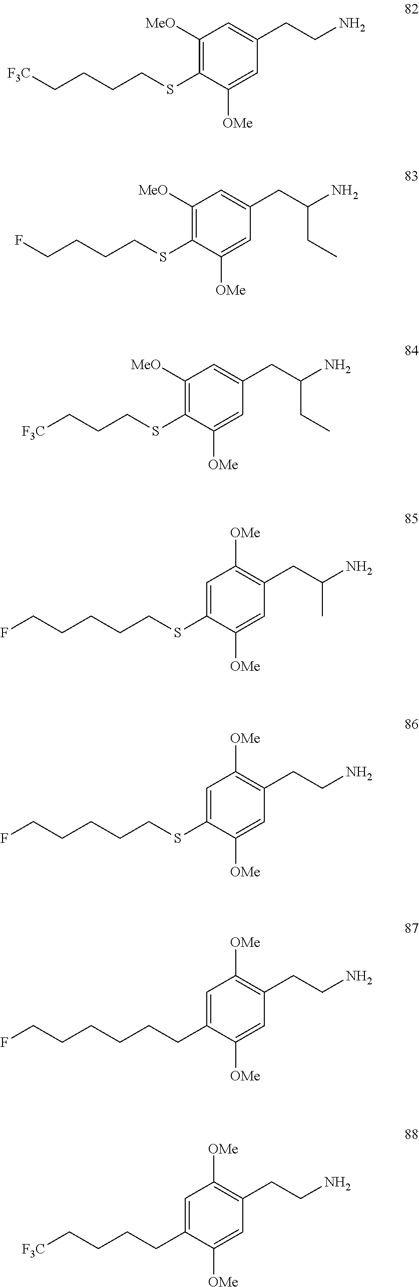 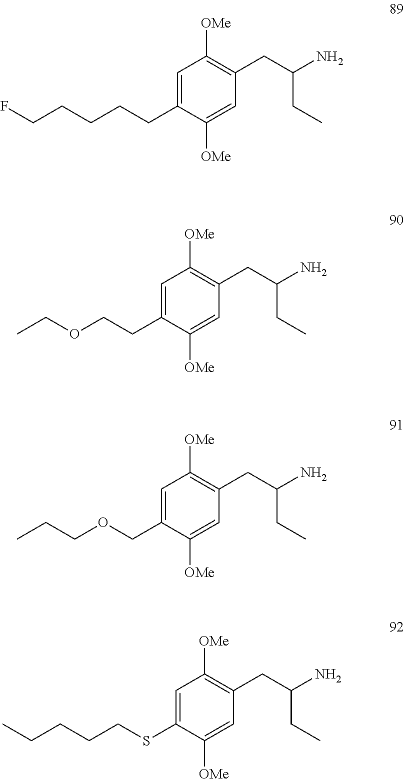
or a pharmaceutically acceptable salt thereof.
